(19) United States
(12) Patent Application Publication
(10) Pub. No.: US 2026/0327295 A1
(43) Pub. Date: Oct. 22, 2026

(54) ANTI-C-REACTIVE PROTEIN SINGLE-DOMAIN ANTIBODIES

(71) Applicants: Christopher REES, Cobham (GB); SEERAVE VENTURES LIMITED, St Helier (JE)

(72) Inventor: Manuel FANKHAUSER, St Helier (GB)

(73) Assignees: Christoper REES, Cobham (GB); SEERAVE ENTERPRISES LIMITED, St Helier (JE)

(21) Appl. No.: 19/483,602

(22) PCT Filed: May 10, 2025
(86) PCT No.: PCT/EP2024/062989

§ 371 Date: Nov. 11, 2025

(30) Foreign Application Priority Data
May 12, 2023 (EP) 23173246.2

(51) Int. Cl. C07K 16/18; A61B 5/1473; G01N 33/543; G01N 33/68

(52) U.S. Cl. CPC C07K 16/18; A61B 5/14735; G01N 33/5438; G01N 33/6893; C07K 2317/565; C07K 2317/567; C07K 2317/569; C07K 2317/76; C07K 2317/92; G01N 2333/4737

(57) ABSTRACT

The present invention relates to single-domain antibodies (sdAbs) binding C-reactive protein (CRP). In particular, the invention relates to sdAbs that bind CRP within a specific range of an on-rate constant and off-rate constant. The sdAbs of the present invention can associate and dissociate with CRP in a continuous manner. The invention further relates to nucleic acids encoding anti-CRP sdAbs, methods for detecting CRP, methods of treatment and diagnosis making use of the anti-CRP sdAbs, methods for monitoring CRP levels and apparatuses comprising the anti-CRP sdAbs.

The present invention relates to single-domain antibodies (sdAbs) binding C-reactive protein (CRP). In particular, the invention relates to sdAbs that bind CRP within a specific range of an on-rate constant and off-rate constant. The sdAbs of the present invention can associate and dissociate with CRP in a continuous manner. The invention further relates to nucleic acids encoding anti-CRP sdAbs, methods for detecting CRP, treatments and diagnoses making use of the anti-CRP sdAbs, methods for monitoring CRP levels and apparatuses comprising the anti-CRP sdAbs.

Single-domain antibodies (sdAb) are a type of antibody fragment consisting of a single monomeric variable antibody domain only. Similarly, to their larger counterparts they are able to selectively bind to a specific antigen. sdAb can, e.g. be engineered from heavy-chain antibodies in camelids, referred to as VHH fragments. An advantage when using sdAbs is that they can be designed to not affect the biological activity or structure of a bound molecule. However, they may also be specifically designed to block biological activity. Further, sdAbs are particularly robust which is advantageous for e.g. application as biosensors or as components of biosensors. Thus, sdAbs are advantageous to specifically detect a molecule of interest with or without interfering with biological functions, e.g. as or in a biosensor. C-reactive protein (CRP) is an annular, pentameric protein found in blood plasma, whose circulating concentrations rise in response to inflammation. It is an acute-phase protein of hepatic origin that increases following interleukin 6 secretion by macrophages and T cells. The pathological significance of CRP for various diseases has been indicated in numerous studies, including metabolic syndrome, cardiovascular disease, active inflammatory bowel disease, cholecystitis, chronic obstructive pulmonary disease, acute bronchitis, sepsis, active advanced cancer, metastasis, pancreatitis, pneumonia, severe infection (lung, urinary, skin, gastrointestinal, blood), rheumatologic causes, and advanced tumors. The treatment of such diseases with drugs preferably results in reduced inflammation, i.e. a reduced level of CRP in a subject, for example reduced to the upper limit of normal (ULN) in a healthy non-inflammatory subject avoiding excessive tissue damage. To achieve this and to avoid overdosing or underdosing of drugs that may invoke adverse effects or result in an ineffective therapy, precise monitoring of CRP levels in a subject is desired. In addition, monitoring of CRP levels is useful to assess the therapeutic outcome of a potential treatment and whether a subject is for example incompatible with a treatment resulting in inflammation after drug administration. To achieve the above, precise measurement of CRP levels, preferably over an extended period of time, i.e. continuous monitoring of CRP levels is needed. However, the current means for detecting CRP are limited to antibodies with exceptionally high affinities to CRP which do however not consider the dynamic range of CRP levels in a subject and are not feasible for use in a biosensor. For example, Oloketuyi, et al. (2021) discloses anti-CRP nanobodies. HyTest (“HyTest, TechNotes: Human C-Reactive Protein) discloses anti-CRP monoclonal antibodies. A very high affinity CRP antibody results in a permanent binding of CRP which would saturate a biosensor. A saturated biosensor is unable to continuously monitor levels of CRP thereby rendering the same useless for measurements over time. When using a very high affinity CRP antibody, detection of CRP is limited to a yes, no type of spot measurement of CRP which does not allow for continuously monitoring dynamic levels of CRP that may be specific for certain diseases and therapies.

Accordingly, there is a need for continuous monitoring of dynamic CRP levels, e.g. in a subject undergoing therapy or in a subject suffering from a disease. The technical problem underlying the present invention is the provision of means and methods for the detection, quantification and/or the monitoring of CRP, particularly continuous detection, quantification and/or monitoring. The technical problem is solved by provision of the embodiments characterized in the claims and as provided herein below. Specifically, the technical problem is solved, and the above-mentioned difficulties are overcome by the provision of an anti-CRP sdAb.

The inventors have predicted the dynamic range of CRP levels in the context of disease and homeostasis, i.e. in a healthy context. They have shown that means for detecting, quantifying and monitoring CRP should cover said dynamic range to allow for an unambiguous conclusion about the inflammation status in a subject. The inventors have used the dynamic range of CRP to specifically generate sdAb that are capable of detecting CRP throughout the entire range, which allows detection and monitoring of the CRP level and thus the inflammation status in a subject or sample. In addition, the sdAb of the present invention embody a specific on-rate constant and off-rate constant, allowing for continuous association and dissociation with CRP, thereby enabling continuous measurement of CRP levels over time.

The invention, inter alia, provides the following advantages:
Anti-CRP sdAb specifically associate with CRP over the entire dynamic range of CRP allowing for precise detection and quantification of the CRP level and thus inflammation status that is relevant in therapy.
The anti-CRP sdAbs can detect CRP from baseline CRP levels to CRP levels being present in severe inflammation.
The anti-CRP sdAbs can continuously and repeatedly bind CRP allowing for continuous detection, quantification and monitoring of CRP levels, e.g. when applied as a biosensor or as an implanted biosensor in a sample or subject, thereby overcoming extensive and laborious CRP spot measurements.
The dynamic detection of CRP levels contributes to the assessment of the therapeutic outcome of a treatment, or a possible adverse effect invoked by an intolerance to a drug.
The detection and quantification of CRP levels over time allow for targeted therapy of inflammatory diseases, thereby reducing the amount of medication and potential side effects of said medication
The anti-CRP sdAbs can be used as a biosensor in a device that allows for continuous monitoring of CRP levels in a subject.

The invention provides the following items:
1. An anti-C-reactive protein (CRP) single-domain antibody (sdAb) comprising an on-rate constant (kₒₙ) of about 3.30×10\hat{ }4 s⁻¹M⁻¹−1.70×10\hat{ }7 s⁻¹M⁻¹ and an off-rate constant (k_{off}) of about 3×10\hat{ }−9 s⁻¹−2.5×10\hat{ }−1 s⁻¹ (preferably an off-rate constant (k_{off}) of about 3.20×10\hat{ }−9 s⁻¹−2.20×10\hat{ }−1 s⁻¹, preferably wherein said sdAb is capable of detecting and/or quantifying CRP concentrations of about 0.01 μM or less to about 1 μM or more.
2. The anti-CRP sdAb of item 1, wherein the sdAb comprises a first complementary determining region (CDR1), a second CDR (CDR2) and third CDR (CDR3), wherein:
   (i) CDR1 comprises SEQ ID NO: 3 or a variant thereof having up to two amino acid substitutions, CDR2 comprises SEQ ID NO: 5 or a variant thereof having up to two amino acid substitutions, CDR3 comprises SEQ ID NO: 7 or a variant thereof having up to five amino acid substitutions; or
   (ii) CDR1 comprises SEQ ID NO: 11 or a variant thereof having up to two amino acid substitutions, CDR2 comprises SEQ ID NO: 13 or a variant thereof having up to two amino acid substitutions, CDR3 comprises SEQ ID NO: 15 or a variant thereof having up to five amino acid substitutions; or
   (iii) CDR1 comprises SEQ ID NO: 19 or a variant thereof having up to two amino acid substitutions, CDR2 comprises SEQ ID NO: 21 or a variant thereof having up to two amino acid substitutions, CDR3 comprises SEQ ID NO: 23 or a variant thereof having up to five amino acid substitutions; or
(iv) CDR1 comprises SEQ ID NO: 27 or a variant thereof having up to two amino acid substitutions, CDR2 comprises SEQ ID NO: 29 or a variant thereof having up to two amino acid substitutions, CDR3 comprises SEQ ID NO: 31 or a variant thereof having up to five amino acid substitutions; or
   (v) CDR1 comprises SEQ ID NO: 35 or a variant thereof having up to two amino acid substitutions, CDR2 comprises SEQ ID NO: 37 or a variant thereof having up to two amino acid substitutions, CDR3 comprises SEQ ID NO: 39 or a variant thereof having up to five amino acid substitutions; or
   (vi) CDR1 comprises SEQ ID NO: 43 or a variant thereof having up to two amino acid substitutions, CDR2 comprises SEQ ID NO: 45 or a variant thereof having up to two amino acid substitutions, CDR3 comprises SEQ ID NO: 47 or a variant thereof having up to five amino acid substitutions;
   (vii) CDR1 comprises SEQ ID NO: 51 or a variant thereof having up to two amino acid substitutions, CDR2 comprises SEQ ID NO: 53 or a variant thereof having up to two amino acid substitutions, CDR3 comprises SEQ ID NO: 55 or a variant thereof having up to five amino acid substitutions; or
   (viii) CDR1 comprises SEQ ID NO: 67 or a variant thereof having up to two amino acid substitutions, CDR2 comprises SEQ ID NO: 69 or a variant thereof having up to two amino acid substitutions, CDR3 comprises SEQ ID NO: 71 or a variant thereof having up to five amino acid substitutions; or
   (ix) CDR1 comprises SEQ ID NO: 75 or a variant thereof having up to two amino acid substitutions, CDR2 comprises SEQ ID NO: 77 or a variant thereof having up to two amino acid substitutions, CDR3 comprises SEQ ID NO: 79 or a variant thereof having up to five amino acid substitutions; or
   (x) CDR1 comprises SEQ ID NO: 83 or a variant thereof having up to two amino acid substitutions, CDR2 comprises SEQ ID NO: 85 or a variant thereof having up to two amino acid substitutions, CDR3 comprises SEQ ID NO: 87 or a variant thereof having up to five amino acid substitutions; or
   (xi) CDR1 comprises SEQ ID NO: 91 or a variant thereof having up to two amino acid substitutions, CDR2 comprises SEQ ID NO: 93 or a variant thereof having up to two amino acid substitutions, CDR3 comprises SEQ ID NO: 95 or a variant thereof having up to five amino acid substitutions.
3. The anti-CRP sdAb of item 1 or 2, wherein the sdAb comprises a first framework region (FR1), a second framework region (FR2) and third framework region (FR3), wherein:
   (i) FR1 comprises SEQ ID NO: 2 or a variant thereof having up to six amino acid substitutions, FR2 comprises SEQ ID NO: 4 or a variant thereof having up to four amino acid substitutions, FR3 comprises SEQ ID NO: 6 or a variant thereof having up to ten amino acid substitutions, and FR4 comprises SEQ ID NO: 8 or a variant thereof having up to three amino acid substitutions; or
   (ii) FR1 comprises SEQ ID NO: 10 or a variant thereof having up to six amino acid substitutions, FR2 comprises SEQ ID NO: 12 or a variant thereof having up to four amino acid substitutions, FR3 comprises SEQ ID NO: 14 or a variant thereof having up to ten amino acid substitutions, and FR4 comprises SEQ ID NO: 16 or a variant thereof having up to three amino acid substitutions; or
   (iii) FR1 comprises SEQ ID NO: 18 or a variant thereof having up to six amino acid substitutions, FR2 comprises SEQ ID NO: 20 or a variant thereof having up to four amino acid substitutions, FR3 comprises SEQ ID NO: 22 or a variant thereof having up to ten amino acid substitutions, and FR4 comprises SEQ ID NO: 24 or a variant thereof having up to three amino acid substitutions; or
   (iv) FR1 comprises SEQ ID NO: 26 or a variant thereof having up to six amino acid substitutions, FR2 comprises SEQ ID NO: 28 or a variant thereof having up to four amino acid substitutions, FR3 comprises SEQ ID NO: 30 or a variant thereof having up to ten amino acid substitutions, and FR4 comprises SEQ ID NO: 32 or a variant thereof having up to three amino acid substitutions; or
   (v) FR1 comprises SEQ ID NO: 34 or a variant thereof having up to six amino acid substitutions, FR2 comprises SEQ ID NO: 36 or a variant thereof having up to four amino acid substitutions, FR3 comprises SEQ ID NO: 38 or a variant thereof having up to ten amino acid substitutions, and FR4 comprises SEQ ID NO: 40 or a variant thereof having up to three amino acid substitutions; or
   (vi) FR1 comprises SEQ ID NO: 42 or a variant thereof having up to six amino acid substitutions, FR2 comprises SEQ ID NO: 44 or a variant thereof having up to four amino acid substitutions, FR3 comprises SEQ ID NO: 46 or a variant thereof having up to ten amino acid substitutions, and FR4 comprises SEQ ID NO: 48 or a variant thereof having up to three amino acid substitutions; or
   (vii) FR1 comprises SEQ ID NO: 50 or a variant thereof having up to six amino acid substitutions, FR2 comprises SEQ ID NO: 52 or a variant thereof having up to four amino acid substitutions, FR3 comprises SEQ ID NO: 54 or a variant thereof having up to ten amino acid substitutions, and FR4 comprises SEQ ID NO: 56 or a variant thereof having up to three amino acid substitutions;
   (viii) FR1 comprises SEQ ID NO: 58 or a variant thereof having up to six amino acid substitutions, FR2 comprises SEQ ID NO: 60 or a variant thereof having up to four amino acid substitutions, FR3 comprises SEQ ID NO: 62 or a variant thereof having up to ten amino acid substitutions, and FR4 comprises SEQ ID NO: 64 or a variant thereof having up to three amino acid substitutions; or
   (ix) FR1 comprises SEQ ID NO: 66 or a variant thereof having up to six amino acid substitutions, FR2 comprises SEQ ID NO: 68 or a variant thereof having up to four amino acid substitutions, FR3 comprises SEQ ID NO: 70 or a variant thereof having up to ten amino acid substitutions, and FR4 comprises SEQ ID NO: 72 or a variant thereof having up to three amino acid substitutions; or
   (x) FR1 comprises SEQ ID NO: 74 or a variant thereof having up to six amino acid substitutions, FR2 comprises SEQ ID NO: 76 or a variant thereof having up to four amino acid substitutions, FR3 comprises SEQ ID NO: 78 or a variant thereof having up to ten amino acid substitutions, and FR4 comprises SEQ ID NO: 80 or a variant thereof having up to three amino acid substitutions; or
   (xi) FR1 comprises SEQ ID NO: 82 or a variant thereof having up to six amino acid substitutions, FR2 comprises SEQ ID NO: 84 or a variant thereof having up to four amino acid substitutions, FR3 comprises SEQ ID NO: 86 or a variant thereof having up to ten amino acid substitutions, and FR4 comprises SEQ ID NO: 88 or a variant thereof having up to three amino acid substitutions; or
   (xii) FR1 comprises SEQ ID NO: 90 or a variant thereof having up to six amino acid substitutions, FR2 comprises SEQ ID NO: 92 or a variant thereof having up to four amino acid substitutions, FR3 comprises SEQ ID NO: 94 or a variant thereof having up to ten amino acid substitutions, and FR4 comprises SEQ ID NO: 96 or a variant thereof having up to three amino acid substitutions.
4. The anti-CRP sdAb of any one of items 1 to 3, wherein the sdAb comprises any one of
   (i) SEQ ID NO: 1 or a variant thereof having at least about 90% sequence identity to SEQ ID NO: 1; or
   (ii) SEQ ID NO: 9 or a variant thereof having at least about 90% sequence identity to SEQ ID NO: 9; or
   (iii) SEQ ID NO: 17 or a variant thereof having at least about 90% sequence identity to SEQ ID NO: 17; or
   (iv) SEQ ID NO: 25 or a variant thereof having at least about 90% sequence identity to SEQ ID NO: 25; or
   (v) SEQ ID NO: 33 or a variant thereof having at least about 90% sequence identity to SEQ ID NO: 33; or
   (vi) SEQ ID NO: 41 or a variant thereof having at least about 90% sequence identity to SEQ ID NO: 41; or
   (vii) SEQ ID NO: 49 or a variant thereof having at least about 90% sequence identity to SEQ ID NO: 49; or
   (viii) SEQ ID NO: 57 or a variant thereof having at least about 90% sequence identity to SEQ ID NO: 57; or
   (ix) SEQ ID NO: 65 or a variant thereof having at least about 90% sequence identity to SEQ ID NO: 65; or
   (x) SEQ ID NO: 73 or a variant thereof having at least about 90% sequence identity to SEQ ID NO: 73; or
   (xi) SEQ ID NO: 81 or a variant thereof having at least about 90% sequence identity to SEQ ID NO: 81; or
   (xii) SEQ ID NO: 89 or a variant thereof having at least about 90% sequence identity to SEQ ID NO: 89.
5. The anti-CRP sdAb of any one of items 1-4, wherein the anti-CRP sdAb comprises an on-rate constant (kₒₙ) of about 3.80×10\hat{ }5 s⁻¹M⁻¹−1.70×10\hat{ }7 s⁻¹M⁻¹ and an off-rate constant (k_{off}) of about 3.20×10\hat{ }−9 s⁻¹−1.30×10\hat{ }−2 s⁻¹.
6. The anti-CRP sdAb of any one of items 1-4, wherein the anti-CRP sdAb comprises an on-rate constant (kₒₙ) of about 3.30×10\hat{ }4 s⁻¹M⁻¹−4.60×10\hat{ }6 s⁻¹M⁻¹ and an off-rate constant (k_{off}) of about 2.60×10\hat{ }−4 s⁻¹−2.20×10\hat{ }−1 s⁻¹.
7. The anti-CRP sdAb of any one of items 1-4, wherein the anti-CRP sdAb comprises an on-rate constant (kₒₙ) of about 4.10×10\hat{ }4 s⁻¹M⁻¹−3.80×10\hat{ }5 s⁻¹M⁻¹ and an off-rate constant (k_{off}) of about 2.00×10\hat{ }−5 s⁻¹−6.90×10\hat{ }−3 s⁻¹.
8. The anti-CRP sdAb of any one of items 1-4, wherein the anti-CRP sdAb comprises an on-rate constant (kₒₙ) of about 3.80×10\hat{ }5 s⁻¹M⁻¹−1.70×10\hat{ }7 s⁻¹M⁻¹±9.06×10\hat{ }2 s 1M⁻¹−3.00×10\hat{ }5 s⁻¹M⁻¹ and an off-rate constant (k_{off}) of about 3.20×10\hat{ }−9 s⁻¹−1.30×10\hat{ }−2 s⁻¹±1.34×10\hat{ }−7 s⁻¹−8.82×10\hat{ }−5 s⁻¹ and optionally a K_{D} of about 1.00×10\hat{ }−2 M−9.62 M, optionally wherein the values are obtained by a SPR Biacore binding kinetic measurement, preferably wherein the sdAb is defined in any one of SEQ ID NOs: 1, 9, 17, 25, 33, 41, 49, 57, 65, 73, 81, 89.
9. The anti-CRP sdAb of any one of items 1-4, wherein the anti-CRP sdAb comprises an on-rate constant (kₒₙ) of about 4.10×10\hat{ }4 s⁻¹M⁻¹−3.80×10\hat{ }5 s⁻¹M−1=3.31×10\hat{ }2 s⁻¹ M⁻¹−1.48×10\hat{ }4 s⁻¹M⁻¹ and an off-rate constant (k_{off}) of about 2.00×10\hat{ }−5 s⁻¹−6.90×10\hat{ }−3 s-1±1.41×10\hat{ }−6 s⁻¹−8.61×10\hat{ }−5 s⁻¹ and optionally a K_{D} of about 5.80×10\hat{ }−11 M−6.50×10\hat{ }−8 M, optionally wherein the values are obtained by an Octet BLI binding kinetic measurement, preferably wherein the sdAb is defined in any one of SEQ ID NOs: 1, 9, 17, 25, 33, 49, 57, 65, 73, 81, 89.
10. The anti-CRP sdAb of any one of items 1-9, wherein the anti-CRP sdAb continuously and reversibly binds CRP.
11. The anti-CRP sdAb of any one of items 1-10, wherein the anti-CRP sdAb binds a second or further CRP after dissociating from a first CRP.
12. The anti-CRP sdAb of any one of items 1-11, wherein the anti-CRP sdAb inhibits the biological function of CRP.
13. The anti-CRP sdAb of any one of items 1-11, wherein the anti-CRP sdAb does not inhibit the biological function of CRP.
14. The anti-CRP sdAb of item 12 or 13, wherein the biological function of CRP is CRP signalling or the CRP signalling pathway.
15. The anti-CRP sdAb of any one of items 1-14, wherein the anti-CRP sdAb is conjugated to a biosensor electrode, such as a working electrode.
16. The anti-CRP sdAb of item 15, wherein the anti-CRP sdAb is conjugated to a biosensor electrode via a linker, such as a nucleic acid.
17. The anti-CRP sdAb of item 15 or 16, wherein the biosensor comprises one or more electrode, such as a working electrode and/or a counter electrode and/or a reference electrode, and a substrate.
18. The anti-CRP sdAb of any one of items 15-17, wherein the biosensor comprises at least a working electrode and a substrate.
19. The anti-CRP sdAb of any one of items 15-18, wherein the biosensor comprises an electrode comprising a conductive material such as carbon, metal, or metallic compound.
20. The anti-CRP sdAb of any one of items 1-19, wherein the anti-CRP sdAb further comprises a detection label or tag.
21. The anti-CRP sdAb of any one of items 1-19, wherein the anti-CRP sdAb does not comprise a detection label or tag.
22. A nucleic acid encoding the anti-CRP sdAb of any one of items 1-21.
23. A vector comprising the nucleic acid of item 22.
24. The vector of item 23, wherein the vector is an expression vector.
25. A method for detecting and/or quantifying CRP in a sample, the method comprising:
   (i) contacting a sample with the anti-CRP sdAb of any one of items 1-21, and
   (ii) detecting binding of the anti-CRP sdAb.
26. An in situ method for detecting and/or quantifying CRP in a subject, the method comprising
   (i) contacting a subject's biological fluid with the anti-CRP sdAb of any one of items 1-21; and
   (ii) detecting binding of the anti-CRP sdAb.
27. The method of item 25 or 26, further comprising detecting the binding of the anti-CRP sdAb over time.
28. The method of any one of items 25-27, further comprising detecting the repeated binding of the anti-CRP sdAb over time.
29. The method of any one of items 25-28, wherein binding of the anti-CRP sdAb is detected by a biosensor.
30. The method of any one of items 25-29, wherein the sample comprises a biological fluid, such as a bodily fluid, for example interstitial fluid (ISF), blood, urine, stool, serum, tear fluid, sweat or saliva; or wherein the biological fluid is a bodily fluid, for example interstitial fluid (ISF), blood, urine, stool, serum, tear fluid, sweat or saliva.
31. The method of any one of items 25-30, wherein the sample comprises human interstitial fluid (ISF), blood, urine, stool, serum, tear, sweat or saliva, or wherein the biological fluid is a human bodily fluid, for example human interstitial fluid (ISF), blood, urine, stool, serum, tear fluid, sweat or saliva.
32. The method of any one of items 25 and 27-29, wherein the sample comprises a sample from a biological production system such as a culture sample.
33. The anti-CRP sdAb of any one of items 1-21 for use as a medicament.
34. The anti-CRP sdAb of any one of items 1-21 for use in in vivo diagnosis.
35. The anti-CRP sdAb of any one of items 1-21 for use in the treatment of a disease associated with an inflammation, optionally a systemic inflammation.
36. A method of treatment a disease associated with an inflammation, optionally a systemic inflammation comprising a step of administering the anti-CRP sdAb of any one of items 1-21 to a subject in need thereof.
37. The anti-CRP sdAb for use of item 35, or the method of item 36, wherein the disease is associated with an increased production or release of inflammatory markers.
38. The anti-CRP sdAb for use of item 35 or 37, or the method of item 36 or 37, wherein the disease is associated with an increased production or release of CRP.
39. The anti-CRP sdAb for use of any one of items 35 or 37-38, or the method of any one of items 36-38, wherein the disease is rheumatoid arthritis, inflammatory bowel disease, IgA nephropathy, Psoriasis, lupus, multiple sclerosis, Endometriosis, Sepsis, cardiovascular disease, metabolic syndrome, hypertension, diabetes, cancer, bacterial or viral infections, COVID19, asthma, familial Mediterranean fever, Alzheimer's disease, Parkinson's diseases, diabetic macular edema (DME), diabetic retinopathy, uveitis, glaucoma, dry eye (e.g., dry eye disease or dry eye syndrome), allergic conjunctivitis, ocular pain, rhegmatogenous retinal detachment (RRD), age-related macular degeneration (AMD), proliferative diabetic retinopathy (PDR), retinal vein occlusion (RVO), neuromyelitis optica (NMO), corneal transplant, corneal abrasion, physical injury to the eye, non-alcoholic fat liver disease, chronic kidney disease, or tumour.
40. The anti-CRP sdAb of any one of items 1-21 for use in a method of in vivo diagnosis of a disease associated with an inflammation, optionally a systemic inflammation, the method comprising:
   (i) contacting a subject with the anti-CRP sdAb of any one of items 1-21, and
   (ii) detecting the binding of the anti-CRP sdAb.
41. A method of in vivo diagnosis of a disease associated with an inflammation, optionally a systemic inflammation, the method comprising:
   (i) contacting a subject with the anti-CRP sdAb of any one of items 1-21, and
   (ii) detecting binding of the anti-CRP sdAb.
42. An ex vivo or in vitro method of monitoring the level of CRP comprising:
   (i) contacting a sample of a subject with the anti-CRP sdAb of any one of items 1-21, and
   (ii) detecting binding of the anti-CRP sdAb.
43. A method of monitoring the level of CRP in a subject comprising:
   (i) contacting the subject with the anti-CRP sdAb of any one of items 1-21, and
   (ii) detecting binding of the anti-CRP sdAb.
44. The anti-CRP sdAb for use of items 35 or 37-40, or the method of any one of items 36-39 or 41-43, wherein binding of the anti-CRP sdAb is repeatedly detected over time.
45. An apparatus comprising:
   (i) the anti-CRP sdAb of any one of items 1-21, and
   (ii) a contacting unit for contacting a sample or a subject with the anti-CRP sdAb, and
   (iii) a detecting unit for detecting and/or quantifying binding of the anti-CRP sdAb, and optionally
   (iv) a dispensing unit for dispensing a drug, optionally wherein the drug is the anti-CRP sdAb of any one of items 1-21.
46. The apparatus of item 45 for use in the treatment or in vivo diagnosis of a disease associated with an inflammation, optionally a systemic inflammation.
47. The apparatus of item 45 or the apparatus for use of item 46, wherein the detecting unit for detecting and/or quantifying binding of the anti-CRP sdAb comprises a biosensor.
48. The apparatus or the apparatus for use of item 47, wherein the biosensor comprises one or more electrode, such as a working electrode and/or a counter electrode and/or a reference electrode, and a substrate, optionally wherein the sdAb is conjugated to the working electrode.
49. The apparatus or the apparatus for use of item 47 or 48, wherein the biosensor comprises at least a working electrode and a substrate.
50. The apparatus or the apparatus for use of any one of items 47-49, wherein the biosensor comprises an electrode comprising a conductive material such as carbon, a metal or metallic compound.
51. The apparatus or the apparatus for use of any one of items 48-50, wherein the anti-CRP sdAb is layered on the one or more electrode or conjugated to the one or more electrode, preferably the working electrode and optionally, wherein the anti-CRP sdAb is conjugated to said electrode via a linker.
52. The anti-CRP sdAb for use of item 35 or 40, or the method of item 36 or 41, or the apparatus for use of any one of items 46-51, wherein the disease is associated with an increased production or release of inflammatory markers.
53. The anti-CRP sdAb for use of any one of items 35, 40 or 52, or the method of any one of items 36, 41 or 52, or the apparatus for use of any one of items 46-52, wherein the disease is associated with an increased production or release of CRP.
54. The anti-CRP sdAb for use of any one of items 35, 40 or 52-53, or the method of any one of items 36, 41 or 52-53, or the apparatus of any one of items 46-53, wherein the disease is any one of rheumatoid arthritis, inflammatory bowel disease, IgA nephropathy, Psoriasis, lupus, multiple sclerosis, Endometriosis, Sepsis, cardiovascular disease, metabolic syndrome, hypertension, diabetes, cancer, bacterial or viral infections, COVID19, asthma, familial Mediterranean fever, Alzheimer's disease, Parkinson's diseases, diabetic macular edema (DME), diabetic retinopathy, uveitis, glaucoma, dry eye (e.g., dry eye disease or dry eye syndrome), allergic conjunctivitis, ocular pain, rhegmatogenous retinal detachment (RRD), age-related macular degeneration (AMD), proliferative diabetic retinopathy (PDR), retinal vein occlusion (RVO), neuromyelitis optica (NMO), corneal transplant, corneal abrasion, physical injury to the eye, non-alcoholic fat liver disease, chronic kidney disease, or tumour.
55. A method of producing an anti-CRP sdAb comprising:
   (i) contacting a subject with a recombinant human CRP, and
   (ii) isolating a sdAb which successfully binds to CRP, and
   (iii) selecting for an on-rate constant (kₒₙ) of about 3.30×10\hat{ }4 s⁻¹M⁻¹−1.70×10\hat{ }7 s⁻¹M⁻¹ and an off-rate constant (k_{off}) of about 3.20×10\hat{ }−9 s⁻¹−2.20×10\hat{ }−1 s⁻¹.
56. The method of item 55, wherein the method comprises selecting for an on-rate constant (kₒₙ) of about 3.80×10\hat{ }5 s⁻¹M⁻¹−1.70×10\hat{ }7 s⁻¹M⁻¹ and an off-rate constant (k_{off}) of about 3.20×10\hat{ }−9 s⁻¹−1.30×10\hat{ }−2 s⁻¹.
57. The method of item 55, wherein the method comprises selecting for an on-rate constant (kₒₙ) of about 3.30×10\hat{ }4 s⁻¹M⁻¹−4.60×10\hat{ }6 s⁻¹M⁻¹ and an off-rate constant (k_{off}) of about 2.60×10\hat{ }−4 s⁻¹−2.20×10\hat{ }−1 s⁻¹.
58. The method of item 55, wherein the method comprises selecting for an on-rate constant (kₒₙ) of about 4.10×10\hat{ }4 s⁻¹M⁻¹−3.80×10\hat{ }5 s⁻¹M⁻¹ and an off-rate constant (k_{off}) of about 2.00×10\hat{ }−5 s⁻¹−6.90×10\hat{ }−3 s⁻¹.
59. The method of any one of items 55-58, wherein the method further comprises a step of selecting for continuous and reversible binding to CRP.
60. The method of any one of items 55-59, wherein the method further comprises a step of selecting for binding to a second or further CRP after dissociation from a first CRP.
61. The method of any one of items 55-60, wherein the method further comprises a step of selecting for inhibiting the biological function of CRP or not inhibiting the biological function of CRP.
62. The method of item 61, wherein the biological function of CRP is CRP signalling or the CRP signalling pathway.
63. An anti-CRP sdAb produced by the method of any one of items 55-62.
64. A biosensor comprising the anti-CRP sdAb of any one of items 1-21.
65. The biosensor of item 64, wherein the biosensor comprises one or more biosensor electrodes.
66. The biosensor of item 64 or 65, wherein the biosensor comprises a working electrode and a substrate 67. The biosensor of item 66, wherein the biosensor further comprises a counter electrode and/or a reference electrode.
68. The biosensor of any one of items 64-67, wherein the anti-CRP sdAb is conjugated to a biosensor electrode, optionally to a working electrode.
69. The biosensor of item 68, wherein the anti-CRP sdAb is conjugated to a biosensor electrode, optionally to a working electrode, via a linker, such as a nucleic acid linker, amino acid/peptide linker, peptide nucleic acid linker, polymer linker, polyethylenglycol (PEG) linker, protein-tag linker, or conventional chemistry technique linker.
70. The biosensor of any one of items 66-69, wherein the working electrode comprises a conductive material, such as carbon, metal or metallic compound.
71. The biosensor of any one of items 64-70, wherein the biosensor, the anti-CRP sdAb or the linker comprises a detection label, a tag, or redox reporters/indicators.
72. The method of any one of items 25-32, 36-39 or 41-44, or the anti-CRP sdAb for use of any one of items 35, 37-40 or 44, wherein binding of the anti-CRP sdAb to CRP is detected and/or quantified by electrochemical ELISA.

The present invention provides single-domain antibodies (sdAb) that specifically bind to C-reactive protein. The sdAb specifically binding CRP of the present invention may be referred to anti-CRP sdAb or anti-CRP VHH. Single-domain antibodies are also known as nanobodies. In the sense of the present invention a sdAb is the heavy chain variable domain of a single chain of a heavy-chain antibody. A heavy-chain antibody is an antibody which consists only of two heavy chains, each of which contain a heavy chain variable domain (V_{H}). The sdAb of the present invention is preferably engineered from the heavy chain variable domain of a camelid heavy-chain antibody.

The sdAbs of the present invention are particularly useful for detecting and monitoring the CRP level over time since they embody a kinetic binding range and allow for continuous association and dissociation within the dynamic range of CRP levels. The sdAb of the present invention have specifically been developed to be capable of detecting CRP throughout the relevant inflammation ranges. Thus, the inventors have found sdAb that can detect CRP levels from a baseline, e.g. in a healthy subject, to CRP levels present during severe inflammation. Consequently, the sdAb of the present invention allow for precise single or continuous, quantitative detection of CRP levels in all clinically relevant ranges of CRP levels.

The sdAbs of the present invention can embody a specific on-rate constant (kₒₙ) and off-rate constant (k_{off}). The kₒₙ (k_{d}) describes the rate of complex formation, i.e. the number of sdAb-CRP complexes formed per second in a one molar solution of sdAb and CRP. The k_{off} (k_{d}) describes the rate or speed at which e.g. a ligand dissociates from a protein, such as a sdAb from CRP. The binding constant (also affinity constant or association constant) Kₐ is defined as kₒₙ/k_{off}. The dissociation constant K_{D} is defined as 1/Kₐ.

Unless otherwise defined, the herein provided kₒₙ (kₐ) values are displayed/provided in the unit [s⁻¹M⁻¹]. Unless otherwise defined, the herein provided k_{off} (k_{d}) values are displayed/provided in the unit [s⁻¹]. Unless otherwise defined, the herein provided dissociation constant K_{D} values are displayed/provided in the unit [nM].

In the sense of the present invention, a sdAb can have a kₒₙ and k_{off} in a range that allows for continuous association and dissociation to CRP. The invention is not particularly limited to any sdAb having specific kₒₙ or k_{off} values, rather any sdAb having kₒₙ and k_{off} values that allow for association and dissociation to CRP can be used.

Accordingly, the present invention provides an anti-C-reactive protein (CRP) single-domain antibody (sdAb) comprising an on-rate constant (kₒₙ) of about 3.30×10\hat{ }4 s⁻¹M⁻¹−1.70×10\hat{ }7 s⁻¹M⁻¹ and an off-rate constant (k_{off}) of about 3×10\hat{ }−9 s⁻¹−2.5×10\hat{ }−1 s⁻¹ (preferably an off-rate constant (k_{off}) of about 3.20×10\hat{ }−9 s⁻¹−2.20×10\hat{ }−1 s⁻¹, preferably wherein said values are obtained by an Octet BLI kinetic measurement, and preferably wherein said sdAb is capable of detecting and/or quantifying CRP concentrations of about 0.01 μM or less to about 1 μM or more.

In the context of the present invention an anti-C-reactive protein (CRP) single-domain antibody (sdAb) can comprise an on-rate constant (kₒₙ) of about or more than about 2.1×10\hat{ }5 s⁻¹M⁻¹ and/or an off-rate constant (k_{off}) of about 2.5×10\hat{ }−9 s⁻¹−2×10\hat{ }−2 s⁻¹, particularly wherein the values are obtained by a SPR Biacore binding kinetic measurement (preferably on samples comprising purified sdAbs).

Accordingly, the present invention relates to an anti-C-reactive protein (CRP) single-domain antibody (sdAb) preferably comprising an on-rate constant (kₒₙ) about or more than about 2.1×10\hat{ }5 s⁻¹M⁻¹.

Preferably the lower limit is about 2.2×10\hat{ }5 s⁻¹M⁻¹, 2.3×10\hat{ }5 s⁻¹M⁻¹, 2.4×10\hat{ }5 s⁻¹M⁻¹, 2.5×10\hat{ }5 s⁻¹M⁻¹, 2.6×10\hat{ }5 s⁻¹M⁻¹, 2.6×10\hat{ }5 s⁻¹M⁻¹, 2.8×10\hat{ }5 s⁻¹M⁻¹, 2.9×10\hat{ }5 s⁻¹M⁻¹, more preferably about 3.0×10\hat{ }5 s⁻¹M⁻¹, 3.1×10\hat{ }5 s⁻¹M⁻¹, 3.2×10\hat{ }5 s⁻¹M⁻¹, 3.3×10\hat{ }5 s⁻¹M⁻¹, 3.4×10\hat{ }5 s⁻¹M⁻¹, 3.5×10\hat{ }5 s⁻¹M⁻¹, 3.6×10\hat{ }5 s⁻¹M⁻¹, 3.7×10\hat{ }5 s⁻¹M⁻¹, and most preferred about 3.8×10\hat{ }5 s⁻¹M⁻¹ (specifically about 3.80×10\hat{ }5 s⁻¹M⁻¹).

The lower limit may be about 4×10\hat{ }5 s⁻¹M⁻¹, 5×10\hat{ }5 s⁻¹M⁻¹, 6×10\hat{ }5 s⁻¹M⁻¹ (specifically about 6.00×10\hat{ }5 s⁻¹M⁻¹), 7×10\hat{ }5 s⁻¹M⁻¹, 8×10\hat{ }5 s⁻¹M⁻¹ (specifically about 8.00×10\hat{ }5 s⁻¹M-1), 9×10\hat{ }5 s 1M−1, 1×10\hat{ }6 s⁻¹M⁻¹ (e.g., about 1.1×10\hat{ }6 s⁻¹M⁻¹, specifically about 1.30×10\hat{ }6 s⁻¹M⁻¹, specifically about 1.50×10\hat{ }6 s⁻¹M⁻¹, specifically about 1.60×10\hat{ }6 s⁻¹M⁻¹, specifically about 1.70×10\hat{ }6 s⁻¹M⁻¹), 2×10\hat{ }6 s⁻¹M⁻¹ (e.g., about 2.05×10\hat{ }6 s⁻¹M⁻¹, specifically about 2.10×10\hat{ }6 s⁻¹M⁻¹, specifically about 2.40×10\hat{ }6 s⁻¹M⁻¹, specifically about 2.70×10\hat{ }6 s⁻¹M-1), 3×10\hat{ }6 s⁻¹M⁻¹, 4×10\hat{ }6 s⁻¹M⁻¹, 5×10\hat{ }6 s⁻¹M⁻¹, 6×10\hat{ }6 s⁻¹M⁻¹, 7×10\hat{ }6 s⁻¹M⁻¹, 8×10\hat{ }6 s⁻¹M⁻¹, 9×10\hat{ }6 s⁻¹M⁻¹, 1×10\hat{ }7 s⁻¹M⁻¹, (e.g., about 1.1×10\hat{ }7 s⁻¹M⁻¹, specifically about 1.70×10\hat{ }7 s⁻¹M⁻¹).

As also mentioned herein above and below, the herein provided sdAbs are, inter alia, particularly useful for their application in (in vitro and/or in vivo) diagnosis and/or diagnostic devices (such as biosensors) or for continuous monitoring of CRP levels. In this context, it is particularly useful that the sdAbs comprise fast association to and dissociation from the antigen (e.g., CRP). Accordingly, in the context of the present invention a particularly high on-rate constant (kₒₙ) is particularly advantageous. Accordingly, the upper limit of the on-rate constant (kₒₙ) of the herein provided anti-CRP sdAbs is not particularly limited.

Nonetheless, the on-rate constant (kₒₙ) of the herein provided anti-CRP sdAbs may comprise an upper limit of about 3×10\hat{ }7 s⁻¹M⁻¹. Accordingly, an anti-CRP sdAb in the sense of the present invention can comprise an on-rate constant (kₒₙ) of about 3×10\hat{ }5 s⁻¹M⁻¹−9×10\hat{ }9 s⁻¹ M⁻¹ and/or an off-rate constant (k_{off}) of about 2.5×10\hat{ }−9 s⁻¹−2×10\hat{ }−2 s⁻¹, particularly wherein the values are obtained by a SPR Biacore binding kinetic measurement (preferably on samples comprising purified sdAbs).

More preferably, the upper limit is about 9×10\hat{ }9 s⁻¹M⁻¹, 8×10\hat{ }9 s⁻¹M⁻¹, 7×10\hat{ }9 s⁻¹M⁻¹, 6×10\hat{ }9 s⁻¹M⁻¹, 5×10\hat{ }9 s⁻¹M⁻¹, 4×10\hat{ }9 s⁻¹M⁻¹, 3×10\hat{ }9 s⁻¹M⁻¹, 2×10\hat{ }9 s⁻¹M⁻¹, 1×10\hat{ }9 s⁻¹M⁻¹, 9×10\hat{ }8 s⁻¹M⁻¹, 8×10\hat{ }8 s⁻¹M⁻¹, 7×10\hat{ }8 s⁻¹M⁻¹, 6×10\hat{ }8 s⁻¹M⁻¹, 5×10\hat{ }8 s⁻¹M⁻¹, 4×10\hat{ }8 s⁻¹ M−1, 3×10\hat{ }8 s⁻¹M⁻¹, 2×10\hat{ }8 s⁻¹M⁻¹, 1×10\hat{ }8 s⁻¹M⁻¹, 9×10\hat{ }7 s⁻¹M⁻¹, 8×10\hat{ }7 s⁻¹M⁻¹, 7×10\hat{ }7 s⁻¹M⁻¹, 6×10\hat{ }7 s⁻¹M⁻¹, 5×10\hat{ }7 s⁻¹M⁻¹, 4×10\hat{ }7 s⁻¹M⁻¹, 3×10\hat{ }7 s⁻¹M⁻¹, 2×10\hat{ }7 s⁻¹M⁻¹, even more preferably about 1.9×10\hat{ }7 s⁻¹M⁻¹, 1.8×10\hat{ }7 s⁻¹M⁻¹, and most preferably about 1.7×10\hat{ }7 s⁻¹M⁻¹.

In this context, the on-rate constant (kₒₙ) values are preferably determined using an SPR Biacore measurement (preferably on samples comprising purified sdAbs).

Accordingly, the present invention relates to an anti-C-reactive protein (CRP) single-domain antibody (sdAb) preferably comprising an off-rate constant (k_{off}) of about 2.5×10\hat{ }−9 s⁻¹−2×10\hat{ }−2 s⁻¹.

Preferably the lower limit is about 2.6×10\hat{ }−9 s¹, 2.7×10\hat{ }−9 s¹, 2.8×10\hat{ }−9 s¹, 2.9×10\hat{ }−9 s¹, 3×10\hat{ }−9 s¹, 3.1×10\hat{ }−9 s⁻¹, most preferred about 3.2×10\hat{ }−9 s⁻¹.

The lower limit may be about 3.3×10\hat{ }−9 s¹, 4×10\hat{ }−9 s¹, 5×10\hat{ }−9 s¹, 6.0×10\hat{ }−9 s¹, 5.0×10\hat{ }−9 s¹, 1.0×10\hat{ }−8 s¹, 1.0×10\hat{ }−7 s⁻¹ (e.g., 1.1×10\hat{ }−7 s⁻¹, specifically about 1.3×10\hat{ }−7 s 1), 1.0×10\hat{ }−6 s¹, 1.0×10\hat{ }−5 s⁻¹ (e.g., about 1.1×10\hat{ }−5 s⁻¹, specifically about 2×10\hat{ }−5 s⁻¹), 6.0×10\hat{ }−5 s⁻¹ (e.g., about 6.1×10\hat{ }−5 s⁻¹, specifically about 6.2×10\hat{ }−5 s⁻¹), 7.0×10\hat{ }−5 s¹, 8.0×10\hat{ }−5 s⁻¹ (e.g., about 8.01×10\hat{ }−5 s⁻¹, specifically about 8.1×10\hat{ }−5 s⁻¹), 9.0×10\hat{ }−5 s⁻¹ (e.g., about 9.1×10\hat{ }−5 s⁻¹, specifically about 9.8×10\hat{ }−5 s⁻¹).

More preferably, the upper limit is about 2×10\hat{ }−2 s¹, 1.9×10\hat{ }−2 s¹, 1.8×10\hat{ }−2 s¹, 1.7×10\hat{ }−2 s¹, 1.6×10\hat{ }−2 s¹, 1.5×10\hat{ }−2 s¹, 1.4×10\hat{ }−2 s⁻¹, (preferably about 1.3×10\hat{ }−2 s⁻¹).

Even more preferably, the upper limit is about 1×10\hat{ }−2 s¹, 9×10\hat{ }−3 s¹, 8×10\hat{ }−3 s¹, 7×10\hat{ }−3 s¹, 6×10\hat{ }−3 s¹, 5×10\hat{ }−3 s¹, 4×10\hat{ }−3 s¹, 3×10\hat{ }−3 s¹, 2×10\hat{ }−3 s⁻¹, (specifically about 1.9×10\hat{ }−3 s⁻¹ or specifically about 1.3×10\hat{ }−3 s⁻¹), 1×10\hat{ }−3 s¹, 9×10\hat{ }−4 s¹, 8×10\hat{ }−4 s¹, 7×10\hat{ }−4 s¹, 6×10\hat{ }−4 s¹, 5×10\hat{ }−4 s¹, 4×10\hat{ }−4 s⁻¹ (specifically about 3×10\hat{ }−4 s⁻¹), 2.9×10\hat{ }−4 s¹, 2.8×10\hat{ }−4 s¹, 2.7×10\hat{ }−4 s¹, 2.6×10\hat{ }−4 s⁻¹ (specifically about 2.5×10\hat{ }−4 s⁻¹), 2.4×10\hat{ }−4 s¹, 2.3×10\hat{ }−4 s¹, 2.2×10\hat{ }−4 s¹, 2.1×10\hat{ }−4 s¹, 2.0×10\hat{ }−4 s¹, 1.9×10\hat{ }−4 s⁻¹, 1.8×10\hat{ }−4 s⁻¹ (specifically about 1.7×10\hat{ }−4 s⁻¹).

In this context, the off-rate constant (k_{off}) values are preferably determined using an SPR Biacore measurement (preferably on samples comprising purified sdAbs).

In particular, the present invention provides for anti-CRP sdAb SCR-3E4 (or variants thereof), comprising an on-rate constant (kₒₙ) of about 3.8×10\hat{ }5 s⁻¹M⁻¹ and/or an off-rate constant (k_{off}) of about 6.2×10\hat{ }−5 s⁻¹.

In particular, the present invention provides for anti-CRP sdAb SCR-3G7 (or variants thereof), comprising an on-rate constant (kₒₙ) of about 6×10\hat{ }5 s⁻¹M⁻¹ and/or an off-rate constant (k_{off}) of about 9.8×10\hat{ }−5 s⁻¹.

In particular, the present invention provides for anti-CRP sdAb SCR-2D1 (or variants thereof), comprising an on-rate constant (kₒₙ) of about 8×10\hat{ }5 s⁻¹M⁻¹ and/or an off-rate constant (k_{off}) of about 3×10\hat{ }−4 s⁻¹

In particular, the present invention provides for anti-CRP sdAb SCR-1A1 (or variants thereof), comprising an on-rate constant (kₒₙ) of about 1.3×10\hat{ }6 s⁻¹M⁻¹ and/or an off-rate constant (k_{off}) of about 1.3×10\hat{ }−2 s⁻¹.

In particular, the present invention provides for anti-CRP sdAb SCR-3B7 (or variants thereof), comprising an on-rate constant (kₒₙ) of about 1.5×10\hat{ }6 s⁻¹M⁻¹ and/or an off-rate constant (k_{off}) of about 1.3×10\hat{ }−7 s⁻¹.

In particular, the present invention provides for anti-CRP sdAb SCR-2D2 (or variants thereof), comprising an on-rate constant (kₒₙ) of about 1.6×10\hat{ }6 s⁻¹M⁻¹ and/or an off-rate constant (k_{off}) of about 2×10\hat{ }−5 s⁻¹

In particular, the present invention provides for anti-CRP sdAb SCR-3E5 (or variants thereof), comprising an on-rate constant (kₒₙ) of about 1.7×10\hat{ }6 s⁻¹M⁻¹ and/or an off-rate constant (k_{off}) of about 3.2×10\hat{ }−9 s⁻¹.

In particular, the present invention provides for anti-CRP sdAb SCR-2B4 (or variants thereof), comprising an on-rate constant (kₒₙ) of about 1.7×10\hat{ }6 s⁻¹M⁻¹ and/or an off-rate constant (k_{off}) of about 1.7×10\hat{ }−4 s⁻¹.

In particular, the present invention provides for anti-CRP sdAb SCR-3H1 (or variants thereof), comprising an on-rate constant (kₒₙ) of about 2.1×10\hat{ }6 s⁻¹M⁻¹ and/or an off-rate constant (k_{off}) of about 2.5×10\hat{ }−4 s⁻¹.

In particular, the present invention provides for anti-CRP sdAb SCR-3H6 (or variants thereof), comprising an on-rate constant (kₒₙ) of about 2.4×10\hat{ }6 s⁻¹M⁻¹ and/or an off-rate constant (k_{off}) of about 8.1×10\hat{ }−5 s⁻¹.

In particular, the present invention provides for anti-CRP sdAb SCR-3A4 (or variants thereof), comprising an on-rate constant (kₒₙ) of about 2.7×10\hat{ }6 s⁻¹M⁻¹ and/or an off-rate constant (k_{off}) of about 1.9×10\hat{ }−3 s⁻¹.

In particular, the present invention provides for anti-CRP sdAb SCR-3A5 (or variants thereof), comprising an on-rate constant (kₒₙ) of about 1.7×10\hat{ }7 s⁻¹M⁻¹ and/or an off-rate constant (k_{off}) of about 1.3×10\hat{ }−3 s⁻¹.

In this context, the on-rate constant (kₒₙ) values and/or the off-rate constant (k_{off}) values are preferably determined using an SPR Biacore measurement (preferably on samples comprising purified sdAbs).

Generally, the lower the K_{D}, the higher the affinity of the corresponding sdAb, meaning the sdAb is capable of detecting a lower limit of CRP concentrations in a biological fluid. This is particularly useful in cases where a very low concentration of analyte e.g. IL6 is present in such a biological fluid. In contrast, CRP is highly concentrated in e.g. human samples, thus, low K_{D} (high affinity) may be less preferred given that in a biosensor setting the biosensor comprising the sdAb with a very low K_{D} will saturate faster.

Accordingly the present invention relates to an anti-C-reactive protein (CRP) single-domain antibody (sdAb) preferably comprising a dissociation constant (K_{D}) of about or less than about 1×10\hat{ }−8 M.

Accordingly, the upper limit is about 1×10\hat{ }−8 M, preferably 9.9×10\hat{ }−9 M, more preferably 9.8×10\hat{ }−9 M, even more preferably 9.7×10\hat{ }−9 M, even more preferably 9.69×10\hat{ }−9 M, even more preferably 9.68×10\hat{ }−9 M, even more preferably 9.67×10\hat{ }−9 M, even more preferably 9.66×10\hat{ }−9 M, even more preferably 9.65×10\hat{ }−9 M, even more preferably 9.64×10\hat{ }−9 M, even more preferably 9.63×10\hat{ }−9 M, most preferred about 9.62×10\hat{ }−9 M.

The lower limit of the dissociation constant (K_{D}) of the herein provided anti-CRP sdAbs is not particularly limited.

Nonetheless, the dissociation constant (K_{D}) of the herein provided anti-CRP sdAbs may comprise a lower limit of about 1×10\hat{ }−16 M, preferably 2×10\hat{ }−16 M, more preferably 3×10\hat{ }−16 M, even more preferably 4×10\hat{ }−16 M, even more preferably 5×10\hat{ }−16 M, even more preferably 6×10\hat{ }−16 M, even more preferably 7×10\hat{ }−16 M, even more preferably 8×10\hat{ }−16 M, even more preferably 9×10\hat{ }−16 M, even more preferably 1×10\hat{ }−15 M, most preferably 1.8×10\hat{ }−15 M. Accordingly, an anti-CRP sdAb in the sense of the present invention can comprise an dissociation constant (K_{D}) of about 1×10\hat{ }−9 M−1×10\hat{ }−8 M.

In this context, the off-rate constant (k_{off}) values are preferably determined using an SPR Biacore measurement (preferably on samples comprising purified sdAbs).

Accordingly, the present invention provides for an anti-C-reactive protein (CRP) single-domain antibody (sdAb) comprising an on-rate constant (kₒₙ) of about or more than about 2.1×10\hat{ }5 s⁻¹ M⁻¹, an off-rate constant (k_{off}) of about 2.5×10\hat{ }−9 s⁻¹−2×10\hat{ }−1 s⁻¹, and/or a dissociation constant (K_{D}) of about less than about 1×10\hat{ }−8 M, preferably wherein said sdAb is capable of detecting and/or quantifying CRP concentrations of about 0.01 μM or less to 1 μM or more, preferably wherein kₒₙ, k_{off}, and K_{D} values are obtained by a SPR Biacore binding kinetic measurement (preferably on samples comprising purified sdAbs).

In the context of the present invention, the herein provided anti-CRP sdAbs may be particularly useful in diagnostic applications or in monitoring of CRP levels. Such diagnostic applications or monitoring may be in vivo and/or in vitro and are not particularly limited. They may include continuous monitoring of CRP concentrations, may rely on dilution, may include a forced release mechanism (e.g. via heat, light/optogenetics, vibration, redox potential or pH), and/or a multisensory chip. All of the herein provided sdAbs may be suitable for any of the above listed applications.

Accordingly, the present invention provides for anti-CRP sdAbs that are, inter alia, useful in in vitro or in vivo diagnostic applications or continuous monitoring of CRP levels e.g. when being comprised in a biosensor which can be used either in vitro or in vivo, i.e. being partially or fully implanted into a subject. Such anti-CRP sdAbs may be any of the herein provided sdAbs. Preferably, the anti-CRP sdAbs (that are particularly useful in in vitro or in vivo diagnostic applications or applications monitoring CRP levels such as, for example in vitro or in vivo biosensors employing such sdAbs in the continuous monitoring/continuous detection/continuous quantification of CRP concentrations) comprise an on-rate constant (kₒₙ) of about 2×10\hat{ }5 s⁻¹M⁻¹−1×10\hat{ }6 s⁻¹M⁻¹, more preferably of about 3×10\hat{ }5 s⁻¹M⁻¹−9×10\hat{ }5 s⁻¹M⁻¹, even more preferably of about 3.5×10\hat{ }5 s⁻¹M⁻¹−8.5×10\hat{ }5 s⁻¹M⁻¹, even more preferably of about 3.8×10\hat{ }5 s⁻¹M⁻¹−8×10\hat{ }5 s⁻¹M⁻¹. In this context, the on-rate constant (kₒₙ) values are preferably determined using a SPR Biacore measurement (preferably on samples comprising purified sdAbs). As is further detailed in the enclosed examples, anti-CRP sdAbs SCR-3E4, SCR-2D1, and SCR-3G7 are particularly useful for in vitro or in vivo diagnostic applications (such as in vivo biosensors). In this context SCR-3E4 and SCR-2D1 may be especially useful as they cover a surprisingly broad inflammation range, comprise a surprisingly high affinity towards CRP, (these advantages are further detailed in Table 13).

Anti-CRP sdAbs SCR-2D2, SCR-3H6, SCR-3A5, SCR-2B4, SCR-3H1, SCR-2B4, and SCR-3A4 may be particularly useful in diagnostic applications that include dilution and/or a forced release mechanism (e.g. via heat, light/optogenetics, vibration, redox potential or pH). Sample dilution allows the antigen concentration within a sample to be aligned with the available kₒₙ/k_{off}/K_{D} characteristics of the sdAbs such that continuous monitoring/continuous detection/continuous quantification of CRP concentrations can be achieved using the herein described thermodynamic equilibrium model. Dilution would reduce the slope of the reaction rate (decrease C₀ in Equation 6) and allow to measure a higher number of datapoints to extract C₀.

Alternatively, serial dilution could be used to reduce the input concentration and reach a linear binding regime between CRP and the sdAbs. The optimal dilution factor will depend on the kinetic rate of the sdAbs used as target molecule and the extent of the targeted CRP concentration range. A forced release mechanism (alone or in combination with dilution) such as by an optogenetic release mechanism, increasing temperature, inducing vibration, pH modifications, or changing the redox potential can further allow for continuous monitoring/continuous detection/continuous quantification of CRP concentrations. Combining multi-sensor chips with those approaches could further increase the inflammation ranges to be monitored (i.e. by using different dilutions on separate chips and/or release mechanisms and/or different sdAbs within the same biosensor).

In the context of the present invention, the herein provided anti-CRP sdAbs may also be useful in therapeutic applications, which may include the treatment and/or prevention of CRP-associated diseases and/or diseases and/or conditions that are associated with elevated CRP-concentrations (for example in a biological fluid, such as the anti-CRP sdAbs for use in the treatment of a disease associated with an inflammation as provided herein or the corresponding method of treatment as provided herein. Preferred sdAbs useful in one or more of such therapeutic applications may, inter alia, be found in Table 13. Anti-CRP sdAbs SCR-3E5, SCR-3B7, SCR-3G7, SCR-2D2, SCR-3H6, SCR-3A5, SCR-2B4, SCR-3H1, SCR-2B4, and SCR-3A4 may be particularly useful in such therapeutic applications given their sub-nanomolar affinity. Anti-CRP sdAbs SCR-2D2, SCR-3H6, SCR-3A5, SCR-2B4, SCR-3H1, SCR-2B4, and SCR-3A4 may be preferred for the herein provided therapeutic applications given their sub-nanomolar affinity.

As is detailed in the herein provided non-limiting examples instead of determining or quantifying the binding affinities (such as kₒₙ, k_{off}, and K_{D} values) using SPR Biacore measurements (as can illustratively be seen in Table 10), such values can also be determined by other means, such as by Octet BLI kinetic measurements (as can, inter alia, illustratively be seen in Table 8).

Accordingly, in the context of the present invention an anti-C-reactive protein (CRP) single-domain antibody (sdAb) can comprise an on-rate constant (kₒₙ) of about or less than about 1.9×10\hat{ }5 s⁻¹M⁻¹ and/or an off-rate constant (k_{off}) of about 1×10\hat{ }−5 s⁻¹−7×10\hat{ }−3 s⁻¹, preferably wherein said values are obtained by an Octet BLI kinetic measurement (preferably on samples comprising purified sdAbs)

Accordingly, the present invention relates to an anti-C-reactive protein (CRP) single-domain antibody (sdAb) preferably comprising an on-rate constant (kₒₙ) about or less than about 1.9×10\hat{ }5 s⁻¹M⁻¹.

Preferably the upper limit is about or less than about 1.9×10\hat{ }5 s⁻¹M⁻¹, more preferably 1.8×10\hat{ }5 s⁻¹M⁻¹, even more preferably 1.7×10\hat{ }5 s⁻¹M⁻¹, even more preferably 1.6×10\hat{ }5 s⁻¹M⁻¹, even more preferably 1.5×10\hat{ }5 s⁻¹M⁻¹, even more preferably 1.4×10\hat{ }5 s⁻¹M⁻¹, even more preferably 1.3×10\hat{ }5 s⁻¹M⁻¹ (specifically 1.30×10\hat{ }5 s⁻¹M⁻¹). It may also be preferred herein that the upper limit is about or less than about 1.2×10\hat{ }5 s⁻¹M⁻¹, preferably 1.1×10\hat{ }5 s⁻¹M⁻¹ (specifically 1.0×10\hat{ }5 s⁻¹M⁻¹).

In this context, the lower limit of the on-rate constant (kₒₙ) of the herein provided anti-CRP sdAbs is not particularly limited.

Nonetheless, the on-rate constant (kₒₙ) of the herein provided anti-CRP sdAbs may comprise a lower limit of about 1×10\hat{ }3 s⁻¹M⁻¹, preferably 2×10\hat{ }3 s⁻¹M⁻¹, more preferably 3×10\hat{ }3 s⁻¹M⁻¹, even more preferably 4×10\hat{ }3 s⁻¹M⁻¹, even more preferably 5×10\hat{ }3 s⁻¹M⁻¹, even more preferably 6×10\hat{ }3 s⁻¹M⁻¹, even more preferably 7×10\hat{ }3 s⁻¹M⁻¹, even more preferably 8×10\hat{ }3 s⁻¹M⁻¹, even more preferably 9×10\hat{ }3 s⁻¹M⁻¹, even more preferably 1×10\hat{ }4 s⁻¹M⁻¹, even more preferably 2×10\hat{ }4 s⁻¹M⁻¹, even more preferably 3×10\hat{ }4 s⁻¹M⁻¹, most preferably 4×10\hat{ }4 s⁻¹M⁻¹ (specifically about 4.1×10\hat{ }4 s⁻¹M⁻¹). Accordingly, an anti-CRP sdAb in the sense of the present invention can comprise an on-rate constant (kₒₙ) of about 4×10\hat{ }4 s⁻¹M⁻¹−1.9×10\hat{ }5 s⁻¹M⁻¹ and/or an off-rate constant (k_{off}) of about 1×10\hat{ }−5 s⁻¹−7×10\hat{ }−3 s⁻¹, particularly wherein the values are obtained by an Octet BLI kinetic measurement (preferably on samples comprising purified sdAbs), however any other herein above (or below) defined upper and/or lower limit is also envisaged herein.

In the context of the present invention an anti-C-reactive protein (CRP) single-domain antibody (sdAb) can comprise an on-rate constant (kₒₙ) of about or more than about 2×10\hat{ }5 s⁻¹M⁻¹ and/or an off-rate constant (k_{off}) of about 1×10\hat{ }−5 s⁻¹−7×10\hat{ }−3 s⁻¹, preferably wherein said values are obtained by an Octet BLI kinetic measurement (preferably on samples comprising purified sdAbs)

Accordingly, the present invention relates to an anti-C-reactive protein (CRP) single-domain antibody (sdAb) preferably comprising an on-rate constant (kₒₙ) about or more than about 2×10\hat{ }5 s⁻¹M⁻¹.

Preferably the lower limit is about or more than about 2×10\hat{ }5 s⁻¹M⁻¹, more preferably 2.1×10\hat{ }5 s⁻¹M⁻¹ (specifically 2.2×10\hat{ }5 s⁻¹M⁻¹).

In this context, the upper limit of the on-rate constant (kₒₙ) of the herein provided anti-CRP sdAbs is not particularly limited.

Nonetheless, the on-rate constant (kₒₙ) of the herein provided anti-CRP sdAbs may comprise a upper limit of about 9×10\hat{ }6 s⁻¹M⁻¹, preferably 8×10\hat{ }6 s⁻¹M⁻¹, more preferably 7×10\hat{ }6 s⁻¹M⁻¹, even more preferably 6×10\hat{ }6 s⁻¹M⁻¹, even more preferably 5×10\hat{ }6 s⁻¹M⁻¹, even more preferably 4×10\hat{ }6 s⁻¹M⁻¹, even more preferably 3×10\hat{ }6 s⁻¹M⁻¹, even more preferably 2×10\hat{ }6 s⁻¹M⁻¹, even more preferably 1×10\hat{ }6 s⁻¹M⁻¹, even more preferably 9×10\hat{ }5 s⁻¹M⁻¹, even more preferably 8×10\hat{ }5 s⁻¹M⁻¹, even more preferably 7×10\hat{ }5 s⁻¹M⁻¹, even more preferably 6×10\hat{ }5 s⁻¹M⁻¹, even more preferably 5×10\hat{ }5 s⁻¹M⁻¹, most preferably 4×10\hat{ }5 s⁻¹M⁻¹ (specifically about 3.8×10\hat{ }5 s⁻¹M⁻¹). Accordingly, an anti-CRP sdAb in the sense of the present invention can comprise an on-rate constant (kₒₙ) of about 2×10\hat{ }5 s⁻¹M⁻¹−4×10\hat{ }5 s″ 1M⁻¹ and/or an off-rate constant (k_{off}) of about 1×10\hat{ }−5 s⁻¹−7×10\hat{ }−3 s⁻¹, particularly wherein the values are obtained by an Octet BLI kinetic measurement (preferably on samples comprising purified sdAbs), however any other herein above (or below) defined upper and/or lower limit is also envisaged herein.

In the context of the present invention an anti-C-reactive protein (CRP) single-domain antibody (sdAb) can comprise an off-rate constant (k_{off}) of about or less than about 2×10\hat{ }−3 s⁻¹ and/or an on-rate constant (kₒₙ) of about 4×10\hat{ }4 s⁻¹M⁻¹−4×10\hat{ }5 s⁻¹M⁻¹, preferably wherein said values are obtained by an Octet BLI kinetic measurement (preferably on samples comprising purified sdAbs)

Accordingly, the present invention relates to an anti-C-reactive protein (CRP) single-domain antibody (sdAb) preferably comprising an off-rate constant (k_{off}) about or less than about 2×10\hat{ }−3 s⁻¹.

Preferably the upper limit is about or less than about 2×10\hat{ }−3 s⁻¹, more preferably 1×10\hat{ }−3 s″ 1, even more preferably 9×10\hat{ }−4 s⁻¹, even more preferably 8×10\hat{ }−4 s⁻¹, even more preferably 7×10\hat{ }−4 s⁻¹ (specifically 6,8×10\hat{ }−4 s⁻¹).

In this context, the lower limit of the off-rate constant (k_{off}) of the herein provided anti-CRP sdAbs is not particularly limited.

Nonetheless, the off-rate constant (k_{off}) of the herein provided anti-CRP sdAbs may comprise a lower limit of about 1×10\hat{ }−6 s⁻¹, preferably 2×10\hat{ }−6 s⁻¹, more preferably 3×10\hat{ }−6 s⁻¹, even more preferably 4×10\hat{ }−6 s⁻¹, even more preferably 5×10\hat{ }−6 s⁻¹, even more preferably 6×10\hat{ }−6 s⁻¹, even more preferably 7×10\hat{ }−6 s⁻¹, even more preferably 8×10\hat{ }−6 s⁻¹, even more preferably 9×10\hat{ }−6 s⁻¹, even more preferably 1×10\hat{ }−5 s⁻¹, most preferably 2×10\hat{ }−5 s⁻¹ (specifically about 2×10\hat{ }−5 s⁻¹). Accordingly, an anti-CRP sdAb in the sense of the present invention can comprise an off-rate constant (k_{off}) of 2×10\hat{ }−7 s⁻¹−2×10\hat{ }−3 s⁻¹ and/or an on-rate constant (kₒₙ) of about 4×10\hat{ }4 s⁻¹M⁻¹−4×10\hat{ }5 s⁻¹M⁻¹, particularly wherein the values are obtained by an Octet BLI kinetic measurement (preferably on samples comprising purified sdAbs), however any other herein above (or below) defined upper and/or lower limit is also envisaged herein.

In the context of the present invention an anti-C-reactive protein (CRP) single-domain antibody (sdAb) can comprise an off-rate constant (k_{off}) of about or more than about 3×10\hat{ }−3 s⁻¹ and/or an on-rate constant (kₒₙ) of about 4×10\hat{ }4 s⁻¹M⁻¹−4×10\hat{ }5 s⁻¹M⁻¹, preferably wherein said values are obtained by an Octet BLI kinetic measurement (preferably on samples comprising purified sdAbs)

Accordingly, the present invention relates to an anti-C-reactive protein (CRP) single-domain antibody (sdAb) preferably comprising an off-rate constant (k_{off}) about or more than about 3×10\hat{ }−3 s⁻¹.

Preferably the lower limit is about or less than about 3×10\hat{ }−3 s⁻¹, more preferably 4×10\hat{ }−3 s⁻¹, most preferably 5×10\hat{ }−3 s⁻¹ (specifically 5,8×10\hat{ }−3 s⁻¹).

In this context, the upper limit of the off-rate constant (k_{off}) of the herein provided anti-CRP sdAbs is not particularly limited.

Nonetheless, the off-rate constant (k_{off}) of the herein provided anti-CRP sdAbs may comprise an upper limit of about 9×10\hat{ }−2 s⁻¹, preferably 8×10\hat{ }−2 s⁻¹, more preferably 7×10\hat{ }−2 s⁻¹, even more preferably 6×10\hat{ }−2 s⁻¹, even more preferably 5×10\hat{ }−2 s⁻¹, even more preferably 4×10\hat{ }−2 s⁻¹, even more preferably 3×10\hat{ }−2 s⁻¹, even more preferably 2×10\hat{ }−2 s⁻¹, even more preferably 1×10\hat{ }−2 s⁻¹, even more preferably 9×10\hat{ }−3 s⁻¹, even more preferably 8×10\hat{ }−3 s⁻¹, most preferably 7×10\hat{ }−3 s⁻¹ (specifically about 6.9×10\hat{ }−3 s⁻¹). Accordingly, an anti-CRP sdAb in the sense of the present invention can comprise an off-rate constant (k_{off}) of 3×10\hat{ }−3−7×10\hat{ }−3 s⁻¹ s⁻¹ and/or an on-rate constant (kₒₙ) of about 4×10\hat{ }4 s⁻¹M⁻¹−4×10\hat{ }5 s⁻¹M⁻¹, particularly wherein the values are obtained by an Octet BLI kinetic measurement (preferably on samples comprising purified sdAbs), however any other herein above (or below) defined upper and/or lower limit is also envisaged herein.

In the context of the present invention an anti-C-reactive protein (CRP) single-domain antibody (sdAb) can comprise a dissociation constant (K_{D}) about or less than about 1.2×10\hat{ }−8 M, preferably wherein said value is obtained by an Octet BLI kinetic measurement (preferably on samples comprising purified sdAbs)

Preferably the upper limit is about or less than about 1.2×10\hat{ }−8 M, more preferably 1.1×10\hat{ }−8 M, even more preferably 1×10\hat{ }−8 M, even more preferably 9×10\hat{ }−9 M, even more preferably 8×10\hat{ }−9 M, even more preferably 7×10\hat{ }−9 M, even more preferably 6×10\hat{ }−9 M (specifically 5.1×10\hat{ }−9 M).

In this context, the lower limit of the dissociation constant (K_{D}) of the herein provided anti-CRP sdAbs is not particularly limited.

Nonetheless, the dissociation constant (K_{D}) of the herein provided anti-CRP sdAbs may comprise a lower limit of about 5×10\hat{ }−12 M, preferably 6×10\hat{ }−12 M, more preferably 7×10\hat{ }−12 M, even more preferably 8×10\hat{ }−12 M, even more preferably 9×10\hat{ }−12 M, even more preferably 1×10\hat{ }−11 M, even more preferably 2×10\hat{ }−11 M, even more preferably 3×10\hat{ }−11 M, even more preferably 4×10\hat{ }−11 M, most preferably 5×10\hat{ }−11 M (specifically about 5.8×10\hat{ }−11 M). Accordingly, an anti-CRP sdAb in the sense of the present invention can comprise a dissociation constant (K_{D}) about 5×10\hat{ }−11 M−1.2×10\hat{ }−8 M, preferably wherein said value is obtained by an Octet BLI kinetic measurement (preferably on samples comprising purified sdAbs), however any other herein above (or below) defined upper and/or lower limit is also envisaged herein.

In the context of the present invention an anti-C-reactive protein (CRP) single-domain antibody (sdAb) can comprise a dissociation constant (K_{D}) about 1.4×10\hat{ }−8 M−5×10\hat{ }−8 M, preferably about 1.5×10\hat{ }−8 M−4×10\hat{ }−8 M, more preferably about 1.6×10\hat{ }−8 M−3×10\hat{ }−8 M, even more preferably about 1.7×10\hat{ }−8 M−2×10\hat{ }−8 M, most preferably about 1.7×10\hat{ }−8 M−1.9×10\hat{ }−8 M, preferably wherein said value is obtained by an Octet BLI kinetic measurement (preferably on samples comprising purified sdAbs).

In the context of the present invention an anti-C-reactive protein (CRP) single-domain antibody (sdAb) can comprise a dissociation constant (K_{D}) about 5.2×10\hat{ }−8 M−1×10\hat{ }−7 M, preferably about 5.3×10\hat{ }−8 M−9×10\hat{ }−8 M, more preferably 5.4×10\hat{ }−8 M−8×10\hat{ }−8 M, even more preferably about 5.5×10\hat{ }−8 M−7×10\hat{ }−8 M, most preferably about 5.5×10\hat{ }−8 M−6.5×10\hat{ }−8 M, preferably wherein said value is obtained by an Octet BLI kinetic measurement (preferably on samples comprising purified sdAbs).

In particular, the present invention provides for anti-CRP sdAb SCR-3E4 (or variants thereof), comprising an on-rate constant (kₒₙ) of about 4.1×10\hat{ }4 s⁻¹M⁻¹ and/or an off-rate constant (k_{off}) of about 6.8×10\hat{ }−4 s⁻¹.

In particular, the present invention provides for anti-CRP sdAb SCR-3G7 (or variants thereof), comprising an on-rate constant (kₒₙ) of about 1.0×10\hat{ }5 s⁻¹M⁻¹ and/or an off-rate constant (k_{off}) of about 6.8×10\hat{ }−3 s⁻¹.

In particular, the present invention provides for anti-CRP sdAb SCR-2D1 (or variants thereof), comprising an on-rate constant (kₒₙ) of about 6.4×10\hat{ }4 s⁻¹M⁻¹ and/or an off-rate constant (k_{off}) of about 3.3×10\hat{ }−4 s⁻¹.

In particular, the present invention provides for anti-CRP sdAb SCR-1A1 (or variants thereof), comprising an on-rate constant (kₒₙ) of about 1.3×10\hat{ }5 s⁻¹M⁻¹ and/or an off-rate constant (k_{off}) of about 6.9×10\hat{ }−3 s⁻¹.

In particular, the present invention provides for anti-CRP sdAb SCR-3B7 (or variants thereof), comprising an on-rate constant (kₒₙ) of about 3.0×10\hat{ }5 s⁻¹M⁻¹ and/or an off-rate constant (k_{off}) of about 5.9×10\hat{ }−5 s⁻¹.

In particular, the present invention provides for anti-CRP sdAb SCR-2D2 (or variants thereof), comprising an on-rate constant (kₒₙ) of about 3.8×10\hat{ }5 s⁻¹M⁻¹ and/or an off-rate constant (k_{off}) of about 1.3×10\hat{ }−4 s⁻¹.

In particular, the present invention provides for anti-CRP sdAb SCR-3E5 (or variants thereof), comprising an on-rate constant (kₒₙ) of about 2.2×10\hat{ }5 s⁻¹M⁻¹ and/or an off-rate constant (k_{off}) of about 3.0×10\hat{ }−5 s⁻¹.

In particular, the present invention provides for anti-CRP sdAb SCR-2B4 (or variants thereof), comprising an on-rate constant (kₒₙ) of about 2.7×10\hat{ }5 s⁻¹M⁻¹ and/or an off-rate constant (k_{off}) of about 4.0×10\hat{ }−4 s⁻¹.

In particular, the present invention provides for anti-CRP sdAb SCR-3H1 (or variants thereof), comprising an on-rate constant (kₒₙ) of about 3.0×10\hat{ }5 s⁻¹M⁻¹ and/or an off-rate constant (k_{off}) of about 8.1×10\hat{ }−5 s⁻¹.

In particular, the present invention provides for anti-CRP sdAb SCR-3H6 (or variants thereof), comprising an on-rate constant (kₒₙ) of about 3.5×10\hat{ }5 s⁻¹M⁻¹ and/or an off-rate constant (k_{off}) of about 2.0×10\hat{ }−5 s⁻¹.

In particular, the present invention provides for anti-CRP sdAb SCR-3A4 (or variants thereof), comprising an on-rate constant (kₒₙ) of about 3.1×10\hat{ }5 s⁻¹M⁻¹ and/or an off-rate constant (k_{off}) of about 5.8×10\hat{ }−3 s⁻¹.

In this context, the on-rate constant (kₒₙ) values and/or the off-rate constant (k_{off}) values are preferably determined using an Octet BLI kinetic measurement (preferably on samples comprising purified sdAbs).

In particular, the present invention provides for anti-CRP sdAb SCR-3A5 (or variants thereof), comprising an on-rate constant (kₒₙ) of about 1.3×10\hat{ }6 s⁻¹M⁻¹ and/or an off-rate constant (k_{off}) of about 2.2×10\hat{ }−1 s⁻¹, preferably wherein the on-rate constant (kₒₙ) values and/or the off-rate constant (k_{off}) values are preferably determined using an Octet BLI kinetic measurement (preferably on samples comprising non-purified or crude sdAbs).

It is understood that the anti-C-reactive protein (CRP) single-domain antibody (sdAb) of the invention can comprise any of the above disclosed on-rate constant (kₒₙ) and/or any of the above disclosed off-rate constant (k_{off}). Any combinations of the disclosed on-rate constant (kₒₙ) and off-rate constant (k_{off}) are envisaged.

In the sense of the present invention, an anti-CRP sdAb can comprise an on-rate constant (kₒₙ) of about or more than about 2.1×10\hat{ }5 s⁻¹M⁻¹ and/or an off-rate constant (k_{off}) of about 3×10\hat{ }−9 s⁻¹−2×10\hat{ }−2 s⁻¹, preferably wherein said values are obtained by a SPR Biacore measurement.

In the sense of the present invention, an anti-CRP sdAb can comprise an on-rate constant (kₒₙ) of about or more than about 3.80×10\hat{ }5 s⁻¹M⁻¹ and/or an off-rate constant (k_{off}) of about 3.2×10\hat{ }−9 s⁻¹−1.3×10\hat{ }−2 s⁻¹, preferably wherein said values are obtained by a SPR Biacore kinetic measurement.

In the sense of the present invention, an anti-CRP sdAb can comprise an on-rate constant (kₒₙ) of about 2.1×10\hat{ }5 s⁻¹M⁻¹−3×10\hat{ }7 s⁻¹M⁻¹ and/or an off-rate constant (k_{off}) of about 3×10\hat{ }−9 s⁻¹−2×10\hat{ }−2 s⁻¹, preferably wherein said values are obtained by a SPR Biacore measurement.

In the sense of the present invention, an anti-CRP sdAb can comprise an on-rate constant (kₒₙ) of about 3.80×10\hat{ }5 s⁻¹M⁻¹−1.7×10\hat{ }7 s⁻¹M⁻¹ and/or an off-rate constant (k_{off}) of about 3.2×10\hat{ }−9 s⁻¹−1.3×10\hat{ }−2 s⁻¹, preferably wherein said values are obtained by a SPR Biacore kinetic measurement.

In the sense of the present invention, an anti-CRP sdAb can comprise an on-rate constant (kₒₙ) of about 3×10\hat{ }4 s⁻¹M⁻¹−5×10\hat{ }6 s⁻¹M⁻¹ and/or an off-rate constant (k_{off}) of about 2×10\hat{ }−4 s⁻¹−3×10\hat{ }−1 s⁻¹, preferably wherein said values are obtained by an Octet BLI kinetic measurement.

In the sense of the present invention, an anti-CRP sdAb can comprise an on-rate constant (kₒₙ) of about 3.3×10\hat{ }4 s⁻¹M⁻¹−4.6×10\hat{ }6 s⁻¹M⁻¹ and/or an off-rate constant (k_{off}) of about 2.4×10\hat{ }−4 s⁻¹−2.2×10\hat{ }−1 s⁻¹, preferably wherein said values are obtained by an Octet BLI kinetic measurement.

In the sense of the present invention, an anti-CRP sdAb can comprise an on-rate constant (kₒₙ) of about 3×10\hat{ }5 s⁻¹M⁻¹−5×10\hat{ }6 s⁻¹M⁻¹ and/or an off-rate constant (k_{off}) of about 3×10\hat{ }−4 s⁻¹−3×10\hat{ }−1 s⁻¹, preferably wherein said values are obtained by an Octet BLI kinetic measurement.

In the sense of the present invention, an anti-CRP sdAb can comprise an on-rate constant (kₒₙ) of about 3.9×10\hat{ }5 s⁻¹M⁻¹−4.6×10\hat{ }6 s⁻¹M⁻¹ and/or an off-rate constant (k_{off}) of about 3.7×10\hat{ }−4 s⁻¹−2.2×10\hat{ }−1 s⁻¹, preferably wherein said values are obtained by an Octet BLI kinetic measurement.

In the sense of the present invention, an anti-CRP sdAb can comprise a kₒₙ of about 3.30×10\hat{ }4−1.70×10\hat{ }7 and a k_{off} of about 3.20×10\hat{ }−9−2.20×10\hat{ }−1. An anti-CRP sdAb can comprise a kₒₙ of about 3.80×10\hat{ }5−1.70×10\hat{ }7 and k_{off} of about 3.20×10\hat{ }−9−1.30×10\hat{ }−2. An anti-CRP sdAb can comprise a kₒₙ of about 3.30×10\hat{ }4−4.60×10\hat{ }6 and k_{off} of about 2.60×10\hat{ }−4−2.20×10\hat{ }−1. An anti-CRP sdAb can comprise a kₒₙ of about 4.10×10\hat{ }4−3.80×10\hat{ }5 and K_{off} of about 2.00×10\hat{ }−5−6.90×10\hat{ }−3.

An anti-CRP sdAb can have a kₒₙ of about 3.80×10\hat{ }5−1.70×10\hat{ }7 and a k_{off} of about 3.20×10\hat{ }−9−1.30×10\hat{ }−2, wherein the values are obtained by a SPR Biacore binding kinetic measurement. An anti-CRP sdAb can have a kₒₙ of about 3.80×10\hat{ }5−1.70×10\hat{ }7 and a k_{off} of about 3.20×10\hat{ }−9−1.30×10\hat{ }−2 and optionally a K_{D} of about 1.00×10\hat{ }−2−9.62, wherein the values are obtained by a SPR Biacore binding kinetic measurement.

An anti-CRP sdAb can comprise a kₒₙ of about 3.30×10\hat{ }4−4.60×10\hat{ }6 and a k_{off} of about 2.60×10\hat{ }−4−2.20×10\hat{ }−1, wherein the values are obtained by an Octet BLI binding kinetic measurement. An anti-CRP sdAb can comprise a kₒₙ of about 3.30×10\hat{ }4−4.60×10\hat{ }6 and a K_{off} of about 2.60×10\hat{ }−4−2.20×10\hat{ }−1, and optionally a K_{D} of about 1.50×10\hat{ }−10 M−1.80×10\hat{ }−7 M, wherein the values are obtained by an Octet BLI binding kinetic measurement.

An anti-CRP sdAb can comprise a kₒₙ of about 4.10×10\hat{ }4−3.80×10\hat{ }5 and a k_{off} of about 2.00×10\hat{ }−5−6.90×10\hat{ }−3, wherein the values are obtained by an Octet BLI binding kinetic measurement. An anti-CRP sdAb can comprise a kₒₙ of about 4.10×10\hat{ }4−3.80×10\hat{ }5 and a K_{off} of about 2.00×10\hat{ }−5−6.90×10\hat{ }−3, and optionally a K_{D} of about 5.80×10\hat{ }−11 M−6.50×10\hat{ }−8 M, wherein the values are obtained by an Octet BLI binding kinetic measurement.

The kₒₙ and k_{off} values of a sdAb of the present invention can vary depending on the sdAb, the method of measurement and the measurement setup used. Accordingly, the skilled person is aware that a sdAb of the present invention may not embody strict kₒₙ and k_{off} values, but these may be in a range defined by the above parameters. Accordingly, it is possible to define a certain range of a kₒₙ and k_{off} value by repeating measurements and forming a standard deviation (SD). For example, a SD can be based on repeated measurements, deviations reported in the prior art, and/or minimum and maximum values obtained for SDs from repeated measurements and SDs reported in the prior art. Different means to arrive at standard deviations for the sdAbs of the present application are described in detail in the appended examples.

Accordingly, the invention provides in one aspect an anti-CRP sdAb comprising an on-rate constant (kₒₙ) of about 3.80×10\hat{ }5−1.70×10\hat{ }7±3.53×10\hat{ }5−1.58×10\hat{ }7 and an off-rate constant (k_{off}) of about 3.20×10\hat{ }−9−1.30×10\hat{ }−2±1.34×10\hat{ }−7−1.85×10\hat{ }−2 and optionally a K_{D} of about 1.00×10\hat{ }−2−9.62, optionally wherein the values are obtained by a SPR Biacore binding kinetic measurement, preferably wherein the sdAb is defined in any one of SEQ ID NOs: 1, 9, 17, 25, 33, 41, 49, 57, 65, 73, 81, 89.

Terms like “on-rate constant (kₒₙ) of about 3.80×10\hat{ }5−1.70×10\hat{ }7±3.53×10\hat{ }5−1.58×10\hat{ }7 and an off-rate constant (k_{off}) of about 3.20×10\hat{ }−9−1.30×10\hat{ }−2±1.34×10\hat{ }−7−1.85×10\hat{ }−2” as disclosed herein are to be understood to relate to “on-rate constant (kₒₙ) of about between (from) 3.80×10\hat{ }5 to 1.70×10\hat{ }7±3.53×10\hat{ }5−1.58×10\hat{ }7 (wherein the “±3.53×10\hat{ }5−1.58×10\hat{ }7” is the range of the SD of “between (from) 3.53×10\hat{ }5−1.58×10\hat{ }7” associated with the indicated range of the on-rate constant (kₒₙ)) and an off-rate constant (k_{off}) of between (from) 3.20×10\hat{ }−9−1.30×10\hat{ }−2±1.34×10\hat{ }−7−1.85×10\hat{ }−2 (wherein the “+1.34×10\hat{ }−7−1.85×10\hat{ }−2” is the range of the SD of “between (from) 1.34×10\hat{ }−7 to 1.85×10\hat{ }−2” associated with the indicated range of the off-rate constant (k_{off})”. This explanation applies to all similar terms disclosed herein.

In some embodiments, the invention provides an anti-CRP sdAb comprising an on-rate constant (kₒₙ) of about 3.80×10\hat{ }5−1.70×10\hat{ }7±3.53×10\hat{ }5−1.58×10\hat{ }7 and an off-rate constant (k_{off}) of about 3.20×10\hat{ }−9−1.30×10\hat{ }−2±4.56×10\hat{ }−9−1.85×10\hat{ }−2 and optionally a K_{D} of about 1.00×10\hat{ }−2−9.62, optionally wherein the values are obtained by a SPR Biacore binding kinetic measurement, preferably wherein the sdAb is defined in any one of SEQ ID NOs: 1, 9, 17, 25, 33, 41, 49, 57, 65, 73, 81, 89.

In some embodiments, the invention provides an anti-CRP sdAb comprising an on-rate constant (kₒₙ) of about 3.80×10\hat{ }5−1.70×10\hat{ }7±9.06×10\hat{ }2−3.00×10\hat{ }5 and an off-rate constant (k_{off}) of about 3.20×10\hat{ }−9−1.30×10\hat{ }−2±1.34×10\hat{ }−7−8.82×10\hat{ }−5 and optionally a K_{D} of about 1.00×10\hat{ }−2−9.62, optionally wherein the values are obtained by a SPR Biacore binding kinetic measurement, preferably wherein the sdAb is defined in any one of SEQ ID NOs: 1, 9, 17, 25, 33, 41, 49, 57, 65, 73, 81, 89.

In some embodiments, the invention provides an anti-CRP sdAb comprising an on-rate constant (kₒₙ) of about 1.30×10\hat{ }6±1.21×10\hat{ }6 and an off-rate constant (k_{off}) of about 1.30×10\hat{ }−2±1.85×10\hat{ }−2 and optionally a K_{D} of about 9.62, optionally wherein the values are obtained by a SPR Biacore binding kinetic measurement, preferably wherein the sdAb is defined in SEQ ID NO: 1.

In some embodiments, the invention provides an anti-CRP sdAb comprising an on-rate constant (kₒₙ) of about 1.30×10\hat{ }6±9.31×10\hat{ }3 and an off-rate constant (k_{off}) of about 1.30×10\hat{ }−2±8.82×10\hat{ }−5 and optionally a K_{D} of about 9.62, optionally wherein the values are obtained by a SPR Biacore binding kinetic measurement, preferably wherein the sdAb is defined in SEQ ID NO: 1.

In some embodiments, the invention provides an anti-CRP sdAb comprising an on-rate constant (kₒₙ) of about 1.70×10\hat{ }6±1.58×10\hat{ }6 and an off-rate constant (k_{off}) of about 1.70×10\hat{ }−4±2.42×10\hat{ }−4 and optionally a K_{D} of about 0.10, optionally wherein the values are obtained by a SPR Biacore binding kinetic measurement, preferably wherein the sdAb is defined in SEQ ID NO: 9.

In some embodiments, the invention provides an anti-CRP sdAb comprising an on-rate constant (kₒₙ) of about 1.70×10\hat{ }6±5.00×10\hat{ }3 and an off-rate constant (k_{off}) of about 1.70×10\hat{ }−4±2.20×10\hat{ }−6 and optionally a K_{D} of about 0.10, optionally wherein the values are obtained by a SPR Biacore binding kinetic measurement, preferably wherein the sdAb is defined in SEQ ID NO: 9.

In some embodiments, the invention provides an anti-CRP sdAb comprising an on-rate constant (kₒₙ) of about 8.00×10\hat{ }5±7.43×10\hat{ }5 and an off-rate constant (k_{off}) of about 3.00×10\hat{ }−4±4.28×10\hat{ }−4 and optionally a K_{D} of about 0.38, optionally wherein the values are obtained by a SPR Biacore binding kinetic measurement, preferably wherein the sdAb is defined in SEQ ID NO: 17.

In some embodiments, the invention provides an anti-CRP sdAb comprising an on-rate constant (kₒₙ) of about 8.00×10\hat{ }5±1.26×10\hat{ }3 and an off-rate constant (k_{off}) of about 3.00×10\hat{ }−4±1.20×10\hat{ }−6 and optionally a K_{D} of about 0.38, optionally wherein the values are obtained by a SPR Biacore binding kinetic measurement, preferably wherein the sdAb is defined in SEQ ID NO: 17.

In some embodiments, the invention provides an anti-CRP sdAb comprising an on-rate constant (kₒₙ) of about 1.60×10\hat{ }6±1.49×10\hat{ }6 and an off-rate constant (k_{off}) of about 2.00×10\hat{ }−5±2.85×10\hat{ }−5 and optionally a K_{D} of about 0.01, optionally wherein the values are obtained by a SPR Biacore binding kinetic measurement, preferably wherein the sdAb is defined in SEQ ID NO: 25.

In some embodiments, the invention provides an anti-CRP sdAb comprising an on-rate constant (kₒₙ) of about 1.60×10\hat{ }6±3.80×10\hat{ }3 and an off-rate constant (k_{off}) of about 2.00×10\hat{ }−5±1.79×10\hat{ }−6 and optionally a K_{D} of about 0.01, optionally wherein the values are obtained by a SPR Biacore binding kinetic measurement, preferably wherein the sdAb is defined in SEQ ID NO: 25.

In some embodiments, the invention provides an anti-CRP sdAb comprising an on-rate constant (kₒₙ) of about 2.70×10\hat{ }6±2.51×10\hat{ }6 and an off-rate constant (k_{off}) of about 1.90×10\hat{ }−3±2.71×10\hat{ }−3 and optionally a K_{D} of about 0.70, optionally wherein the values are obtained by a SPR Biacore binding kinetic measurement, preferably wherein the sdAb is defined in SEQ ID NO: 33.

In some embodiments, the invention provides an anti-CRP sdAb comprising an on-rate constant (kₒₙ) of about 2.70×10\hat{ }6±4.47×10\hat{ }4 and an off-rate constant (k_{off}) of about 1.90×10\hat{ }−3±2.91×10\hat{ }−5 and optionally a K_{D} of about 0.70, optionally wherein the values are obtained by a SPR Biacore binding kinetic measurement, preferably wherein the sdAb is defined in SEQ ID NO: 33.

In some embodiments, the invention provides an anti-CRP sdAb comprising an on-rate constant (kₒₙ) of about 1.70×10\hat{ }7±1.58×10\hat{ }7 and an off-rate constant (k_{off}) of about 1.30×10\hat{ }−3±1.85×10\hat{ }−3 and optionally a K_{D} of about 0.08, optionally wherein the values are obtained by a SPR Biacore binding kinetic measurement, preferably wherein the sdAb is defined in SEQ ID NO: 41.

In some embodiments, the invention provides an anti-CRP sdAb comprising an on-rate constant (kₒₙ) of about 1.70×10\hat{ }7±3.00×10\hat{ }5 and an off-rate constant (k_{off}) of about 1.30×10\hat{ }−3±2.00×10\hat{ }−5 and optionally a K_{D} of about 0.08, optionally wherein the values are obtained by a SPR Biacore binding kinetic measurement, preferably wherein the sdAb is defined in SEQ ID NO: 41.

In some embodiments, the invention provides an anti-CRP sdAb comprising an on-rate constant (kₒₙ) of about 1.50×10\hat{ }6±1.39×10\hat{ }6 and an off-rate constant (k_{off}) of about 1.30×10\hat{ }−7±1.88×10\hat{ }−7 and optionally a K_{D} of about 7.60×10\hat{ }−5, optionally wherein the values are obtained by a SPR Biacore binding kinetic measurement, preferably wherein the sdAb is defined in SEQ ID NO: 49.

In some embodiments, the invention provides an anti-CRP sdAb comprising an on-rate constant (kₒₙ) of about 1.50×10\hat{ }6±1.39×10\hat{ }6 and an off-rate constant (k_{off}) of about 1.30×10\hat{ }−7±1.85×10\hat{ }−7 and optionally a K_{D} of about 7.60×10\hat{ }−5, optionally wherein the values are obtained by a SPR Biacore binding kinetic measurement, preferably wherein the sdAb is defined in SEQ ID NO: 49.

In some embodiments, the invention provides an anti-CRP sdAb comprising an on-rate constant (kₒₙ) of about 1.50×10\hat{ }6±3.80×10\hat{ }3 and an off-rate constant (k_{off}) of about 1.30×10\hat{ }−7±1.88×10\hat{ }−7 and optionally a K_{D} of about 7.60×10\hat{ }−5, optionally wherein the values are obtained by a SPR Biacore binding kinetic measurement, preferably wherein the sdAb is defined in SEQ ID NO: 49.

In some embodiments, the invention provides an anti-CRP sdAb comprising an on-rate constant (kₒₙ) of about 3.80×10\hat{ }5±3.53×10\hat{ }5 and an off-rate constant (k_{off}) of about 6.20×10\hat{ }−5±8.84×10\hat{ }−5 and optionally a K_{D} of about 0.16, optionally wherein the values are obtained by a SPR Biacore binding kinetic measurement, preferably wherein the sdAb is defined in SEQ ID NO: 57.

In some embodiments, the invention provides an anti-CRP sdAb comprising an on-rate constant (kₒₙ) of about 3.80×10\hat{ }5±9.06×10\hat{ }2 and an off-rate constant (k_{off}) of about 6.20×10\hat{ }−5±2.28×10\hat{ }−6 and optionally a K_{D} of about 0.16, optionally wherein the values are obtained by a SPR Biacore binding kinetic measurement, preferably wherein the sdAb is defined in SEQ ID NO: 57.

In some embodiments, the invention provides an anti-CRP sdAb comprising an on-rate constant (kₒₙ) of about 1.70×10\hat{ }6±1.58×10\hat{ }6 and an off-rate constant (k_{off}) of about 3.20×10\hat{ }−9±1.34×10\hat{ }−7 and optionally a K_{D} of about 1.80×10\hat{ }−6, optionally wherein the values are obtained by a SPR Biacore binding kinetic measurement, preferably wherein the sdAb is defined in SEQ ID NO: 65.

In some embodiments, the invention provides an anti-CRP sdAb comprising an on-rate constant (kₒₙ) of about 1.70×10\hat{ }6±1.58×10\hat{ }6 and an off-rate constant (k_{off}) of about 3.20×10\hat{ }9±4.56×10\hat{ }−9 and optionally a K_{D} of about 1.80×10\hat{ }−6, optionally wherein the values are obtained by a SPR Biacore binding kinetic measurement, preferably wherein the sdAb is defined in SEQ ID NO: 65.

In some embodiments, the invention provides an anti-CRP sdAb comprising an on-rate constant (kₒₙ) of about 1.70×10\hat{ }6±2.46×10\hat{ }3 and an off-rate constant (k_{off}) of about 3.20×10\hat{ }−9±1.34×10\hat{ }−7 and optionally a K_{D} of about 1.80×10\hat{ }−6, optionally wherein the values are obtained by a SPR Biacore binding kinetic measurement, preferably wherein the sdAb is defined in SEQ ID NO: 65.

In some embodiments, the invention provides an anti-CRP sdAb comprising an on-rate constant (kₒₙ) of about 6.00×10\hat{ }5±5.57×10\hat{ }5 and an off-rate constant (k_{off}) of about 9.80×10\hat{ }−5±1.40×10\hat{ }−4 and optionally a K_{D} of about 0.16, optionally wherein the values are obtained by a SPR Biacore binding kinetic measurement, preferably wherein the sdAb is defined in SEQ ID NO: 73.

In some embodiments, the invention provides an anti-CRP sdAb comprising an on-rate constant (kₒₙ) of about 6.00×10\hat{ }5±6.24×10\hat{ }3 and an off-rate constant (k_{off}) of about 9.80×10\hat{ }−5±1.11×10\hat{ }−6 and optionally a K_{D} of about 0.16, optionally wherein the values are obtained by a SPR Biacore binding kinetic measurement, preferably wherein the sdAb is defined in SEQ ID NO: 73.

In some embodiments, the invention provides an anti-CRP sdAb comprising an on-rate constant (kₒₙ) of about 2.10×10\hat{ }6±1.95×10\hat{ }6 and an off-rate constant (k_{off}) of about 2.50×10\hat{ }−4±3.57×10\hat{ }−4 and optionally a K_{D} of about 0.12, optionally wherein the values are obtained by a SPR Biacore binding kinetic measurement, preferably wherein the sdAb is defined in SEQ ID NO: 81.

In some embodiments, the invention provides an anti-CRP sdAb comprising an on-rate constant (kₒₙ) of about 2.10×10\hat{ }6±4.47×10\hat{ }3 and an off-rate constant (k_{off}) of about 2.50×10\hat{ }−4±1.86×10\hat{ }−6 and optionally a K_{D} of about 0.12, optionally wherein the values are obtained by a SPR Biacore binding kinetic measurement, preferably wherein the sdAb is defined in SEQ ID NO: 81.

In some embodiments, the invention provides an anti-CRP sdAb comprising an on-rate constant (kₒₙ) of about 2.40×10\hat{ }6±2.23×10\hat{ }6 and an off-rate constant (k_{off}) of about 8.10×10\hat{ }−5±1.16×10\hat{ }−4 and optionally a K_{D} of about 0.03, optionally wherein the values are obtained by a SPR Biacore binding kinetic measurement, preferably wherein the sdAb is defined in SEQ ID NO: 89.

In some embodiments, the invention provides an anti-CRP sdAb comprising an on-rate constant (kₒₙ) of about 2.40×10\hat{ }6±4.90×10\hat{ }3 and an off-rate constant (k_{off}) of about 8.10×10\hat{ }−5±2.69×10\hat{ }−7 and optionally a K_{D} of about 0.03, optionally wherein the values are obtained by a SPR Biacore binding kinetic measurement, preferably wherein the sdAb is defined in SEQ ID NO: 89.

In some embodiments, the invention provides an anti-CRP sdAb comprising a first complementary determining region (CDR1) or a variant thereof having up to two amino acid substitutions, a second CDR (CDR2) or a variant thereof having up to two amino acid substitutions and a third CDR (CDR3) or a variant thereof having up to five amino acid substitutions, comprising an on-rate constant (kₒₙ) of about 3.30×10\hat{ }4−1.70×10\hat{ }7 and an off-rate constant (k_{off}) of about 3.20×10\hat{ }−9−2.20×10\hat{ }−1, wherein CDR1 comprises any one of SEQ ID NOs: 3, 11, 19, 27, 35, 43, 51, 59, 67, 75, 83, or 91, wherein CDR2 comprises any one of SEQ ID NOs: 5, 13, 21, 29, 37, 45, 53, 61, 69, 77, 85, or 93 and wherein CDR3 comprises any one of SEQ ID NOs: 7, 15, 23, 31, 39, 47, 55, 63, 71, 79, 87, or 95.

In some embodiments, the invention provides an anti-CRP sdAb comprising a first complementary determining region (CDR1) or a variant thereof having up to two amino acid substitutions, a second CDR (CDR2) or a variant thereof having up to two amino acid substitutions and a third CDR (CDR3) or a variant thereof having up to five amino acid substitutions, comprising an on-rate constant (kₒₙ) of about 3.80×10\hat{ }5−1.70×10\hat{ }7 and an off-rate constant (k_{off}) of about 3.20×10\hat{ }−9−1.30×10\hat{ }−2, wherein CDR1 comprises any one of SEQ ID NOs: 3, 11, 19, 27, 35, 43, 51, 59, 67, 75, 83, or 91, wherein CDR2 comprises any one of SEQ ID NOs: 5, 13, 21, 29, 37, 45, 53, 61, 69, 77, 85, or 93 and wherein CDR3 comprises any one of SEQ ID NOs: 7, 15, 23, 31, 39, 47, 55, 63, 71, 79, 87, or 95, optionally wherein the values are obtained by a SPR Biacore binding kinetic measurement.

In some embodiments, the invention provides an anti-CRP sdAb comprising a first complementary determining region (CDR1) or a variant thereof having up to two amino acid substitutions, a second CDR (CDR2) or a variant thereof having up to two amino acid substitutions and a third CDR (CDR3) or a variant thereof having up to five amino acid substitutions, comprising an on-rate constant (kₒₙ) of about 3.80×10\hat{ }5−1.70×10\hat{ }7=3.53×10\hat{ }5−1.58×10\hat{ }7 and an off-rate constant (k_{off}) of about 3.20×10\hat{ }−9−1.30×10\hat{ }−2±1.34×10\hat{ }−7−1.85×10\hat{ }−2 and optionally a K_{D} of about 1.00×10\hat{ }−2−9.62, wherein CDR1 comprises any one of SEQ ID NOs: 3, 11, 19, 27, 35, 43, 51, 59, 67, 75, 83, or 91, wherein CDR2 comprises any one of SEQ ID NOs: 5, 13, 21, 29, 37, 45, 53, 61, 69, 77, 85, or 93 and wherein CDR3 comprises any one of SEQ ID NOs: 7, 15, 23, 31, 39, 47, 55, 63, 71, 79, 87, or 95, optionally wherein the values are obtained by a SPR Biacore binding kinetic measurement. In some embodiments, the invention provides an anti-CRP sdAb comprising a first complementary determining region (CDR1) or a variant thereof having up to two amino acid substitutions, a second CDR (CDR2) or a variant thereof having up to two amino acid substitutions and a third CDR (CDR3) or a variant thereof having up to five amino acid substitutions, comprising an on-rate constant (kₒₙ) of about 3.80×10\hat{ }5−1.70×10\hat{ }7±3.53×10\hat{ }5−1.58×10\hat{ }7 and an off-rate constant (k_{off}) of about 3.20×10\hat{ }−9−1.30×10\hat{ }−2=4.56×10\hat{ }−9−1.85×10\hat{ }−2 and optionally a K_{D} of about 1.00×10\hat{ }−2−9.62, wherein CDR1 comprises any one of SEQ ID NOs: 3, 11, 19, 27, 35, 43, 51, 59, 67, 75, 83, or 91, wherein CDR2 comprises any one of SEQ ID NOs: 5, 13, 21, 29, 37, 45, 53, 61, 69, 77, 85, or 93 and wherein CDR3 comprises any one of SEQ ID NOs: 7, 15, 23, 31, 39, 47, 55, 63, 71, 79, 87, or 95, optionally wherein the values are obtained by a SPR Biacore binding kinetic measurement. In some embodiments, the invention provides an anti-CRP sdAb comprising a first complementary determining region (CDR1) or a variant thereof having up to two amino acid substitutions, a second CDR (CDR2) or a variant thereof having up to two amino acid substitutions and a third CDR (CDR3) or a variant thereof having up to five amino acid substitutions, comprising an on-rate constant (kₒₙ) of about 3.80×10\hat{ }5−1.70×10\hat{ }7±9.06×10\hat{ }2−3.00×10\hat{ }5 and an off-rate constant (k_{off}) of about 3.20×10\hat{ }−9−1.30×10\hat{ }−2±1.34×10\hat{ }−7−8.82×10\hat{ }−5 and optionally a K_{D} of about 1.00×10\hat{ }−2−9.62, wherein CDR1 comprises any one of SEQ ID NOs: 3, 11, 19, 27, 35, 43, 51, 59, 67, 75, 83, or 91, wherein CDR2 comprises any one of SEQ ID NOs: 5, 13, 21, 29, 37, 45, 53, 61, 69, 77, 85, or 93 and wherein CDR3 comprises any one of SEQ ID NOs: 7, 15, 23, 31, 39, 47, 55, 63, 71, 79, 87, or 95, optionally wherein the values are obtained by a SPR Biacore binding kinetic measurement.

In some embodiments, the invention provides an anti-CRP sdAb comprising a CDR1 comprising SEQ ID NO: 3 or a variant thereof having up to two amino acid substitutions, CDR2 comprising SEQ ID NO: 5 or a variant thereof having up to two amino acid substitutions, CDR3 comprising SEQ ID NO: 7 or a variant thereof having up to five amino acid substitutions, comprising an on-rate constant (kₒₙ) of about 1.30×10\hat{ }6±1.21×10\hat{ }6 and an off-rate constant (K_{off}) of about 1.30×10\hat{ }−2±1.85×10\hat{ }−2 and optionally a K_{D} of about 9.62, optionally wherein the values are obtained by a SPR Biacore binding kinetic measurement.

In some embodiments, the invention provides an anti-CRP sdAb comprising a CDR1 comprising SEQ ID NO: 3 or a variant thereof having up to two amino acid substitutions, CDR2 comprising SEQ ID NO: 5 or a variant thereof having up to two amino acid substitutions, CDR3 comprising SEQ ID NO: 7 or a variant thereof having up to five amino acid substitutions, comprising an on-rate constant (kₒₙ) of about 1.30×10\hat{ }6±9.31×10\hat{ }3 and an off-rate constant (k_{off}) of about 1.30×10\hat{ }−2±8.82×10\hat{ }−5 and optionally a K_{D} of about 9.62, optionally wherein the values are obtained by a SPR Biacore binding kinetic measurement.

In some embodiments, the invention provides an anti-CRP sdAb comprising a CDR1 comprising SEQ ID NO: 11 or a variant thereof having up to two amino acid substitutions, CDR2 comprising SEQ ID NO: 13 or a variant thereof having up to two amino acid substitutions, CDR3 comprising SEQ ID NO: 15 or a variant thereof having up to five amino acid substitutions, comprising an on-rate constant (kₒₙ) of about 1.70×10\hat{ }6±1.58×10\hat{ }6 and an off-rate constant (k_{off}) of about 1.70×10\hat{ }−4±2.42×10\hat{ }−4 and optionally a K_{D} of about 0.10, optionally wherein the values are obtained by a SPR Biacore binding kinetic measurement.

In some embodiments, the invention provides an anti-CRP sdAb comprising a CDR1 comprising SEQ ID NO: 11 or a variant thereof having up to two amino acid substitutions, CDR2 comprising SEQ ID NO: 13 or a variant thereof having up to two amino acid substitutions, CDR3 comprising SEQ ID NO: 15 or a variant thereof having up to five amino acid substitutions, comprising an on-rate constant (kₒₙ) of about 1.70×10\hat{ }6±5.00×10\hat{ }3 and an off-rate constant (k_{off}) of about 1.70×10\hat{ }−4±2.20×10\hat{ }−6 and optionally a K_{D} of about 0.10, optionally wherein the values are obtained by a SPR Biacore binding kinetic measurement.

In some embodiments, the invention provides an anti-CRP sdAb comprising a CDR1 comprising SEQ ID NO: 19 or a variant thereof having up to two amino acid substitutions, CDR2 comprising SEQ ID NO: 21 or a variant thereof having up to two amino acid substitutions, CDR3 comprising SEQ ID NO: 23 or a variant thereof having up to five amino acid substitutions, comprising an on-rate constant (kₒₙ) of about 8.00×10\hat{ }5±7.43×10\hat{ }5 and an off-rate constant (k_{off}) of about 3.00×10\hat{ }−4±4.28×10\hat{ }−4 and optionally a K_{D} of about 0.38, optionally wherein the values are obtained by a SPR Biacore binding kinetic measurement.

In some embodiments, the invention provides an anti-CRP sdAb comprising a CDR1 comprising SEQ ID NO: 19 or a variant thereof having up to two amino acid substitutions, CDR2 comprising SEQ ID NO: 21 or a variant thereof having up to two amino acid substitutions, CDR3 comprising SEQ ID NO: 23 or a variant thereof having up to five amino acid substitutions, comprising an on-rate constant (kₒₙ) of about 8.00×10\hat{ }5±1.26×10\hat{ }3 and an off-rate constant (k_{off}) of about 3.00×10\hat{ }−4±1.20×10\hat{ }−6 and optionally a K_{D} of about 0.38, optionally wherein the values are obtained by a SPR Biacore binding kinetic measurement.

In some embodiments, the invention provides an anti-CRP sdAb comprising a CDR1 comprising SEQ ID NO: 27 or a variant thereof having up to two amino acid substitutions, CDR2 comprising SEQ ID NO: 29 or a variant thereof having up to two amino acid substitutions, CDR3 comprising SEQ ID NO: 31 or a variant thereof having up to five amino acid substitutions, comprising an on-rate constant (kₒₙ) of about 1.60×10\hat{ }6±1.49×10\hat{ }6 and an off-rate constant (k_{off}) of about 2.00×10\hat{ }−5±2.85×10\hat{ }−5 and optionally a K_{D} of about 0.01, optionally wherein the values are obtained by a SPR Biacore binding kinetic measurement.

In some embodiments, the invention provides an anti-CRP sdAb comprising a CDR1 comprising SEQ ID NO: 27 or a variant thereof having up to two amino acid substitutions, CDR2 comprising SEQ ID NO: 29 or a variant thereof having up to two amino acid substitutions, CDR3 comprising SEQ ID NO: 31 or a variant thereof having up to five amino acid substitutions, comprising an on-rate constant (kₒₙ) of about 1.60×10\hat{ }6±3.80×10\hat{ }3 and an off-rate constant (k_{off}) of about 2.00×10\hat{ }−5±1.79×10\hat{ }−6 and optionally a K_{D} of about 0.01, optionally wherein the values are obtained by a SPR Biacore binding kinetic measurement.

In some embodiments, the invention provides an anti-CRP sdAb comprising a CDR1 comprising SEQ ID NO: 35 or a variant thereof having up to two amino acid substitutions, CDR2 comprising SEQ ID NO: 37 or a variant thereof having up to two amino acid substitutions, CDR3 comprising SEQ ID NO: 39 or a variant thereof having up to five amino acid substitutions, comprising an on-rate constant (kₒₙ) of about 2.70×10\hat{ }6=2.51×10\hat{ }6 and an off-rate constant (k_{off}) of about 1.90×10\hat{ }−3±2.71×10\hat{ }−3 and optionally a K_{D} of about 0.70, optionally wherein the values are obtained by a SPR Biacore binding kinetic measurement.

In some embodiments, the invention provides an anti-CRP sdAb comprising a CDR1 comprising SEQ ID NO: 35 or a variant thereof having up to two amino acid substitutions, CDR2 comprising SEQ ID NO: 37 or a variant thereof having up to two amino acid substitutions, CDR3 comprising SEQ ID NO: 39 or a variant thereof having up to five amino acid substitutions, comprising an on-rate constant (kₒₙ) of about 2.70×10\hat{ }6±4.47×10\hat{ }4 and an off-rate constant (k_{off}) of about 1.90×10\hat{ }−3±2.91×10\hat{ }−5 and optionally a K_{D} of about 0.70, optionally wherein the values are obtained by a SPR Biacore binding kinetic measurement.

In some embodiments, the invention provides an anti-CRP sdAb comprising a CDR1 comprising SEQ ID NO: 43 or a variant thereof having up to two amino acid substitutions, CDR2 comprising SEQ ID NO: 45 or a variant thereof having up to two amino acid substitutions, CDR3 comprising SEQ ID NO: 47 or a variant thereof having up to five amino acid substitutions, comprising an on-rate constant (kₒₙ) of about 1.70×10\hat{ }7=1.58×10\hat{ }7 and an off-rate constant (k_{off}) of about 1.30×10\hat{ }−3±1.85×10\hat{ }−3 and optionally a K_{D} of about 0.08, optionally wherein the values are obtained by a SPR Biacore binding kinetic measurement.

In some embodiments, the invention provides an anti-CRP sdAb comprising a CDR1 comprising SEQ ID NO: 43 or a variant thereof having up to two amino acid substitutions, CDR2 comprising SEQ ID NO: 45 or a variant thereof having up to two amino acid substitutions, CDR3 comprising SEQ ID NO: 47 or a variant thereof having up to five amino acid substitutions, comprising an on-rate constant (kₒₙ) of about 1.70×10\hat{ }7±3.00×10\hat{ }5 and an off-rate constant (k_{off}) of about 1.30×10\hat{ }−3±2.00×10\hat{ }−5 and optionally a K_{D} of about 0.08, optionally wherein the values are obtained by a SPR Biacore binding kinetic measurement.

In some embodiments, the invention provides an anti-CRP sdAb comprising a CDR1 comprising SEQ ID NO: 51 or a variant thereof having up to two amino acid substitutions, CDR2 comprising SEQ ID NO: 53 or a variant thereof having up to two amino acid substitutions, CDR3 comprising SEQ ID NO: 55 or a variant thereof having up to five amino acid substitutions, comprising an on-rate constant (kₒₙ) of about 1.50×10\hat{ }6=1.39×10\hat{ }6 and an off-rate constant (k_{off}) of about 1.30×10\hat{ }−7±1.88×10\hat{ }−7 and optionally a K_{D} of about 7.60×10\hat{ }−5, optionally wherein the values are obtained by a SPR Biacore binding kinetic measurement.

In some embodiments, the invention provides an anti-CRP sdAb comprising a CDR1 comprising SEQ ID NO: 51 or a variant thereof having up to two amino acid substitutions, CDR2 comprising SEQ ID NO: 53 or a variant thereof having up to two amino acid substitutions, CDR3 comprising SEQ ID NO: 55 or a variant thereof having up to five amino acid substitutions, comprising an on-rate constant (kₒₙ) of about 1.50×10\hat{ }6=1.39×10\hat{ }6 and an off-rate constant (k_{off}) of about 1.30×10\hat{ }−7±1.85×10\hat{ }−7 and optionally a K_{D} of about 7.60×10\hat{ }−5, optionally wherein the values are obtained by a SPR Biacore binding kinetic measurement.

In some embodiments, the invention provides an anti-CRP sdAb comprising a CDR1 comprising SEQ ID NO: 51 or a variant thereof having up to two amino acid substitutions, CDR2 comprising SEQ ID NO: 53 or a variant thereof having up to two amino acid substitutions, CDR3 comprising SEQ ID NO: 55 or a variant thereof having up to five amino acid substitutions, comprising an on-rate constant (kₒₙ) of about 1.50×10\hat{ }6±3.80×10\hat{ }3 and an off-rate constant (k_{off}) of about 1.30×10\hat{ }−7±1.88×10\hat{ }−7 and optionally a K_{D} of about 7.60×10\hat{ }−5, optionally wherein the values are obtained by a SPR Biacore binding kinetic measurement.

In some embodiments, the invention provides an anti-CRP sdAb comprising a CDR1 comprising SEQ ID NO: 59 or a variant thereof having up to two amino acid substitutions, CDR2 comprising SEQ ID NO: 61 or a variant thereof having up to two amino acid substitutions, CDR3 comprising SEQ ID NO: 63or a variant thereof having up to five amino acid substitutions, comprising an on-rate constant (kₒₙ) of about 3.80×10\hat{ }5±3.53×10\hat{ }5 and an off-rate constant (k_{off}) of about 6.20×10\hat{ }−5±8.84×10\hat{ }−5 and optionally a K_{D} of about 0.16, optionally wherein the values are obtained by a SPR Biacore binding kinetic measurement.

In some embodiments, the invention provides an anti-CRP sdAb comprising a CDR1 comprising SEQ ID NO: 59 or a variant thereof having up to two amino acid substitutions, CDR2 comprising SEQ ID NO: 61 or a variant thereof having up to two amino acid substitutions, CDR3 comprising SEQ ID NO: 63 or a variant thereof having up to five amino acid substitutions, comprising an on-rate constant (kₒₙ) of about 3.80×10\hat{ }5±9.06×10\hat{ }2 and an off-rate constant (k_{off}) of about 6.20×10\hat{ }−5±2.28×10\hat{ }−6 and optionally a K_{D} of about 0.16, optionally wherein the values are obtained by a SPR Biacore binding kinetic measurement.

In some embodiments, the invention provides an anti-CRP sdAb comprising a CDR1 comprising SEQ ID NO: 67 or a variant thereof having up to two amino acid substitutions, CDR2 comprising SEQ ID NO: 69 or a variant thereof having up to two amino acid substitutions, CDR3 comprising SEQ ID NO: 71 or a variant thereof having up to five amino acid substitutions, comprising an on-rate constant (kₒₙ) of about 1.70×10\hat{ }6±1.58×10\hat{ }6 and an off-rate constant (k_{off}) of about 3.20×10\hat{ }−9±1.34×10\hat{ }−7 and optionally a K_{D} of about 1.80×10\hat{ }−6, optionally wherein the values are obtained by a SPR Biacore binding kinetic measurement.

In some embodiments, the invention provides an anti-CRP sdAb comprising a CDR1 comprising SEQ ID NO: 67 or a variant thereof having up to two amino acid substitutions, CDR2 comprising SEQ ID NO: 69 or a variant thereof having up to two amino acid substitutions, CDR3 comprising SEQ ID NO: 71 or a variant thereof having up to five amino acid substitutions, comprising an on-rate constant (kₒₙ) of about 1.70×10\hat{ }6±1.58×10\hat{ }6 and an off-rate constant (k_{off}) of about 3.20×10\hat{ }−9±4.56×10\hat{ }−9 and optionally a K_{D} of about 1.80×10\hat{ }−6, optionally wherein the values are obtained by a SPR Biacore binding kinetic measurement.

In some embodiments, the invention provides an anti-CRP sdAb comprising a CDR1 comprising SEQ ID NO: 67 or a variant thereof having up to two amino acid substitutions, CDR2 comprising SEQ ID NO: 69 or a variant thereof having up to two amino acid substitutions, CDR3 comprising SEQ ID NO: 71 or a variant thereof having up to five amino acid substitutions, comprising an on-rate constant (kₒₙ) of about 1.70×10\hat{ }6±2.46×10\hat{ }3 and an off-rate constant (k_{off}) of about 3.20×10\hat{ }−9±1.34×10\hat{ }−7 and optionally a K_{D} of about 1.80×10\hat{ }−6, optionally wherein the values are obtained by a SPR Biacore binding kinetic measurement.

In some embodiments, the invention provides an anti-CRP sdAb comprising a CDR1 comprising SEQ ID NO: 75 or a variant thereof having up to two amino acid substitutions, CDR2 comprising SEQ ID NO: 77 or a variant thereof having up to two amino acid substitutions, CDR3 comprising SEQ ID NO: 79 or a variant thereof having up to five amino acid substitutions, comprising an on-rate constant (kₒₙ) of about 6.00×10\hat{ }5±5.57×10\hat{ }5 and an off-rate constant (k_{off}) of about 9.80×10\hat{ }−5±1.40×10\hat{ }−4 and optionally a K_{D} of about 0.16, optionally wherein the values are obtained by a SPR Biacore binding kinetic measurement.

In some embodiments, the invention provides an anti-CRP sdAb comprising a CDR1 comprising SEQ ID NO: 75 or a variant thereof having up to two amino acid substitutions, CDR2 comprising SEQ ID NO: 77 or a variant thereof having up to two amino acid substitutions, CDR3 comprising SEQ ID NO: 79 or a variant thereof having up to five amino acid substitutions, comprising an on-rate constant (kₒₙ) of about 6.00×10\hat{ }5±6.24×10\hat{ }3 and an off-rate constant (k_{off}) of about 9.80×10\hat{ }−5±1.11×10\hat{ }−6 and optionally a K_{D} of about 0.16, optionally wherein the values are obtained by a SPR Biacore binding kinetic measurement.

In some embodiments, the invention provides an anti-CRP sdAb comprising a CDR1 comprising SEQ ID NO: 83 or a variant thereof having up to two amino acid substitutions, CDR2 comprising SEQ ID NO: 85 or a variant thereof having up to two amino acid substitutions, CDR3 comprising SEQ ID NO: 87 or a variant thereof having up to five amino acid substitutions, comprising an on-rate constant (kₒₙ) of about 2.10×10\hat{ }6±1.95×10\hat{ }6 and an off-rate constant (k_{off}) of about 2.50×10\hat{ }−4±3.57×10\hat{ }−4 and optionally a K_{D} of about 0.12, optionally wherein the values are obtained by a SPR Biacore binding kinetic measurement.

In some embodiments, the invention provides an anti-CRP sdAb comprising a CDR1 comprising SEQ ID NO: 83 or a variant thereof having up to two amino acid substitutions, CDR2 comprising SEQ ID NO: 85 or a variant thereof having up to two amino acid substitutions, CDR3 comprising SEQ ID NO: 87 or a variant thereof having up to five amino acid substitutions, comprising an on-rate constant (kₒₙ) of about 2.10×10\hat{ }6=4.47×10\hat{ }3 and an off-rate constant (k_{off}) of about 2.50×10\hat{ }−4±1.86×10\hat{ }−6 and optionally a K_{D} of about 0.12, optionally wherein the values are obtained by a SPR Biacore binding kinetic measurement.

In some embodiments, the invention provides an anti-CRP sdAb comprising a CDR1 comprising SEQ ID NO: 91 or a variant thereof having up to two amino acid substitutions, CDR2 comprising SEQ ID NO: 93 or a variant thereof having up to two amino acid substitutions, CDR3 comprising SEQ ID NO: 95 or a variant thereof having up to five amino acid substitutions, comprising an on-rate constant (kₒₙ) of about 2.40×10\hat{ }6±2.23×10\hat{ }6 and an off-rate constant (k_{off}) of about 8.10×10\hat{ }−5±1.16×10\hat{ }−4 and optionally a K_{D} of about 0.03, optionally wherein the values are obtained by a SPR Biacore binding kinetic measurement.

In some embodiments, the invention provides an anti-CRP sdAb comprising a CDR1 comprising SEQ ID NO: 91 or a variant thereof having up to two amino acid substitutions, CDR2 comprising SEQ ID NO: 93 or a variant thereof having up to two amino acid substitutions, CDR3 comprising SEQ ID NO: 95 or a variant thereof having up to five amino acid substitutions, comprising an on-rate constant (kₒₙ) of about 2.40×10\hat{ }6±4.90×10\hat{ }3 and an off-rate constant (k_{off}) of about 8.10×10\hat{ }−5±2.69×10\hat{ }−7 and optionally a K_{D} of about 0.03, optionally wherein the values are obtained by a SPR Biacore binding kinetic measurement.

Accordingly, the invention provides in one aspect an anti-CRP sdAb comprising an on-rate constant (kₒₙ) of about 4.10×10\hat{ }4−3.80×10\hat{ }5±3.81×10\hat{ }4−3.53×10\hat{ }5 and an off-rate constant (k_{off}) of about 2.00×10\hat{ }−5−6.90×10\hat{ }−3±2.85×10\hat{ }−5−9.84×10\hat{ }−3 and optionally a K_{D} of about 5.80×10\hat{ }−11 M−6.50×10\hat{ }−8 M, optionally wherein the values are obtained by an Octet BLI binding kinetic measurement, preferably wherein the sdAb is defined in any one of SEQ ID NOs: 1, 9, 17, 25, 33, 49, 57, 65, 73, 81, 89.

In some embodiments, the invention provides an anti-CRP sdAb comprising an on-rate constant (kₒₙ) of about 4.10×10\hat{ }4−3.80×10\hat{ }5±3.31×10\hat{ }2−1.48×10\hat{ }4 and an off-rate constant (k_{off}) of about 2.00×10\hat{ }−5−6.90×10\hat{ }−3±1.41×10\hat{ }−6−8.61×10\hat{ }−5 and optionally a K_{D} of about 5.80×10\hat{ }−11 M−6.50×10\hat{ }−8 M, optionally wherein the values are obtained by an Octet BLI binding kinetic measurement, preferably wherein the sdAb is defined in any one of SEQ ID NOs: 1, 9, 17, 25, 33, 49, 57, 65, 73, 81, 89.

In some embodiments, the invention provides an anti-CRP sdAb comprising an on-rate constant (kₒₙ) of about 1.30×10\hat{ }5±1.21×10\hat{ }5 and an off-rate constant (k_{off}) of about 6.90×10\hat{ }−3±9.84×10\hat{ }−3 and optionally a K_{D} of about 5.50×10\hat{ }−8 M, optionally wherein the values are obtained by an Octet BLI binding kinetic measurement, preferably wherein the sdAb is defined in SEQ ID NO: 1.

In some embodiments, the invention provides an anti-CRP sdAb comprising an on-rate constant (kₒₙ) of about 1.30×10\hat{ }5±3.02×10\hat{ }3 and an off-rate constant (k_{off}) of about 6.90×10\hat{ }−3±4.29×10\hat{ }−5 and optionally a K_{D} of about 5.50×10\hat{ }−8 M, optionally wherein the values are obtained by an Octet BLI binding kinetic measurement, preferably wherein the sdAb is defined in SEQ ID NO: 1.

In some embodiments, the invention provides an anti-CRP sdAb comprising an on-rate constant (kₒₙ) of about 2.70×10\hat{ }5±2.51×10\hat{ }5 and an off-rate constant (k_{off}) of about 4.00×10\hat{ }−4±5.70×10\hat{ }−4 and optionally a K_{D} of about 1.50×10\hat{ }−9 M, optionally wherein the values are obtained by an Octet BLI binding kinetic measurement, preferably wherein the sdAb is defined in SEQ ID NO: 9.

In some embodiments, the invention provides an anti-CRP sdAb comprising an on-rate constant (kₒₙ) of about 2.70×10\hat{ }5±1.80×10\hat{ }3 and an off-rate constant (k_{off}) of about 4.00×10\hat{ }−4±3.44×10\hat{ }−6 and optionally a K_{D} of about 1.50×10\hat{ }−9 M, optionally wherein the values are obtained by an Octet BLI binding kinetic measurement, preferably wherein the sdAb is defined in SEQ ID NO: 9.

In some embodiments, the invention provides an anti-CRP sdAb comprising an on-rate constant (kₒₙ) of about 6.40×10\hat{ }4±5.94×10\hat{ }4 and an off-rate constant (k_{off}) of about 3.30×10\hat{ }−4±4.71×10\hat{ }−4 and optionally a K_{D} of about 5.10×10\hat{ }−9 M, optionally wherein the values are obtained by an Octet BLI binding kinetic measurement, preferably wherein the sdAb is defined in SEQ ID NO: 17.

In some embodiments, the invention provides an anti-CRP sdAb comprising an on-rate constant (kₒₙ) of about 6.40×10\hat{ }4±4.22×10\hat{ }2 and an off-rate constant (k_{off}) of about 3.30×10\hat{ }−4±2.48×10\hat{ }−6 and optionally a K_{D} of about 5.10×10\hat{ }−9 M, optionally wherein the values are obtained by an Octet BLI binding kinetic measurement, preferably wherein the sdAb is defined in SEQ ID NO: 17.

In some embodiments, the invention provides an anti-CRP sdAb comprising an on-rate constant (kₒₙ) of about 3.80×10\hat{ }5±3.53×10\hat{ }5 and an off-rate constant (k_{off}) of about 1.30×10\hat{ }−4±1.85×10\hat{ }−4 and optionally a K_{D} of about 3.40×10\hat{ }−10 M, optionally wherein the values are obtained by an Octet BLI binding kinetic measurement, preferably wherein the sdAb is defined in SEQ ID NO: 25.

In some embodiments, the invention provides an anti-CRP sdAb comprising an on-rate constant (kₒₙ) of about 3.80×10\hat{ }5±4.34×10\hat{ }3 and an off-rate constant (k_{off}) of about 1.30×10\hat{ }−4±5.53×10\hat{ }−6 and optionally a K_{D} of about 3.40×10\hat{ }−10 M, optionally wherein the values are obtained by an Octet BLI binding kinetic measurement, preferably wherein the sdAb is defined in SEQ ID NO: 25.

In some embodiments, the invention provides an anti-CRP sdAb comprising an on-rate constant (kₒₙ) of about 3.10×10\hat{ }5±2.88×10\hat{ }5 and an off-rate constant (k_{off}) of about 5.80×10\hat{ }−3±8.27×10\hat{ }−3 and optionally a K_{D} of about 1.90×10\hat{ }−8 M, optionally wherein the values are obtained by an Octet BLI binding kinetic measurement, preferably wherein the sdAb is defined in SEQ ID NO: 33.

In some embodiments, the invention provides an anti-CRP sdAb comprising an on-rate constant (kₒₙ) of about 3.10×10\hat{ }5±1.48×10\hat{ }4 and an off-rate constant (k_{off}) of about 5.80×10\hat{ }−3±8.61×10\hat{ }−5 and optionally a K_{D} of about 1.90×10\hat{ }−8 M, optionally wherein the values are obtained by an Octet BLI binding kinetic measurement, preferably wherein the sdAb is defined in SEQ ID NO: 33.

In some embodiments, the invention provides an anti-CRP sdAb comprising an on-rate constant (kₒₙ) of about 3.00×10\hat{ }5±2.79×10\hat{ }5 and an off-rate constant (k_{off}) of about 5.90×10\hat{ }−5±8.41×10\hat{ }−5 and optionally a K_{D} of about 1.90×10\hat{ }−10 M, optionally wherein the values are obtained by an Octet BLI binding kinetic measurement, preferably wherein the sdAb is defined in SEQ ID NO: 49.

In some embodiments, the invention provides an anti-CRP sdAb comprising an on-rate constant (kₒₙ) of about 3.00×10\hat{ }5±8.37×10\hat{ }2 and an off-rate constant (k_{off}) of about 5.90×10\hat{ }−5±1.41×10\hat{ }−6 and optionally a K_{D} of about 1.90×10\hat{ }−10 M, optionally wherein the values are obtained by an Octet BLI binding kinetic measurement, preferably wherein the sdAb is defined in SEQ ID NO: 49.

In some embodiments, the invention provides an anti-CRP sdAb comprising an on-rate constant (kₒₙ) of about 4.10×10\hat{ }4±3.81×10\hat{ }4 and an off-rate constant (k_{off}) of about 6.80×10\hat{ }−4±9.70×10\hat{ }−4 and optionally a K_{D} of about 1.70×10\hat{ }−8 M, optionally wherein the values are obtained by an Octet BLI binding kinetic measurement, preferably wherein the sdAb is defined in SEQ ID NO: 57.

In some embodiments, the invention provides an anti-CRP sdAb comprising an on-rate constant (kₒₙ) of about 4.10×10\hat{ }4±3.31×10\hat{ }2 and an off-rate constant (k_{off}) of about 6.80×10\hat{ }−4±3.27×10\hat{ }−6 and optionally a K_{D} of about 1.70×10\hat{ }−8 M, optionally wherein the values are obtained by an Octet BLI binding kinetic measurement, preferably wherein the sdAb is defined in SEQ ID NO: 57.

In some embodiments, the invention provides an anti-CRP sdAb comprising an on-rate constant (kₒₙ) of about 2.20×10\hat{ }5±2.04×10\hat{ }5 and an off-rate constant (k_{off}) of about 3.00×10\hat{ }−5±4.28×10\hat{ }−5 and optionally a K_{D} of about 1.40×10\hat{ }−10 M, optionally wherein the values are obtained by an Octet BLI binding kinetic measurement, preferably wherein the sdAb is defined in SEQ ID NO: 65.

In some embodiments, the invention provides an anti-CRP sdAb comprising an on-rate constant (kₒₙ) of about 2.20×10\hat{ }5±1.09×10\hat{ }3 and an off-rate constant (k_{off}) of about 3.00×10\hat{ }−5±2.65×10\hat{ }−6 and optionally a K_{D} of about 1.40×10\hat{ }−10 M, optionally wherein the values are obtained by an Octet BLI binding kinetic measurement, preferably wherein the sdAb is defined in SEQ ID NO: 65.

In some embodiments, the invention provides an anti-CRP sdAb comprising an on-rate constant (kₒₙ) of about 1.00×10\hat{ }5±9.29×10\hat{ }4 and an off-rate constant (k_{off}) of about 6.80×10\hat{ }−3±9.70×10\hat{ }−3 and optionally a K_{D} of about 6.50×10\hat{ }−8 M, optionally wherein the values are obtained by an Octet BLI binding kinetic measurement, preferably wherein the sdAb is defined in SEQ ID NO: 73.

In some embodiments, the invention provides an anti-CRP sdAb comprising an on-rate constant (kₒₙ) of about 1.00×10\hat{ }5±4.84×10\hat{ }3 and an off-rate constant (k_{off}) of about 6.80×10\hat{ }−3±8.57×10\hat{ }−5 and optionally a K_{D} of about 6.50×10\hat{ }−8 M, optionally wherein the values are obtained by an Octet BLI binding kinetic measurement, preferably wherein the sdAb is defined in SEQ ID NO: 73.

In some embodiments, the invention provides an anti-CRP sdAb comprising an on-rate constant (kₒₙ) of about 3.00×10\hat{ }5±2.79×10\hat{ }5 and an off-rate constant (k_{off}) of about 8.10×10\hat{ }−5±1.16×10\hat{ }−4 and optionally a K_{D} of about 2.70×10\hat{ }−10 M, optionally wherein the values are obtained by an Octet BLI binding kinetic measurement, preferably wherein the sdAb is defined in SEQ ID NO: 81.

In some embodiments, the invention provides an anti-CRP sdAb comprising an on-rate constant (kₒₙ) of about 3.00×10\hat{ }5±1.58×10\hat{ }3 and an off-rate constant (k_{off}) of about 8.10×10\hat{ }−5±2.73×10\hat{ }−6 and optionally a K_{D} of about 2.70×10\hat{ }−10 M, optionally wherein the values are obtained by an Octet BLI binding kinetic measurement, preferably wherein the sdAb is defined in SEQ ID NO: 81.

In some embodiments, the invention provides an anti-CRP sdAb comprising an on-rate constant (kₒₙ) of about 3.50×10\hat{ }5±3.25×10\hat{ }5 and an off-rate constant (k_{off}) of about 2.00×10\hat{ }−5±2.85×10\hat{ }−5 and optionally a K_{D} of about 5.80×10\hat{ }−11 M, optionally wherein the values are obtained by an Octet BLI binding kinetic measurement, preferably wherein the sdAb is defined in SEQ ID NO: 89.

In some embodiments, the invention provides an anti-CRP sdAb comprising an on-rate constant (kₒₙ) of about 3.50×10\hat{ }5±2.36×10\hat{ }3 and an off-rate constant (k_{off}) of about 2.00×10\hat{ }−5±3.39×10\hat{ }−6 and optionally a K_{D} of about 5.80×10\hat{ }−11 M, optionally wherein the values are obtained by an Octet BLI binding kinetic measurement, preferably wherein the sdAb is defined in SEQ ID NO: 89.

In some embodiments, the invention provides an anti-CRP sdAb comprising a first complementary determining region (CDR1) or a variant thereof having up to two amino acid substitutions, a second CDR (CDR2) or a variant thereof having up to two amino acid substitutions and a third CDR (CDR3) or a variant thereof having up to five amino acid substitutions, comprising an on-rate constant (kₒₙ) of about 3.30×10\hat{ }4−4.60×10\hat{ }6 and an off-rate constant (k_{off}) of about 2.60×10\hat{ }−4−2.20×10\hat{ }−1, wherein CDR1 comprises any one of SEQ ID NOs: 3, 11, 19, 27, 35, 43, 51, 59, 67, 75, 83, or 91, wherein CDR2 comprises any one of SEQ ID NOs: 5, 13, 21, 29, 37, 45, 53, 61, 69, 77, 85, or 93 and wherein CDR3 comprises any one of SEQ ID NOs: 7, 15, 23, 31, 39, 47, 55, 63, 71, 79, 87, or 95, optionally wherein the values are obtained by an Octet BLI binding kinetic measurement.

In some embodiments, the invention provides an anti-CRP sdAb comprising a first complementary determining region (CDR1) or a variant thereof having up to two amino acid substitutions, a second CDR (CDR2) or a variant thereof having up to two amino acid substitutions and a third CDR (CDR3) or a variant thereof having up to five amino acid substitutions, comprising an on-rate constant (kₒₙ) of about 4.10×10\hat{ }4−3.80×10\hat{ }5 and an off-rate constant (k_{off}) of about 2.00×10\hat{ }−5−6.90×10\hat{ }−3, wherein CDR1 comprises any one of SEQ ID NOs: 3, 11, 19, 27, 35, 43, 51, 59, 67, 75, 83, or 91, wherein CDR2 comprises any one of SEQ ID NOs: 5, 13, 21, 29, 37, 45, 53, 61, 69, 77, 85, or 93 and wherein CDR3 comprises any one of SEQ ID NOs: 7, 15, 23, 31, 39, 47, 55, 63, 71, 79, 87, or 95, optionally wherein the values are obtained by an Octet BLI binding kinetic measurement.

In some embodiments, the invention provides an anti-CRP sdAb comprising a first complementary determining region (CDR1) or a variant thereof having up to two amino acid substitutions, a second CDR (CDR2) or a variant thereof having up to two amino acid substitutions and a third CDR (CDR3) or a variant thereof having up to five amino acid substitutions, comprising an on-rate constant (kₒₙ) of about 4.10×10\hat{ }4−3.80×10\hat{ }5±3.81×10\hat{ }4−3.53×10\hat{ }5 and an off-rate constant (k_{off}) of about 2.00×10\hat{ }−5−6.90×10\hat{ }−3=2.85×10\hat{ }−5−9.84×10\hat{ }−3 and optionally a K_{D} of about 5.80×10\hat{ }−11 M−6.50×10\hat{ }−8 M, wherein CDR1 comprises any one of SEQ ID NOs: 3, 11, 19, 27, 35, 43, 51, 59, 67, 75, 83, or 91, wherein CDR2 comprises any one of SEQ ID NOs: 5, 13, 21, 29, 37, 45, 53, 61, 69, 77, 85, or 93 and wherein CDR3 comprises any one of SEQ ID NOs: 7, 15, 23, 31, 39, 47, 55, 63, 71, 79, 87, or 95, optionally wherein the values are obtained by an Octet BLI binding kinetic measurement.

In some embodiments, the invention provides an anti-CRP sdAb comprising a first complementary determining region (CDR1) or a variant thereof having up to two amino acid substitutions, a second CDR (CDR2) or a variant thereof having up to two amino acid substitutions and a third CDR (CDR3) or a variant thereof having up to five amino acid substitutions, comprising an on-rate constant (kₒₙ) of about 4.10×10\hat{ }4−3.80×10\hat{ }5±3.31×10\hat{ }2−1.48×10\hat{ }4 and an off-rate constant (k_{off}) of about 2.00×10\hat{ }−5−6.90×10\hat{ }−3=1.41×10\hat{ }−6−8.61×10\hat{ }−5 and optionally a K_{D} of about 5.80×10\hat{ }−11 M−6.50×10\hat{ }−8 M, wherein CDR1 comprises any one of SEQ ID NOs: 3, 11, 19, 27, 35, 43, 51, 59, 67, 75, 83, or 91, wherein CDR2 comprises any one of SEQ ID NOs: 5, 13, 21, 29, 37, 45, 53, 61, 69, 77, 85, or 93 and wherein CDR3 comprises any one of SEQ ID NOs: 7, 15, 23, 31, 39, 47, 55, 63, 71, 79, 87, or 95, optionally wherein the values are obtained by an Octet BLI binding kinetic measurement.

In some embodiments, the invention provides an anti-CRP sdAb comprising a CDR1 comprising SEQ ID NO: 3 or a variant thereof having up to two amino acid substitutions, CDR2 comprising SEQ ID NO: 5 or a variant thereof having up to two amino acid substitutions, CDR3 comprising SEQ ID NO: 7 or a variant thereof having up to five amino acid substitutions, comprising an on-rate constant (kₒₙ) of about 1.30×10\hat{ }5=1.21×10\hat{ }5 and an off-rate constant (k_{off}) of about 6.90×10\hat{ }−3±9.84×10\hat{ }−3 and optionally a K_{D} of about 5.50×10\hat{ }−8 M, optionally wherein the values are obtained by an Octet BLI binding kinetic measurement.

In some embodiments, the invention provides an anti-CRP sdAb comprising a CDR1 comprising SEQ ID NO: 3 or a variant thereof having up to two amino acid substitutions, CDR2 comprising SEQ ID NO: 5 or a variant thereof having up to two amino acid substitutions, CDR3 comprising SEQ ID NO: 7 or a variant thereof having up to five amino acid substitutions, comprising an on-rate constant (kₒₙ) of about 1.30×10\hat{ }5±3.02×10\hat{ }3 and an off-rate constant (k_{off}) of about 6.90×10\hat{ }−3±4.29×10\hat{ }−5 and optionally a K_{D} of about 5.50×10\hat{ }−8 M, optionally wherein the values are obtained by an Octet BLI binding kinetic measurement.

In some embodiments, the invention provides an anti-CRP sdAb comprising a CDR1 comprising SEQ ID NO: 11 or a variant thereof having up to two amino acid substitutions, CDR2 comprising SEQ ID NO: 13 or a variant thereof having up to two amino acid substitutions, CDR3 comprising SEQ ID NO: 15 or a variant thereof having up to five amino acid substitutions, comprising an on-rate constant (kₒₙ) of about 2.70×10\hat{ }5±2.51×10\hat{ }5 and an off-rate constant (k_{off}) of about 4.00×10\hat{ }−4±5.70×10\hat{ }−4 and optionally a K_{D} of about 1.50×10\hat{ }−9 M, optionally wherein the values are obtained by an Octet BLI binding kinetic measurement.

In some embodiments, the invention provides an anti-CRP sdAb comprising a CDR1 comprising SEQ ID NO: 11 or a variant thereof having up to two amino acid substitutions, CDR2 comprising SEQ ID NO: 13 or a variant thereof having up to two amino acid substitutions, CDR3 comprising SEQ ID NO: 15 or a variant thereof having up to five amino acid substitutions, comprising an on-rate constant (kₒₙ) of about 2.70×10\hat{ }5±1.80×10\hat{ }3 and an off-rate constant (k_{off}) of about 4.00×10\hat{ }−4±3.44×10\hat{ }−6 and optionally a K_{D} of about 1.50×10\hat{ }−9 M, optionally wherein the values are obtained by an Octet BLI binding kinetic measurement.

In some embodiments, the invention provides an anti-CRP sdAb comprising a CDR1 comprising SEQ ID NO: 19 or a variant thereof having up to two amino acid substitutions, CDR2 comprising SEQ ID NO: 21 or a variant thereof having up to two amino acid substitutions, CDR3 comprising SEQ ID NO: 23 or a variant thereof having up to five amino acid substitutions, comprising an on-rate constant (kₒₙ) of about 6.40×10\hat{ }4±5.94×10\hat{ }4 and an off-rate constant (k_{off}) of about 3.30×10\hat{ }−4±4.71×10\hat{ }−4 and optionally a K_{D} of about 5.10×10\hat{ }−9 M, optionally wherein the values are obtained by an Octet BLI binding kinetic measurement.

In some embodiments, the invention provides an anti-CRP sdAb comprising a CDR1 comprising SEQ ID NO: 19 or a variant thereof having up to two amino acid substitutions, CDR2 comprising SEQ ID NO: 21 or a variant thereof having up to two amino acid substitutions, CDR3 comprising SEQ ID NO: 23 or a variant thereof having up to five amino acid substitutions, comprising an on-rate constant (kₒₙ) of about 6.40×10\hat{ }4=4.22×10\hat{ }2 and an off-rate constant (k_{off}) of about 3.30×10\hat{ }−4±2.48×10\hat{ }−6 and optionally a K_{D} of about 5.10×10\hat{ }−9 M, optionally wherein the values are obtained by an Octet BLI binding kinetic measurement.

In some embodiments, the invention provides an anti-CRP sdAb comprising a CDR1 comprising SEQ ID NO: 27 or a variant thereof having up to two amino acid substitutions, CDR2 comprising SEQ ID NO: 29 or a variant thereof having up to two amino acid substitutions, CDR3 comprising SEQ ID NO: 31 or a variant thereof having up to five amino acid substitutions, comprising an on-rate constant (kₒₙ) of about 3.80×10\hat{ }5±3.53×10\hat{ }5 and an off-rate constant (k_{off}) of about 1.30×10\hat{ }−4±1.85×10\hat{ }−4 and optionally a K_{D} of about 3.40×10\hat{ }−10 M, optionally wherein the values are obtained by an Octet BLI binding kinetic measurement.

In some embodiments, the invention provides an anti-CRP sdAb comprising a CDR1 comprising SEQ ID NO: 27 or a variant thereof having up to two amino acid substitutions, CDR2 comprising SEQ ID NO: 29 or a variant thereof having up to two amino acid substitutions, CDR3 comprising SEQ ID NO: 31 or a variant thereof having up to five amino acid substitutions, comprising an on-rate constant (kₒₙ) of about 3.80×10\hat{ }5±4.34×10\hat{ }3 and an off-rate constant (k_{off}) of about 1.30×10\hat{ }−4±5.53×10\hat{ }−6 and optionally a K_{D} of about 3.40×10\hat{ }−10 M, optionally wherein the values are obtained by an Octet BLI binding kinetic measurement.

In some embodiments, the invention provides an anti-CRP sdAb comprising a CDR1 comprising SEQ ID NO: 35 or a variant thereof having up to two amino acid substitutions, CDR2 comprising SEQ ID NO: 37 or a variant thereof having up to two amino acid substitutions, CDR3 comprising SEQ ID NO: 39 or a variant thereof having up to five amino acid substitutions, comprising an on-rate constant (kₒₙ) of about 3.10×10\hat{ }5=2.88×10\hat{ }5 and an off-rate constant (k_{off}) of about 5.80×10\hat{ }−3±8.27×10\hat{ }−3 and optionally a K_{D} of about 1.90×10\hat{ }−8 M, optionally wherein the values are obtained by an Octet BLI binding kinetic measurement.

In some embodiments, the invention provides an anti-CRP sdAb comprising a CDR1 comprising SEQ ID NO: 35 or a variant thereof having up to two amino acid substitutions, CDR2 comprising SEQ ID NO: 37 or a variant thereof having up to two amino acid substitutions, CDR3 comprising SEQ ID NO: 39 or a variant thereof having up to five amino acid substitutions, comprising an on-rate constant (kₒₙ) of about 3.10×10\hat{ }5±1.48×10\hat{ }4 and an off-rate constant (k_{off}) of about 5.80×10\hat{ }−3±8.61×10\hat{ }−5 and optionally a K_{D} of about 1.90×10\hat{ }−8 M, optionally wherein the values are obtained by an Octet BLI binding kinetic measurement.

In some embodiments, the invention provides an anti-CRP sdAb comprising a CDR1 comprising SEQ ID NO: 51 or a variant thereof having up to two amino acid substitutions, CDR2 comprising SEQ ID NO: 53 or a variant thereof having up to two amino acid substitutions, CDR3 comprising SEQ ID NO: 55 or a variant thereof having up to five amino acid substitutions, comprising an on-rate constant (kₒₙ) of about 3.00×10\hat{ }5±2.79×10\hat{ }5 and an off-rate constant (k_{off}) of about 5.90×10\hat{ }−5±8.41×10\hat{ }−5 and optionally a K_{D} of about 1.90×10\hat{ }−10 M, optionally wherein the values are obtained by an Octet BLI binding kinetic measurement.

In some embodiments, the invention provides an anti-CRP sdAb comprising a CDR1 comprising SEQ ID NO: 51 or a variant thereof having up to two amino acid substitutions, CDR2 comprising SEQ ID NO: 53 or a variant thereof having up to two amino acid substitutions, CDR3 comprising SEQ ID NO: 55 or a variant thereof having up to five amino acid substitutions, comprising an on-rate constant (kₒₙ) of about 3.00×10\hat{ }5±8.37×10\hat{ }2 and an off-rate constant (k_{off}) of about 5.90×10\hat{ }−5±1.41×10\hat{ }−6 and optionally a K_{D} of about 1.90×10\hat{ }−10 M, optionally wherein the values are obtained by an Octet BLI binding kinetic measurement.

In some embodiments, the invention provides an anti-CRP sdAb comprising a CDR1 comprising SEQ ID NO: 59 or a variant thereof having up to two amino acid substitutions, CDR2 comprising SEQ ID NO: 61 or a variant thereof having up to two amino acid substitutions, CDR3 comprising SEQ ID NO: 63 or a variant thereof having up to five amino acid substitutions, comprising an on-rate constant (kₒₙ) of about 4.10×10\hat{ }4±3.81×10\hat{ }4 and an off-rate constant (k_{off}) of about 6.80×10\hat{ }−4±9.70×10\hat{ }−4 and optionally a K_{D} of about 1.70×10\hat{ }−8 M, optionally wherein the values are obtained by an Octet BLI binding kinetic measurement.

In some embodiments, the invention provides an anti-CRP sdAb comprising a CDR1 comprising SEQ ID NO: 59 or a variant thereof having up to two amino acid substitutions, CDR2 comprising SEQ ID NO: 61 or a variant thereof having up to two amino acid substitutions, CDR3 comprising SEQ ID NO: 63 or a variant thereof having up to five amino acid substitutions, comprising an on-rate constant (kₒₙ) of about 4.10×10\hat{ }4±3.31×10\hat{ }2 and an off-rate constant (k_{off}) of about 6.80×10\hat{ }−4±3.27×10\hat{ }−6 and optionally a K_{D} of about 1.70×10\hat{ }−8 M, optionally wherein the values are obtained by an Octet BLI binding kinetic measurement.

In some embodiments, the invention provides an anti-CRP sdAb comprising a CDR1 comprising SEQ ID NO: 67 or a variant thereof having up to two amino acid substitutions, CDR2 comprising SEQ ID NO: 69 or a variant thereof having up to two amino acid substitutions, CDR3 comprising SEQ ID NO: 71 or a variant thereof having up to five amino acid substitutions, comprising an on-rate constant (kₒₙ) of about 2.20×10\hat{ }5=2.04×10\hat{ }5 and an off-rate constant (k_{off}) of about 3.00×10\hat{ }−5±4.28×10\hat{ }−5 and optionally a K_{D} of about 1.40×10\hat{ }−10 M, optionally wherein the values are obtained by an Octet BLI binding kinetic measurement.

In some embodiments, the invention provides an anti-CRP sdAb comprising a CDR1 comprising SEQ ID NO: 67 or a variant thereof having up to two amino acid substitutions, CDR2 comprising SEQ ID NO: 69 or a variant thereof having up to two amino acid substitutions, CDR3 comprising SEQ ID NO: 71 or a variant thereof having up to five amino acid substitutions, comprising an on-rate constant (kₒₙ) of about 2.20×10\hat{ }5±1.09×10\hat{ }3 and an off-rate constant (k_{off}) of about 3.00×10\hat{ }−5±2.65×10\hat{ }−6 and optionally a K_{D} of about 1.40×10\hat{ }−10 M, optionally wherein the values are obtained by an Octet BLI binding kinetic measurement.

In some embodiments, the invention provides an anti-CRP sdAb comprising a CDR1 comprising SEQ ID NO: 75 or a variant thereof having up to two amino acid substitutions, CDR2 comprising SEQ ID NO: 77 or a variant thereof having up to two amino acid substitutions, CDR3 comprising SEQ ID NO: 79 or a variant thereof having up to five amino acid substitutions, comprising an on-rate constant (kₒₙ) of about 1.00×10\hat{ }5±9.29×10\hat{ }4 and an off-rate constant (k_{off}) of about 6.80×10\hat{ }−3±9.70×10\hat{ }−3 and optionally a K_{D} of about 6.50×10\hat{ }−8 M, optionally wherein the values are obtained by an Octet BLI binding kinetic measurement.

In some embodiments, the invention provides an anti-CRP sdAb comprising a CDR1 comprising SEQ ID NO: 75 or a variant thereof having up to two amino acid substitutions, CDR2 comprising SEQ ID NO: 77 or a variant thereof having up to two amino acid substitutions, CDR3 comprising SEQ ID NO: 79 or a variant thereof having up to five amino acid substitutions, comprising an on-rate constant (kₒₙ) of about 1.00×10\hat{ }5±4.84×10\hat{ }3 and an off-rate constant (k_{off}) of about 6.80×10\hat{ }−3±8.57×10\hat{ }−5 and optionally a K_{D} of about 6.50×10\hat{ }−8 M, optionally wherein the values are obtained by an Octet BLI binding kinetic measurement.

In some embodiments, the invention provides an anti-CRP sdAb comprising a CDR1 comprising SEQ ID NO: 83 or a variant thereof having up to two amino acid substitutions, CDR2 comprising SEQ ID NO: 85 or a variant thereof having up to two amino acid substitutions, CDR3 comprising SEQ ID NO: 87 or a variant thereof having up to five amino acid substitutions, comprising an on-rate constant (kₒₙ) of about 3.00×10\hat{ }5±2.79×10\hat{ }5 and an off-rate constant (k_{off}) of about 8.10×10\hat{ }−5±1.16×10\hat{ }−4 and optionally a K_{D} of about 2.70×10\hat{ }−10 M, optionally wherein the values are obtained by an Octet BLI binding kinetic measurement.

In some embodiments, the invention provides an anti-CRP sdAb comprising a CDR1 comprising SEQ ID NO: 83 or a variant thereof having up to two amino acid substitutions, CDR2 comprising SEQ ID NO: 85 or a variant thereof having up to two amino acid substitutions, CDR3 comprising SEQ ID NO: 87 or a variant thereof having up to five amino acid substitutions, comprising an on-rate constant (kₒₙ) of about 3.00×10\hat{ }5±1.58×10\hat{ }3 and an off-rate constant (k_{off}) of about 8.10×10\hat{ }−5±2.73×10\hat{ }−6 and optionally a K_{D} of about 2.70×10\hat{ }−10 M, optionally wherein the values are obtained by an Octet BLI binding kinetic measurement.

In some embodiments, the invention provides an anti-CRP sdAb comprising a CDR1 comprising SEQ ID NO: 91 or a variant thereof having up to two amino acid substitutions, CDR2 comprising SEQ ID NO: 93 or a variant thereof having up to two amino acid substitutions, CDR3 comprising SEQ ID NO: 95 or a variant thereof having up to five amino acid substitutions, comprising an on-rate constant (kₒₙ) of about 3.50×10\hat{ }5±3.25×10\hat{ }5 and an off-rate constant (k_{off}) of about 2.00×10\hat{ }−5±2.85×10\hat{ }−5 and optionally a K_{D} of about 5.80×10\hat{ }−11 M, optionally wherein the values are obtained by an Octet BLI binding kinetic measurement.

In some embodiments, the invention provides an anti-CRP sdAb comprising a CDR1 comprising SEQ ID NO: 91 or a variant thereof having up to two amino acid substitutions, CDR2 comprising SEQ ID NO: 93 or a variant thereof having up to two amino acid substitutions, CDR3 comprising SEQ ID NO: 95 or a variant thereof having up to five amino acid substitutions, comprising an on-rate constant (kₒₙ) of about 2.00×10\hat{ }−5±3.39×10\hat{ }−6 and optionally a K_{D} of about 5.80×10\hat{ }−11 M, optionally wherein the values are obtained by an Octet BLI binding kinetic measurement.

In some embodiments, the invention provides an anti-C-reactive protein (CRP) single-domain antibody (sdAb) comprising an on-rate constant (kₒₙ) of about 1.30×10\hat{ }6 and an off-rate constant (k_{off}) of about 1.30×10\hat{ }−2 and optionally a K_{D} of about 9.62, optionally wherein the values are obtained by a SPR Biacore binding kinetic measurement, preferably wherein the sdAb is defined in SEQ ID NO: 1.

In some embodiments, the invention provides an anti-C-reactive protein (CRP) single-domain antibody (sdAb) comprising an on-rate constant (kₒₙ) of about 1.70×10\hat{ }6 and an off-rate constant (k_{off}) of about 1.70×10\hat{ }−4 and optionally a K_{D} of about 0.10, optionally wherein the values are obtained by a SPR Biacore binding kinetic measurement, preferably wherein the sdAb is defined in SEQ ID NO: 9.

In some embodiments, the invention provides an anti-C-reactive protein (CRP) single-domain antibody (sdAb) comprising an on-rate constant (kₒₙ) of about 8.00×10\hat{ }5 and an off-rate constant (k_{off}) of about 3.00×10\hat{ }−4 and optionally a K_{D} of about 0.38, optionally wherein the values are obtained by a SPR Biacore binding kinetic measurement, preferably wherein the sdAb is defined in SEQ ID NO: 17.

In some embodiments, the invention provides an anti-C-reactive protein (CRP) single-domain antibody (sdAb) comprising an on-rate constant (kₒₙ) of about 1.60×10\hat{ }6 and an off-rate constant (k_{off}) of about 2.00×10\hat{ }−5 and optionally a K_{D} of about 0.01, optionally wherein the values are obtained by a SPR Biacore binding kinetic measurement, preferably wherein the sdAb is defined in SEQ ID NO: 25.

In some embodiments, the invention provides an anti-C-reactive protein (CRP) single-domain antibody (sdAb) comprising an on-rate constant (kₒₙ) of about 2.70×10\hat{ }6 and an off-rate constant (k_{off}) of about 1.90×10\hat{ }−3 and optionally a K_{D} of about 0.70, optionally wherein the values are obtained by a SPR Biacore binding kinetic measurement, preferably wherein the sdAb is defined in SEQ ID NO: 33.

In some embodiments, the invention provides an anti-C-reactive protein (CRP) single-domain antibody (sdAb) comprising an on-rate constant (kₒₙ) of about 1.70×10\hat{ }7 and an off-rate constant (k_{off}) of about 1.30×10\hat{ }−3 and optionally a K_{D} of about 0.08, optionally wherein the values are obtained by a SPR Biacore binding kinetic measurement, preferably wherein the sdAb is defined in SEQ ID NO: 41.

In some embodiments, the invention provides an anti-C-reactive protein (CRP) single-domain antibody (sdAb) comprising an on-rate constant (kₒₙ) of about 1.50×10\hat{ }6 and an off-rate constant (k_{off}) of about 1.30×10\hat{ }−7 and optionally a K_{D} of about 7.60×10\hat{ }−5, optionally wherein the values are obtained by a SPR Biacore binding kinetic measurement, preferably wherein the sdAb is defined in SEQ ID NO: 49.

In some embodiments, the invention provides an anti-C-reactive protein (CRP) single-domain antibody (sdAb) comprising an on-rate constant (kₒₙ) of about 3.80×10\hat{ }5 and an off-rate constant (k_{off}) of about 6.20×10\hat{ }−5 and optionally a K_{D} of about 0.16, optionally wherein the values are obtained by a SPR Biacore binding kinetic measurement, preferably wherein the sdAb is defined in SEQ ID NO: 57.

In some embodiments, the invention provides an anti-C-reactive protein (CRP) single-domain antibody (sdAb) comprising an on-rate constant (kₒₙ) of about 1.70×10\hat{ }6 and an off-rate constant (k_{off}) of about 3.20×10\hat{ }−9 and optionally a K_{D} of about 1.8×10\hat{ }−6, optionally wherein the values are obtained by a SPR Biacore binding kinetic measurement, preferably wherein the sdAb is defined in SEQ ID NO: 65.

In some embodiments, the invention provides an anti-C-reactive protein (CRP) single-domain antibody (sdAb) comprising an on-rate constant (kₒₙ) of about 6.00×10\hat{ }5 and an off-rate constant (k_{off}) of about 9.80×10\hat{ }−5 and optionally a K_{D} of about 0.16, optionally wherein the values are obtained by a SPR Biacore binding kinetic measurement, preferably wherein the sdAb is defined in SEQ ID NO: 73.

In some embodiments, the invention provides an anti-C-reactive protein (CRP) single-domain antibody (sdAb) comprising an on-rate constant (kₒₙ) of about 2.10×10\hat{ }6 and an off-rate constant (k_{off}) of about 2.50×10\hat{ }−4 and optionally a K_{D} of about 0.12, optionally wherein the values are obtained by a SPR Biacore binding kinetic measurement, preferably wherein the sdAb is defined in SEQ ID NO: 81.

In some embodiments, the invention provides an anti-C-reactive protein (CRP) single-domain antibody (sdAb) comprising an on-rate constant (kₒₙ) of about 2.40×10\hat{ }6 and an off-rate constant (k_{off}) of about 8.10×10\hat{ }−5 and optionally a K_{D} of about 0.03, optionally wherein the values are obtained by a SPR Biacore binding kinetic measurement, preferably wherein the sdAb is defined in SEQ ID NO: 89.

In some embodiments, the invention provides an anti-C-reactive protein (CRP) single-domain antibody (sdAb) comprising an on-rate constant (kₒₙ) of about 6.20×10\hat{ }5 and an off-rate constant (k_{off}) of about 9.30×10\hat{ }−2 and optionally a K_{D} of about 1.50×10\hat{ }−8 M, optionally wherein the values are obtained by an Octet BLI binding kinetic measurement, preferably wherein the sdAb is defined in SEQ ID NO: 1.

In some embodiments, the invention provides an anti-C-reactive protein (CRP) single-domain antibody (sdAb) comprising an on-rate constant (kₒₙ) of about 6.20×10\hat{ }5 and an off-rate constant (k_{off}) of about 1.90×10\hat{ }−3 and optionally a K_{D} of about 3.10×10\hat{ }−9 M, optionally wherein the values are obtained by an Octet BLI binding kinetic measurement, preferably wherein the sdAb is defined in SEQ ID NO: 9.

In some embodiments, the invention provides an anti-C-reactive protein (CRP) single-domain antibody (sdAb) comprising an on-rate constant (kₒₙ) of about 4.10×10\hat{ }4 and an off-rate constant (k_{off}) of about 1.20×10\hat{ }−3 and optionally a K_{D} of about 2.90×10\hat{ }−8 M, optionally wherein the values are obtained by an Octet BLI binding kinetic measurement, preferably wherein the sdAb is defined in SEQ ID NO: 17.

In some embodiments, the invention provides an anti-C-reactive protein (CRP) single-domain antibody (sdAb) comprising an on-rate constant (kₒₙ) of about 5.70×10\hat{ }5 and an off-rate constant (k_{off}) of about 5.70×10\hat{ }−4 and optionally a K_{D} of about 1.00×10\hat{ }−9 M, optionally wherein the values are obtained by an Octet BLI binding kinetic measurement, preferably wherein the sdAb is defined in SEQ ID NO: 25.

In some embodiments, the invention provides an anti-C-reactive protein (CRP) single-domain antibody (sdAb) comprising an on-rate constant (kₒₙ) of about 1.00×10\hat{ }6 and an off-rate constant (k_{off}) of about 8.60×10\hat{ }−3 and optionally a K_{D} of about 8.50×10\hat{ }−9 M, optionally wherein the values are obtained by an Octet BLI binding kinetic measurement, preferably wherein the sdAb is defined in SEQ ID NO: 33.

In some embodiments, the invention provides an anti-C-reactive protein (CRP) single-domain antibody (sdAb) comprising an on-rate constant (kₒₙ) of about 1.30×10\hat{ }6 and an off-rate constant (k_{off}) of about 2.20×10\hat{ }−1 and optionally a K_{D} of about 1.80×10\hat{ }−7 M, optionally wherein the values are obtained by an Octet BLI binding kinetic measurement, preferably wherein the sdAb is defined in SEQ ID NO: 41.

In some embodiments, the invention provides an anti-C-reactive protein (CRP) single-domain antibody (sdAb) comprising an on-rate constant (kₒₙ) of about 1.80×10\hat{ }6 and an off-rate constant (k_{off}) of about 3.70×10\hat{ }−4 and optionally a K_{D} of about 2.00×10\hat{ }−10 M, optionally wherein the values are obtained by an Octet BLI binding kinetic measurement, preferably wherein the sdAb is defined in SEQ ID NO: 49.

In some embodiments, the invention provides an anti-C-reactive protein (CRP) single-domain antibody (sdAb) comprising an on-rate constant (kₒₙ) of about 3.30×10\hat{ }4 and an off-rate constant (k_{off}) of about 2.60×10\hat{ }−4 and optionally a K_{D} of about 7.80×10\hat{ }−9 M, optionally wherein the values are obtained by an Octet BLI binding kinetic measurement, preferably wherein the sdAb is defined in SEQ ID NO: 57.

In some embodiments, the invention provides an anti-C-reactive protein (CRP) single-domain antibody (sdAb) comprising an on-rate constant (kₒₙ) of about 2.70×10\hat{ }6 and an off-rate constant (k_{off}) of about 3.90×10\hat{ }−4 and optionally a K_{D} of about 1.50×10\hat{ }−10 M, optionally wherein the values are obtained by an Octet BLI binding kinetic measurement, preferably wherein the sdAb is defined in SEQ ID NO: 65.

In some embodiments, the invention provides an anti-C-reactive protein (CRP) single-domain antibody (sdAb) comprising an on-rate constant (kₒₙ) of about 3.90×10\hat{ }5 and an off-rate constant (k_{off}) of about 2.80×10\hat{ }−3 and optionally a K_{D} of about 7.30×10\hat{ }−9 M, optionally wherein the values are obtained by an Octet BLI binding kinetic measurement, preferably wherein the sdAb is defined in SEQ ID NO: 73.

In some embodiments, the invention provides an anti-C-reactive protein (CRP) single-domain antibody (sdAb) comprising an on-rate constant (kₒₙ) of about 4.60×10\hat{ }6 and an off-rate constant (k_{off}) of about 1.10×10\hat{ }−3 and optionally a K_{D} of about 2.40×10\hat{ }−10 M, optionally wherein the values are obtained by an Octet BLI binding kinetic measurement, preferably wherein the sdAb is defined in SEQ ID NO: 81.

In some embodiments, the invention provides an anti-C-reactive protein (CRP) single-domain antibody (sdAb) comprising an on-rate constant (kₒₙ) of about 2.40×10\hat{ }6 and an off-rate constant (k_{off}) of about 4.60×10\hat{ }−4 and optionally a K_{D} of about 1.90×10\hat{ }−10 M, optionally wherein the values are obtained by an Octet BLI binding kinetic measurement, preferably wherein the sdAb is defined in SEQ ID NO: 89.

In one aspect, the present invention provides an anti-C-reactive protein (CRP) single-domain antibody (sdAb) comprising an on-rate constant (kₒₙ), an off-rate constant (k_{off}) and optionally a K_{D} as defined in any one of Tables 6, 7, 10 or 11, optionally wherein the values are obtained by a SPR Biacore binding kinetic measurement or an Octet BLI binding kinetic measurement.

Accordingly, the present invention provides an anti-C-reactive protein (CRP) single-domain antibody (sdAb) comprising an on-rate constant (kₒₙ) of about 3.30×10\hat{ }4−1.70×10\hat{ }7 and an off-rate constant (k_{off}) of about 3.2×10\hat{ }−9−2.20×10\hat{ }−1.

A sdAb comprises framework regions (FRs) and complementary determining regions (CDRs). The FRs are structural components, whereas the CDRs determine the specificity. In general, a sdAb of the present invention consists of four FRs and three CDRs. A sdAb of the present invention can be defined by its CDRs which determine the specificity and the epitope bound. The invention is not particularly limited to sdAb comprising specific CDRs. In particular, the invention provides herein below a detailed description how sdAb having desired kₒₙ and k_{off} values may be obtained. Accordingly, the skilled person is in the position to generate and select further anti-CRP sdAb according to the invention. In particular, the skilled person is in the position to select antibodies having CDRs having desired kₒₙ and k_{off} values according to the present invention. The invention provides exemplary sdAb having CRP specific CDRs and specific kₒₙ and k_{off} values allowing for specific, reversible and therefore continuous association and dissociation with CRP over time. Exemplary sdAb of the present invention can comprise a first CDR, a second CDR and a third CDR, wherein the first CDR is selected from SEQ ID NOs: 3, 11, 19, 27, 35, 43, 51, 59, 67, 75, 83, or 91, and wherein the second CDR is selected from SEQ ID NOs: 5, 13, 21, 29, 37, 45, 53, 61, 69, 77, 85, or 93, and wherein the third CDR is selected from SEQ ID NOs: 7, 15, 23, 31, 39, 47, 55, 63, 71, 79, 87, or 95.

It is envisioned herein, that the CDRs of a sdAb also comprise variants thereof having one or more amino acid modifications. An “amino-acid modification” at a specified position, e.g. of the CDR, refers to the substitution or deletion of the specified residue, or the insertion of at least one amino acid residue adjacent the specified residue. Insertion “adjacent” to a specified residue means insertion within one to two residues thereof. The insertion may be N-terminal or C-terminal to the specified residue. The preferred amino acid modification herein is a substitution. Accordingly, the invention provides CDRs or variants thereof having amino acid substitutions. It may be preferred herein, that said one or more amino acid substitutions do not interfere with the ability of the sdAb to continuously associate and dissociate with CRP over time. A CDR of the present invention may have one or more amino acid substitutions. A CDR of the present invention may have 1-10 amino acid substitutions, such as 1, 2, 3, 4, 5, 6, 7, 8, 9, or 10 amino acid substitutions. A first CDR of the invention may have 1-2 amino acid substitutions, such as 1 or 2 amino acid substitutions and/or up to 2 amino acid substitutions. A second CDR may have 1-2 amino acid substitutions, such as 1 or 2 amino acid substitutions and/or up to 2 amino acid substitutions. A third CDR may have 1-5 amino acid substitutions, such as 1, 2, 3, 4, or 5 amino acid substitutions and/or up to 5 amino acid substitutions.

It is preferred herein, that a sdAb of the present invention comprises a first CDR, a second CDR and a third CDR, wherein,
(i) the first CDR comprises SEQ ID NO: 3, the second CDR comprises SEQ ID NO: 5 and the third CDR comprises SEQ ID NO: 7, or
(ii) the first CDR comprises SEQ ID NO: 11, the second CDR comprises SEQ ID NO: 13 and the third CDR comprises SEQ ID NO: 15, or
(iii) the first CDR comprises SEQ ID NO: 19, the second CDR comprises SEQ ID NO: 21 and the third CDR comprises SEQ ID NO: 23, or
(iv) the first CDR comprises SEQ ID NO: 27, the second CDR comprises SEQ ID NO: 29 and the third CDR comprises SEQ ID NO: 31, or
(v) the first CDR comprises SEQ ID NO: 35, the second CDR comprises SEQ ID NO: 37 and the third CDR comprises SEQ ID NO: 39, or
(vi) the first CDR comprises SEQ ID NO: 43, the second CDR comprises SEQ ID NO: 45 and the third CDR comprises SEQ ID NO: 47, or
(vii) the first CDR comprises SEQ ID NO: 51, the second CDR comprises SEQ ID NO: 53 and the third CDR comprises SEQ ID NO: 55, or
(viii) the first CDR comprises SEQ ID NO: 59, the second CDR comprises SEQ ID NO: 61 and the third CDR comprises SEQ ID NO: 63, or
(ix) the first CDR comprises SEQ ID NO: 67, the second CDR comprises SEQ ID NO: 69 and the third CDR comprises SEQ ID NO: 71, or
(x) the first CDR comprises SEQ ID NO: 75, the second CDR comprises SEQ ID NO: 77 and the third CDR comprises SEQ ID NO: 79, or
(xi) the first CDR comprises SEQ ID NO: 83, the second CDR comprises SEQ ID NO: 85 and the third CDR comprises SEQ ID NO: 87, or
(xii) the first CDR comprises SEQ ID NO: 91, the second CDR comprises SEQ ID NO: 93 and the third CDR comprises SEQ ID NO: 95.

In another aspect, it is preferred herein that a sdAb of the present invention comprises a first CDR, a second CDR and a third CDR, wherein,
(i) the first CDR comprises SEQ ID NO: 3 or a variant thereof having one or two amino acid substitutions, the second CDR comprises SEQ ID NO: 5 or a variant thereof having one or two amino acid substitutions, and the third CDR comprises SEQ ID NO: 7 or a variant thereof having one, two, three, four or five amino acid substitutions, or
(ii) the first CDR comprises SEQ ID NO: 11 or a variant thereof having one or two amino acid substitutions, the second CDR comprises SEQ ID NO: 13 or a variant thereof having one or two amino acid substitutions, and the third CDR comprises SEQ ID NO: 15 or a variant thereof having one, two, three, four or five amino acid substitutions, or
(iii) the first CDR comprises SEQ ID NO: 19 or a variant thereof having one or two amino acid substitutions, the second CDR comprises SEQ ID NO: 21 or a variant thereof having one or two amino acid substitutions, and the third CDR comprises SEQ ID NO: 23 or a variant thereof having one, two, three, four or five amino acid substitutions, or
(iv) the first CDR comprises SEQ ID NO: 27 or a variant thereof having one or two amino acid substitutions, the second CDR comprises SEQ ID NO: 29 or a variant thereof having one or two amino acid substitutions, and the third CDR comprises SEQ ID NO: 31 or a variant thereof having one, two, three, four or five amino acid substitutions, or
(v) the first CDR comprises SEQ ID NO: 35 or a variant thereof having one or two amino acid substitutions, the second CDR comprises SEQ ID NO: 37 or a variant thereof having one or two amino acid substitutions, and the third CDR comprises SEQ ID NO: 39 or a variant thereof having one, two, three, four or five amino acid substitutions, or
(vi) the first CDR comprises SEQ ID NO: 43 or a variant thereof having one or two amino acid substitutions, the second CDR comprises SEQ ID NO: 45 or a variant thereof having one or two amino acid substitutions, and the third CDR comprises SEQ ID NO: 47 or a variant thereof having one, two, three, four or five amino acid substitutions, or
(vii) the first CDR comprises SEQ ID NO: 51 or a variant thereof having one or two amino acid substitutions, the second CDR comprises SEQ ID NO: 53 or a variant thereof having one or two amino acid substitutions, and the third CDR comprises SEQ ID NO: 55 or a variant thereof having one, two, three, four or five amino acid substitutions, or
(viii) the first CDR comprises SEQ ID NO: 59 or a variant thereof having one or two amino acid substitutions, the second CDR comprises SEQ ID NO: 61 or a variant thereof having one or two amino acid substitutions, and the third CDR comprises SEQ ID NO: 63 or a variant thereof having one, two, three, four or five amino acid substitutions, or
(ix) the first CDR comprises SEQ ID NO: 67 or a variant thereof having one or two amino acid substitutions, the second CDR comprises SEQ ID NO: 69 or a variant thereof having one or two amino acid substitutions, and the third CDR comprises SEQ ID NO: 71 or a variant thereof having one, two, three, four or five amino acid substitutions, or
(x) the first CDR comprises SEQ ID NO: 75 or a variant thereof having one or two amino acid substitutions, the second CDR comprises SEQ ID NO: 77 or a variant thereof having one or two amino acid substitutions, and the third CDR comprises SEQ ID NO: 79 or a variant thereof having one, two, three, four or five amino acid substitutions, or
(xi) the first CDR comprises SEQ ID NO: 83 or a variant thereof having one or two amino acid substitutions, the second CDR comprises SEQ ID NO: 85 or a variant thereof having one or two amino acid substitutions, and the third CDR comprises SEQ ID NO: 87 or a variant thereof having one, two, three, four or five amino acid substitutions, or
(xii) the first CDR comprises SEQ ID NO: 91 or a variant thereof having one or two amino acid substitutions, the second CDR comprises SEQ ID NO: 93 or a variant thereof having one or two amino acid substitutions, and the third CDR comprises SEQ ID NO: 95 or a variant thereof having one, two, three, four or five amino acid substitutions.

Accordingly, the invention provides in one aspect an anti-C-reactive protein (CRP) single-domain antibody (sdAb) comprising an on-rate constant (kₒₙ) of about 3.30×10\hat{ }4−1.70×10\hat{ }7 and an off-rate constant (k_{off}) of about 3.30×10\hat{ }4−1.70×10\hat{ }7, comprising a first complementary determining region (CDR1), a second CDR (CDR2) and third CDR (CDR3), wherein:
(i) CDR1 comprises SEQ ID NO: 3 or a variant thereof having up to two amino acid substitutions, CDR2 comprises SEQ ID NO: 5 or a variant thereof having up to two amino acid substitutions, CDR3 comprises SEQ ID NO: 7 or a variant thereof having up to five amino acid substitutions; or
(ii) CDR1 comprises SEQ ID NO: 11 or a variant thereof having up to two amino acid substitutions, CDR2 comprises SEQ ID NO: 13 or a variant thereof having up to two amino acid substitutions, CDR3 comprises SEQ ID NO: 15 or a variant thereof having up to five amino acid substitutions; or
(iii) CDR1 comprises SEQ ID NO: 19 or a variant thereof having up to two amino acid substitutions, CDR2 comprises SEQ ID NO: 21 or a variant thereof having up to two amino acid substitutions, CDR3 comprises SEQ ID NO: 23 or a variant thereof having up to five amino acid substitutions; or
(iv) CDR1 comprises SEQ ID NO: 27 or a variant thereof having up to two amino acid substitutions, CDR2 comprises SEQ ID NO: 29 or a variant thereof having up to two amino acid substitutions, CDR3 comprises SEQ ID NO: 31 or a variant thereof having up to five amino acid substitutions; or
(v) CDR1 comprises SEQ ID NO: 35 or a variant thereof having up to two amino acid substitutions, CDR2 comprises SEQ ID NO: 37 or a variant thereof having up to two amino acid substitutions, CDR3 comprises SEQ ID NO: 39 or a variant thereof having up to five amino acid substitutions; or
(vi) CDR1 comprises SEQ ID NO: 43 or a variant thereof having up to two amino acid substitutions, CDR2 comprises SEQ ID NO: 45 or a variant thereof having up to two amino acid substitutions, CDR3 comprises SEQ ID NO: 47 or a variant thereof having up to five amino acid substitutions; or
(vii) CDR1 comprises SEQ ID NO: 51 or a variant thereof having up to two amino acid substitutions, CDR2 comprises SEQ ID NO: 53 or a variant thereof having up to two amino acid substitutions, CDR3 comprises SEQ ID NO: 55 or a variant thereof having up to five amino acid substitutions; or
(viii) CDR1 comprises SEQ ID NO: 59 or a variant thereof having up to two amino acid substitutions, CDR2 comprises SEQ ID NO: 61 or a variant thereof having up to two amino acid substitutions, CDR3 comprises SEQ ID NO: 63 or a variant thereof having up to five amino acid substitutions; or
(ix) CDR1 comprises SEQ ID NO: 67 or a variant thereof having up to two amino acid substitutions, CDR2 comprises SEQ ID NO: 69 or a variant thereof having up to two amino acid substitutions, CDR3 comprises SEQ ID NO: 71 or a variant thereof having up to five amino acid substitutions; or
(x) CDR1 comprises SEQ ID NO: 75 or a variant thereof having up to two amino acid substitutions, CDR2 comprises SEQ ID NO: 77 or a variant thereof having up to two amino acid substitutions, CDR3 comprises SEQ ID NO: 79 or a variant thereof having up to five amino acid substitutions; or
(xi) CDR1 comprises SEQ ID NO: 83 or a variant thereof having up to two amino acid substitutions, CDR2 comprises SEQ ID NO: 85 or a variant thereof having up to two amino acid substitutions, CDR3 comprises SEQ ID NO: 87 or a variant thereof having up to five amino acid substitutions; or
(xii) CDR1 comprises SEQ ID NO: 91 or a variant thereof having up to two amino acid substitutions, CDR2 comprises SEQ ID NO: 93 or a variant thereof having up to two amino acid substitutions, CDR3 comprises SEQ ID NO: 95 or a variant thereof having up to five amino acid substitutions.

As mentioned herein above, a sdAb of the present invention can also comprise structural framework regions (FR). In general, the sdAb of the present invention are not limited to any FRs, rather any FR that results in a functional sdAb according to the present invention may be used. The invention provides herein below a detailed description how feasible FRs can be selected. Accordingly, the skilled person is enabled to select FRs that can be used in the sense of the invention. The invention provides exemplary FRs that can be used with the CDRs described herein to form a sdAb according to the invention. Exemplary FRs can comprise a first FR, a second FR, a third FR and a fourth FR. It may be preferred herein, that a first FR comprises any one of SEQ ID NOs: 2, 10, 18, 26, 34, 42, 50, 58, 66, 74, 82, or 90, a second FR comprises any one of SEQ ID NOs: 4, 12, 20, 28, 36, 44, 52, 60, 68, 76, 84, or 92, a third FR comprises any one of SEQ ID NOs: 6, 14, 22, 30, 38, 46, 54, 62, 70, 78, 86, or 94 and a fourth FR comprises any one of SEQ ID NOs: 8, 16, 24, 32, 40, 48, 56, 64, 72, 80, 88, or 96. The FRs in a sdAb have structural function.

Accordingly, it is envisioned herein, that the FRs of a sdAb also comprise variants thereof having one or more amino acid modifications. The invention provides FRs or variants thereof having amino acid substitutions. It may be preferred herein, that said one or more amino acid substitutions do not interfere with the ability of the sdAb to continuously associate and dissociate with CRP over time. It may further be preferred herein, that said one or more amino acid substitutions do not interfere with the structural integrity of the sdAb. A FR of the present invention may have one or more amino acid substitutions. A FR of the present invention may have 1-10 amino acid substitutions, such as 1, 2, 3, 4, 5, 6, 7, 8, 9, or 10 amino acid substitutions. A first FR of the invention may have 1-6 amino acid substitutions, such as 1, 2, 3, 4, 5, or 6 amino acid substitutions and/or up to 6 amino acid substitutions. A second CDR may have 1-4 amino acid substitutions, such as 1, 2, 3, or 4 amino acid substitutions and/or up to 4 amino acid substitutions. A third CDR may have 1-10 amino acid substitutions, such as 1, 2, 3, 4, 5, 6, 7, 8, 9, or 10 amino acid substitutions and/or up to 10 amino acid substitutions. A third CDR may have 1-3 amino acid substitutions, such as 1, 2, or 3 amino acid substitutions and/or up to 3 amino acid substitutions.

It is preferred herein, that a sdAb of the present invention comprises a first FR, a second FR, a third FR and a fourth FR, wherein,
(i) the first FR comprises SEQ ID NO: 2, the second FR comprises SEQ ID NO: 4, the third FR comprises SEQ ID NO: 6, and the fourth FR comprises SEQ ID NO: 8, or
(ii) the first FR comprises SEQ ID NO: 10, the second FR comprises SEQ ID NO: 12, the third FR comprises SEQ ID NO: 14, and the fourth FR comprises SEQ ID NO: 16, or
(iii) the first FR comprises SEQ ID NO: 18, the second FR comprises SEQ ID NO: 20, the third FR comprises SEQ ID NO: 22, and the fourth FR comprises SEQ ID NO: 24, or
(iv) the first FR comprises SEQ ID NO: 26, the second FR comprises SEQ ID NO: 28, the third FR comprises SEQ ID NO: 30, and the fourth FR comprises SEQ ID NO: 32, or
(v) the first FR comprises SEQ ID NO: 34, the second FR comprises SEQ ID NO: 36, the third FR comprises SEQ ID NO: 38, and the fourth FR comprises SEQ ID NO: 40, or
(vi) the first FR comprises SEQ ID NO: 42, the second FR comprises SEQ ID NO: 44, the third FR comprises SEQ ID NO: 46, and the fourth FR comprises SEQ ID NO: 48, or
(vii) the first FR comprises SEQ ID NO: 50, the second FR comprises SEQ ID NO: 52, the third FR comprises SEQ ID NO: 54, and the fourth FR comprises SEQ ID NO: 56, or
(viii) the first FR comprises SEQ ID NO: 58, the second FR comprises SEQ ID NO: 60, the third FR comprises SEQ ID NO: 62, and the fourth FR comprises SEQ ID NO: 64, or
(ix) the first FR comprises SEQ ID NO: 66, the second FR comprises SEQ ID NO: 68, the third FR comprises SEQ ID NO: 70, and the fourth FR comprises SEQ ID NO: 72, or
(x) the first FR comprises SEQ ID NO: 74, the second FR comprises SEQ ID NO: 76, the third FR comprises SEQ ID NO: 78, and the fourth FR comprises SEQ ID NO: 80, or
(xi) the first FR comprises SEQ ID NO: 82, the second FR comprises SEQ ID NO: 84, the third FR comprises SEQ ID NO: 86, and the fourth FR comprises SEQ ID NO: 88, or
(xii) the first FR comprises SEQ ID NO: 90, the second FR comprises SEQ ID NO: 92, the third FR comprises SEQ ID NO: 94, and the fourth FR comprises SEQ ID NO: 96.

In another aspect, it is preferred herein, that a sdAb of the present invention comprises a first FR, a second FR and a third FR, wherein,
(i) the first FR comprises SEQ ID NO: 2 or a variant thereof having one, two, three, four, five or six amino acid substitutions, the second FR comprises SEQ ID NO: 4 or a variant thereof having one, two, three, or four amino acid substitutions, the third FR comprises SEQ ID NO: 6 or a variant thereof having one, two, three, four, five, six, seven, eight, nine, or ten amino acid substitutions, and the fourth FR comprises SEQ ID NO: 8 or a variant thereof having one, two, or three amino acid substitutions, or
(ii) the first FR comprises SEQ ID NO: 10 or a variant thereof having one, two, three, four, five or six amino acid substitutions, the second FR comprises SEQ ID NO: 12 or a variant thereof having one, two, three, or four amino acid substitutions, the third FR comprises SEQ ID NO: 14 or a variant thereof having one, two, three, four, five, six, seven, eight, nine, or ten amino acid substitutions, and the fourth FR comprises SEQ ID NO: 16 or a variant thereof having one, two, or three amino acid substitutions, or
(iii) the first FR comprises SEQ ID NO: 18 or a variant thereof having one, two, three, four, five or six amino acid substitutions, the second FR comprises SEQ ID NO: 20 or a variant thereof having one, two, three, or four amino acid substitutions, the third FR comprises SEQ ID NO: 22 or a variant thereof having one, two, three, four, five, six, seven, eight, nine, or ten amino acid substitutions, and the fourth FR comprises SEQ ID NO: 24 or a variant thereof having one, two, or three amino acid substitutions, or
(iv) the first FR comprises SEQ ID NO: 26 or a variant thereof having one, two, three, four, five or six amino acid substitutions, the second FR comprises SEQ ID NO: 28 or a variant thereof having one, two, three, or four amino acid substitutions, the third FR comprises SEQ ID NO: 30 or a variant thereof having one, two, three, four, five, six, seven, eight, nine, or ten amino acid substitutions, and the fourth FR comprises SEQ ID NO: 32 or a variant thereof having one, two, or three amino acid substitutions, or
(v) the first FR comprises SEQ ID NO: 34 or a variant thereof having one, two, three, four, five or six amino acid substitutions, the second FR comprises SEQ ID NO: 36 or a variant thereof having one, two, three, or four amino acid substitutions, the third FR comprises SEQ ID NO: 38 or a variant thereof having one, two, three, four, five, six, seven, eight, nine, or ten amino acid substitutions, and the fourth FR comprises SEQ ID NO: 40 or a variant thereof having one, two, or three amino acid substitutions, or
(vi) the first FR comprises SEQ ID NO: 42 or a variant thereof having one, two, three, four, five or six amino acid substitutions, the second FR comprises SEQ ID NO: 44 or a variant thereof having one, two, three, or four amino acid substitutions, the third FR comprises SEQ ID NO: 46 or a variant thereof having one, two, three, four, five, six, seven, eight, nine, or ten amino acid substitutions, and the fourth FR comprises SEQ ID NO: 48 or a variant thereof having one, two, or three amino acid substitutions, or
(vii) the first FR comprises SEQ ID NO: 50 or a variant thereof having one, two, three, four, five or six amino acid substitutions, the second FR comprises SEQ ID NO: 52 or a variant thereof having one, two, three, or four amino acid substitutions, the third FR comprises SEQ ID NO: 54 or a variant thereof having one, two, three, four, five, six, seven, eight, nine, or ten amino acid substitutions, and the fourth FR comprises SEQ ID NO: 56 or a variant thereof having one, two, or three amino acid substitutions, or
(viii) the first FR comprises SEQ ID NO: 58 or a variant thereof having one, two, three, four, five or six amino acid substitutions, the second FR comprises SEQ ID NO: 60 or a variant thereof having one, two, three, or four amino acid substitutions, the third FR comprises SEQ ID NO: 62 or a variant thereof having one, two, three, four, five, six, seven, eight, nine, or ten amino acid substitutions, and the fourth FR comprises SEQ ID NO: 64 or a variant thereof having one, two, or three amino acid substitutions, or
(ix) the first FR comprises SEQ ID NO: 66 or a variant thereof having one, two, three, four, five or six amino acid substitutions, the second FR comprises SEQ ID NO: 68 or a variant thereof having one, two, three, or four amino acid substitutions, the third FR comprises SEQ ID NO: 70 or a variant thereof having one, two, three, four, five, six, seven, eight, nine, or ten amino acid substitutions, and the fourth FR comprises SEQ ID NO: 72 or a variant thereof having one, two, or three amino acid substitutions, or
(x) the first FR comprises SEQ ID NO: 74 or a variant thereof having one, two, three, four, five or six amino acid substitutions, the second FR comprises SEQ ID NO: 76 or a variant thereof having one, two, three, or four amino acid substitutions, the third FR comprises SEQ ID NO: 78 or a variant thereof having one, two, three, four, five, six, seven, eight, nine, or ten amino acid substitutions, and the fourth FR comprises SEQ ID NO: 80 or a variant thereof having one, two, or three amino acid substitutions, or
(xi) the first FR comprises SEQ ID NO: 82 or a variant thereof having one, two, three, four, five or six amino acid substitutions, the second FR comprises SEQ ID NO: 84 or a variant thereof having one, two, three, or four amino acid substitutions, the third FR comprises SEQ ID NO: 86 or a variant thereof having one, two, three, four, five, six, seven, eight, nine, or ten amino acid substitutions, and the fourth FR comprises SEQ ID NO: 88 or a variant thereof having one, two, or three amino acid substitutions, or
(xii) the first FR comprises SEQ ID NO: 90 or a variant thereof having one, two, three, four, five or six amino acid substitutions, the second FR comprises SEQ ID NO: 92 or a variant thereof having one, two, three, or four amino acid substitutions, the third FR comprises SEQ ID NO: 94 or a variant thereof having one, two, three, four, five, six, seven, eight, nine, or ten amino acid substitutions, and the fourth FR comprises SEQ ID NO: 96 or a variant thereof having one, two, or three amino acid substitutions.

Accordingly, the invention provides in one aspect an anti-C-reactive protein (CRP) single-domain antibody (sdAb) comprising an on-rate constant (kₒₙ) of about 3.30×10\hat{ }4−1.70×10\hat{ }7 and an off-rate constant (k_{off}) of about 3.20×10\hat{ }−9−2.20×10\hat{ }−1, comprising a first framework region (FR1), a second framework region (FR2), a third framework region (FR3), and a fourth framework region (FR3) wherein,
(i) FR1 comprises SEQ ID NO: 2 or a variant thereof having up to six amino acid substitutions, FR2 comprises SEQ ID NO: 4 or a variant thereof having up to four amino acid substitutions, FR3 comprises SEQ ID NO: 6 or a variant thereof having up to ten amino acid substitutions, and FR4 comprises SEQ ID NO: 8 or a variant thereof having up to three amino acid substitutions; or
(ii) FR1 comprises SEQ ID NO: 10 or a variant thereof having up to six amino acid substitutions, FR2 comprises SEQ ID NO: 12 or a variant thereof having up to four amino acid substitutions, FR3 comprises SEQ ID NO: 14 or a variant thereof having up to ten amino acid substitutions, and FR4 comprises SEQ ID NO: 16 or a variant thereof having up to three amino acid substitutions; or
(iii) FR1 comprises SEQ ID NO: 18 or a variant thereof having up to six amino acid substitutions, FR2 comprises SEQ ID NO: 20 or a variant thereof having up to four amino acid substitutions, FR3 comprises SEQ ID NO: 22 or a variant thereof having up to ten amino acid substitutions, and FR4 comprises SEQ ID NO: 24 or a variant thereof having up to three amino acid substitutions; or
(iv) FR1 comprises SEQ ID NO: 26 or a variant thereof having up to six amino acid substitutions, FR2 comprises SEQ ID NO: 28 or a variant thereof having up to four amino acid substitutions, FR3 comprises SEQ ID NO: 30 or a variant thereof having up to ten amino acid substitutions, and FR4 comprises SEQ ID NO: 32 or a variant thereof having up to three amino acid substitutions; or
(v) FR1 comprises SEQ ID NO: 34 or a variant thereof having up to six amino acid substitutions, FR2 comprises SEQ ID NO: 36 or a variant thereof having up to four amino acid substitutions, FR3 comprises SEQ ID NO: 38 or a variant thereof having up to ten amino acid substitutions, and FR4 comprises SEQ ID NO: 40 or a variant thereof having up to three amino acid substitutions; or
(vi) FR1 comprises SEQ ID NO: 42 or a variant thereof having up to six amino acid substitutions, FR2 comprises SEQ ID NO: 44 or a variant thereof having up to four amino acid substitutions, FR3 comprises SEQ ID NO: 46 or a variant thereof having up to ten amino acid substitutions, and FR4 comprises SEQ ID NO: 48 or a variant thereof having up to three amino acid substitutions; or
(vii) FR1 comprises SEQ ID NO: 50 or a variant thereof having up to six amino acid substitutions, FR2 comprises SEQ ID NO: 52 or a variant thereof having up to four amino acid substitutions, FR3 comprises SEQ ID NO: 54 or a variant thereof having up to ten amino acid substitutions, and FR4 comprises SEQ ID NO: 56 or a variant thereof having up to three amino acid substitutions; or
(viii) FR1 comprises SEQ ID NO: 58 or a variant thereof having up to six amino acid substitutions, FR2 comprises SEQ ID NO: 60 or a variant thereof having up to four amino acid substitutions, FR3 comprises SEQ ID NO: 62 or a variant thereof having up to ten amino acid substitutions, and FR4 comprises SEQ ID NO: 64 or a variant thereof having up to three amino acid substitutions; or
(ix) FR1 comprises SEQ ID NO: 66 or a variant thereof having up to six amino acid substitutions, FR2 comprises SEQ ID NO: 68 or a variant thereof having up to four amino acid substitutions, FR3 comprises SEQ ID NO: 70 or a variant thereof having up to ten amino acid substitutions, and FR4 comprises SEQ ID NO: 72 or a variant thereof having up to three amino acid substitutions; or
(x) FR1 comprises SEQ ID NO: 74 or a variant thereof having up to six amino acid substitutions, FR2 comprises SEQ ID NO: 76 or a variant thereof having up to four amino acid substitutions, FR3 comprises SEQ ID NO: 78 or a variant thereof having up to ten amino acid substitutions, and FR4 comprises SEQ ID NO: 80 or a variant thereof having up to three amino acid substitutions; or
(xi) FR1 comprises SEQ ID NO: 82 or a variant thereof having up to six amino acid substitutions, FR2 comprises SEQ ID NO: 84 or a variant thereof having up to four amino acid substitutions, FR3 comprises SEQ ID NO: 86 or a variant thereof having up to ten amino acid substitutions, and FR4 comprises SEQ ID NO: 88 or a variant thereof having up to three amino acid substitutions; or
(xii) FR1 comprises SEQ ID NO: 90 or a variant thereof having up to six amino acid substitutions, FR2 comprises SEQ ID NO: 92 or a variant thereof having up to four amino acid substitutions, FR3 comprises SEQ ID NO: 94 or a variant thereof having up to ten amino acid substitutions, and FR4 comprises SEQ ID NO: 96 or a variant thereof having up to three amino acid substitutions.

As mentioned herein above, a sdAb of the present invention can preferably comprise three CDRs and four FRs. Accordingly, a sdAb preferably comprises a first FR, a first CDR, a second FR, a second CDR, a third FR, a third CDR and a fourth FR. A sdAb of the present invention can consist of a first FR, a first CDR, a second FR, a second CDR, a third FR, a third CDR and a fourth FR. A sdAb of the present invention can comprise any one of SEQ ID NOs: 1, 9, 17, 25, 33, 41, 49, 57, 65, 73, 81, or 89. A sdAb of the present invention can also consist of SEQ ID NOs: 1, 9, 17, 25, 33, 41, 49, 57, 65, 73, 81, or 89. As described herein above for the individual CDRs and FRs, the sdAb can also comprise amino acid substitutions that essentially do not interfere with its function or structural integrity. A sdAb of the present invention can comprise any number of amino acid substitutions as long as it can still be used according to the invention. Accordingly, the present invention also provides variants of a sdAb of the present invention having 80, 81, 82, 83, 84, 85, 86, 87, 88, 89, 90, 91, 92, 93, 94, 95, 96, 97, 98, or 99% sequence identity when compared to the sdAb. Preferably, the invention provides variants of a sdAb of the invention having more than 90%, such as 91, 92, 93, 94, 95, 96, 97, 98, or 99%, or at least 90%, such as 90, 91, 92, 93, 94, 95, 96, 97, 98, or 99%, or 90−99%, such as 90, 91, 92, 93, 94, 95, 96, 97, 98, or 99% sequence identity when compared to the sdAb.

Accordingly, the present invention provides in another aspect an anti-C-reactive protein (CRP) single-domain antibody (sdAb) comprising an on-rate constant (kₒₙ) of about 3.30×10\hat{ }4−1.70×10\hat{ }7 and an off-rate constant (k_{off}) of about 3.20×10\hat{ }−9−2.20×10\hat{ }−1, wherein the sdAb comprises any one of
(i) SEQ ID NO: 1 or a variant thereof having at least about 90% sequence identity to SEQ ID NO: 1; or
(ii) SEQ ID NO: 9 or a variant thereof having at least about 90% sequence identity to SEQ ID NO: 9; or
(iii) SEQ ID NO: 17 or a variant thereof having at least about 90% sequence identity to SEQ ID NO: 17; or
(iv) SEQ ID NO: 25 or a variant thereof having at least about 90% sequence identity to SEQ ID NO: 25; or
(v) SEQ ID NO: 33 or a variant thereof having at least about 90% sequence identity to SEQ ID NO: 33; or
(vi) SEQ ID NO: 41 or a variant thereof having at least about 90% sequence identity to SEQ ID NO: 41; or
(vii) SEQ ID NO: 49 or a variant thereof having at least about 90% sequence identity to SEQ ID NO: 49; or
(viii) SEQ ID NO: 57 or a variant thereof having at least about 90% sequence identity to SEQ ID NO: 57; or
(ix) SEQ ID NO: 65 or a variant thereof having at least about 90% sequence identity to SEQ ID NO: 65; or
(x) SEQ ID NO: 73 or a variant thereof having at least about 90% sequence identity to SEQ ID NO: 73; or
(xi) SEQ ID NO: 81 or a variant thereof having at least about 90% sequence identity to SEQ ID NO: 81; or
(xii) SEQ ID NO: 89 or a variant thereof having at least about 90% sequence identity to SEQ ID NO: 89.

As mentioned herein above, the sdAbs of the invention can embody a specific on-rate constant (kₒₙ) and off-rate constant (k_{off}). sdAbs within the ranges defined herein have the advantage that they can associate and dissociate with CRP over time. This means that a continuous and repeated association followed by dissociation with CRP is achieved. This is particularly desired when the sdAbs are used in a measurement over time where the dynamic level of CRP is to be determined. As described herein, determining and monitoring the dynamic level of CRP is particularly advantageous for determining inflammation spikes, such as during the course of a disease or to monitor inflammatory reactions, e.g. to a treatment or drug. Accordingly, it is preferred herein, that the anti-CRP sdAbs of the present invention continuously and reversibly bind CRP. The reversible binding of CRP is particularly desired when the sdAbs are used on a sensor, e.g. biosensor. A sdAb having exceptionally high affinity cannot dissociate from CRP and thus is not feasible in a measurement of dynamic CRP levels over time. The present invention provides anti-CRP sdAbs within kₒₙ and k_{off} ranges allowing continuous and reversible binding of CRP. For example, a sdAb of the invention can bind or associate with an CRP, followed by dissociation from said CRP. After dissociation from the first CRP, the same sdAb can bind a second (other) CRP. After dissociation from the second CRP, the same sdAb can bind a third (other) CRP. Association and dissociation of any further CRP can be repeated as described before. Thus, the sdAbs of the present invention can associate and dissociate a multitude of CRP (reversible binding), enabling measurement of dynamic CRP levels over time (continuous binding).

Accordingly, the invention provides in one aspect an anti-C-reactive protein (CRP) single-domain antibody (sdAb) comprising an on-rate constant (kₒₙ) of about 3.30×10\hat{ }4−1.70×10\hat{ }7 and an off-rate constant (k_{off}) of about 3.20×10\hat{ }−9−2.20×10\hat{ }−1, wherein the anti-CRP sdAb continuously and reversibly binds CRP.

The invention also provides an anti-C-reactive protein (CRP) single-domain antibody (sdAb) comprising an on-rate constant (kₒₙ) of about 3.80×10\hat{ }5−1.70×10\hat{ }7 and an off-rate constant (k_{off}) of about 3.20×10\hat{ }−9−1.30×10\hat{ }−2, wherein the anti-CRP sdAb continuously and reversibly binds CRP.

The invention also provides an anti-C-reactive protein (CRP) single-domain antibody (sdAb) comprising an on-rate constant (kₒₙ) of about 3.30×10\hat{ }4−4.60×10\hat{ }6 and an off-rate constant (k_{off}) of about 2.60×10\hat{ }−4−2.20×10\hat{ }−1, wherein the anti-CRP sdAb continuously and reversibly binds CRP. The invention also provides an anti-C-reactive protein (CRP) single-domain antibody (sdAb) comprising an on-rate constant (kₒₙ) of about 4.10×10\hat{ }4−3.80×10\hat{ }5 and an off-rate constant (k_{off}) of about 2.00×10\hat{ }−5−6.90×10\hat{ }−3, wherein the anti-CRP sdAb continuously and reversibly binds CRP.

The invention also provides in one aspect an anti-C-reactive protein (CRP) single-domain antibody (sdAb) comprising an on-rate constant (kₒₙ) of about 3.30×10\hat{ }4−1.70×10\hat{ }7 and an off-rate constant (k_{off}) of about 3.20×10\hat{ }−9−2.20×10\hat{ }−1, wherein the anti-CRP sdAb binds a second or further CRP after dissociating from a first CRP.

The invention also provides in one aspect an anti-C-reactive protein (CRP) single-domain antibody (sdAb) comprising an on-rate constant (kₒₙ) of about 3.80×10\hat{ }5−1.70×10\hat{ }7 and an off-rate constant (k_{off}) of about 3.20×10\hat{ }−9−1.30×10\hat{ }−2, wherein the anti-CRP sdAb binds a second or further CRP after dissociating from a first CRP.

The invention also provides in one aspect an anti-C-reactive protein (CRP) single-domain antibody (sdAb) comprising an on-rate constant (kₒₙ) of about 3.30×10\hat{ }4−4.60×10\hat{ }6 and an off-rate constant (k_{off}) of about 2.60×10\hat{ }−4−2.20×10\hat{ }−1, wherein the anti-CRP sdAb binds a second or further CRP after dissociating from a first CRP.

The invention also provides in one aspect an anti-C-reactive protein (CRP) single-domain antibody (sdAb) comprising an on-rate constant (kₒₙ) of about 4.10×10\hat{ }4−3.80×10\hat{ }5 and an off-rate constant (k_{off}) of about 2.00×10\hat{ }−5−6.90×10\hat{ }−3, wherein the anti-CRP sdAb binds a second or further CRP after dissociating from a first CRP.

As mentioned herein above, the herein provided anti-CRP sdAbs can associate and dissociate with CRP (over time). Accordingly, the present invention provides for anti-CRP sdAbs that are, inter alia, particularly useful in the detection and/or quantification of CRP across a broad range of CRP concentrations. As is further detailed herein below, due to the high (i.e., μM) CRP concentrations in a biological fluid, e.g. human blood even at baseline inflammation (see, for example, Table 1), in the context of the present invention it is particularly advantageous that the herein provided anti-CRP sdAbs, due to their advantageous binding affinities (and kₒₙ and k_{off} values), are able to detect and/or quantify CRP concentrations throughout the entire inflammation range from baseline to severe inflammation (i.e., μM) in a biological fluid, e.g. human blood (see, for example, Table 12 or 13).

Accordingly, the present invention provides for an anti-C-reactive protein (CRP) single-domain antibody (sdAb) capable of detecting and/or quantifying CRP concentrations of about 0.01 μM or less to 1 μM or more, such as 0.01 μM or less and/or 0.01 μM to 0.1 μM and/or 0.1 μM to 1 μM and/or 1 μM or more, optionally in a human biological fluid. Accordingly, the present invention provides for an anti-C-reactive protein (CRP) single-domain antibody (sdAb) capable of detecting and/or quantifying baseline to severe inflammation, such as baseline inflammation and/or low inflammation and/or moderate inflammation and/or severe inflammation, optionally in a human biological fluid.

In particular, the present invention provides for anti-CRP sdAb SCR-3H6 (or variants thereof), capable of detecting and/or quantifying CRP concentrations of about 0.01 μM or less to 1 M or more, such as 0.01 μM or less and/or 0.01 μM to 0.1 μM and/or 0.1 μM to 1 μM and/or 1 μM or more, optionally in a human biological fluid. Accordingly, the present invention provides for anti-CRP sdAb SCR-3H6 (or variants thereof), capable of detecting and/or quantifying baseline to severe inflammation, such as baseline inflammation and/or low inflammation and/or moderate inflammation and/or severe inflammation, optionally in a human biological fluid.

In particular, the present invention provides for anti-CRP sdAb SCR-3A4 (or variants thereof), detecting and/or quantifying CRP concentrations of about 0.01 μM or less to 1 μM or more, such as 0.01 μM or less and/or 0.01 μM to 0.1 μM and/or 0.1 μM to 1 μM and/or 1 μM or more, optionally in a human biological fluid. Accordingly, the present invention provides for anti-CRP sdAb SCR-3A4 (or variants thereof), capable of detecting and/or quantifying baseline to severe inflammation, such as baseline inflammation and/or low inflammation and/or moderate inflammation and/or severe inflammation, optionally in a human biological fluid.

In particular, the present invention provides for anti-CRP sdAb SCR-1A1 (or variants thereof), detecting and/or quantifying CRP concentrations of about 0.01 μM or less to 1 μM or more, such as 0.01 μM or less and/or 0.01 μM to 0.1 μM and/or 0.1 μM to 1 μM and/or 1 μM or more, optionally in a human biological fluid. Accordingly, the present invention provides for anti-CRP sdAb SCR-1A1 (or variants thereof), capable of detecting and/or quantifying baseline to severe inflammation, such as baseline inflammation and/or low inflammation and/or moderate inflammation and/or severe inflammation, optionally in a human biological fluid.

In particular, the present invention provides for anti-CRP sdAb SCR-3E5 (or variants thereof), detecting and/or quantifying CRP concentrations of about 0.01 μM or less to 1 μM or more, such as 0.01 μM or less and/or 0.01 μM to 0.1 μM and/or 0.1 μM to 1 μM and/or 1 μM or more, optionally in a human biological fluid. Accordingly, the present invention provides for anti-CRP sdAb SCR-3E5 (or variants thereof), capable of detecting and/or quantifying baseline to severe inflammation, such as baseline inflammation and/or low inflammation and/or moderate inflammation and/or severe inflammation, optionally in a human biological fluid.

In particular, the present invention provides for anti-CRP sdAb SCR-3B7 (or variants thereof), detecting and/or quantifying CRP concentrations of about 0.01 μM or less to 1 μM or more, such as 0.01 μM or less and/or 0.01 μM to 0.1 μM and/or 0.1 μM to 1 μM and/or 1 μM or more, optionally in a human biological fluid. Accordingly, the present invention provides for anti-CRP sdAb SCR-3B7 (or variants thereof), capable of detecting and/or quantifying baseline to severe inflammation, such as baseline inflammation and/or low inflammation and/or moderate inflammation and/or severe inflammation, optionally in a human biological fluid.

In particular, the present invention provides for anti-CRP sdAb SCR-3H1 (or variants thereof), detecting and/or quantifying CRP concentrations of about 0.01 μM or less to 1 μM or more, such as 0.01 μM or less and/or 0.01 μM to 0.1 μM and/or 0.1 μM to 1 μM and/or 1 μM or more, optionally in a human biological fluid. Accordingly, the present invention provides for anti-CRP sdAb SCR-3H1 (or variants thereof), capable of detecting and/or quantifying baseline to severe inflammation, such as baseline inflammation and/or low inflammation and/or moderate inflammation and/or severe inflammation, optionally in a human biological fluid.

In particular, the present invention provides for anti-CRP sdAb SCR-2D2 (or variants thereof), detecting and/or quantifying CRP concentrations of about 0.01 μM or less to 1 μM or more, such as 0.01 μM or less and/or 0.01 μM to 0.1 μM and/or 0.1 μM to 1 μM and/or 1 μM or more, optionally in a human biological fluid. Accordingly, the present invention provides for anti-CRP sdAb SCR-2D2 (or variants thereof), capable of detecting and/or quantifying baseline to severe inflammation, such as baseline inflammation and/or low inflammation and/or moderate inflammation and/or severe inflammation, optionally in a human biological fluid.

In particular, the present invention provides for anti-CRP sdAb SCR-2B4 (or variants thereof), detecting and/or quantifying CRP concentrations of about 0.01 μM or less to 1 μM or more, such as 0.01 μM or less and/or 0.01 μM to 0.1 μM and/or 0.1 μM to 1 μM and/or 1 μM or more, optionally in a human biological fluid. Accordingly, the present invention provides for anti-CRP sdAb SCR-2B4 (or variants thereof), capable of detecting and/or quantifying baseline to severe inflammation, such as baseline inflammation and/or low inflammation and/or moderate inflammation and/or severe inflammation, optionally in a human biological fluid.

In particular, the present invention provides for anti-CRP sdAb SCR-2D1 (or variants thereof), detecting and/or quantifying CRP concentrations of about 0.01 μM or less to 1 μM or more, such as 0.01 μM or less and/or 0.01 μM to 0.1 μM and/or 0.1 μM to 1 μM and/or 1 μM or more, optionally in a human biological fluid. Accordingly, the present invention provides for anti-CRP sdAb SCR-2D1 (or variants thereof), capable of detecting and/or quantifying baseline to severe inflammation, such as baseline inflammation and/or low inflammation and/or moderate inflammation and/or severe inflammation, optionally in a human biological fluid.

In particular, the present invention provides for anti-CRP sdAb SCR-3E4 (or variants thereof), detecting and/or quantifying CRP concentrations of about 0.01 μM or less to 1 μM or more, such as 0.01 μM or less and/or 0.01 μM to 0.1 μM and/or 0.1 μM to 1 μM and/or 1 μM or more, optionally in a human biological fluid. Accordingly, the present invention provides for anti-CRP sdAb SCR-3E4 (or variants thereof), capable of detecting and/or quantifying baseline to severe inflammation, such as baseline inflammation and/or low inflammation and/or moderate inflammation and/or severe inflammation, optionally in a human biological fluid.

In particular, the present invention provides for anti-CRP sdAb SCR-3G7 (or variants thereof), detecting and/or quantifying CRP concentrations of about 0.01 μM or less to 1 μM or more, such as 0.01 μM or less and/or 0.01 μM to 0.1 μM and/or 0.1 μM to 1 μM and/or 1 μM or more, optionally in a human biological fluid. Accordingly, the present invention provides for anti-CRP sdAb SCR-3G7 (or variants thereof), capable of detecting and/or quantifying baseline to severe inflammation, such as baseline inflammation and/or low inflammation and/or moderate inflammation and/or severe inflammation, optionally in a human biological fluid.

In particular, the present invention provides for anti-CRP sdAb SCR-3A5 (or variants thereof), detecting and/or quantifying CRP concentrations of about 0.01 μM or less to 1 μM or more, such as 0.01 μM or less and/or 0.01 μM to 0.1 μM and/or 0.1 μM to 1 μM and/or 1 μM or more, optionally in a human biological fluid. Accordingly, the present invention provides for anti-CRP sdAb SCR-3A5 (or variants thereof), capable of detecting and/or quantifying baseline to severe inflammation, such as baseline inflammation and/or low inflammation and/or moderate inflammation and/or severe inflammation, optionally in a human biological fluid.

As is detailed in Table 12 and 13, the herein provided anti-CRP sdAbs can all, inter alia, can detect the entire inflammatory range of CRP and can, accordingly, quantify and/or detect CRP concentrations of, inter alia, 0.01 μM or less to 1 μM or more, such as 0.01 μM or less and/or 0.01 μM to 0.1 μM and/or 0.1 μM to 1 μM and/or 1 μM or more, optionally in a human biological fluid. Accordingly, in the context of the present invention, if any herein provided sdAb detects and/or quantifies moderate inflammation, this means that said antibody detects and/or quantifies baseline inflammation to moderate inflammation (i.e., baseline inflammation, low inflammation, and moderate inflammation). This similarly applies to baseline, low, moderate and severe inflammation.

Accordingly, in the context of the present invention, if any herein provided sdAb detects and/or quantifies CRP concentrations of about or more than about 0.1 μM, this means that said antibody detects and/or quantifies CRP concentrations of about 0.01 μM (or less than about 0.01 μM) to about or more than about 0.1 μM.

The herein provided anti-CRP sdAbs SCR-3E4, SCR-3G7, SCR-2D1, SCR-1A1, SCR-3B7, SCR-2D2, SCR-3E5, SCR-2B4, SCR-3H1, SCR-3H6, SCR-3A4, SCR-3A5 may in the context of the present invention be defined as detailed herein above or below, by any function characterization detailed in, inter alia, Table 13, and/or by any of the sequences (e.g., CDRs, CDRs and FRs, full length sequences) summarized in Table 14 and provided in the enclosed sequence listing. As is also mentioned herein, such sdAbs (or the respective CDR, FR, or full length sequences) may comprise up to five amino acid substitutions. Accordingly, they may comprise about 1, about 2, about 3, about 4, or about 5 amino acid substitutions.

TABLE 13
Summary characteristics of exemplary sdAbs provided herein.
The column “Covered inflammation range” in the table below indicates
the respective inflammation range that a given sdAb can at least partly
cover (i.e., any inflammation range that is indicated with “*” in
Table 12) or fully cover (i.e., any inflammation range that is indicated
with “**” in Table 12). All other columns summarize data also already
detailed herein above.

| Clone | kₐ[s⁻¹M⁻¹] | k_{d} [s⁻¹] | K_{D} [nM] | Covered inflammation range |
|---|---|---|---|---|
| SCR-3H6 | 2.4E6 | 8.1E−5 | 0.03 | Baseline to severe |
| SCR-3E5 | 1.7E6 | 3.2E−9* | 1.8E−6* | Baseline to severe |
| SCR-3B7 | 1.5E6 | 1.3E−7* | 7.6E−5* | Baseline to severe |
| SCR-3H1 | 2.1E6 | 2.5E−4 | 0.12 | Baseline to severe |
| SCR-2D2 | 1.6E6 | 2.0E−5 | 0.01 | Baseline to severe |
| SCR-2B4 | 1.7E6 | 1.7E−4 | 0.1 | Baseline to severe |
| SCR-2D1 | 8.0E5 | 3.0E−4 | 0.38 | Baseline to severe |
| SCR-3E4 | 3.8E5 | 6.2E−5 | 0.16 | Baseline to severe |
| SCR-3A4 | 2.7E6 | 1.9E−3 | 0.70 | Baseline to severe |
| SCR-1A1 | 1.3E6 | 1.3E−2 | 9.62 | Baseline to severe |
| SCR-3G7 | 6.0E5 | 9.8E−5* | 0.16* | Baseline to severe |
| SCR-3A5 | 1.7E7 | 1.3E−3 | 0.08 | Baseline to severe |

*k^{d} too slow to be accurately calculated

Antibodies in general can be used to inhibit the biological function of their targets. One advantage of sdAb is that they do not necessarily inhibit the biological function of their targets. In the sense of the present invention, both can be desired, i.e. inhibition of biological function in the sense of a treatment or a non-inhibiting sdAb, in the sense of monitoring CRP levels together with a treatment or independent thereof. Accordingly, the present invention provides sdAbs that do not inhibit the biological function of CRP. In another aspect, the present invention provides sdAbs that do inhibit the biological function of CRP. As used herein “biological function of CRP” relates in general to any effect of CRP, i.e. relates to anything that CRP “does”. This can include any function related to CRP or CRP signalling or the CRP signalling pathway. The biological function of CRP also includes upstream and downstream effects of and on CRP that may be related to CRP or the corresponding inflammation response.

Accordingly, the invention provides in one aspect an anti-C-reactive protein (CRP) single-domain antibody (sdAb) comprising an on-rate constant (kₒₙ) of about 3.30×10\hat{ }4−1.70×10\hat{ }7 and an off-rate constant (k_{off}) of about 3.20×10\hat{ }−9−2.20×10\hat{ }−1, wherein the anti-CRP sdAb inhibits the biological function of CRP.

The invention also provides an anti-C-reactive protein (CRP) single-domain antibody (sdAb) comprising an on-rate constant (kₒₙ) of about 3.30×10\hat{ }4−1.70×10\hat{ }7 and an off-rate constant (k_{off}) of about 3.20×10\hat{ }−9−2.20×10\hat{ }−1, wherein the anti-CRP sdAb inhibits the biological function of CRP, wherein the biological function of CRP is CRP signalling or the CRP signalling pathway.

In another aspect, the invention provides an anti-C-reactive protein (CRP) single-domain antibody (sdAb) comprising an on-rate constant (kₒₙ) of about 3.30×10\hat{ }4−1.70×10\hat{ }7 and an off-rate constant (k_{off}) of about 3.20×10\hat{ }−9−2.20×10\hat{ }−1, wherein the anti-CRP sdAb does not inhibit the biological function of CRP.

The invention also provides an anti-C-reactive protein (CRP) single-domain antibody (sdAb) comprising an on-rate constant (kₒₙ) of about 3.30×10\hat{ }4−1.70×10\hat{ }7 and an off-rate constant (k_{off}) of about 3.20×10\hat{ }−9−2.20×10\hat{ }−1, wherein the anti-CRP sdAb does not inhibit the biological function of CRP, wherein the biological function of CRP is CRP signalling or the CRP signalling pathway.

As mentioned above, a sdAb of the present invention is particularly useful as a sensor, such as a biosensor. In general, a biosensor is an object used for the detection of a substance. A biosensor can combine a biological component, e.g. sdAb of the invention, with a physiochemical detector. When used in a biosensor, the biological component (sdAb) can associate with CRP, and the physiochemical detector then detects the association/binding. As mentioned above, the sdAb of the invention can continuously and reversibly associate with and dissociate from CRP allowing the physiochemical detector to measure association/dissociation and therefore CRP levels over time. In the sense of the present invention, the physiochemical detector can be an electrochemical detector. In general, the invention is not particularly limited to the use of any physiochemical detector, rather any physiochemical detector that allows a quantitative readout of the association/dissociation of anti-CRP sdAb of the present invention can be used. Exemplary physiochemical detectors that can be used to quantify anti-CRP sdAb association/dissociation with CRP are described herein below. The biosensor of the present invention comprises at least, a sdAb of the present invention, at least one working electrode (also known as the sensing or redox electrode) and a substrate (structural element). Association of CRP with the sdAb induces an electrical signal in the working electrode which allows for a quantitative measurement of CRP association. The electric signal that is generated can be readout by any electrochemical detection techniques commonly used, such as amperometry, chronoamperometry, potentiometry, conductometry, voltammetry, cyclic voltammetry, galvanic cell, impedance spectroscopy, and the like. A working electrode used in the biosensor may comprise any conductive material, such as carbon, metal or alloy, or a metallic compound. An exemplary carbon-based material that can be used in the working electrode is graphite. An exemplary metal or alloy that can be used in the working electrode is platinum, gold or a gold alloy. Exemplary metallic compounds are silicon, ruthenium dioxide or titanium dioxide. In some embodiments, a biosensor electrode can be or comprise a glassy carbon electrode, a carbon nanotube-modified electrode, an indium tin oxide (ITO) electrode, a platinum electrode, a silver electrode, a gold electrode, or a palladium electrode. In some embodiments, a biosensor electrode can be or comprise a gold nanostructured microelectrode or a gold wire electrode. In the sense of the present invention a biosensor electrode can be or comprise a working electrode and/or a counter electrode and/or a reference electrode. In some embodiments a biosensor electrode can be or can comprise a working electrode. In some embodiments a biosensor electrode can be or can comprise a counter electrode. In some embodiments a biosensor electrode can be or can comprise a reference electrode.

In a non-limiting example, a biosensor can comprise a working electrode (known as transduction element, where the biochemical reaction happens), a counter electrode (to close the circuit and allow current to flow between working and counter electrode), a substrate (where the electrodes are deposited on) and a reference electrode (e.g. made from Ag/AgCl), kept at a distance from the reaction site to maintain a known and stable potential.

The sdAbs of the present invention can be immobilized on the surface of the working electrode directly, e.g. by physisorption, or indirectly, e.g. by a linker. In the sense of the present invention a linker can be any conventional protein-tag, such as a His-tag (linking a sdAb to metal ions immobilized on working electrode), Strep-tag (linking a sdAb to Strep-tactin immobilized on working electrode), SpyTag (linking a sdAb to SpyCatcher immobilized on working electrode) or FLAG-tag (linking a sdAb to anti-FLAG-tag monoclonal antibodies immobilized on working electrode). A linker can also be any conventional protein-tag suitable for chemo-enzymatic conjugation, such as for Sortase-mediated linkage (linking an LPXTG-tagged sdAb to amines/lysines modified working electrode interface), BirA-mediated linkage (introducing biotin on sdAb, which then links to a strepdavidin or avidin modified working electrode interface), Lipoic Acid Ligase (LpIA)-mediated linkage (covalently attaching an azide bearing lipoic acid derivative on sdAb, which can then can be immobilized on sensor through azide-alkyne click chemistry), Tubulin tyrosine ligase (TTL)-mediated linkage (catalyzes the addition of tyrosine derivatives to an engineered Tub-tag, which enables subsequent bioorthogonal reactions with a great variety of probes or effector molecules immobilized on working electrode), or Transglutaminase-mediated linkage (catalyses the formation of stable isopeptide bond between glutamine on c-myc-tag and various primary amine functionalized proteins/substrates). A linker can also be any conventional chemistry techniques including click chemistry and copper-free click chemistry, such as EDC/NHS chemistry (linking primary amines presents on sdAb to a carboxylic acid terminated working electrode), maleimide/cysteine chemistry (linking cysteines presents on sdAb to a maleimide terminated working electrode), or DBCO chemistry (forming covalent triazole linkage to azide modified molecules). A linker can further comprise an amino acid sequence (“peptide tether”), a single-or double-stranded nucleic acid sequence (“ssDNA or dsDNA tether”), Polyethylenglycol (PEG) chains, or combinations thereof, acting as a bridge between the linker on the sdAb to the working electrode. A linker can comprise double-stranded DNA (dsDNA), single-stranded DNA (ssDNA), charged polymers, uncharged polymers, or any combination thereof. A linker can be negatively charged and comprise a DNA/DNA duplex, a peptide nucleic acid (PNA)/DNA duplex, a PNA/PNA duplex where one or both of the PNA are modified with negative charged amino acids, a rigid anionic polyelectrolyte, a rigid negatively charged peptide, or any combination thereof. A linker can also be positively charged and comprise a PNA/PNA duplex with lysines, a rigid cationic polyelectrolyte, a rigid positively charged peptide, or any combination thereof. A linker can have a length ranging from about 5 nm to about 20 nm. In some embodiments, a linker comprises ssDNA having a length ranging from about 10mer to about 100mer. In some embodiments, a linker comprises a dsDNA having a length ranging from about 15mer to about 60mer. In some embodiments a linker is a rigid linker. Alternatively, it may be preferred that a sdAb is directly immobilized (e.g.: via thiol-gold chemistry) on a gold working electrode surface. A DNA tether may also be used to bind the sdAb to the working electrode. In this case sdAbs are coupled to one end of a DNA tether via maleimide-cysteine chemistry. While the other end of the DNA strand is attached to the gold working electrode via thiol-gold chemistry. The linker can further comprise a detection label or a tag as defined herein, e.g. a redox reporter or an enzyme. A redox reporter can be bound to the linker.

A linker can have a first end and a second end, the first end of the linker can be bound to a surface of a biosensor electrode, e.g. working electrode, the second end can be bound to an anti-CRP sdAb of the present invention.

The sequence of the sdAb of the present invention, i.e. SEQ ID NOs: 1, 9, 17, 25, 33, 41, 49, 57, 65, 73, 81, or 89 may be modified to include a linker sequence. The sdAb and working electrode may be attached to a substrate as a structural element. Exemplary substrates that can be used in the biosensor are glass, or ceramic or plastic (e.g.: flexible/semirigid polyimide). The biosensor of the present invention can further comprise a counter electrode and/or a reference electrode. The reference electrode, e.g. made from Ag/AgCl, is kept at a distance from the reaction site in order to maintain a known and stable potential. The working electrode serves as the transduction element in the biochemical reaction, while the counter electrode establishes a connection to the electrolytic solution so that a current can be applied to the working electrode. These electrodes preferably are conductive and chemically stable (see e.g. Grieshaber, Dorothee, et al. “Electrochemical biosensors-sensor principles and architectures.” Sensors 8.3 (2008): 1400−1458.). The biosensor of the present invention can further comprise a detection label or a tag, e.g. a redox reporter or an enzyme. Exemplary redox reporters/indicators are ferrocene, [Co(GA)₂ (phen)] (GA=glycolic acid, phen=1,10-phenathroline), methylene blue, metal nanoparticles (e.g., Au, Pt, Pd, Ag, Cu), pyrroloquinoline quinone (PQQ), benzoquine, Osmium (III) complexes such as Os(bpy)Cl₂³⁺, diphenylamine, or any combination thereof. These can e.g. be incorporated into a DNA linker and can be oxidized at an electrode potential, generating a measurable faraday current (see e.g. Das, Jagotamoy, et al. “Reagentless biomolecular analysis using a molecular pendulum.” Nature chemistry 13.5 (2021): 428-434.) Such a tag can be comprised in or fused e.g. to the sdAb of the present invention. A tag can be fused for example to the C-terminus or the N-terminus of a sdAb of the invention. A tag can also be comprised in a linker as disclosed herein. A sdAb of the invention can for example comprise a Cysteine-tag, a fluorescence tag, a metal tag or a poly-histidine tag (His-tag). Use of such a tag is optional for the biosensor as described herein. As used herein “conjugated” preferably means that a sdAb of the present invention is linked to/bound to/layered on to/attached to a surface of a biosensor electrode, such as a working electrode. In the sense of the present invention, an anti-CRP sdAb can be comprised in a biosensor or can be part of a biosensor. A sdAb of the present invention may be attached to a surface of a biosensor electrode via a linker as disclosed herein or can physically be deposited (e.g. primarily by van der Waals forces) onto the surface of a biosensor electrode via adsorption physiosorption. Accordingly, “conjugated” as used herein can also mean physical deposition onto the surface of a biosensor electrode via adsorption physiosorption. As used herein “conjugated to a working electrode” can also be “conjugated to a biosensor electrode”.

Accordingly, the invention provides in one aspect a biosensor comprising a sdAb of the present invention and a working electrode.

The invention also provides a biosensor comprising a sdAb of the present invention and a working electrode, wherein the sdAb is conjugated to the working electrode.

The invention also provides a biosensor comprising a sdAb of the present invention and a working electrode, wherein the sdAb is conjugated to the working electrode via a linker.

The invention also provides a biosensor comprising a sdAb of the present invention and a working electrode, wherein the sdAb is conjugated to the working electrode via a linker, wherein the linker is selected from the group of:
(i) protein-tag, such as His-tag, Strep-tag, SpyTag, or FLAG-tag,
(ii) protein-tag suitable for chemo-enzymatic conjugation, such as for Sortase-mediated linkage, BirA-mediated linkage, Lipoic Acid Ligase (LpIA)-mediated linkage, Tubulin tyrosine ligase (TTL)-mediated linkage, Transglutaminase-mediated linkage,
(iii) conventional chemistry techniques, such as click chemistry, copper-free click chemistry, EDC/NHS chemistry, maleimide/cysteine chemistry, or DBCO chemistry, amino acid sequence, such as a peptide tether, (iv)
(v) nucleic acid sequence, such as a single stranded DNA or a double stranded DNA,
(vi) Polyethylenglycol (PEG).

The invention also provides a biosensor comprising a sdAb of the present invention and a working electrode, wherein the sdAb is conjugated to the working electrode, and a counter electrode and/or a reference electrode, and a substrate.

The invention also provides a biosensor comprising at least a sdAb of the present invention and a working electrode, and a substrate.

The invention also provides a biosensor comprising a sdAb of the present invention and a working electrode, wherein the working electrode comprises a conductive material, such as carbon, metal or metallic compound.

The invention also provides a biosensor comprising a sdAb of the present invention and a working electrode, wherein the sdAb comprises a detection label or tag.

In another aspect, the invention provides an anti-C-reactive protein (CRP) single-domain antibody (sdAb) comprising an on-rate constant (kₒₙ) of about 3.30×10\hat{ }4−1.70×10\hat{ }7 and an off-rate constant (k_{off}) of about 3.20×10\hat{ }−9−2.20×10\hat{ }−1, wherein the anti-CRP sdAb is conjugated a working electrode.

The invention also provides an anti-C-reactive protein (CRP) single-domain antibody (sdAb) comprising an on-rate constant (kₒₙ) of about 3.30×10\hat{ }4−1.70×10\hat{ }7 and an off-rate constant (k_{off}) of about 3.20×10\hat{ }−9−2.20×10\hat{ }−1, wherein the anti-CRP sdAb is conjugated to a working electrode via a nucleic acid (linker).

The invention also provides an anti-C-reactive protein (CRP) single-domain antibody (sdAb) comprising an on-rate constant (kₒₙ) of about 3.30×10\hat{ }4−1.70×10\hat{ }7 and an off-rate constant (k_{off}) of about 3.20×10\hat{ }−9−2.20×10\hat{ }−1, wherein the anti-CRP sdAb is conjugated to a working electrode, further comprising a counter electrode and/or a reference electrode, and a substrate.

The invention also provides an anti-C-reactive protein (CRP) single-domain antibody (sdAb) comprising an on-rate constant (kₒₙ) of about 3.30×10\hat{ }4−1.70×10\hat{ }7 and an off-rate constant (k_{off}) of about 3.20×10\hat{ }−9−2.20×10\hat{ }−1 in a biosensor, comprising at least an anti-CRP sdAb of the present invention, a working electrode, and a substrate.

The invention also provides an anti-C-reactive protein (CRP) single-domain antibody (sdAb) comprising an on-rate constant (kₒₙ) of about 3.30×10\hat{ }4−1.70×10\hat{ }7 and an off-rate constant (K_{off}) of about 3.20×10\hat{ }−9−2.20×10\hat{ }−1, wherein the anti-CRP sdAb is conjugated a working electrode, wherein the working electrode comprises a conductive material, such as carbon, metal or metallic compound.

The invention also provides an anti-C-reactive protein (CRP) single-domain antibody (sdAb) comprising an on-rate constant (kₒₙ) of about 3.30×10\hat{ }4−1.70×10\hat{ }7 and an off-rate constant (k_{off}) of about 3.20×10\hat{ }−9−2.20×10\hat{ }−1, wherein the anti-CRP sdAb is conjugated a working electrode, wherein the sdAb comprises a detection label or tag.

It is envisioned herein that a sdAb of the invention can be combined with other antibodies e.g. other antibodies/sdAbs binding CRP or other antibodies/sdAbs binding any other/further targets. It is also envisioned herein, that one or more anti-CRP sdAb of the invention can be combined in the products, methods, uses and purpose limited products provided herein. Accordingly, one or more sdAbs of the present invention can be any one of the sdAbs defined in SEQ ID NOs: 1, 9, 17, 25, 33, 41, 49, 57, 65, 73, 81, or 89. Similarly, two or more sdAbs of the present invention can be any one of the sdAbs defined in SEQ ID NOs: 1, 9, 17, 25, 33, 41, 49, 57, 65, 73, 81, or 89. In the sense of the present invention “one or more sdAb of the present invention” preferably means one, two, three, four, five, six, seven, eight, nine, ten, eleven or twelve anti-CRP sdAbs selected from the group of SEQ ID NOs: 1, 9, 17, 25, 33, 41, 49, 57, 65, 73, 81, or 89. In the sense of the present invention “two or more sdAb of the present invention” preferably means two, three, four, five, six, seven, eight, nine, ten, eleven or twelve anti-CRP sdAbs selected from the group of SEQ ID NOs: 1, 9, 17, 25, 33, 41, 49, 57, 65, 73, 81, or 89.

The present invention also provides a nucleic acid encoding an anti-CRP sdAb of the invention. A “nucleic acid” in the sense of the present invention is a polymer of nucleotides, such as a polynucleotide. A nucleic acid can be any nucleic acid, preferably a deoxyribonucleic acid (DNA) or a ribonucleic acid (RNA). A nucleic acid encoding an anti-CRP sdAb of the invention is particularly useful for producing the sdAb of the invention. For example, a nucleic acid can comprise a DNA plasmid or DNA vector encoding an anti-CRP sdAb of the invention. A DNA plasmid or DNA vector can comprise additional elements that aid the transcription and translation of the sdAb in a host cell, such as artificial or viral promoters. For example, a DNA plasmid or DNA vector can further comprise a strong viral promoter, such as the simian vacuolating virus 40 (SV40) promoter, the Rous Sarcoma Virus (RSV) promoter, the cytomegalovirus (CMV) immediate early promoter or a retroviral cis-acting transcriptional element. A DNA plasmid or DNA vector can further comprise strong polyadenylation/transcriptional termination signals, such as bovine growth hormone or rabbit beta-globulin polyadenylation sequences. A DNA plasmid or DNA vector can further comprise modifications, such as insertion of enhancer sequences, synthetic introns, adenovirus tripartite leader (TPL) sequences and modifications to the polyadenylation and transcriptional termination sequences. The DNA plasmid or DNA vector of the present invention can be codon optimized for expression in a host, host cell or organism. Useful hosts, host cells or organisms for sdAb production are for example viruses or bacteria, such as bacteriophages, Escherichia coli, E. coli strain BL21, E. coli strain TG1, yeast, insect cells, or mammalian cells. For example the E. coli strain BL21 or E. coli strain TG1 can be used to produce sdAbs of the present invention. The DNA of the present invention can be single-stranded DNA or double-stranded DNA. The DNA of the present invention is preferably double stranded. The nucleic acid, such as a DNA, of the present invention can be present in a host cell, e.g. for the purpose of expressing or producing a sdAb of the invention. Accordingly, the invention provides a host cell comprising a nucleic acid encoding an anti-C-reactive protein (CRP) single-domain antibody (sdAb) comprising an on-rate constant (kₒₙ) of about 3.30×10\hat{ }4−1.70×10\hat{ }7 and an off-rate constant (k_{off}) of about 3.20×10\hat{ }−9−2.20×10\hat{ }−1.

The invention also provides a nucleic acid encoding an anti-C-reactive protein (CRP) single-domain antibody (sdAb) comprising an on-rate constant (kₒₙ) of about 3.30×10\hat{ }4−1.70×10\hat{ }7 and an off-rate constant (k_{off}) of about 3.20×10\hat{ }−9−2.20×10\hat{ }−1.

In another aspect, the invention provides a DNA encoding an anti-C-reactive protein (CRP) single-domain antibody (sdAb) comprising an on-rate constant (kₒₙ) of about 3.30×10\hat{ }4−1.70×10\hat{ }7 and an off-rate constant (k_{off}) of about 3.20×10\hat{ }−9−2.20×10\hat{ }−1.

In another aspect, the invention provides a DNA vector encoding an anti-C-reactive protein (CRP) single-domain antibody (sdAb) comprising an on-rate constant (kₒₙ) of about 3.30×10\hat{ }4−1.70×10\hat{ }7 and an off-rate constant (k_{off}) of about 3.20×10\hat{ }−9−2.20×10\hat{ }−1.

In another aspect, the invention provides a DNA expression vector encoding an anti-C-reactive protein (CRP) single-domain antibody (sdAb) comprising an on-rate constant (kₒₙ) of about 3.30×10\hat{ }4−1.70×10\hat{ }7 and an off-rate constant (k_{off}) of about 3.20×10\hat{ }−9−2.20×10\hat{ }−1.

In another aspect the invention provides a RNA encoding an anti-CRP sdAb of the invention. A sdAb of the present invention can be encoded by e.g. a messenger ribonucleic acid (mRNA) which is typically a single-stranded molecule of RNA that can be used by a ribosome for peptide synthesis. In the sense of the present invention a RNA encoding an anti-CRP sdAb of the invention is preferably a mRNA. The mRNA encoding an anti-CRP sdAb of the invention is delivered to a host cell where it is translated into an anti-CRP sdAb or can be used in in vitro transcription. In general, any mRNA construct encoding an anti-CRP sdAb that is capable of expressing an anti-CRP sdAb can be used in the sense of the present invention.

Accordingly, the invention provides a RNA encoding an anti-C-reactive protein (CRP) single-domain antibody (sdAb) comprising an on-rate constant (kₒₙ) of about 3.30×10\hat{ }4−1.70×10\hat{ }7 and an off-rate constant (k_{off}) of about 3.20×10\hat{ }−9−2.20×10\hat{ }−1.

In another aspect, the invention provides a mRNA encoding an anti-C-reactive protein (CRP) single-domain antibody (sdAb) comprising an on-rate constant (kₒₙ) of about 3.30×10\hat{ }4−1.70×10\hat{ }7 and an off-rate constant (k_{off}) of about 3.20×10\hat{ }−9−2.20×10\hat{ }−1.

The skilled person is aware that, except where otherwise noted, polynucleotide sequences set forth in the present application will recite “T”s in a representative DNA sequence but where the sequence represents RNA (e.g., mRNA), the “T”s would be substituted for “U”s. Thus, any of the RNA polynucleotides encoded by a DNA identified by a particular sequence identification number may also comprise the corresponding RNA (e.g., mRNA) sequence encoded by the DNA, where each “T” of the DNA sequence is substituted with “U”.

A mRNA can comprise at least an open reading frame encoding an anti-CRP sdAb of the present invention. The basic components of an mRNA molecule typically include at least one coding regions, a 5′ untranslated region (UTR), a 3′ UTR, a 5′ cap and a poly-A tail. Polynucleotides of the present invention may function as mRNA but can be distinguished from wild-type mRNA in their functional and/or structural design features which serve to overcome existing problems of effective polypeptide expression e.g. when using nucleic-acid based therapeutics or when used for optimized expression/production of sdAbs. The mRNAs of the present invention may be codon optimized for expression in a host, host cell or organism. The nucleic acid, such as a RNA, of the present invention can be present in a host cell, e.g. for the purpose of expressing or producing a sdAb of the invention. Accordingly, the invention provides a host cell comprising a RNA encoding an anti-C-reactive protein (CRP) single-domain antibody (sdAb) comprising an on-rate constant (kₒₙ) of about 3.30×10\hat{ }4−1.70×10\hat{ }7 and an off-rate constant (k_{off}) of about 3.20×10\hat{ }−9−2.20×10\hat{ }−1.

The sample and/or biological fluid to be used and/or analyzed herein preferably have a physiological pH, e.g. about 7.0 to about 7.5, preferably about 7.2 to 7.4. For example, the pH of (arterial) blood normally is within a range of about 7.35−7.45. Intracellular pH (e.g. pH of interstitial fluid) can range between about 7.03 to 7.46.

It is preferred herein that he sample and/or biological fluid is blood or interstitial fluid.

The invention also provides a method for detecting and/or quantifying CRP, e.g. in a sample. Such a method makes use of the sdAbs provided herein which specifically bind CRP within the entire dynamic range of the CRP level. As described herein above, it is especially useful to detect and/or quantify CRP levels in a sample to evaluate the inflammation status of a subject or monitor efficacy and tolerability of a treatment. Such a method can comprise contacting a sample with an anti-CRP sdAb of the invention and detecting and/or quantifying binding of the anti-CRP sdAb. A sample used in the sense of the present invention can be any sample that allows for detection and/or quantification of CRP levels. A sample can be for example wherein a biological fluid, such as a bodily fluid. Exemplary samples are interstitial fluid (ISF), blood, urine, stool, serum, tear fluid, sweat, saliva and the like.

Accordingly, the invention provides in one aspect a method for detecting and/or quantifying CRP in a sample, the method comprising:
(i) contacting a sample with the anti-CRP sdAb of the invention, and
(ii) detecting binding of the anti-CRP sdAb,
wherein the sample comprises interstitial fluid (ISF), blood, urine, stool, serum, tear fluid, sweat, or saliva.

Similarly, the invention is not limited to means of contacting a sample, any means that are feasible for contacting an CRP with an anti-CRP sdAb of the invention can be used. Exemplary means of contacting a sample with an anti-CRP sdAb of the invention are collection through a finger stick/prick or syringe and deposition on the anti-CRP sdAb and/or biosensor, collection through microfluidic or capillary-based system to the anti-CRP sdAb and/or biosensor surface, collection through needle or microneedle-based system, direct implantation of the biosensor comprising the anti-CRP sdAb in contact with the bodily fluid of interest. Other means of contacting a sample with an anti-CRP sdAb and/or biosensor of the invention comprise determining a body site where to collect a sample (e.g. bodily fluid) from (ex vivo) and/or contacting a sample (e.g. body fluid) with the anti-CRP sdAb and/or the biosensor (in vivo). In the sense of the present invention, a sample may be contacted with an anti-CRP sdAb conjugated to a working electrode in a biosensor. Similarly, a sdAb conjugated to a working electrode in a biosensor may be contacted with an CRP or a sample comprising CRP.

Accordingly, the invention provides in one aspect a method for detecting and/or quantifying CRP in a sample, the method comprising:
(i) contacting a sample with the anti-CRP sdAb of the invention, and
(ii) detecting binding of the anti-CRP sdAb,
wherein the contacting step comprises contacting a sample via a syringe, a needle, a microfluidic or capillary-based system with the anti-CRP sdAb of the invention.

In another aspect, the invention provides a method for detecting and/or quantifying CRP in a sample, the method comprising:
(i) contacting a sample with the anti-CRP sdAb of the invention, and
(ii) detecting binding of the anti-CRP sdAb,
wherein the contacting step comprises contacting the sample with a biosensor of the invention, optionally wherein the biosensor comprises a sdAb of the invention conjugated to a working electrode.

In general, the invention is not limited to any means of detecting a sdAb, an CRP and/or an CRP bound by a sdAb of the present invention. Any means allowing for detection/quantification can be used in the sense of the invention. Exemplary antibody-based detection methods are well known in the art, and include, for example, ELISA, electrochemical ELISA, immunoblots, radioimmunoassays, immunofluorescence, immunoprecipitation, and other related techniques. Association of a sdAb of the invention and CRP can also be physiochemically such as electrochemically detected/quantified using physiochemical or electrochemical techniques as described herein. The electric signal that is generated can be readout by any electrochemical techniques commonly used, such as amperometry, chronoamperometry, potentiometry, conductometry, voltammetry, cyclic voltammetry, galvanic cell, impedance spectroscopy, and the like resulting in detection/quantification of sdAb and CRP association. It is preferred herein, that CRP levels are detected/quantified making use of the biosensor as described herein above. Accordingly, the method for detecting and/or quantifying CRP in a sample can be performed using a biosensor as described herein, such as a biosensor comprising a sdAb of the invention conjugated to a working electrode.

Accordingly, the invention provides in one aspect a method for detecting and/or quantifying CRP in a sample, the method comprising:
(i) contacting a sample with the anti-CRP sdAb of the invention, and
(ii) detecting binding of the anti-CRP sdAb,
wherein the detection step comprises physiochemical or electrochemical detection of the binding of the anti-CRP sdAb and CRP.

In another aspect, the invention provides a method for detecting and/or quantifying CRP in a sample, the method comprising:
(i) contacting a sample with the anti-CRP sdAb of the invention, and
(ii) detecting binding of the anti-CRP sdAb,
wherein the detection step comprises detection of the binding of the anti-CRP sdAb and CRP via an electrical signal produced by the binding.

In another aspect, the invention provides a method for detecting and/or quantifying CRP in a sample, the method comprising:
(i) contacting a sample with the anti-CRP sdAb of the invention, and
(ii) detecting binding of the anti-CRP sdAb,
wherein the detection step comprises detection of the binding of the anti-CRP sdAb and CRP with a biosensor of the invention, optionally, wherein the biosensor comprises a sdAb of the invention conjugated to a working electrode.

In a preferred aspect, the invention provides a method for detecting and/or quantifying CRP in a sample, the method comprising:
(i) contacting a sample with the anti-CRP sdAb of the invention, and
(ii) detecting binding of the anti-CRP sdAb,
wherein the contacting step comprises contacting the sample with a biosensor of the invention, optionally wherein the biosensor comprises a sdAb of the invention conjugated to a working electrode and,
wherein the detection step comprises detecting the binding of the binding of the anti-CRP sdAb and CRP with a biosensor of the invention, optionally, wherein the biosensor comprises a sdAb of the invention conjugated to a working electrode.

Electrochemical ELISA can be used as an exemplary means for detecting and/or quantifying CRP or in a detecting unit for detecting and/or quantifying CRP. In brief, the anti-CRP sdAb can be directly or indirectly immobilized/conjugated to a working electrode. The working electrode can be incubated with samples (e.g. interstitial fluid (ISF), blood, urine, stool, serum, tear fluid, sweat, or saliva) to be tested for CRP levels. During incubation, CRP can bind to the anti-CRP sdAb. After incubation, the working electrode can be washed, e.g. with phosphate buffered saline (PBS). A solution with a secondary antibody (i.e. antibody binding the anti-CRP sdAb) modified with biotin can be added and incubated, followed by another washing step of the working electrode. A solution containing streptavidin modified enzyme, such as alkaline phosphatase (ALP), polymeric alkaline phosphatease (PALP), or Horseradish Peroxidase (HRP), can be added to the working electrode, followed by another washing step of the working electrode. A solution containing enzyme substrate such as 4-APP, ABTS, AEC, DAB, ECL, TMB, Amplex Red, BCIP/NBT (5-Bromo-4-chloro-3-indolyl phosphate along with nitro blue tetrazolium), pNPP (para-Nitrophenylphosphate), or Fast Red, can then be added, and the electrochemical oxidation signal can be recorded, e.g. by using differential pulse voltammetry. An alternative approach of applying electrochemical ELISA is to directly or indirectly immobilize/conjugate the anti-CRP sdAb to a working electrode. The working electrode can be incubated with samples (e.g. interstitial fluid (ISF), blood, urine, stool, serum, tear fluid, sweat, or saliva) to be tested for CRP levels. During incubation, CRP can bind to the anti-CRP sdAb. After incubation, the working electrode can be washed, e.g. with phosphate buffered saline (PBS). A solution with a secondary antibody (i.e. antibody binding the anti-CRP sdAb) modified with an enzyme, such as alkaline phosphatase (ALP), polymeric alkaline phosphatease (PALP), or Horseradish Peroxidase (HRP), can be added and incubated, followed by another washing step of the working electrode. A solution containing enzyme substrate such as 4-APP, ABTS, AEC, DAB, ECL, TMB, Amplex Red, BCIP/NBT (5-Bromo-4-chloro-3-indolyl phosphate along with nitro blue tetrazolium), pNPP (para-Nitrophenylphosphate), or Fast Red, can then be added, and the electrochemical oxidation signal can be recorded, e.g. by using differential pulse voltammetry.

Biosensing via electrochemical ELISA is for example disclosed in Arya, Sunil K., and Pedro Estrela. “Electrochemical ELISA protein biosensing in undiluted serum using a polypyrrole-based platform.” Sensors 20.10 (2020): 2857.

Accordingly, the invention provides in one aspect, a method for detecting and/or quantifying CRP in a sample, the method comprising:
(i) contacting a sample with the anti-CRP sdAb of the invention, and
(ii) detecting binding of the anti-CRP sdAb,
wherein the detection step comprises detection of the binding of the anti-CRP sdAb and CRP via electrochemical ELISA.

The method for detecting and/or quantifying CRP in a sample as described above can be ex vivo or in vitro. Accordingly, the invention provides in one aspect an ex vivo or in vitro method for detecting and/or quantifying CRP in a sample, the method comprising:
(i) contacting a sample with an anti-CRP sdAb of the present invention, and
(ii) detecting binding of the anti-CRP sdAb.

In some aspects of the invention, the methods described herein can further comprise quantifying the CRP level in a sample or a subject and comparing the CRP level with a reference sample or value. A reference sample may be a sample comprising an CRP level corresponding to any specific inflammation conditions, such as inactive and active IBD, COVID, cancer, sepsis, and septic shock. A reference sample may be a standardized sample, i.e. a sample comprising CRP levels that have been standardized to certain CRP levels. Such CRP levels may correspond to values or ranges of CRP levels indicating e.g. baseline inflammation, low inflammation, moderate inflammation, high inflammation or severe inflammation in a sample or subject. Accordingly, the CRP level in a sample or a subject may also be compared to a CRP level value. An CRP level value can be obtained from a reference sample as described herein or e.g. from the literature. CRP level reference sample or values and thereby the inflammation status they indicate may vary dependent on the subject, the disease a subject may be suffering from and/or the treatment a subject is receiving. The skilled practitioner is aware and knows what reference samples or values can be used to properly assess a subject's inflammation status. Exemplary reference values may be obtained from Devran et al., 2012, Dinh et al., 2019, Hart et al., 2020, Herieka & Erridge, 2014, Keegan et al., 2020, Landry et al., 2017, Mavropoulou et al., 2020, Póvoa et al., 1998, Ridker et al., 2003, Ruiz-González et al., 2016, Sproston & Ashworth, 2018 . . .

For example, the following reference values may be used:

TABLE 1
CRP concentrations in humans during
homeostasis and disease.

| | Concentration in a | |
|---|---|---|
| | biological fluid, | Example |
| Range | such as blood | indications/disease |
| Baseline | <0.01 μM | |
| Low | 0.01 μM-0.1 μM | Metabolic Syndrome |
| elevation | | (incl obesity) |
| | | CVD risk (low: 1 mg/ml, |
| | | interm: 1-3, high: >3) |
| | | Low self rated |
| | | physical health score |
| | | Active IBD |
| Moderate | 0.1 μM-1 μM | Cholecystitis |
| elevation | | Chronic obstructive |
| | | pulmonary disease (COPD) |
| | | Acute bronchitis |
| | | Sepsis |
| | | Active advanced cancer, |
| | | metastasis (>50 mg/ml) |
| | | Prediction of |
| | | Immunotherapy |
| | | efficiency |
| Severe | >1 μM | Pancreatitis |
| elevation | | Pneumonia |
| | | Sepsis related mortality |
| | | Severe infection (lung, |
| | | urinary, skin, GI, blood), |
| | | Rheumatologic causes |
| | | and/or Advanced tumors |

Detection and/or quantification of CRP in a sample as described herein above can for example be realized by a spot measurement of the CRP levels making use of the sdAb of the present invention. For example, the method for detecting and/or quantifying CRP can comprise obtaining a sample from a subject, contacting the sample with an anti-CRP sdAb of the invention, and detecting binding of the anti-CRP sdAb. The method can further comprise quantifying the CRP level in the sample, which correlates with the binding kinetics between sdAb and CRP. In an exemplary embodiment, a spot measurement can be realized in the method for detecting and/or quantifying CRP comprising obtaining a sample from a subject e.g. obtaining a drop of blood by fingerpick, contacting the sample with an anti-CRP sdAb of the invention e.g. collecting the sample through capillary mechanisms or deposition onto an anti-CRP sdAb or a biosensor comprising the same as disclosed herein, detecting binding of the anti-CRP sdAb, thereby quantifying the CRP level in the sample and the subject. In some embodiments of the method, the anti-CRP sdAb may be conjugated to a working electrode in a biosensor as described herein.

Accordingly, the invention provides a method for detecting and/or quantifying CRP in a sample, the method comprising:
(i) contacting a sample with the anti-CRP sdAb of the invention, and
(ii) detecting binding of the anti-CRP sdAb.

The method can be repeated for samples from individual subjects. This is particularly desired when the development or dynamics of the CRP levels in a subject are relevant for a therapy of the subject. Rising levels of CRP over time correlate with increased inflammation, while declining levels of CRP over time correlate with reduced inflammation. For example, a rising level of CRP can indicate an infectious disease. Particularly, the CRP levels can be useful to indicate whether a subject suffers from a bacterial or viral infection. CRP levels are elevated in subjects suffering from infection, when treated with antibiotics, CRP levels will decline if the subject suffers from a bacterial infection. In contrast, when treated with antibiotics, CRP levels will not decline if the subject suffers from viral infection. Accordingly, it is especially useful to monitor the CRP levels in a subject over time, e.g. to arrive at a clinical conclusion of the best treatment of a subject. By repeating the spot measurement as described above, the dynamic CRP level in a subject can be detected over time allowing the skilled practitioner to make reasoned treatment decisions, e.g. in case of bacterial vs. viral infection. In addition, CRP levels can correlate with inflammation spikes in inflammatory diseases and/or autoimmune diseases. Exemplary inflammatory and/or autoimmune diseases in the sense of the invention are rheumatoid arthritis, inflammatory bowel disease, IgA nephropathy, Psoriasis, lupus, multiple sclerosis, Endometriosis, Sepsis, cardiovascular disease, metabolic syndrome, hypertension, diabetes, cancer, bacterial or viral infections, COVID19, asthma, familial Mediterranean fever, Alzheimer's disease, Parkinson's diseases, diabetic macular edema (DME), diabetic retinopathy, uveitis, glaucoma, dry eye (e.g., dry eye disease or dry eye syndrome), allergic conjunctivitis, ocular pain, rhegmatogenous retinal detachment (RRD), age-related macular degeneration (AMD), proliferative diabetic retinopathy (PDR), retinal vein occlusion (RVO), neuromyelitis optica (NMO), corneal transplant, corneal abrasion, physical injury to the eye, non-alcoholic fat liver disease, chronic kidney disease, tumour and the like. Inflammatory and/or autoimmune diseases may require close medical attention and medication. During the course of an inflammatory and/or autoimmune disease it is especially useful to monitor the CRP levels in a subject. By doing so, inflammation spikes can be detected enabling targeted therapy and medication which reduces the total amount of administered medication needed and thereby potential side effects compared to a continuous treatment. Accordingly, the detection and/or quantification of the CRP levels in a subject enables the skilled practitioner to modulate and personalize the treatment of a subject while reducing the total amount of drugs administered, particularly, if the CRP levels are detected/quantified over time. Thus, the detection and/or quantification of CRP levels over time is desired for improved disease management. In some embodiments, the method for detecting and/or quantifying CRP in a sample does not comprise a step of obtaining a sample from a subject.

Accordingly, in one aspect, the invention provides a method for detecting and/or quantifying CRP in a sample, the method comprising:
(i) contacting a sample with the anti-CRP sdAb of the invention, and
(ii) detecting binding of the anti-CRP sdAb,
further comprising detecting the binding of the anti-CRP sdAb over time.

In another aspect, the invention provides a method for detecting and/or quantifying CRP in a sample, the method comprising:
(i) contacting a sample with the anti-CRP sdAb of the invention, and
(ii) detecting binding of the anti-CRP sdAb,
further comprising detecting the binding of the anti-CRP sdAb over time, wherein steps (i) and
(ii) are performed on a further sample, optionally wherein the sample is from the same subject.

In yet another aspect, the invention provides a method for detecting and/or quantifying CRP in a sample, the method comprising:
(i) contacting a sample with the anti-CRP sdAb of the invention, and
(ii) detecting binding of the anti-CRP sdAb,
further comprising contacting and/or detecting the binding of the anti-CRP sdAb over time, wherein steps (i) and (ii) are performed on a further sample, optionally wherein the sample is from the same subject, and
wherein the contacting step comprises contacting the sample with a contacting a sample with a biosensor of the invention, optionally wherein the biosensor comprises a sdAb of the invention conjugated to a working electrode, and
wherein the detection step comprises detecting the binding of the binding of the anti-CRP sdAb and CRP with a biosensor of the invention, optionally, wherein the biosensor comprises a sdAb of the invention conjugated to a working electrode.

In another preferred aspect, the invention provides a method for detecting and/or quantifying CRP in a sample, the method comprising:
(i) contacting a sample with the anti-CRP sdAb of the invention, and
(ii) detecting binding of the anti-CRP sdAb,
further comprising contacting and/or detecting the binding of the anti-CRP sdAb over time,
wherein steps (i) and (ii) are performed on a further sample, optionally wherein the sample is from the same subject, and
wherein the contacting step comprises contacting the sample with a contacting a sample with a biosensor of the invention, optionally wherein the biosensor comprises a sdAb of the invention conjugated to a working electrode, and
wherein the detection step comprises detecting the binding of the binding of the anti-CRP sdAb and CRP with a biosensor of the invention, optionally, wherein the biosensor comprises a sdAb of the invention conjugated to a working electrode.

In another preferred aspect, the invention provides a method for detecting and/or quantifying CRP in a sample, the method comprising:
(i) contacting a sample comprising interstitial fluid (ISF), blood, urine, stool, serum, tear fluid, sweat, or saliva with the anti-CRP sdAb of the invention, and
(ii) detecting binding of the anti-CRP sdAb,
further comprising contacting and/or detecting the binding of the anti-CRP sdAb over time, wherein steps (i) and (ii) are performed on a further sample, optionally wherein the sample is from the same subject, and
wherein the contacting step comprises contacting the sample with a contacting a sample with a biosensor of the invention, optionally wherein the biosensor comprises a sdAb of the invention conjugated to a working electrode, and
wherein the detection step comprises detecting the binding of the binding of the anti-CRP sdAb and CRP with a biosensor of the invention, optionally, wherein the biosensor comprises a sdAb of the invention conjugated to a working electrode.

In another preferred aspect, the invention provides a method for detecting and/or quantifying CRP in a sample, the method comprising:
(i) contacting a sample comprising interstitial fluid (ISF), blood, urine, stool, serum, tear fluid, sweat, or saliva with the anti-CRP sdAb of the invention, and
(ii) detecting binding of the anti-CRP sdAb,
wherein the detection step comprises detection of the binding of the anti-CRP sdAb and CRP via electrochemical ELISA.

It is also envisioned herein to use more than one sdAb of the invention in a method for detecting and/or quantifying CRP in a sample. Two or more anti-CRP sdAb can be used to combine specific desired ranges of on-rate constant (kₒₙ) and off-rate constant (k_{off}) to arrive at the desired association and dissociation dynamics. Accordingly, the method for detecting and/or quantifying CRP in a sample of the present invention can comprise contacting a sample with one or more, such as one, two, three, four, five, six, seven, eight, nine, ten, eleven or twelve anti-CRP sdAb of the present invention. In some embodiments, the method for detecting and/or quantifying CRP in a sample of the present invention can comprise contacting a sample with two or more, such as two, three, four, five, six, seven, eight, nine, ten, eleven or twelve anti-CRP sdAb of the present invention.

Accordingly, the invention provides in one aspect a method for detecting and/or quantifying CRP in a sample, the method comprising:
(i) contacting a sample with one or more anti-CRP sdAb of the invention, and
(ii) detecting binding of the one or more anti-CRP sdAb.

In another aspect, the invention provides a method for detecting and/or quantifying CRP in a sample, the method comprising:
(i) contacting a sample with one or more, such as one, two, three, four, five, six, seven, eight, nine, ten, eleven or twelve anti-CRP sdAbs of the invention, and
(ii) detecting binding of the one or more, such as one, two, three, four, five, six, seven, eight, nine, ten, eleven or twelve anti-CRP sdAbs.

In another aspect, the invention provides a method for detecting and/or quantifying CRP in a sample, the method comprising:
(i) contacting a sample with two or more anti-CRP sdAb of the invention, and
(ii) detecting binding of the two or more anti-CRP sdAb.

In another aspect, the invention provides a method for detecting and/or quantifying CRP in a sample, the method comprising:
(i) contacting a sample with two or more, such as two, three, four, five, six, seven, eight, nine, ten, eleven or twelve anti-CRP sdAb of the invention, and
(ii) detecting binding of the two or more, such as two, three, four, five, six, seven, eight, nine, ten, eleven or twelve anti-CRP sdAb.

As described herein, a particular advantage of the sdAb of the invention is the reversible and continuous binding of CRP. Spot measurements as described herein are particularly useful to determine levels of CRP. In another aspect, it is also desired to continuously monitor the levels of CRP in a subject, e.g. avoiding obtaining samples from a subject at multiple time points. Reducing the sample collection steps, e.g. blood draws on a subject, is especially advantageous, since it reduces treatment burden on the subject. For this, the measurement of CRP levels can be in or on a subject, i.e. in vivo.

Accordingly, in one aspect the invention provides a method for detecting and/or quantifying CRP in a subject, the method comprising:
(i) contacting a subject with the anti-CRP sdAb of the invention, and
(ii) detecting binding of the anti-CRP sdAb.

In general, the features provided for the method for detecting and/or quantifying CRP in a sample herein above can be applied to the method for detecting and/or quantifying CRP in a subject. In some aspects, it is not necessary to obtain a sample from a subject when a subject's interstitial fluid (ISF), blood, urine, stool, serum, tear fluid, sweat, saliva and the like is contacted with an anti-CRP sdAb of the invention. When the CRP level is detected on or in the subject, a subject's interstitial fluid (ISF), blood, urine, stool, serum, tear fluid, sweat, saliva and the like may be contacted directly with an anti-CRP sdAb of the invention, or e.g. a biosensor comprising a sdAb of the invention conjugated to a working electrode. The invention is not particularly limited to any means of contacting, rather any means of contacting that allow a sdAb of the invention to come in close proximity with a CRP of the subject (i.e. that binding/association of an anti-CRP sdAb of the invention and an CRP of the subject is realized) can be used. Exemplary forms of contacting a subject's interstitial fluid (ISF), blood, urine, stool, serum, tear fluid, sweat, saliva and the like can be via a syringe, a needle, a microfluidic or capillary-based system or the like.

In one aspect, the invention provides a method for detecting and/or quantifying CRP in a subject, the method comprising:
(i) contacting a subject's interstitial fluid (ISF), blood, urine, stool, serum, tear fluid, sweat, or saliva with an anti-CRP sdAb of the invention, and
(ii) detecting binding of the anti-CRP sdAb.

In another aspect, the invention provides a method for detecting and/or quantifying CRP in a subject, the method comprising:
(i) contacting a subject's interstitial fluid (ISF) with an anti-CRP sdAb of the invention, and
(ii) detecting binding of the anti-CRP sdAb.

In the sense of the present invention, a subject may be contacted with an anti-CRP sdAb conjugated to a working electrode in a biosensor. Similarly, a sdAb conjugated to a working electrode in a biosensor may be contacted with a subject's interstitial fluid (ISF), blood, urine, stool, serum, tear fluid, sweat, saliva and the like comprising CRP.

Accordingly, the invention provides in one aspect a method for detecting and/or quantifying CRP in a subject, the method comprising:
(i) contacting a subject with the anti-CRP sdAb of the invention, and
(ii) detecting binding of the anti-CRP sdAb,
wherein the contacting step comprises contacting the subject with a biosensor of the invention, optionally wherein the biosensor comprises a sdAb of the invention conjugated to a working electrode.

As described herein above, the method for detecting and/or quantifying CRP can be performed using a biosensor as described herein, e.g. a biosensor comprising a sdAb of the invention conjugated to a working electrode. This can also be applied to the method for detecting and/or quantifying CRP in a subject of the invention.

Accordingly, in another aspect the invention provides a method for detecting and/or quantifying CRP in a subject, the method comprising:
(i) contacting a subject with the anti-CRP sdAb of the invention, and
(ii) detecting binding of the anti-CRP sdAb,
wherein the contacting step comprises contacting the subject with a biosensor of the invention, optionally wherein the biosensor comprises a sdAb of the invention conjugated to a working electrode, and
wherein the detection step comprises detecting the binding of the binding of the anti-CRP sdAb and CRP with a biosensor of the invention, optionally, wherein the biosensor comprises a sdAb of the invention conjugated to a working electrode.

In another aspect the invention provides a method for detecting and/or quantifying CRP in a subject, the method comprising:
(i) contacting a subject with the anti-CRP sdAb of the invention, and
(ii) detecting binding of the anti-CRP sdAb,
wherein the detection step comprises detection of the binding of the anti-CRP sdAb and CRP via electrochemical ELISA.

The method of the invention for detecting and/or quantifying CRP in a subject, can further comprise quantifying the CRP level in a subject and comparing the CRP level with a reference sample or value. Thus, the statements described herein above regarding further comprising quantifying the CRP level and comparing the CRP level with a reference sample or value also apply to the method of the invention for detecting and/or quantifying CRP in a subject.

Similarly, to the spot type measurements in a sample as described above, the invention also provides a means to measure the CRP levels in or on a subject over time, i.e. a method for detecting and/or quantifying CRP in a subject over time. Measuring CRP levels over time is especially advantageous when done in or on a subject since no additional samples have to be obtained from the subject reducing the burden on the subject. This allows to reduce the time steps in which the CRP levels are measured in the subject resulting in a superior resolution of the dynamic CRP level in the subject over time. The thereby increased resolution of the dynamic CRP levels in the subject allow the skilled practitioner to precisely modulate and personalize the treatment of a subject while reducing the total amount of drugs administered, particularly. Thus, the detection and/or quantification of CRP levels over time in a subject result in improved disease management. The method for detecting and/or quantifying CRP in or on a subject as described herein can be realizes within a device or apparatus in form of an applicator like appliance, such as a handheld or stationary device comprising the biosensor of the present invention, or in form of a permanently or reversibly implanted or attached (to the subject's body) like appliance, such as an implanted, wearable device comprising the biosensor of the present invention. As used herein “detecting binding of the anti-CRP sdAb to CRP” in the context of a sdAb comprised in a biosensor or apparatus of the present invention can include or be “detecting an electrochemical signal (optionally where detection of said signal indicates the presence of CRP and/or wherein the signal strength indicates the amount of CRP)”. Devices and apparatuses useful for performing the methods provided herein are described below in more detail.

Accordingly, in one aspect the invention provides a method for detecting and/or quantifying CRP in a subject over time, the method comprising:
(i) contacting a subject with the anti-CRP sdAb of the invention, and
(ii) detecting binding of the anti-CRP sdAb.

In another aspect, the invention provides a method for detecting and/or quantifying CRP in a subject, the method comprising:
(i) contacting a subject with the anti-CRP sdAb of the invention, and
(ii) detecting binding of the anti-CRP sdAb,
further comprising contacting and/or detecting the binding of the anti-CRP sdAb over time,
wherein steps (i) and (ii) are repeatedly performed in or on the subject.

In a preferred aspect, the invention provides a method for detecting and/or quantifying CRP in a subject, the method comprising:
(i) contacting a subject with the anti-CRP sdAb of the invention, and
(ii) detecting binding of the anti-CRP sdAb,
further comprising contacting and/or detecting the binding of the anti-CRP sdAb over time,
wherein steps (i) and (ii) are repeatedly performed in or on the subject, and
wherein the contacting step comprises contacting the subject with a biosensor of the invention, optionally wherein the biosensor comprises a sdAb of the invention conjugated to a working electrode, and
wherein the detection step comprises detecting the binding of the binding of the anti-CRP sdAb and CRP with a biosensor of the invention, optionally, wherein the biosensor comprises a sdAb of the invention conjugated to a working electrode.

In another preferred aspect, the invention provides a method for detecting and/or quantifying CRP in a subject, the method comprising:
(i) contacting a subject's interstitial fluid (ISF), blood, urine, stool, serum, tear fluid, sweat, or saliva with the anti-CRP sdAb of the invention, and
(ii) detecting binding of the anti-CRP sdAb,
further comprising contacting and/or detecting the binding of the anti-CRP sdAb over time, wherein steps (i) and (ii) are repeatedly performed in or on the subject, and
wherein the contacting step comprises contacting the subject with a biosensor of the invention, optionally wherein the biosensor comprises a sdAb of the invention conjugated to a working electrode, and
wherein the detection step comprises detecting the binding of the binding of the anti-CRP sdAb and CRP with a biosensor of the invention, optionally, wherein the biosensor comprises a sdAb of the invention conjugated to a working electrode.

It is also envisioned herein to use more than one sdAb of the invention in a method for detecting and/or quantifying CRP in a subject. Two or more anti-CRP sdAb can be used to combine specific desired ranges of on-rate constant (kₒₙ) and off-rate constant (k_{off}) to arrive at the desired association and dissociation dynamics. Accordingly, the method for detecting and/or quantifying CRP in a subject of the present invention can comprise contacting a subject with one or more, such as one, two, three, four, five, six, seven, eight, nine, ten, eleven or twelve anti-CRP sdAb of the present invention. In some embodiments, the method for detecting and/or quantifying CRP in a subject of the present invention can comprise contacting a subject with two or more, such as two, three, four, five, six, seven, eight, nine, ten, eleven or twelve anti-CRP sdAb of the present invention.

Accordingly, the invention provides in one aspect a method for detecting and/or quantifying CRP in a subject, the method comprising:
(i) contacting a subject with one or more anti-CRP sdAb of the invention, and
(ii) detecting binding of one or more anti-CRP sdAb.

In another aspect, the invention provides a method for detecting and/or quantifying CRP in a subject, the method comprising:
(i) contacting a subject with one or more, such as one, two, three, four, five, six, seven, eight, nine, ten, eleven or twelve anti-CRP sdAb of the invention, and
(ii) detecting binding of one or more, such as one, two, three, four, five, six, seven, eight, nine, ten, eleven or twelve anti-CRP sdAb.

In another aspect, the invention provides a method for detecting and/or quantifying CRP in a subject, the method comprising:
(i) contacting a subject with two or more anti-CRP sdAb of the invention, and
(ii) detecting binding of two or more anti-CRP sdAb.

In another aspect, the invention provides a method for detecting and/or quantifying CRP in a subject, the method comprising:
(i) contacting a subject with two or more, such as two, three, four, five, six, seven, eight, nine, ten, eleven or twelve anti-CRP sdAb of the invention, and
(ii) detecting binding of two or more, such as two, three, four, five, six, seven, eight, nine, ten, eleven or twelve anti-CRP sdAb.

It is also encompassed herein that a level of CRP can be detected and/or quantified in sample from a subject or in a subject in its original place, i.e. at the original location of the sample or at the location in a subject (in situ). Thus, the anti-CRP sdAb, the biosensor comprising the same and the apparatuses comprising the same of the present invention are particularly useful for in situ detection and/or quantification of CRP. An in situ method as disclosed herein can comprise or be an in vivo method. Accordingly, the invention provides in one aspect an in situ method for detecting and/or quantifying CRP in a subject, the method comprising:
(i) contacting a subject's biological fluid or sample e.g. a bodily fluid with an anti-CRP sdAb of the invention, and
(ii) detecting binding of the anti-CRP sdAb.

In another aspect, the invention provides an in situ method for detecting and/or quantifying CRP in a subject, the method comprising:
(i) contacting a subject's bodily fluid e.g. interstitial fluid (ISF), blood, urine, stool, serum, tear fluid, sweat or saliva with an anti-CRP sdAb of the invention, and
(ii) detecting binding of the anti-CRP sdAb.

In general, the features provided for the method for detecting and/or quantifying CRP in a subject herein above can also be applied mutatis mutandis to the in situ method for detecting and/or quantifying CRP in a subject.

The features described herein in context of the method for detecting and/or quantifying CRP in a sample apply mutatis mutandis to the method for detecting and/or quantifying CRP in a subject. In some aspect, the method for detecting and/or quantifying CRP in a subject may not comprise a step of obtaining a sample from the subject.

The methods for detecting and/or quantifying CRP in a sample or in/on a subject can be performed for example in an apparatus or performed by an apparatus. An apparatus comprising components for performing the methods described herein is especially useful for efficient and quick measurement of CRP levels in a sample or in/on a subject. Thus, an apparatus according to the present invention can be used in any spot measurement or any continuous measurement of CRP as described herein. An apparatus of the present invention is not particularly limited, in general, an apparatus comprising an anti-CRP sdAb of the invention and any means of measuring/detecting/quantifying association/binding of the anti-CRP sdAb of the invention to CRP can be used.

The apparatus of the present invention can comprise one or more anti-CRP sdAb of the present invention. Using more than one sdAb can be preferred herein to take advantage of the individual properties of anti-CRP sdAb provided herein. For example, a biosensor or apparatus provided herein comprising two or more anti-CRP sdAb can be used to combine specific desired ranges of on-rate constant (kₒₙ) and off-rate constant (k_{off}) to arrive at the desired association and dissociation dynamics. In some embodiments, the apparatus of the present invention comprises one or more, such as one, two, three, four, five, six, seven, eight, nine, ten, eleven or twelve anti-CRP sdAb of the present invention. In some embodiments, the biosensor of the present invention comprises one or more, such as one, two, three, four, five, six, seven, eight, nine, ten, eleven or twelve anti-CRP sdAb of the present invention. In some embodiments, one or more, such as one, two, three, four, five, six, seven, eight, nine, ten, eleven or twelve anti-CRP sdAb of the present invention can be used in any of the products, methods or uses of the present invention.

Accordingly, the invention provides in one aspect an apparatus comprising:
(i) an anti-CRP sdAb of the invention, and
(ii) a contacting unit for contacting a sample with the anti-CRP sdAb, and
(iii) a detecting unit for detecting and/or quantifying binding of the anti-CRP sdAb.

In another aspect, the invention provides an apparatus comprising:
(i) an anti-CRP sdAb of the invention, and
(ii) a contacting unit for contacting a subject with the anti-CRP sdAb, and
(iii) a detecting unit for detecting and/or quantifying binding of the anti-CRP sdAb.

In another aspect, the invention provides an apparatus comprising:
(i) one or more, such as one, two, three, four, five, six, seven, eight, nine, ten, eleven or twelve anti-CRP sdAb of the invention, and
(ii) a contacting unit for contacting a sample with the one or more anti-CRP sdAb, and
(iii) a detecting unit for detecting and/or quantifying binding of the one or more anti-CRP sdAb.

In another aspect, the invention provides an apparatus comprising:
(i) one or more, such as one, two, three, four, five, six, seven, eight, nine, ten, eleven or twelve anti-CRP sdAb of the invention, and
(ii) a contacting unit for contacting a subject with the one or more anti-CRP sdAb, and
(iii) a detecting unit for detecting and/or quantifying binding of the one or more anti-CRP sdAb.

The detecting unit for detecting and/or quantifying binding of the anti-CRP sdAb can comprise for example a contacting unit for contacting a sample or a subject with the anti-CRP sdAb of the invention. Such means can be comprised in a first unit which allows a sample or a subject to be contacted. As described above, exemplary forms of contacting a sample or a subject can be via a syringe, a needle, a microfluidic or capillary-based system or the like. A sample to be contacted with the first unit can comprise interstitial fluid (ISF), blood, urine, stool, serum, tear fluid, sweat, saliva and the like. The contacting step is in a subject, a subject's interstitial fluid (ISF), blood, urine, stool, serum, tear fluid, sweat, saliva and the like can be contacted by the first unit. In some embodiments it is preferred that a sample comprises interstitial fluid or blood. In some embodiments it is preferred that a subject's interstitial fluid or blood is contacted.

The detecting unit for detecting and/or quantifying binding of the anti-CRP sdAb can comprise any means that allow a quantitative readout of the association of anti-CRP sdAb and CRP in the sample or subject. The detecting unit for detecting and/or quantifying binding of the anti-CRP sdAb can be comprised in a second unit which allows to detect and/or quantify binding of the anti-CRP sdAb. Accordingly, the detecting unit for detecting and/or quantifying binding of the anti-CRP sdAb of the present invention described in the context of the methods herein also apply to the detecting unit for detecting and/or quantifying binding of the anti-CRP sdAb in the apparatus. For example, an anti-CRP sdAb conjugated to a working electrode, as described herein, can be used as a detecting unit for detecting and/or quantifying binding of the anti-CRP sdAb in the apparatus. Similarly, a biosensor as described herein can be used as a detecting unit for detecting and/or quantifying binding of the anti-CRP sdAb in the apparatus. Other exemplary detecting units for detecting and/or quantifying binding of the anti-CRP sdAb in the apparatus can include electrochemical, optical, thermal, electronic, gravimetric, or acoustic means of detection. For example, a detecting unit for detecting and/or quantifying binding of the anti-CRP sdAb to CRP in the apparatus can comprise electrochemical ELISA. In some embodiments, the first and the second units can be combined in a single unit. In this case, the single unit may be the first unit.

Accordingly, the invention provides in one aspect an apparatus comprising:
(i) an anti-CRP sdAb of the invention, and
(ii) a first unit comprising a contacting unit for contacting a sample with the anti-CRP sdAb, and
(iii) a second unit comprising a detecting unit for detecting and/or quantifying binding of the anti-CRP sdAb.

In another aspect, the invention provides an apparatus comprising:
(i) an anti-CRP sdAb of the invention, and
(ii) a first unit comprising a contacting unit for contacting a subject with the anti-CRP sdAb, and
(iii) a second unit comprising a detecting unit for detecting and/or quantifying binding of the anti-CRP sdAb.

In another aspect, the invention provides an apparatus comprising:
(i) an anti-CRP sdAb of the invention, and
(ii) a first unit comprising a contacting unit for contacting a sample with the anti-CRP sdAb and comprising a detecting unit for detecting and/or quantifying binding of the anti-CRP sdAb.

In another aspect, the invention provides an apparatus comprising:
(i) an anti-CRP sdAb of the invention, and
(ii) a first unit comprising a contacting unit for contacting a subject with the anti-CRP sdAb and comprising a detecting unit for detecting and/or quantifying binding of the anti-CRP sdAb.

In another aspect, the invention provides an apparatus comprising:
(i) an anti-CRP sdAb of the invention, and
(ii) a first unit comprising a contacting unit for contacting a sample comprising interstitial fluid (ISF), blood, urine, stool, serum, tear fluid, sweat, or saliva with the anti-CRP sdAb, and
(iii) a second unit comprising a detecting unit for detecting and/or quantifying binding of the anti-CRP sdAb.

In another aspect, the invention provides an apparatus comprising:
(i) an anti-CRP sdAb of the invention, and
(ii) a first unit comprising a contacting unit for contacting a subject's interstitial fluid (ISF), blood, urine, stool, serum, tear fluid, sweat, or saliva with the anti-CRP sdAb, and
(iii) a second unit comprising a detecting unit for detecting and/or quantifying binding of the anti-CRP sdAb.

In another aspect, the invention provides an apparatus comprising:
(i) an anti-CRP sdAb of the invention, and
(ii) a first unit comprising a contacting unit for contacting a sample with the anti-CRP sdAb, and
(iii) a second unit comprising a detecting unit for detecting and/or quantifying binding of the anti-CRP sdAb, wherein the second unit comprises the anti-CRP sdAb conjugated to a working electrode.

In another aspect, the invention provides an apparatus comprising:
(i) an anti-CRP sdAb of the invention, and
(ii) a first unit comprising a contacting unit for contacting a subject with the anti-CRP sdAb, and
(iii) a second unit comprising a detecting unit for detecting and/or quantifying binding of the anti-CRP sdAb, wherein the second unit comprises the anti-CRP sdAb conjugated to a working electrode.

In another aspect, the invention provides an apparatus comprising:
(i) an anti-CRP sdAb of the invention, and
(ii) a first unit comprising a contacting unit for contacting a sample with the anti-CRP sdAb, and
(iii) a second unit comprising a detecting unit for detecting and/or quantifying binding of the anti-CRP sdAb, wherein the second unit comprises a biosensor of the present invention.

In another aspect, the invention provides an apparatus comprising:
(i) an anti-CRP sdAb of the invention, and
(ii) a first unit comprising a contacting unit for contacting a subject with the anti-CRP sdAb, and
(iii) a second unit comprising a detecting unit for detecting and/or quantifying binding of the anti-CRP sdAb, wherein the second unit comprises a biosensor of the present invention.

In another aspect, the invention provides an apparatus comprising:
(i) an anti-CRP sdAb of the invention, and
(ii) a first unit comprising a contacting unit for contacting a sample comprising interstitial fluid (ISF), blood, urine, stool, serum, tear fluid, sweat, or saliva with the anti-CRP sdAb, and
(iii) a second unit comprising a detecting unit for detecting and/or quantifying binding of the anti-CRP sdAb, wherein the second unit comprises a biosensor of the present invention.

In another aspect, the invention provides an apparatus comprising:
(i) an anti-CRP sdAb of the invention, and
(ii) a first unit comprising a contacting unit for contacting a subject's interstitial fluid (ISF), blood, urine, stool, serum, tear fluid, sweat, or saliva with the anti-CRP sdAb, and
(iii) a second unit comprising a detecting unit for detecting and/or quantifying binding of the anti-CRP sdAb, wherein the second unit comprises a biosensor of the present invention.

In another aspect, the invention provides an apparatus comprising:
(i) an anti-CRP sdAb of the invention, and
(ii) a first unit comprising a contacting unit for contacting a sample comprising interstitial fluid (ISF), blood, urine, stool, serum, tear fluid, sweat, or saliva with the anti-CRP sdAb, and
(iii) a second unit comprising a detecting unit for detecting and/or quantifying binding of the anti-CRP sdAb, wherein the second unit comprises a biosensor comprising the anti-CRP sdAb and a working electrode.

In another aspect, the invention provides an apparatus comprising:
(i) an anti-CRP sdAb of the invention, and
(ii) a first unit comprising a contacting unit for contacting a subject's interstitial fluid (ISF), blood, urine, stool, serum, tear fluid, sweat, or saliva with the anti-CRP sdAb, and
(iii) a second unit comprising a detecting unit for detecting and/or quantifying binding of the anti-CRP sdAb, wherein the second unit comprises a biosensor comprising the anti-CRP sdAb and a working electrode.

In another aspect, the invention provides an apparatus comprising:
(i) an anti-CRP sdAb of the invention, and
(ii) a first unit comprising a contacting unit for contacting a sample comprising interstitial fluid (ISF), blood, urine, stool, serum, tear fluid, sweat, or saliva with the anti-CRP sdAb, and
(iii) a second unit comprising a detecting unit for detecting and/or quantifying binding of the anti-CRP sdAb, wherein the second unit comprises a biosensor comprising the anti-CRP sdAb and a working electrode, wherein the anti-CRP sdAb is conjugated to the working electrode, optionally via a nucleic acid (linker).

In another aspect, the invention provides an apparatus comprising:
(i) an anti-CRP sdAb of the invention, and
(ii) a first unit comprising a contacting unit for contacting a subject's interstitial fluid (ISF), blood, urine, stool, serum, tear fluid, sweat, or saliva with the anti-CRP sdAb, and
(iii) a second unit comprising a detecting unit for detecting and/or quantifying binding of the anti-CRP sdAb, wherein the second unit comprises a biosensor comprising the anti-CRP sdAb and a working electrode, wherein the anti-CRP sdAb is conjugated to the working electrode, optionally via a nucleic acid (linker).

In another aspect, the invention provides an apparatus comprising:
(i) an anti-CRP sdAb of the invention, and
(ii) a first unit comprising a contacting unit for contacting a sample comprising interstitial fluid (ISF), blood, urine, stool, serum, tear fluid, sweat, or saliva with the anti-CRP sdAb, and
(iii) a second unit comprising a detecting unit for detecting and/or quantifying binding of the anti-CRP sdAb, wherein the second unit comprises a biosensor comprising the anti-CRP sdAb and a working electrode, a counter electrode and/or a reference electrode, and a substrate, wherein the anti-CRP sdAb is conjugated to the working electrode, optionally via a nucleic acid (linker).

In another aspect, the invention provides an apparatus comprising:
(i) an anti-CRP sdAb of the invention, and
(ii) a first unit comprising a contacting unit for contacting a subject's interstitial fluid (ISF), blood, urine, stool, serum, tear fluid, sweat, or saliva with the anti-CRP sdAb, and
(iii) a second unit comprising a detecting unit for detecting and/or quantifying binding of the anti-CRP sdAb, wherein the second unit comprises a biosensor comprising the anti-CRP sdAb and a working electrode, a counter electrode and/or a reference electrode, and a substrate, wherein the anti-CRP sdAb is conjugated to the working electrode, optionally via a nucleic acid (linker).

In another aspect, the invention provides an apparatus comprising:
(i) an anti-CRP sdAb of the invention, and
(ii) a first unit comprising a contacting unit for contacting a sample comprising interstitial fluid (ISF), blood, urine, stool, serum, tear fluid, sweat, or saliva with the anti-CRP sdAb, and
(iii) a second unit comprising a detecting unit for detecting and/or quantifying binding of the anti-CRP sdAb, wherein the second unit comprises a biosensor comprising the anti-CRP sdAb and a working electrode, a counter electrode and/or a reference electrode, and a substrate, wherein the anti-CRP sdAb is conjugated to the working electrode, optionally via a nucleic acid (linker), and wherein the anti-CRP sdAb comprises a detection label or tag.

In another aspect, the invention provides an apparatus comprising:
(i) an anti-CRP sdAb of the invention, and
(ii) a first unit comprising a contacting unit for contacting a subject's interstitial fluid (ISF), blood, urine, stool, serum, tear fluid, sweat, or saliva with the anti-CRP sdAb, and
(iii) a second unit comprising a detecting unit for detecting and/or quantifying binding of the anti-CRP sdAb, wherein the second unit comprises a biosensor comprising the anti-CRP sdAb and a working electrode, a counter electrode and/or a reference electrode, and a substrate, wherein the anti-CRP sdAb is conjugated to the working electrode, optionally via a nucleic acid (linker), and wherein the anti-CRP sdAb comprises a detection label or tag.

It is also envisioned herein that the apparatus of the present invention can comprise additional components, such as means for dispensing a drug, e.g. a dispensing unit for dispensing a drug. The means for dispensing a drug/dispensing unit for dispensing a drug can be comprised in a third unit which is further comprised the apparatus of the invention. For example, the apparatus as described herein can comprise a third unit comprising means for dispensing a drug/a dispensing unit for dispensing a drug. This is advantageous when it is desired to combine the measurement of CRP levels in a subject with a therapy. For example, the measured level of CRP in a subject can be compared with a reference value as described herein above. Depending on the difference of the CRP level in the subject and the reference value, the third unit can dispense a drug, e.g. to counter inflammation or to decrease or increase levels of CRP. In the sense of the present invention, a drug can be any drug used in the treatment of an CRP related disease, such as an inflammatory disease and/or autoimmune disease. Exemplary inflammatory diseases and/or autoimmune diseases are rheumatoid arthritis, inflammatory bowel disease, IgA nephropathy, Psoriasis, lupus, multiple sclerosis, Endometriosis, Sepsis, cardiovascular disease, metabolic syndrome, hypertension, diabetes, cancer, bacterial or viral infections, COVID19, asthma, familial Mediterranean fever, Alzheimer's disease, Parkinson's diseases, diabetic macular edema (DME), diabetic retinopathy, uveitis, glaucoma, dry eye (e.g., dry eye disease or dry eye syndrome), allergic conjunctivitis, ocular pain, rhegmatogenous retinal detachment (RRD), age-related macular degeneration (AMD), proliferative diabetic retinopathy (PDR), retinal vein occlusion (RVO), neuromyelitis optica (NMO), corneal transplant, corneal abrasion, physical injury to the eye, non-alcoholic fat liver disease, chronic kidney disease, tumour and the like. In general, the invention is not limited to a third unit dispensing a specific drug, the skilled practitioner is aware that any drug suitable for the means of dispensing the drug and for the therapy of the disease to be treated in the subject can be used. Exemplary drugs that can be used in the sense of the invention are remdevisir, nirmatrelvir, molnupiravir, baricitinib, bebtelovimab, etesevimab, casirivimab, imdevimab, aspirin, celecoxib, prednisone, methotrexate, hydroxychloroquine, leflunomide, sulfasalazine, abatacept, adalimumab, certolizumab, etanecept, golimumab, infliximab, rituximab, sarilumab, tocilizumab, baricitinib, tofacitinib, upadacitinib, anakinra, minocycline, azathioprine, cyclosporine, penicillamine, Statins, angiotensin-converting enzyme inhibitors, angiotensin receptor blockers, dapagliflozin, ibuprofen, naproxen, voclosporin, anifrolumab, mycophenolate, belimumab, glatiramer acetate, ocrelizumab, fingolimod, dimethyl fumarate, teriflunomide, methylprednisone, natalizumab, alemtuzumab, diroximel fumarate, siponimod, cladribine, Broad-spectrum antibiotics, amlodipine, diltiazem, Metformin, Insulin, SGLT2 inhibitors, leuprolide, anastrozole, letrozole, tamoxifen, fulvestrant, bevacizumab, alectinib, ibrutinib, imatinib, palbociclib, fluoxymesterone, methyltestosterone, tamoxifen citrate, estramustine, interferon alfa-2b recombinant, goserelin, flutamide, aldesleukin, bicalutamide, porfimer, nilutamide, imiquimod, toremifene, thalidomide, trastuzumab, alitretinoin, bexarotene, denileukin dfititox, exemestane, gemtuzumab, triptorelin, imatinib mesylate, peginterferon alfa-2b, ibritumomab tiuxetan, leuprolide acteate, abarelix, bortezomib, gefitinib, tositumomab, cetuximab, erlotinib hydrochloride, azacitidine, lenalidomide, sorafenib tosylate, dastinib, decitabine, panitumumab, sunitinib malate, vorinostat, lapatinib ditosylate, nilotinib, temsirolimus, degarelix, everolimus, ofatumumab, pazopanib hydrochloride, romodepsin, denosumab, hydroxiurea, sipuleucel-T, abiraterone acetate, brentuximab vedotin, crizotinib, ipilumab, ruxolinitib phosphate, vandetanib, vemurafenig, pertuzumab, axitinib, bosutinib, cabozantinib, cafilzomib, enzalutamide, ponatinib hydrochloride, regorafenib, vismodegib, Ziv-aflibercept, dabrafenib, trametinib, obinutuzumam, ado-trastuzumab emtansine, afatinib, pomalidomide, idelalisib, belinostat, ceritinib, avelumab, durvalumab, cemiplimab, dostarlimab, pembrolizumab, ramuricumab, lanreotide, blinatumomab, nivolumab, olaparib, atezolizumab, ipilimumab, Mechlorethamine, Mercaptopurine, Busulfan, Chlorambucil, Cyclophosphamide, Vincristine sulfate, dactinomycin, vinblastine sulfate, thioguanine, procarbazine hydrochloride, floxuridine, fluorouracil, mitotane, bleomycin, doxorubicin hydrochloride, dacarbazine, lomustine, carmustine, cisplatin, asparaginase, streptozocin, etoposide, ifosfamide, carboplatin, altreatime, fludarabine, pentostatin, paclitaxel, melphalan, teniposide, caldribine, vinorelbine tartrate, pegaspargase, thiotepa, docetaxel, gemcitabine, irinotecan, topotecan hydrochloride, idarubicin, capecitabine, daunorubucin hydrochloride, valrubicin, temozolomide, cytarabine, epirubicin, arsenic trioxide, mitomycin, oxaliplatin, pemetrexed disodium, clorabine, nelarabine, ixabepilone, bendamustine hydrochloride, pralatrexate, cabazitaxel, eribuline mesylate, asparaginase Erwinia chrysanthemi, oamcetaxine mepesuccinate, radium 223 dichloride, Amikacin, Gentamicin, kanamycin, neomycin, netilmicin, tobramycin, paromomycin, streptomycin, spectinomycin, geldanamycin, herbimycin, rifaximin, loracarbef, ertapenem, doripenem, imipenem, cilastatin, meropenem, cefadroxil, cefazolin, cephradine, cephrapirin, cephalothin, cefalexin, cefaclor, cefoxitin, cefotetan, cefamandole, cefmetazole, cefonicid, cefprozil, cefuroxime, cefixime, cefdinir, cefditoren, ceforeprazone, cefotaxime, cefpodoxime, ceftazidime, ceftibuten, ceftizoxime, moxalactam, ceftriaxone, cefepime, ceftaroline fosamil, ceftobirprole, teicoplanin, vancomycin, telavancin, dalbavancin, oritavancin, clindamycin, lincomycin, daptomycin, azithormycin, clarithromycin, erythromycin, roxithromycin, telithromycin, spriamycin, fidaxomicin, aztreonam, furazolidone, nitrodurantoin, linezolid, posizolid, radezolid, torezolid, amoxicillin, ampicillin, azlocillini, dicloxacillin, flucloxacillin, mezlocillin, methicillin, nafcillin, oxacillin, penicillin, piperacillin, temocillin, ticarcillin, bacitracin, colistin, polymyxin, ciprofloxacin, enoxacin, gatifloxacin, gemifloxacin, levofloxacin, lomefloxacin, moxifloxacin, nadifloxacin, nalidixic acid, norfloxacin, ofloxacin, trovafloxacin, grepafloxacin, sparfloxacin, temfloxacin, mafenied, sulfacetamibed, sulfadiazine, silver sulfadiazine, sulfadimethozine, sulfamethizole, sulfamethoxazole, sulfanilimide, sulfisoxazole, trimehoprim-sulfamethoxazole, sulfonamidochrysoidine, demeclocycline, doxycycline, metacycline, minocyclinie, oxytetracycline, tetracycline, clofazimine, dapsone, capreomycin, cycloserine, ethambutol, ethionamide, isoniazid, pyrazinmide, rifampicin, rifabutin, rifapentine, arsphenamine, chloramphenicol, fosfomycin, fusidic acid, metronidazole, mupirocin, platensimycin, quinupristin, thiampehnicol, tigecycline, tinidazole, tremethoprim, teixobactin, malacidins, halicin, fluticasone propionate, budesonide, ciclesonide, c beclomethasone, mometasone, fluticasone furorate, montelukast, zafirlukast, zileuton, fluticasone-salmeterol, budesonide-formoterole, formoterol-mometasone, fluticasone furoate-vilanterol, Theophylline, methylprednisolone, ipratropium, tiotropium, Colchicine, canakinumab, rilonacept, siltuximab, olokizumab, elsilimomab, clazakizumab, sirukumab, levilimab, or any combination thereof. In some embodiments, a drug that can be used in the sense of the invention is one or more of corticosteroids, methotrexate, IL-6 inhibitors, TNF-alpha inhibitors, PD-1/PD-L1 inhibitors, or CTLA-4 inhibitors.

In some embodiments, the drug is an anti-CRP antibody. In some embodiments the drug is an anti-CRP sdAb of the present invention. In some embodiments the drug is one or more of the anti-CRP sdAb as defined in SEQ ID NOs: 1, 9, 17, 25, 33, 41, 49, 57, 65, 73, 81, or 89. In some embodiments the drug is two or more, such as two, three, four, five, six, seven, eight, nine, ten, eleven or twelve of the anti-CRP sdAb as defined in SEQ ID NOs: 1, 9, 17, 25, 33, 41, 49, 57, 65, 73, 81, or 89. In some embodiments, the drug is the anti-CRP sdAb as defined in SEQ ID NO: 1. In some embodiments, the drug is the anti-CRP sdAb as defined in SEQ ID NO: 9. In some embodiments, the drug is the anti-CRP sdAb as defined in SEQ ID NO: 17. In some embodiments, the drug is the anti-CRP sdAb as defined in SEQ ID NO: 25. In some embodiments, the drug is the anti-CRP sdAb as defined in SEQ ID NO: 33. In some embodiments, the drug is the anti-CRP sdAb as defined in SEQ ID NO: 41. In some embodiments, the drug is the anti-CRP sdAb as defined in SEQ ID NO: 49. In some embodiments, the drug is the anti-CRP sdAb as defined in SEQ ID NO: 57. In some embodiments, the drug is the anti-CRP sdAb as defined in SEQ ID NO: 65. In some embodiments, the drug is the anti-CRP sdAb as defined in SEQ ID NO: 73. In some embodiments, the drug is the anti-CRP sdAb as defined in SEQ ID NO: 81. In some embodiments, the drug is the anti-CRP sdAb as defined in SEQ ID NO: 89. The features described herein above regarding the apparatus including the first and second unit apply mutatis mutandis to the apparatus comprising a third unit.

Accordingly, the invention provides in one aspect an apparatus comprising:
(i) an anti-CRP sdAb of the invention, and
(ii) a contacting unit for contacting a sample with the anti-CRP sdAb, and
(iii) a detecting unit for detecting and/or quantifying binding of the anti-CRP sdAb, and
(iv) a dispensing unit for dispensing a drug.

In another aspect, the invention provides an apparatus comprising:
(i) an anti-CRP sdAb of the invention, and
(ii) a contacting unit for contacting a subject with the anti-CRP sdAb, and
(iii) a detecting unit for detecting and/or quantifying binding of the anti-CRP sdAb, and
(iv) a dispensing unit for dispensing a drug.

In another aspect, the invention provides an apparatus comprising:
(i) an anti-CRP sdAb of the invention, and
(ii) a first unit comprising a contacting unit for contacting a sample with the anti-CRP sdAb, and
(iii) a second unit comprising a detecting unit for detecting and/or quantifying binding of the anti-CRP sdAb, and
(iv) a third unit comprising a dispensing unit for dispensing a drug.

In another aspect, the invention provides an apparatus comprising:
(i) an anti-CRP sdAb of the invention, and
(ii) a first unit comprising a contacting unit for contacting a subject with the anti-CRP sdAb, and
(iii) a second unit comprising a detecting unit for detecting and/or quantifying binding of the anti-CRP sdAb, and
(iv) a third unit comprising a dispensing unit for dispensing a drug.

In another aspect, the invention provides an apparatus comprising:
(i) an anti-CRP sdAb of the invention, and
(ii) a first unit comprising a contacting unit for contacting a sample with the anti-CRP sdAb, and
(iii) a second unit comprising a detecting unit for detecting and/or quantifying binding of the anti-CRP sdAb, and
(iv) a third unit comprising a dispensing unit for dispensing a drug, wherein the drug is one or more of an anti-CRP sdAb of the present invention.

In another aspect, the invention provides an apparatus comprising:
(i) an anti-CRP sdAb of the invention, and
(ii) a first unit comprising a contacting unit for contacting a subject with the anti-CRP sdAb, and
(iii) a second unit comprising a detecting unit for detecting and/or quantifying binding of the anti-CRP sdAb, and
(iv) a third unit comprising a dispensing unit for dispensing a drug, wherein the drug is one or more of an anti-CRP sdAb of the present invention.

The apparatus of the present invention can be in form of an applicator like appliance, such as a handheld or stationary device comprising the biosensor of the present invention, or can be permanently or reversibly implanted or attached to the subject's body, such as an implanted, wearable device comprising the biosensor of the present invention. The implanted or attached apparatus may have a shape of lancet and may be implanted in the dermis layer of the skin e.g. of a subject for detecting binding of the anti-CRP sdAb of the present invention to CRP, e.g. by contacting a sample, e.g. interstitial fluid (ISF) with the anti-CRP sdAb. The thereby detected level of CRP may be correlated and/or converted to overall CRP levels in e.g. blood or other bodily fluids. The site and depth of implantation may affect the shape of the implanted or attached apparatus, components, and configuration of the apparatus. The apparatus may be attached or implanted using an insertion mechanism. The signal readout, i.e. detection of CRP level, can be performed by the apparatus. The apparatus can send the results, i.e. detected CRP levels, to any external device, e.g. a server or smartphone, optionally via an application. The advantage of having a reversibly or permanently implanted apparatus is that continuous measurement of CRP is enabled which can increase the reliability of the obtained CRP dynamics over time. This can be especially useful if the apparatus is to be used for disease management and treatment decisions in a subject, e.g. in combination with the in vivo diagnosis or methods of monitoring the level of CRP described herein below. A reversibly or permanently implanted apparatus can for example detect inflammation spikes in a subject and indicate when medication is necessary and what amount of medication is necessary, thereby greatly improving management of an inflammatory disease in a subject. In a further embodiment, an apparatus as described above can detect when medication is necessary and what amount of medication is necessary and dispense the necessary amount at the relevant time into the subject's circulatory system. The apparatus can establish the temporal variable (time when medication is necessary) by detecting that a certain threshold of CRP level is overcome, such as a reference value as described herein. The apparatus can establish the quantitative variable (necessary amount of medication) by detecting the difference of the CRP level measured in the subject and the reference value as described herein.

The above similarly applies to an apparatus of the present invention in form of an applicator like appliance. Applicator like appliance means that the apparatus is not reversibly or permanently attached to or implanted into a subject. It is rather used as a device on the subject's body to obtain a single measurement at a time (spot measurement). The apparatus used in this way can indicate when and if the measurement is indicative that medication is necessary and can indicate what amount of medication is necessary like explained above, i.e. by comparing the measurements with a reference value.

As mentioned herein, the apparatus of the present invention can comprise the biosensor of the present invention. In such a case, the biosensor could be seen as a subsystem within the apparatus. The apparatus of the present invention can also be partially implanted. For example the biosensor subsystem of the apparatus may be the implanted part, i.e. being within the body of a subject (under the skin layer). The entire apparatus does not necessarily have to be entirely within the body (under the skin layer) but can also be superficially attached e.g. on the skin. In such a setup the signal readout electronics is wearable (e.g. being superficially attached on the skin of the subject) and connected with the implanted part, e.g. the biosensor.

When the apparatus is an applicator like appliance, e.g. for the “spot measurement system” it can comprise a biosensor strip (e.g. on a bench), and a portable electronics reader that can be connected to the biosensor strip or vice versa to provide a readout.

Accordingly, the invention provides in one aspect an apparatus comprising:
(i) an anti-CRP sdAb of the present invention, and
(ii) a contacting unit for contacting a sample or a subject with the anti-CRP sdAb, and
(iii) a detecting unit for detecting and/or quantifying binding of the anti-CRP sdAb, and optionally
(iv) a dispensing unit for dispensing a drug, optionally wherein the drug is the anti-CRP sdAb of the present invention,
wherein the apparatus is reversibly attached to or implanted into the subject.

In another aspect, the invention provides an apparatus comprising:
(i) an anti-CRP sdAb of the present invention, and
(ii) a contacting unit for contacting a sample or a subject with the anti-CRP sdAb, and
(iii) a detecting unit for detecting and/or quantifying binding of the anti-CRP sdAb, and optionally
(iv) a dispensing unit for dispensing a drug, optionally wherein the drug is the anti-CRP sdAb of the present invention, wherein the apparatus is permanently attached to or implanted into the subject.

In another aspect, the invention provides an apparatus comprising:
(i) an anti-CRP sdAb of the present invention, and
(ii) a contacting unit for contacting a sample or a subject with the anti-CRP sdAb, and
(iii) a detecting unit for detecting and/or quantifying binding of the anti-CRP sdAb, and optionally
(iv) a dispensing unit for dispensing a drug, optionally wherein the drug is the anti-CRP sdAb of the present invention,
wherein the apparatus is fully or partially implantable.

In another aspect, the invention provides an apparatus comprising:
(i) an anti-CRP sdAb of the present invention, and
(ii) a contacting unit for contacting a sample or a subject with the anti-CRP sdAb, and
(iii) a detecting unit for detecting and/or quantifying binding of the anti-CRP sdAb, and optionally
(iv) a dispensing unit for dispensing a drug, optionally wherein the drug is the anti-CRP sdAb of the present invention,
wherein the apparatus is fully or partially implanted into a subject.

Any of apparatuses as described herein can be permanent, reusable, single use, disposable or for temporary use (e.g. implant). For example, a biosensor of the present invention can be implanted for a limited time e.g. 14 days for a continuous measurement of CRP level.

In yet another aspect, the invention provides the following items:
1. An apparatus (for detecting a target analyte) comprising:
   (i) a collector for collecting a sample,
   (ii) a sensing component comprising a biosensor comprising an anti-CRP sdAb of the present invention,
   (iii) a connector for connecting the sensing component to an electrochemical measurement device.
2. The apparatus of item 1, wherein the collector is designed to at least partially encase the sensing component.
3. The apparatus of item 1 or 2, wherein the collector comprises a first portion and a second portion, the first portion being able to contain the collected sample and the second portion encasing the sensing component.
4. The apparatus of item 3, wherein the first portion comprises a nozzle and a sampling reservoir.
5. The apparatus of item 4, wherein the nozzle comprises microfluidic channels optionally treated with a hydrophilic coating.
6. The apparatus of item 4, wherein the nozzle comprises silica capillary tubes.
7. The apparatus of any one of items 1-6, wherein the collector and the connector are provided with complementary locking means to secure the sensing component within the device.
8. The apparatus of any one of items 1-7, wherein the sensing component further comprises two working electrodes, a counter electrode and a reference electrode.
9. The apparatus of item 8, wherein the electrode of the biosensor and the two working electrodes comprise gold.
10. The apparatus of items 8 or 9, wherein the counter electrode comprises platinum and the reference electrode comprises silver.
11. The apparatus of items 8-10, wherein each electrode is in the form of a wire.
12. The apparatus of item 11, wherein the sensing component has a cylindrical form and the wires are positioned spaced apart within the sensing component along a length thereof.
13. The apparatus of any one of items 8-12, wherein the electrodes are held in a matrix of a non-conductive material.
14. The apparatus of item 13, wherein the non-conductive material comprises a silicon resin.
15. The apparatus of item any one of items 11-14, wherein the anti-CRP sdAb is bound to a first end of the biosensor electrode wire and the anti-CRP sdAb is exposed to the sample when the first end is in contact with the collected sample.
16. The apparatus of any one of items 11-15, wherein a first end of each electrode wire is in contact with the sample when collected in the collector.
17. The apparatus of any one of items 11-15, wherein a second end of the wires are in contact with the connector.
18. The apparatus of any one of items 1-17, wherein the sample comprises a biological fluid, such as a bodily fluid e.g. interstitial fluid (ISF), blood, urine, stool, serum, tear fluid, sweat or saliva.
19. The apparatus of any one of items 1-18, wherein the target analyte is CRP.

The sdAbs of the present invention are particularly useful for therapy since they can inhibit the biological function of CRP thereby reducing inflammation. The sdAbs of the present invention do not necessarily inhibit the biological function of CRP. In some embodiments, it is preferred that a sdAbs of the invention do not inhibit the biological function of CRP, e.g. when detection but not treatment is desired. Accordingly, in some embodiments it is preferred that a sdAb of the invention does inhibit the biological function of CRP, e.g. when detection and therapy or only therapy is desired. As used herein “treatment” preferably relates to the acute treatment, prophylactic treatment, or the disease management of an inflammatory disease and/or autoimmune disease related to an increased CRP production, CRP secretion, CRP release or CRP level or increased inflammation values that are treated with the anti-CRP sdAbs of the invention.

Accordingly, the present invention provides in one aspect an anti-CRP sdAb of the invention for use as a medicament.

In another aspect, the invention provides the anti-CRP sdAb as defined in SEQ ID NO: 1 for use as a medicament.

In another aspect, the invention provides the anti-CRP sdAb as defined in SEQ ID NO: 9 for use as a medicament.

In another aspect, the invention provides the anti-CRP sdAb as defined in SEQ ID NO: 17 for use as a medicament.

In another aspect, the invention provides the anti-CRP sdAb as defined in SEQ ID NO: 25 for use as a medicament.

In another aspect, the invention provides the anti-CRP sdAb as defined in SEQ ID NO: 33 for use as a medicament.

In another aspect, the invention provides the anti-CRP sdAb as defined in SEQ ID NO: 41 for use as a medicament.

In another aspect, the invention provides the anti-CRP sdAb as defined in SEQ ID NO: 49 for use as a medicament.

In another aspect, the invention provides the anti-CRP sdAb as defined in SEQ ID NO: 57 for use as a medicament.

In another aspect, the invention provides the anti-CRP sdAb as defined in SEQ ID NO: 65 for use as a medicament.

In another aspect, the invention provides the anti-CRP sdAb as defined in SEQ ID NO: 73 for use as a medicament.

In another aspect, the invention provides the anti-CRP sdAb as defined in SEQ ID NO: 81 for use as a medicament.

In another aspect, the invention provides the anti-CRP sdAb as defined in SEQ ID NO: 89 for use as a medicament.

In another aspect, the invention provides the anti-CRP sdAb as defined in any one of SEQ ID NOs: 1, 9, 17, 25, 33, 41, 49, 57, 65, 73, 81, or 89 for use as a medicament.

In another aspect, the invention provides one or more, such as one, two, three, four, five, six, seven, eight, nine, ten, eleven or twelve anti-CRP sdAbs as defined in SEQ ID NOs: 1, 9, 17, 25, 33, 41, 49, 57, 65, 73, 81, or 89 for use as a medicament.

Secretion of CRP can induce inflammation. Thus, the sdAbs of the invention are preferably used in the treatment of CRP associated diseases, such as diseases associated with inflammation or systemic inflammation. In particular, the disease may be associated with an increased production or release of CRP. Inhibiting the biological function of CRP can thus ameliorate this effect and reduce the resulting inflammation. The invention is not particularly limited to a disease to be treated, rather any disease that results in increased CRP production, CRP secretion, CRP release or CRP level or increased inflammation values can be treated with the sdAbs of the invention. Such a disease can be associated with an increased production, secretion, release, or level of inflammatory markers. Inflammatory markers can indicate an inflammation event which may then be treated with the anti-CRP sdAbs of the present invention. Exemplary inflammatory markers are Adenosine deaminase (ADA), Artemin (ARTN), Axin-1 (AXIN1), Beta-nerve growth factor (NGF), C-reactive protein (CRP), C-C motif chemokine 13 (CCL13), C-C motif chemokine 19 (CCL19), C-C motif chemokine 2 (CCL2), C-C motif chemokine 20 (CCL20), C-C motif chemokine 23 (CCL23), C-C motif chemokine 25 (CCL25), C-C motif chemokine 28 (CCL28), C-C motif chemokine 3 (CCL3), C-C motif chemokine 4 (CCL4), C-C motif chemokine 7 (CCL7), C-C motif chemokine 8 (CCL8), C-X-C motif chemokine 10 (CXCL10), C-X-C motif chemokine 11 (CXCL11), C-X-C motif chemokine 5 (CXCL5), C-X-C motif chemokine 6 (CXCL6), C-X-C motif chemokine 9 (CXCL9), CUB domain-containing protein 1 (CDCP1), Caspase-8 (CASP8), Cystatin-D (CST5), Delta and Notch-like epidermal growth factor-related receptor (DNER), Eotaxin (CCL11), Eukaryotic translation initiation factor 4E-binding protein 1 (EIF4EBP1), Fibroblast growth factor 19 (FGF19), Fibroblast growth factor 21 (FGF21), Fibroblast growth factor 23 (FGF23), Fibroblast growth factor 5 (FGF5), Fms-related tyrosine kinase 3 ligand (FLT3LG), Fractalkine (CX3CL1), Glial cell line-derived neurotrophic factor (GDNF), Growth-regulated alpha protein (CXCL1), Hepatocyte growth factor (HGF), Interferon gamma (IFNG), Interleukin-1 alpha (IL1A), Interleukin-10 (IL10), Interleukin-10 receptor subunit alpha (IL10RA), Interleukin-10 receptor subunit beta (IL10RB), Interleukin-12 subunit beta (IL12B), Interleukin-13 (IL13), Interleukin-15 receptor subunit alpha (IL15RA), Interleukin-17A (IL17A), Interleukin-17C (IL17C), Interleukin-18 (IL18), Interleukin-18 receptor 1 (IL18R1), Interleukin-2 (IL2), Interleukin-2 receptor subunit beta (IL2RB), Interleukin-20 (IL20), Interleukin-20 receptor subunit alpha (IL20RA), Interleukin-22 receptor subunit alpha-1 (IL22RA1), Interleukin-24 (IL24), Interleukin-33 (IL33), Interleukin-4 (IL4), Interleukin-5 (IL5), Interleukin-6 (IL6), Interleukin-7 (IL7), Interleukin-8 (CXCL8), Interstitial collagenase (MMP1), Kit ligand (KITLG), Leukemia inhibitory factor (LIF), Leukemia inhibitory factor receptor (LIFR), Lymphotoxin-alpha (LTA), Macrophage colony-stimulating factor 1 (CSF1), NAD-dependent protein deacetylase sirtuin-2 (SIRT2), Natural killer cell receptor 2B4 (CD244), Neurotrophin-3 (NTF3), Neurturin (NRTN), Oncostatin-M (OSM), Programmed cell death 1 ligand 1 (CD274), Protein S100-A12 (S100A12), Protransforming growth factor alpha (TGFA), STAM-binding protein (STAMBP), Signaling lymphocytic activation (SLAMF1), Stromelysin-2 (MMP10), Sulfotransferase 1A1 (SULT1A1), T-cell differentiation antigen CD6 (CD6), T-cell surface glycoprotein CD5 (CD5), T-cell surface glycoprotein CD8 alpha chain (CD8A), Thymic stromal lymphopoietin (TSLP), Transforming growth factor beta-1 proprotein (TGFB1), Tumor necrosis factor alpha (TNF-a), Tumor necrosis factor ligand superfamily member 10 (TNFSF10), Tumor necrosis factor ligand superfamily member 11 (TNFSF11), Tumor necrosis factor ligand superfamily member 12 (TNFSF12), Tumor necrosis factor ligand superfamily member 14 (TNFSF14), Tumor necrosis factor receptor superfamily member 11B (TNFRSF11B), Tumor necrosis factor receptor superfamily member 5 (CD40), Tumor necrosis factor receptor superfamily member 9 (TNFRSF9), Urokinase-type plasminogen activator (PLAU), Vascular endothelial growth factor A (VEGFA) and the like or any combination thereof.

Inflammatory markers can also include the following markers. Exemplary additional biomarkers are: Adiponectin, CD44var (v6), Elafin, Lactoferrin, MMP-3, NRP-1, RANTES (CCL5), Resistin (ADSF), VCAM-1, CD14, CD62L (L-Selectin), Endoglin, Leptin, MMP-9, Osteopontin (OPN), RBP4, SCGF beta, ZAG, CD44, Cystatin C, ICAM-1, MBL, NGAL, PAI-1 (Serpin), REG-3a, TIMP-1.

Additionally, markers can indicate vascular injuries. Exemplary vascular injury markers are Serum Amyloid A, C-Reactive Protein CRP, Vascular Cell Adhesion Molecule-1 (VCAM-/CD106), Intercellular Adhesion Molecule-1 (ICAM-1/CD54).

In a preferred embodiment an inflammatory marker is CRP. Exemplary diseases are inflammatory diseases and/or autoimmune diseases. Exemplary inflammatory diseases and/or autoimmune diseases are rheumatoid arthritis, inflammatory bowel disease, IgA nephropathy, Psoriasis, lupus, multiple sclerosis, Endometriosis, Sepsis, cardiovascular disease, metabolic syndrome, hypertension, diabetes, cancer, bacterial or viral infections, COVID19, asthma, familial Mediterranean fever, Alzheimer's disease, Parkinson's diseases, diabetic macular edema (DME), diabetic retinopathy, uveitis, glaucoma, dry eye (e.g., dry eye disease or dry eye syndrome), allergic conjunctivitis, ocular pain, rhegmatogenous retinal detachment (RRD), age-related macular degeneration (AMD), proliferative diabetic retinopathy (PDR), retinal vein occlusion (RVO), neuromyelitis optica (NMO), corneal transplant, corneal abrasion, physical injury to the eye, non-alcoholic fat liver disease, chronic kidney disease, tumour and the like. Accordingly, the invention provides in one aspect an anti-CRP sdAb of the invention for use in the treatment of a disease associated with an inflammation.

In another aspect, the invention provides an anti-CRP sdAb of the invention for use in the treatment of a disease associated with an inflammation, optionally a systemic inflammation.

It is also envisioned herein that the anti-CRP sdAb of the invention can be used in co-therapy, wherein more than one, such as two, three, four, five, six, seven, eight, nine, ten, eleven or twelve anti-CRP sdAb are selected from SEQ ID NOs: 1, 9, 17, 25, 33, 41, 49, 57, 65, 73, 81, or 89. Accordingly, the invention provides two or more, such as two, three, four, five, six, seven, eight, nine, ten, eleven or twelve anti-CRP sdAb selected form SEQ ID NOs: 1, 9, 17, 25, 33, 41, 49, 57, 65, 73, 81, or 89 for use in the treatment of a disease associated with an inflammation, optionally a systemic inflammation.

Accordingly, the invention provides in one aspect one or more anti-CRP sdAb selected from the group of SEQ ID NOs: 1, 9, 17, 25, 33, 41, 49, 57, 65, 73, 81, or 89 for use in the treatment of a disease associated with an inflammation, optionally a systemic inflammation. In general, it is envisioned that sdAbs of the present invention can also be used in combination, e.g. sdAbs having specific kₒₙ/k_{off} values can be combined to achieve coverage of continuous and reversible binding over a desired dynamic range of e.g. CRP.

In another aspect, the invention provides an anti-CRP sdAb of the invention for use in the treatment of a disease associated with an inflammation, optionally a systemic inflammation, wherein the disease is associated with an increased production or release of inflammatory markers.

In another aspect, the invention provides an anti-CRP sdAb of the invention for use in the treatment of a disease associated with an inflammation, optionally a systemic inflammation, wherein the disease is associated with an increased production or release of CRP.

In another aspect, the invention provides an anti-CRP sdAb of the invention for use in the treatment of an inflammatory and/or autoimmune disease.

In another aspect, the invention provides an anti-CRP sdAb of the invention for use in the treatment of rheumatoid arthritis, inflammatory bowel disease, IgA nephropathy, Psoriasis, lupus, multiple sclerosis, Endometriosis, Sepsis, cardiovascular disease, metabolic syndrome, hypertension, diabetes, cancer, bacterial or viral infections, COVID19, asthma, familial Mediterranean fever, Alzheimer's disease, Parkinson's diseases, diabetic macular edema (DME), diabetic retinopathy, uveitis, glaucoma, dry eye (e.g., dry eye disease or dry eye syndrome), allergic conjunctivitis, ocular pain, rhegmatogenous retinal detachment (RRD), age-related macular degeneration (AMD), proliferative diabetic retinopathy (PDR), retinal vein occlusion (RVO), neuromyelitis optica (NMO), corneal transplant, corneal abrasion, physical injury to the eye, non-alcoholic fat liver disease, chronic kidney disease, or tumour.

Methods of treatment are also envisioned herein. In general, the features described in context of the anti-CRP sdAb of the present invention for use in the treatment of a disease also apply to the methods of treatment provided herein. The method of treatment can comprise a step of administering one or more anti-CRP sdAb of the invention to a subject. Similarly, co-therapy can be practiced in a method of treatment, wherein more than one, such as two, three, four, five, six, seven, eight, nine, ten, eleven or twelve anti-CRP sdAb selected from SEQ ID NOs: 1, 9, 17, 25, 33, 41, 49, 57, 65, 73, 81, or 89 are administered to a subject.

Accordingly, the invention provides in one aspect a method of treatment comprising a step of administering an anti-CRP sdAb of the invention to a subject in need thereof.

In another aspect, the invention provides a method of treatment comprising a step of administering one or more of an anti-CRP sdAb selected from the group of SEQ ID NOs: 1, 9, 17, 25, 33, 41, 49, 57, 65, 73, 81, or 89 to a subject in need thereof.

In another aspect, the invention provides a method of treatment comprising a step of administering two or more of an anti-CRP sdAb selected from the group of SEQ ID NOs: 1, 9, 17, 25, 33, 41, 49, 57, 65, 73, 81, or 89 to a subject in need thereof.

In another aspect, the invention provides a method of treatment comprising a step of administering an anti-CRP sdAb of the invention to a subject in need thereof, wherein the subject suffers from a disease associated with an increased production or release of inflammatory markers.

In another aspect, the invention provides a method of treatment comprising a step of administering an anti-CRP sdAb of the invention to a subject in need thereof, wherein the subject suffers from a disease associated with an increased production or release of CRP.

In another aspect, the invention provides a method of treatment comprising a step of administering an anti-CRP sdAb of the invention to a subject in need thereof, wherein the subject suffers from an inflammatory and/or autoimmune disease.

In another aspect, the invention provides a method of treatment comprising a step of administering an anti-CRP sdAb of the invention to a subject in need thereof, wherein the subject suffers from rheumatoid arthritis, inflammatory bowel disease, IgA nephropathy, Psoriasis, lupus, multiple sclerosis, Endometriosis, Sepsis, cardiovascular disease, metabolic syndrome, hypertension, diabetes, cancer, bacterial or viral infections, COVID19, asthma, familial Mediterranean fever, Alzheimer's disease, Parkinson's diseases, diabetic macular edema (DME), diabetic retinopathy, uveitis, glaucoma, dry eye (e.g., dry eye disease or dry eye syndrome), allergic conjunctivitis, ocular pain, rhegmatogenous retinal detachment (RRD), age-related macular degeneration (AMD), proliferative diabetic retinopathy (PDR), retinal vein occlusion (RVO), neuromyelitis optica (NMO), corneal transplant, corneal abrasion, physical injury to the eye, non-alcoholic fat liver disease, chronic kidney disease, or tumour.

Any of the treatments described herein can be performed by or in or by an apparatus as described herein.

Accordingly, the invention provides in one aspect a method of treatment comprising contacting or administering an apparatus of the present invention to a subject.

In another aspect, the invention provides a method of treatment comprising contacting or administering an apparatus of the present invention to a subject, wherein the apparatus administers a drug to the subject, optionally wherein the drug is a sdAb of the present invention.

In another aspect, the invention provides a method of treatment comprising contacting or administering an apparatus of the present invention to a subject, wherein the apparatus administers a drug to the subject, optionally wherein the drug is a sdAb of the present invention, wherein the subject suffers from a disease associated with an increased production or release of inflammatory markers.

In another aspect, the invention provides a method of treatment comprising contacting or administering an apparatus of the present invention to a subject, wherein the apparatus administers a drug to the subject, optionally wherein the drug is a sdAb of the present invention, wherein the subject suffers from a disease associated with an increased production or release of CRP.

In another aspect, the invention provides a method of treatment comprising contacting or administering an apparatus of the present invention to a subject, wherein the apparatus administers a drug to the subject, optionally wherein the drug is a sdAb of the present invention, wherein the subject suffers from an inflammatory and/or autoimmune disease.

In another aspect, the invention provides a method of treatment comprising contacting or administering an apparatus of the present invention to a subject, wherein the apparatus administers a drug to the subject, optionally wherein the drug is a sdAb of the present invention, wherein the subject suffers from rheumatoid arthritis, inflammatory bowel disease, IgA nephropathy, Psoriasis, lupus, multiple sclerosis, Endometriosis, Sepsis, cardiovascular disease, metabolic syndrome, hypertension, diabetes, cancer, bacterial or viral infections, COVID19, asthma, familial Mediterranean fever, Alzheimer's disease, Parkinson's diseases, diabetic macular edema (DME), diabetic retinopathy, uveitis, glaucoma, dry eye (e.g., dry eye disease or dry eye syndrome), allergic conjunctivitis, ocular pain, rhegmatogenous retinal detachment (RRD), age-related macular degeneration (AMD), proliferative diabetic retinopathy (PDR), retinal vein occlusion (RVO), neuromyelitis optica (NMO), corneal transplant, corneal abrasion, physical injury to the eye, non-alcoholic fat liver disease, chronic kidney disease, or tumour.

In another aspect, the invention provides a method of treatment comprising contacting or administering an apparatus of the present invention to a subject, wherein the apparatus administers a drug to the subject, optionally wherein the drug is a sdAb of the present invention, wherein the apparatus performs the method for detecting and/or quantifying CRP in or on a subject of the present invention prior to administering the drug.

In another aspect, the invention provides a method of treatment comprising contacting or administering an apparatus of the present invention to a subject, wherein the apparatus administers a drug to the subject, optionally wherein the drug is a sdAb of the present invention, wherein the apparatus performs the method for detecting and/or quantifying CRP in or on a subject of the present invention prior to administering the drug, and wherein the administration of said drug depends on the measurement obtained from the method for detecting and/or quantifying CRP in or on a subject.

In another aspect, the invention provides a method of treatment comprising contacting or administering an apparatus of the present invention to a subject, wherein the apparatus administers a drug to the subject, optionally wherein the drug is a sdAb of the present invention, wherein the apparatus performs the method for detecting and/or quantifying CRP in or on a subject of the present invention and compares the level of CRP to a reference value prior to administering the drug, and wherein the administration of said drug depends on the difference between the measured CRP level and the reference value.

In another aspect, the invention provides a method of treatment comprising contacting or administering an apparatus of the present invention to a subject, wherein the apparatus administers a drug to the subject, optionally wherein the drug is a sdAb of the present invention, wherein the apparatus performs the method for detecting and/or quantifying CRP in or on a subject of the present invention and compares the level of CRP to a reference value prior to administering the drug, and wherein the administration and amount of said drug depends on the difference between the measured CRP level and the reference value.

The anti-CRP sdAb of the invention can also be used in in vivo diagnosis. The anti-CRP sdAb are particularly useful for in vivo diagnosis since the potential of detecting the dynamic range of CRP in a subject, optionally over time, can allow the skilled practitioner to draw conclusions on the underlying disease or condition. As mentioned above, the level of CRP can be for example indicative to distinguish a bacterial infection from a viral infection. Similarly, the level of CRP can indicate that a subject suffers from rheumatoid arthritis, inflammatory bowel disease, IgA nephropathy, Psoriasis, lupus, multiple sclerosis, Endometriosis, Sepsis, cardiovascular disease, metabolic syndrome, hypertension, diabetes, cancer, bacterial or viral infections, COVID19, asthma, familial Mediterranean fever, Alzheimer's disease, Parkinson's diseases, diabetic macular edema (DME), diabetic retinopathy, uveitis, glaucoma, dry eye (e.g., dry eye disease or dry eye syndrome), allergic conjunctivitis, ocular pain, rhegmatogenous retinal detachment (RRD), age-related macular degeneration (AMD), proliferative diabetic retinopathy (PDR), retinal vein occlusion (RVO), neuromyelitis optica (NMO), corneal transplant, corneal abrasion, physical injury to the eye, non-alcoholic fat liver disease, chronic kidney disease, tumour and the like. In the sense of the present invention, a disease diagnosed in the in vivo diagnosis is preferably a disease associated with an inflammation. A disease associated with an inflammation may be an inflammatory disease and/or an autoimmune disease. Exemplary inflammatory diseases and/or autoimmune diseases that can be diagnosed in the in vivo diagnosis described herein are rheumatoid arthritis, inflammatory bowel disease, IgA nephropathy, Psoriasis, lupus, multiple sclerosis, Endometriosis, Sepsis, cardiovascular disease, metabolic syndrome, hypertension, diabetes, cancer, bacterial or viral infections, COVID19, asthma, familial Mediterranean fever, Alzheimer's disease, Parkinson's diseases, diabetic macular edema (DME), diabetic retinopathy, uveitis, glaucoma, dry eye (e.g., dry eye disease or dry eye syndrome), allergic conjunctivitis, ocular pain, rhegmatogenous retinal detachment (RRD), age-related macular degeneration (AMD), proliferative diabetic retinopathy (PDR), retinal vein occlusion (RVO), neuromyelitis optica (NMO), corneal transplant, corneal abrasion, physical injury to the eye, non-alcoholic fat liver disease, chronic kidney disease, tumour and the like. A contacting step in the sense of any in vivo methods or in situ methods disclosed herein can comprise contacting a subject's interstitial fluid (ISF), blood, urine, stool, serum, tear fluid, sweat, or saliva with an anti-CRP sdAb of the invention. In some embodiments a contacting step of a method of in vivo diagnosis of a disease associated with an inflammation of the present invention comprises contacting a subject's interstitial fluid (ISF), blood, urine, stool, serum, tear fluid, sweat, or saliva with an anti-CRP sdAb of the invention.

Accordingly, the invention provides in one aspect an anti-CRP sdAb of the present invention for use in in vivo diagnosis.

In another aspect, the invention provides an anti-CRP sdAb of the present invention for use in a method of in vivo diagnosis of a disease associated with an inflammation, the method comprising:
(i) contacting a subject with an anti-CRP sdAb of the present invention, and
(ii) detecting the binding of the anti-CRP sdAb.

In another aspect, the invention provides an anti-CRP sdAb of the present invention for use in a method of in vivo diagnosis of a disease associated with an inflammation, the method comprising:
(i) contacting a subject's biological fluid or sample, such as bodily fluid, e.g. interstitial fluid (ISF), blood, urine, stool, serum, tear fluid, sweat, or saliva with an anti-CRP sdAb of the invention, and
(ii) detecting the binding of the anti-CRP sdAb.

In another aspect, the invention provides an anti-CRP sdAb of the present invention for use in a method of in vivo diagnosis of a disease associated with an inflammation, optionally a systemic inflammation, the method comprising:
(i) contacting a subject with an anti-CRP sdAb of the invention, and
(ii) detecting the binding of the anti-CRP sdAb.

In another aspect, the invention provides an anti-CRP sdAb of the present invention for use in a method of in vivo diagnosis of a disease associated with an inflammation, optionally a systemic inflammation, the method comprising:
(i) contacting a subject with an anti-CRP sdAb of the invention, and
(ii) detecting the binding of the anti-CRP sdAb,
wherein the disease associated with an inflammation or systemic inflammation is an inflammatory disease and/or an autoimmune disease.

In another aspect, the invention provides an anti-CRP sdAb of the present invention for use in a method of in vivo diagnosis of a disease associated with an inflammation, optionally a systemic inflammation, the method comprising:
(i) contacting a subject with an anti-CRP sdAb of the invention, and
(ii) detecting the binding of the anti-CRP sdAb,
wherein the disease associated with an inflammation or systemic inflammation is rheumatoid arthritis, inflammatory bowel disease, IgA nephropathy, Psoriasis, lupus, multiple sclerosis, Endometriosis, Sepsis, cardiovascular disease, metabolic syndrome, hypertension, diabetes, cancer, bacterial or viral infections, COVID19, asthma, familial Mediterranean fever, Alzheimer's disease, Parkinson's diseases, diabetic macular edema (DME), diabetic retinopathy, uveitis, glaucoma, dry eye (e.g., dry eye disease or dry eye syndrome), allergic conjunctivitis, ocular pain, rhegmatogenous retinal detachment (RRD), age-related macular degeneration (AMD), proliferative diabetic retinopathy (PDR), retinal vein occlusion (RVO), neuromyelitis optica (NMO), corneal transplant, corneal abrasion, physical injury to the eye, non-alcoholic fat liver disease, chronic kidney disease, or tumour.

In particular, the detection of the CRP level over time achieved by repeated spot measurements of CRP over time or by the continuous measurement of CRP, e.g. in vivo as described herein above, can indicate inflammation spikes caused by diseases requiring constant disease management, close medical attention and/or regular medication. This can be advantageous in disease management e.g. of inflammatory bowel disease, rheumatoid arthritis, or to monitor efficacy and tolerability e.g. of corticosteroid therapy or immunotherapy in cancer patients. The spikes can be detected by the in vivo diagnosis provided herein. This is advantageous for the underlying treatment insofar it allows the subject suffering from a disease to precisely target inflammation spikes with drugs e.g. an CRP drug such as an anti-CRP sdAb of the invention, thereby reducing the total amount of drugs taken, the effectiveness of the treatment and reducing potential side effects from an otherwise regular medication. The in vivo diagnosis can for example be performed by an apparatus of the present invention.

Accordingly, the invention provides in one aspect an anti-CRP sdAb of the present invention for use in a method of in vivo diagnosis of a disease associated with an inflammation, optionally a systemic inflammation, the method comprising:
(i) contacting a subject with an anti-CRP sdAb of the invention, and
(ii) detecting the binding of the anti-CRP sdAb,
wherein steps (i) and (ii) are repeatedly performed.

In another aspect, the invention provides an anti-CRP sdAb of the present invention for use in a method of in vivo diagnosis of a disease associated with an inflammation, optionally a systemic inflammation, the method comprising:
(i) contacting a subject with an anti-CRP sdAb of the invention, and
(ii) detecting the binding of the anti-CRP sdAb,
wherein steps (i) and (ii) are repeatedly performed in a subject over time.

In another aspect, the invention provides an anti-CRP sdAb of the present invention for use in a method of in vivo diagnosis of a disease associated with an inflammation, optionally a systemic inflammation, the method comprising:
(i) contacting a subject with an anti-CRP sdAb of the invention, and
(ii) detecting the binding of the anti-CRP sdAb,
wherein steps (i) and (ii) are repeatedly performed by an apparatus of the present invention permanently or reversibly attached to or implanted into a subject.

Diagnostic methods are also envisioned herein. In general, the features described in context of the anti-CRP sdAb of the present invention for use in in vivo diagnosis also apply to the methods of treatment provided herein.

Accordingly, the invention provides in one aspect a method of in vivo diagnosis of a disease associated with an inflammation, the method comprising:
(i) contacting a subject with an anti-CRP sdAb of the present invention, and
(ii) detecting the binding of the anti-CRP sdAb.

In another aspect, the invention provides a method of in vivo diagnosis of a disease associated with an inflammation, the method comprising:
(i) contacting a subject's biological fluid or sample, such as bodily fluid, e.g. interstitial fluid (ISF), blood, urine, stool, serum, tear fluid, sweat, or saliva with an anti-CRP sdAb of the invention, and
(ii) detecting the binding of the anti-CRP sdAb.

In another aspect, the invention provides a method of in vivo diagnosis of a disease associated with an inflammation, optionally a systemic inflammation, the method comprising:
(i) contacting a subject with an anti-CRP sdAb of the invention, and
(ii) detecting the binding of the anti-CRP sdAb.

In another aspect, the invention provides a method of in vivo diagnosis of a disease associated with an inflammation, optionally a systemic inflammation, the method comprising:
(i) contacting a subject with an anti-CRP sdAb of the invention, and
(ii) detecting the binding of the anti-CRP sdAb,
wherein the disease associated with an inflammation or systemic inflammation is an inflammatory disease and/or an autoimmune disease.

In another aspect, the invention provides a method of in vivo diagnosis of a disease associated with an inflammation, optionally a systemic inflammation, the method comprising:
(i) contacting a subject with an anti-CRP sdAb of the invention, and
(ii) detecting the binding of the anti-CRP sdAb,
wherein the disease associated with an inflammation or systemic inflammation is rheumatoid arthritis, inflammatory bowel disease, IgA nephropathy, Psoriasis, lupus, multiple sclerosis, Endometriosis, Sepsis, cardiovascular disease, metabolic syndrome, hypertension, diabetes, cancer, bacterial or viral infections, COVID19, asthma, familial Mediterranean fever, Alzheimer's disease, Parkinson's diseases, diabetic macular edema (DME), diabetic retinopathy, uveitis, glaucoma, dry eye (e.g., dry eye disease or dry eye syndrome), allergic conjunctivitis, ocular pain, rhegmatogenous retinal detachment (RRD), age-related macular degeneration (AMD), proliferative diabetic retinopathy (PDR), retinal vein occlusion (RVO), neuromyelitis optica (NMO), corneal transplant, corneal abrasion, physical injury to the eye, non-alcoholic fat liver disease, chronic kidney disease, or tumour.

In another aspect, the invention provides a method of in vivo diagnosis of a disease associated with an inflammation, optionally a systemic inflammation, the method comprising:
(i) contacting a subject with an anti-CRP sdAb of the invention, and
(ii) detecting the binding of the anti-CRP sdAb,
wherein steps (i) and (ii) are repeatedly performed.

In another aspect, the invention provides a method of in vivo diagnosis of a disease associated with an inflammation, optionally a systemic inflammation, the method comprising:
(i) contacting a subject with an anti-CRP sdAb of the invention, and
(ii) detecting the binding of the anti-CRP sdAb,
wherein steps (i) and (ii) are repeatedly performed in a subject over time.

In another aspect, the invention provides a method of in vivo diagnosis of a disease associated with an inflammation, optionally a systemic inflammation, the method comprising:
(i) contacting a subject with an anti-CRP sdAb of the invention, and
(ii) detecting the binding of the anti-CRP sdAb,
wherein steps (i) and (ii) are repeatedly performed by an apparatus of the present invention permanently or reversibly attached to or implanted into a subject.

Any of the in vivo diagnosis described herein can further comprise a step of comparing the level of anti-CRP with a reference value or reference sample as described herein.

Any of the in vivo diagnosis described herein can be performed by or in an apparatus as described herein. The in vivo diagnosis as described herein can be further indicative for a necessary treatment which can be performed by the same apparatus as described herein.

The invention also provides methods for monitoring the level of CRP in a sample or subject. As described above, the sdAb are especially useful for the detection of the CRP level over time achieved by repeated in vitro or in vivo spot measurements of CRP over time or by the continuous measurement of CRP, e.g. in vitro or preferably in vivo, which can indicate inflammation spikes in a subject (patient). It is desired to monitor these inflammation spikes, i.e. increased CRP levels in a sample of a subject or in a subject over time. This is advantageous since it allows the skilled practitioner or the subject itself to perform targeted treatment of an underlying inflammatory or autoimmune disease. Such a monitoring of inflammation spikes allows for target treatment and reduces the amount of medication/drugs a subject has to take for properly managing their inflammatory or autoimmune disease and consequently reduces the disease burden and potential burden of side effects of the subject. A method of monitoring the level of CRP can be performed similar to the method for detecting and/or quantifying CRP in a sample or a subject as described herein above and to the in vivo diagnosis as described herein above. For monitoring CRP level, a sample of a subject can be contacted with an anti-CRP sdAb of the invention and the binding of the anti-CRP sdAb to CRP can be detected. When monitoring CRP levels in a sample of a subject over time, the method might be repeatedly applied to two or more samples of a subject that have been obtained in time steps. Thus, the method can repeatedly detect the binding of an anti-CRP sdAb of the present invention to CRP over time, i.e. at certain times which can be distinguished by time steps between detection events. Accordingly, the ex vivo or in vitro method of monitoring the level of CRP can be performed similar to the spot measurements in a sample of a subject or the measurements in a sample of a subject over time as described herein above.

Accordingly, the invention provides in one aspect an ex vivo or in vitro method of monitoring the level of CRP comprising:
(i) contacting a sample with an anti-CRP sdAb of the invention, and
(ii) detecting binding of the anti-CRP sdAb.

The ex vivo or in vitro method of monitoring the level of CRP can be performed by or in an apparatus as described herein.

The features described herein in context of the method for detecting and/or quantifying CRP in a sample apply mutatis mutandis to the ex vivo or in vitro method of monitoring the level of CRP. In some aspect, the ex vivo or in vitro method of monitoring the level of CRP may not comprise a step of obtaining a sample from the subject.

Accordingly, the invention provides in another aspect an ex vivo or in vitro method of monitoring the level of CRP comprising:
(i) contacting a sample with an anti-CRP sdAb of the invention, and
(ii) detecting binding of the anti-CRP sdAb,
wherein the sample comprises interstitial fluid (ISF), blood, urine, stool, serum, tear fluid, sweat, or saliva.

In another aspect, the invention provides an ex vivo or in vitro method of monitoring the level of CRP comprising:
(i) contacting a sample with an anti-CRP sdAb of the invention, and
(ii) detecting binding of the anti-CRP sdAb,
wherein the contacting step comprises contacting a sample via a syringe, a needle, a microfluidic or capillary-based system with the anti-CRP sdAb of the invention.

In another aspect, the invention provides an ex vivo or in vitro method of monitoring the level of CRP comprising:
(i) contacting a sample with an anti-CRP sdAb of the invention, and
(ii) detecting binding of the anti-CRP sdAb,
wherein the contacting step comprises contacting the sample with a biosensor of the invention, optionally wherein the biosensor comprises a sdAb of the invention conjugated to a working electrode.

In another aspect, the invention provides an ex vivo or in vitro method of monitoring the level of CRP comprising:
(i) contacting a sample with an anti-CRP sdAb of the invention, and
(ii) detecting binding of the anti-CRP sdAb,
wherein the detection step comprises physiochemical or electrochemical detection of the binding of the anti-CRP sdAb and CRP.

In another aspect, the invention provides an ex vivo or in vitro method of monitoring the level of CRP comprising:
(i) contacting a sample with an anti-CRP sdAb of the invention, and
(ii) detecting binding of the anti-CRP sdAb,
wherein the detection step comprises detection of the binding of the anti-CRP sdAb and CRP via an electrical signal produced by the binding.

In another aspect, the invention provides an ex vivo or in vitro method of monitoring the level of CRP comprising:
(i) contacting a sample with an anti-CRP sdAb of the invention, and
(ii) detecting binding of the anti-CRP sdAb,
wherein the detection step comprises detection of the binding of the anti-CRP sdAb and CRP with a biosensor of the invention, optionally, wherein the biosensor comprises a sdAb of the invention conjugated to a working electrode.

In another aspect, the invention provides an ex vivo or in vitro method of monitoring the level of CRP comprising:
(i) contacting a sample with an anti-CRP sdAb of the invention, and
(ii) detecting binding of the anti-CRP sdAb,
wherein the contacting step comprises contacting the sample with a biosensor of the invention, optionally wherein the biosensor comprises a sdAb of the invention conjugated to a working electrode and,
wherein the detection step comprises detecting the binding of the binding of the anti-CRP sdAb and CRP with a biosensor of the invention, optionally, wherein the biosensor comprises a sdAb of the invention conjugated to a working electrode.

In another aspect, the invention provides an ex vivo or in vitro method of monitoring the level of CRP comprising:
(i) contacting a sample with an anti-CRP sdAb of the invention, and
(ii) detecting binding of the anti-CRP sdAb, further comprising detecting the binding of the anti-CRP sdAb over time.

In another aspect, the invention provides an ex vivo or in vitro method of monitoring the level of CRP comprising:
(i) contacting a sample with an anti-CRP sdAb of the invention, and
(ii) detecting binding of the anti-CRP sdAb, further comprising detecting the binding of the anti-CRP sdAb over time, wherein steps (i) and
(ii) are performed on a further sample, optionally wherein the sample is from the same subject.

In another aspect, the invention provides an ex vivo or in vitro method of monitoring the level of CRP comprising:
(i) contacting a sample with an anti-CRP sdAb of the invention, and
(ii) detecting binding of the anti-CRP sdAb,
further comprising contacting and/or detecting the binding of the anti-CRP sdAb over time, wherein steps (i) and (ii) are performed on a further sample, optionally wherein the sample is from the same subject, and
wherein the contacting step comprises contacting the sample with a contacting a sample with a biosensor of the invention, optionally wherein the biosensor comprises a sdAb of the invention conjugated to a working electrode, and
wherein the detection step comprises detecting the binding of the binding of the anti-CRP sdAb and CRP with a biosensor of the invention, optionally, wherein the biosensor comprises a sdAb of the invention conjugated to a working electrode.

In a preferred aspect, the invention provides an ex vivo or in vitro method of monitoring the level of CRP comprising:
(i) contacting a sample comprising interstitial fluid (ISF), blood, urine, stool, serum, tear fluid, sweat, or saliva with an anti-CRP sdAb of the invention, and
(ii) detecting binding of the anti-CRP sdAb,
further comprising contacting and/or detecting the binding of the anti-CRP sdAb over time, wherein steps (i) and (ii) are performed on a further sample, optionally wherein the sample is from the same subject, and
wherein the contacting step comprises contacting the sample with a contacting a sample with a biosensor of the invention, optionally wherein the biosensor comprises a sdAb of the invention conjugated to a working electrode, and
wherein the detection step comprises detecting the binding of the binding of the anti-CRP sdAb and CRP with a biosensor of the invention, optionally, wherein the biosensor comprises a sdAb of the invention conjugated to a working electrode.

In another aspect, the invention provides an ex vivo or in vitro method of monitoring the level of CRP comprising:
(i) contacting a sample with an anti-CRP sdAb of the invention, and
(ii) detecting binding of the anti-CRP sdAb,
wherein the detection step comprises detection of the binding of the anti-CRP sdAb and CRP via electrochemical ELISA.

The ex vivo or in vitro method of monitoring the level of CRP described herein can be performed by or in an apparatus as described herein.

In another aspect, it may be preferred to monitor the level of CRP in a subject, i.e. in vivo. A method of monitoring the level of CRP in a subject in vivo is especially advantageous since no samples have to be obtained from the subject, e.g. in time steps, which reduces the workload of the practitioner and the burden on a subject, e.g. from multiple consecutive blood draws.

Accordingly, the invention provides in one aspect a method of monitoring the level of CRP in a subject comprising:
(i) contacting the subject with an anti-CRP sdAb of the invention, and
(ii) detecting binding of the anti-CRP sdAb.

The features described herein in context of the method for detecting and/or quantifying CRP in a subject apply mutatis mutandis to the method of monitoring the level of CRP in a subject.

In another aspect, the invention provides a method of monitoring the level of CRP in a subject comprising:
(i) contacting a subject's biological fluid or sample, such as bodily fluid, e.g. interstitial fluid (ISF), blood, urine, stool, serum, tear fluid, sweat, or saliva with an anti-CRP sdAb of the invention, and
(ii) detecting binding of the anti-CRP sdAb.

In another aspect, the invention provides a method of monitoring the level of CRP in a subject comprising:
(i) contacting a subject's interstitial fluid (ISF), blood, urine, stool, serum, tear fluid, sweat, or saliva with an anti-CRP sdAb of the invention, and
(ii) detecting binding of the anti-CRP sdAb.

In another aspect, the invention provides a method of monitoring the level of CRP in a subject comprising:
(i) contacting a subject's interstitial fluid (ISF) with an anti-CRP sdAb of the invention, and
(ii) detecting binding of the anti-CRP sdAb.

In another aspect, the invention provides a method of monitoring the level of CRP in a subject comprising:
(i) contacting the subject with an anti-CRP sdAb of the invention, and
(ii) detecting binding of the anti-CRP sdAb,
wherein the contacting step comprises contacting the subject with a biosensor of the invention, optionally wherein the biosensor comprises a sdAb of the invention conjugated to a working electrode.

In another aspect, the invention provides a method of monitoring the level of CRP in a subject comprising:
(i) contacting the subject with an anti-CRP sdAb of the invention, and
(ii) detecting binding of the anti-CRP sdAb,
wherein the contacting step comprises contacting the subject with a biosensor of the invention, optionally wherein the biosensor comprises a sdAb of the invention conjugated to a working electrode, and
wherein the detection step comprises detecting the binding of the binding of the anti-CRP sdAb and CRP with a biosensor of the invention, optionally, wherein the biosensor comprises a sdAb of the invention conjugated to a working electrode.

In another aspect, the invention provides a method of monitoring the level of CRP in a subject over time comprising:
(i) contacting the subject with an anti-CRP sdAb of the invention, and
(ii) detecting binding of the anti-CRP sdAb.

In another aspect, the invention provides a method of monitoring the level of CRP in a subject comprising:
(i) contacting the subject with an anti-CRP sdAb of the invention, and
(ii) detecting binding of the anti-CRP sdAb,
further comprising contacting and/or detecting the binding of the anti-CRP sdAb over time, wherein steps (i) and (ii) are repeatedly performed in or on the subject.

In another aspect, the invention provides a method of monitoring the level of CRP in a subject comprising:
(i) contacting the subject with an anti-CRP sdAb of the invention, and
(ii) detecting binding of the anti-CRP sdAb,
further comprising contacting and/or detecting the binding of the anti-CRP sdAb over time, wherein steps (i) and (ii) are repeatedly performed in or on the subject, and
wherein the contacting step comprises contacting the subject with a biosensor of the invention, optionally wherein the biosensor comprises a sdAb of the invention conjugated to a working electrode, and
wherein the detection step comprises detecting the binding of the binding of the anti-CRP sdAb and CRP with a biosensor of the invention, optionally, wherein the biosensor comprises a sdAb of the invention conjugated to a working electrode.

In a preferred aspect, the invention provides a method of monitoring the level of CRP in a subject comprising:
(i) contacting the subject's interstitial fluid (ISF), blood, urine, stool, serum, tear fluid, sweat, or saliva with an anti-CRP sdAb of the invention, and
(ii) detecting binding of the anti-CRP sdAb,
further comprising contacting and/or detecting the binding of the anti-CRP sdAb over time, wherein steps (i) and (ii) are repeatedly performed in or on the subject, and
wherein the contacting step comprises contacting the subject with a biosensor of the invention, optionally wherein the biosensor comprises a sdAb of the invention conjugated to a working electrode, and
wherein the detection step comprises detecting the binding of the binding of the anti-CRP sdAb and CRP with a biosensor of the invention, optionally, wherein the biosensor comprises a sdAb of the invention conjugated to a working electrode.

In another aspect, the invention provides a method of monitoring the level of CRP in a subject comprising:
(i) contacting the subject with an anti-CRP sdAb of the invention, and
(ii) detecting binding of the anti-CRP sdAb,
wherein the detection step comprises detection of the binding of the anti-CRP sdAb and CRP via electrochemical ELISA.

The method of monitoring the level of CRP in a subject described herein can be performed by or in an apparatus as described herein.

As described herein above, the apparatus of the present invention can comprise additional components, such as means for dispensing a drug/a dispensing unit for dispensing a drug. Accordingly, it is encompassed that the apparatus, either as an applicator like appliance or as a permanently or reversibly implanted appliance, can be used in the treatment of a disease or in a method of treatment. As mentioned, the apparatus of the present invention can comprise an anti-CRP sdAb of the present invention. Accordingly, the apparatus is especially useful in the treatment of inflammatory and autoimmune diseases provided herein. As mentioned herein above, it is envisioned that the apparatus of the present invention can measure and detect inflammation spikes, e.g. increased CRP levels and dispense a drug, e.g. anti-inflammation drug in response to such inflammation spikes. In general, all features described in the context of the apparatus and treatments of the present invention apply mutatis mutandis to the apparatus for use in the treatment of a disease provided herein. When used in the context of a treatment or therapy, the treatment may further comprise a set of dispensing a drug as described herein, e.g. a sdAb of the invention or any drug described in the context of treatments herein.

Accordingly, the invention provides in one aspect an apparatus comprising:
(i) an anti-CRP sdAb of the present invention, and
(ii) a contacting unit for contacting a subject with the anti-CRP sdAb, and
(iii) a detecting unit for detecting and/or quantifying binding of the anti-CRP sdAb, and
(iv) a dispensing unit for dispensing a drug, for use in therapy.

In another aspect, the invention provides an apparatus comprising:
(i) an anti-CRP sdAb of the present invention, and
(ii) a contacting unit for contacting a subject with the anti-CRP sdAb, and
(iii) a detecting unit for detecting and/or quantifying binding of the anti-CRP sdAb, and
(iv) a dispensing unit for dispensing a drug, for use in the treatment of a disease associated with an inflammation.

In another aspect, the invention provides an apparatus comprising:
(i) an anti-CRP sdAb of the present invention, and
(ii) a contacting unit for contacting a subject with the anti-CRP sdAb, and
(iii) a detecting unit for detecting and/or quantifying binding of the anti-CRP sdAb, and
(iv) a dispensing unit for dispensing a drug,
for use in the treatment of a disease associated with an inflammation, optionally a systemic inflammation.

In another aspect, the invention provides an apparatus comprising:
(i) an anti-CRP sdAb of the present invention, and
(ii) a contacting unit for contacting a subject with the anti-CRP sdAb, and
(iii) a detecting unit for detecting and/or quantifying binding of the anti-CRP sdAb, and
(iv) a dispensing unit for dispensing a drug,
for use in the treatment of a disease associated with an inflammation, optionally a systemic inflammation, wherein the disease is associated with an increased production or release of inflammatory markers.

In another aspect, the invention provides an apparatus comprising:
(i) an anti-CRP sdAb of the present invention, and
(ii) a contacting unit for contacting a subject with the anti-CRP sdAb, and
(iii) a detecting unit for detecting and/or quantifying binding of the anti-CRP sdAb, and
(iv) a dispensing unit for dispensing a drug, for use in the treatment of a disease associated with an inflammation, optionally a systemic inflammation, wherein the disease is associated with an increased production or release of CRP.

In another aspect, the invention provides an apparatus comprising:
(i) an anti-CRP sdAb of the present invention, and
(ii) a contacting unit for contacting a subject with the anti-CRP sdAb, and
(iii) a detecting unit for detecting and/or quantifying binding of the anti-CRP sdAb, and
(iv) a dispensing unit for dispensing a drug,
for use in the treatment of a disease associated with an inflammation, optionally a systemic inflammation, wherein the disease is an inflammatory and/or autoimmune disease.

In another aspect, the invention provides an apparatus comprising:
(i) an anti-CRP sdAb of the present invention, and
(ii) a contacting unit for contacting a subject with the anti-CRP sdAb, and
(iii) a detecting unit for detecting and/or quantifying binding of the anti-CRP sdAb, and
(iv) a dispensing unit for dispensing a drug,
for use in the treatment of rheumatoid arthritis, inflammatory bowel disease, IgA nephropathy, Psoriasis, lupus, multiple sclerosis, Endometriosis, Sepsis, cardiovascular disease, metabolic syndrome, hypertension, diabetes, cancer, bacterial or viral infections, COVID19, asthma, familial Mediterranean fever, Alzheimer's disease, Parkinson's diseases, diabetic macular edema (DME), diabetic retinopathy, uveitis, glaucoma, dry eye (e.g., dry eye disease or dry eye syndrome), allergic conjunctivitis, ocular pain, rhegmatogenous retinal detachment (RRD), age-related macular degeneration (AMD), proliferative diabetic retinopathy (PDR), retinal vein occlusion (RVO), neuromyelitis optica (NMO), corneal transplant, corneal abrasion, physical injury to the eye, non-alcoholic fat liver disease, chronic kidney disease, or tumour.

In another aspect, the invention provides an apparatus comprising:
(i) an anti-CRP sdAb of the present invention, and
(ii) a contacting unit for contacting a subject with the anti-CRP sdAb, and
(iii) a detecting unit for detecting and/or quantifying binding of the anti-CRP sdAb, and
(iv) a dispensing unit for dispensing a drug,
for use in the treatment of a disease associated with an inflammation, wherein the apparatus performs the method for detecting and/or quantifying CRP in or on a subject of the present invention prior to dispensing a drug.

In another aspect, the invention provides an apparatus comprising:
(i) an anti-CRP sdAb of the present invention, and
(ii) a contacting unit for contacting a subject with the anti-CRP sdAb, and
(iii) a detecting unit for detecting and/or quantifying binding of the anti-CRP sdAb, and
(iv) a dispensing unit for dispensing a drug,
for use in the treatment of a disease associated with an inflammation, wherein the apparatus performs the method for detecting and/or quantifying CRP in or on a subject of the present invention prior to dispensing a drug, and wherein the dispensing of said drug depends on the measurement obtained from the method for detecting and/or quantifying CRP in or on a subject.

In another aspect, the invention provides an apparatus comprising:
(i) an anti-CRP sdAb of the present invention, and
(ii) a contacting unit for contacting a subject with the anti-CRP sdAb, and
(iii) a detecting unit for detecting and/or quantifying binding of the anti-CRP sdAb, and
(iv) a dispensing unit for dispensing a drug,
for use in the treatment of a disease associated with an inflammation, wherein the apparatus performs the method for detecting and/or quantifying CRP in or on a subject of the present invention and compares the level of CRP to a reference value prior to dispensing a drug, and wherein the dispensing of said drug depends on the difference between the measured CRP level and the reference value.

In another aspect, the invention provides an apparatus comprising:
(i) an anti-CRP sdAb of the present invention, and
(ii) a contacting unit for contacting a subject with the anti-CRP sdAb, and
(iii) a detecting unit for detecting and/or quantifying binding of the anti-CRP sdAb, and
(iv) a dispensing unit for dispensing a drug,
for use in the treatment of a disease associated with an inflammation, wherein the apparatus performs the method for detecting and/or quantifying CRP in or on a subject of the present invention and compares the level of CRP to a reference value prior to dispensing a drug, and wherein the dispensing and amount of said drug depends on the difference between the measured CRP level and the reference value.

In another aspect, the invention provides an apparatus comprising:
(i) an anti-CRP sdAb of the present invention, and
(ii) a contacting unit for contacting a subject with the anti-CRP sdAb, and
(iii) a detecting unit for detecting and/or quantifying binding of the anti-CRP sdAb, and
(iv) a dispensing unit for dispensing a drug,
for use in the treatment of a disease associated with an inflammation, wherein the apparatus is permanently or reversibly attached to or implanted into a subject.

In another aspect, the invention provides an apparatus comprising:
(i) an anti-CRP sdAb of the present invention, and
(ii) a contacting unit for contacting a subject with the anti-CRP sdAb, and
(iii) a detecting unit for detecting and/or quantifying binding of the anti-CRP sdAb, and
(iv) a dispensing unit for dispensing a drug,
for use in the treatment of a disease associated with an inflammation, wherein the treatment further comprises comparing the level of anti-CRP with a reference value or reference sample as described herein.

In another aspect, the invention provides an apparatus comprising:
(i) an anti-CRP sdAb of the present invention, and
(ii) a contacting unit for contacting a subject with the anti-CRP sdAb, and
(iii) a detecting unit for detecting and/or quantifying binding of the anti-CRP sdAb, and
(iv) a dispensing unit for dispensing a drug,
for use in the treatment of a disease associated with an inflammation, wherein the apparatus repeatedly contacts the subject with the anti-CRP sdAb and repeatedly detects and/or quantifies binding of the anti-CRP sdAb over time.

In another aspect, the invention provides an apparatus comprising:
(i) an anti-CRP sdAb of the present invention, and
(ii) a contacting unit for contacting a subject with the anti-CRP sdAb, and
(iii) a detecting unit for detecting and/or quantifying binding of the anti-CRP sdAb, and
(iv) a dispensing unit for dispensing a drug,
for use in the treatment of a disease associated with an inflammation, wherein the apparatus repeatedly contacts the subject with the anti-CRP sdAb and repeatedly detects and/or quantifies binding of the anti-CRP sdAb over time.

In a preferred aspect, the invention provides an apparatus comprising:
(i) an anti-CRP sdAb of the present invention, and
(ii) a contacting unit for contacting a subject with the anti-CRP sdAb, and
(iii) a detecting unit for detecting and/or quantifying binding of the anti-CRP sdAb, and
(iv) a dispensing unit for dispensing a drug,
for use in the treatment of a disease associated with an inflammation, wherein the apparatus repeatedly contacts the subject with the anti-CRP sdAb and repeatedly detects and/or quantifies binding of the anti-CRP sdAb over time, wherein the treatment further comprises comparing the level of anti-CRP with a reference value or reference sample as described herein after every detection and/or quantification step, and wherein the apparatus is permanently or reversibly attached to or implanted into a subject.

In addition, the invention provides an apparatus for use in in vivo diagnosis of a disease associated with an inflammation. In general, all features described herein in the context of the apparatus and (in vivo) diagnosis of the present invention apply mutatis mutandis to the apparatus for use in in vivo diagnosis of a disease provided herein.

Accordingly, the invention provides in one aspect an apparatus comprising:
(i) an anti-CRP sdAb of the present invention, and
(ii) a contacting unit for contacting a subject with the anti-CRP sdAb, and
(iii) a detecting unit for detecting and/or quantifying binding of the anti-CRP sdAb, for use in in vivo diagnosis.

In another aspect, the invention provides an apparatus comprising:
(i) an anti-CRP sdAb of the present invention, and
(ii) a contacting unit for contacting a subject with the anti-CRP sdAb, and
(iii) a detecting unit for detecting and/or quantifying binding of the anti-CRP sdAb, for use in in vivo diagnosis of a disease associated with inflammation.

In another aspect, the invention provides an apparatus comprising:
(i) an anti-CRP sdAb of the present invention, and
(ii) a contacting unit for contacting a subject with the anti-CRP sdAb, and
(iii) a detecting unit for detecting and/or quantifying binding of the anti-CRP sdAb,
for use in in vivo diagnosis of a disease associated with inflammation, optionally a systemic inflammation.

In another aspect, the invention provides an apparatus comprising:
(i) an anti-CRP sdAb of the present invention, and
(ii) a contacting unit for contacting a subject with the anti-CRP sdAb, and
(iii) a detecting unit for detecting and/or quantifying binding of the anti-CRP sdAb,
for use in in vivo diagnosis of a disease associated with inflammation, optionally a systemic inflammation, wherein the disease associated with an inflammation or systemic inflammation is an inflammatory disease and/or an autoimmune disease.

In another aspect, the invention provides an apparatus comprising:
(i) an anti-CRP sdAb of the present invention, and
(ii) a contacting unit for contacting a subject with the anti-CRP sdAb, and
(iii) a detecting unit for detecting and/or quantifying binding of the anti-CRP sdAb,
for use in in vivo diagnosis of a disease associated with inflammation, optionally a systemic inflammation, wherein the disease associated with an inflammation or systemic inflammation is rheumatoid arthritis, inflammatory bowel disease, IgA nephropathy, Psoriasis, lupus, multiple sclerosis, Endometriosis, Sepsis, cardiovascular disease, metabolic syndrome, hypertension, diabetes, cancer, bacterial or viral infections, COVID19, asthma, familial Mediterranean fever, Alzheimer's disease, Parkinson's diseases, diabetic macular edema (DME), diabetic retinopathy, uveitis, glaucoma, dry eye (e.g., dry eye disease or dry eye syndrome), allergic conjunctivitis, ocular pain, rhegmatogenous retinal detachment (RRD), age-related macular degeneration (AMD), proliferative diabetic retinopathy (PDR), retinal vein occlusion (RVO), neuromyelitis optica (NMO), corneal transplant, corneal abrasion, physical injury to the eye, non-alcoholic fat liver disease, chronic kidney disease, or tumour.

In another aspect, the invention provides an apparatus comprising:
(i) an anti-CRP sdAb of the present invention, and
(ii) a contacting unit for contacting a subject with the anti-CRP sdAb, and
(iii) a detecting unit for detecting and/or quantifying binding of the anti-CRP sdAb,
for use in in vivo diagnosis of a disease associated with inflammation, optionally a systemic inflammation, wherein the apparatus repeatedly contacts the subject with the anti-CRP sdAb and repeatedly detects and/or quantifies binding of the anti-CRP sdAb.

In another aspect, the invention provides an apparatus comprising:
(i) an anti-CRP sdAb of the present invention, and
(ii) a contacting unit for contacting a subject with the anti-CRP sdAb, and
(iii) a detecting unit for detecting and/or quantifying binding of the anti-CRP sdAb,
for use in in vivo diagnosis of a disease associated with inflammation, optionally a systemic inflammation, wherein the apparatus repeatedly contacts the subject with the anti-CRP sdAb and repeatedly detects and/or quantifies binding of the anti-CRP sdAb over time.

In another aspect, the invention provides an apparatus comprising:
(i) an anti-CRP sdAb of the present invention, and
(ii) a contacting unit for contacting a subject with the anti-CRP sdAb, and
(iii) a detecting unit for detecting and/or quantifying binding of the anti-CRP sdAb,
for use in in vivo diagnosis of a disease associated with inflammation, optionally a systemic inflammation, wherein the apparatus is permanently or reversibly attached to or implanted into a subject.

In another aspect, the invention provides an apparatus comprising:
(i) an anti-CRP sdAb of the present invention, and
(ii) a contacting unit for contacting a subject with the anti-CRP sdAb, and
(iii) a detecting unit for detecting and/or quantifying binding of the anti-CRP sdAb,
for use in in vivo diagnosis of a disease associated with inflammation, optionally a systemic inflammation, wherein the diagnosis further comprises comparing the level of anti-CRP with a reference value or reference sample as described herein.

In another aspect, the invention provides an apparatus comprising:
(i) an anti-CRP sdAb of the present invention, and
(ii) a contacting unit for contacting a subject with the anti-CRP sdAb, and
(iii) a detecting unit for detecting and/or quantifying binding of the anti-CRP sdAb,
for use in in vivo diagnosis of a disease associated with inflammation, optionally a systemic inflammation, wherein the apparatus repeatedly contacts the subject with the anti-CRP sdAb and repeatedly detects and/or quantifies binding of the anti-CRP sdAb over time and wherein the diagnosis further comprises comparing the level of anti-CRP with a reference value or reference sample as described herein after every detection and/or quantification step, and wherein the apparatus is permanently or reversibly attached to or implanted into a subject.

The invention also provides a method of producing an anti-CRP sdAb. As mentioned herein above, the anti-CRP sdAb of the present invention are particularly useful since they embody specific on-rate constant and off-rate constant ranges. Accordingly, an anti-CRP sdAb in the sense of the present invention can be produced by specifically selecting for a desired on-rate constant and off-rate constant. An exemplary range of an on-rate constant in the sense of the invention is about 3.30×10\hat{ }4−1.70×10\hat{ }7. An exemplary range of an off-rate constant is about 3.20×10\hat{ }−9−2.20×10\hat{ }−1. Accordingly, an anti-CRP sdAb can be specifically selected for an on-rate constant (kₒₙ) of about 3.30×10\hat{ }4−1.70×10\hat{ }7 and an off-rate constant (k_{off}) of about 3.20×10\hat{ }−9−2.20×10\hat{ }−1. Another exemplary range of range of an on-rate constant in the sense of the invention is about 3.80×10\hat{ }5−1.70×10\hat{ }7. Another exemplary range of an off-rate constant is about 3.20×10\hat{ }−9−1.30×10\hat{ }−2. Accordingly, an anti-CRP sdAb can be specifically selected for an on-rate constant (kₒₙ) of about 3.80×10\hat{ }5−1.70×10\hat{ }7 and an off-rate constant (k_{off}) of about 3.20×10\hat{ }−9−1.30×10\hat{ }−2. Another exemplary range of range of an on-rate constant in the sense of the invention is about 3.30×10\hat{ }4−4.60×10\hat{ }6. Another exemplary range of an off-rate constant is about 2.60×10\hat{ }−4−2.20×10\hat{ }−1. Accordingly, an anti-CRP sdAb can be specifically selected for an on-rate constant (kₒₙ) of about 3.30×10\hat{ }4-4.60×10\hat{ }6 and an off-rate constant (k_{off}) of about 2.60×10\hat{ }−4−2.20×10\hat{ }−1. Another exemplary range of range of an on-rate constant in the sense of the invention is about 4.10×10\hat{ }4−3.80×10\hat{ }5. Another exemplary range of an off-rate constant is about 2.00×10\hat{ }−5-6.90×10\hat{ }−3. Accordingly, an anti-CRP sdAb can be specifically selected for an on-rate constant (kₒₙ) of about 4.10×10\hat{ }4−3.80×10\hat{ }5 and an off-rate constant (k_{off}) of about 2.00×10\hat{ }−5−6.90×10\hat{ }−3. The skilled person is aware how to select for useful on-rate and off-rate constant ranges depending on the desired application.

Accordingly, the invention provides in one aspect a method of producing an anti-CRP sdAb comprising:
(i) contacting a subject with a recombinant human CRP, and
(ii) isolating a sdAb which successfully binds to CRP, and
(iii) selecting for an on-rate constant (kₒₙ) of about 3.30×10\hat{ }4−1.70×10\hat{ }7 and an off-rate constant (k_{off}) of about 3.20×10\hat{ }−9−2.20×10\hat{ }−1.

In another aspect, the invention provides a method of producing an anti-CRP sdAb comprising:
(i) contacting a subject with a recombinant human CRP, and
(ii) isolating a sdAb which successfully binds to CRP, and
(iii) selecting for an on-rate constant (kₒₙ) of about 3.80×10\hat{ }5−1.70×10\hat{ }7 and an off-rate constant (k_{off}) of about 3.20×10\hat{ }−9−1.30×10\hat{ }−2.

In another aspect, the invention provides a method of producing an anti-CRP sdAb comprising:
(i) contacting a subject with a recombinant human CRP, and
(ii) isolating a sdAb which successfully binds to CRP, and
(iii) selecting for an on-rate constant (kₒₙ) of about 3.30×10\hat{ }4−4.60×10\hat{ }6 and an off-rate constant (k_{off}) of about 2.60×10\hat{ }−4−2.20×10\hat{ }−1.

In another aspect, the invention provides a method of producing an anti-CRP sdAb comprising:
(i) contacting a subject with a recombinant human CRP, and
(ii) isolating a sdAb which successfully binds to CRP, and
(iii) selecting for an on-rate constant (kₒₙ) of about 4.10×10\hat{ }4−3.80×10\hat{ }5 and an off-rate constant (k_{off}) of about 2.00×10\hat{ }−5−6.90×10\hat{ }−3.

A particular desired property of an anti-CRP sdAb of the present invention is the ability to continuously and reversibly bind CRP. In addition, it is desired that a sdAb of the present invention can bind to multiple CRP molecules, i.e. binding to a second or a further CRP molecule after dissociating from a first CRP molecule.

Accordingly, the present invention provides in one aspect a method of producing an anti-CRP sdAb comprising:
(i) contacting a subject with a recombinant human CRP, and
(ii) isolating a sdAb which successfully binds to CRP, and
(iii) selecting for an on-rate constant (kₒₙ) of about 3.30×10\hat{ }4−1.70×10\hat{ }7 and an off-rate constant (k_{off}) of about 3.20×10\hat{ }−9−2.20×10\hat{ }−1, and
(iv) selecting for continuous and reversible binding of CRP.

In another aspect, the present invention provides a method of producing an anti-CRP sdAb comprising:
(i) contacting a subject with a recombinant human CRP, and
(ii) isolating a sdAb which successfully binds to CRP, and
(iii) selecting for an on-rate constant (kₒₙ) of about 3.30×10\hat{ }4−1.70×10\hat{ }7 and an off-rate constant (k_{off}) of about 3.20×10\hat{ }−9−2.20×10\hat{ }−1, and
(iv) selecting for continuous and reversible binding of CRP, and
(v) selecting for binding to a second or further CRP after dissociation from a first CRP.

As mentioned herein above, it can in some embodiments be desired for a sdAb to be able to inhibit the biological function, while it is in other embodiments desired that a sdAb does not inhibit the biological function of CRP. Thus, the method of producing an anti-CRP sdAb can further comprise a step of selecting if a sdAb inhibits or does not inhibit the biological function of CRP. The biological function of CRP may be CRP signalling or the CRP signalling pathway, i.e. an inflammation response.

Accordingly, the invention provides in one aspect a method of producing an anti-CRP sdAb comprising:
(i) contacting a subject with a recombinant human CRP, and
(ii) isolating a sdAb which successfully binds to CRP, and
(iii) selecting for an on-rate constant (kₒₙ) of about 3.30×10\hat{ }4−1.70×10\hat{ }7 and an off-rate constant (k_{off}) of about 3.20×10\hat{ }−9−2.20×10\hat{ }−1, and
(iv) selecting for continuous and reversible binding of CRP, and
(v) selecting for inhibiting the biological function of CRP or not inhibiting the biological function of CRP.

In another aspect, the invention provides a method of producing an anti-CRP sdAb comprising:
(i) contacting a subject with a recombinant human CRP, and
(ii) isolating a sdAb which successfully binds to CRP, and
(iii) selecting for an on-rate constant (kₒₙ) of about 3.30×10\hat{ }4−1.70×10\hat{ }7 and an off-rate constant (k_{off}) of about 3.20×10\hat{ }−9−2.20×10\hat{ }−1, and
(iv) selecting for continuous and reversible binding of CRP, and
(v) selecting for inhibiting the biological function of CRP or not inhibiting the biological function of CRP, wherein the biological function of CRP is CRP signalling or the CRP signalling pathway, such as an inflammation response.

A subject in the sense of the method of producing an anti-CRP sdAb can be a camelid. A camelid is an animal of the biological family of Camelidae. Exemplary camelids are dromedary camels, Bactrian camels, wild Bactrian camels, llamas, alpacas, vicuñas, guanacos, and huarizos. In a preferred embodiment a subject in the context of the method of producing an anti-CRP sdAb as described herein is a camelid. In another preferred embodiment a subject in the context of the method of producing an anti-CRP sdAb as described herein is a huarizo.

The invention also provides anti-CRP sdAbs produced by the method of producing an anti-CRP sdAb as described herein. The skilled person is aware how to select and produce desired sdAbs in the sense of the present invention. One way of producing sdAbs in the sense of the present invention is provided in detail in the appended examples. Exemplary anti-CRP sdAbs produced by the method of producing an anti-CRP sdAb described herein and in the appended examples are provided in SEQ ID NOs: 1, 9, 17, 25, 33, 41, 49, 57, 65, 73, 81, or 89.

In any of the embodiments provided herein, an anti-CRP sdAb can also be used in combination with an anti-interleukin 6 (anti-IL6) sdAb.

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art. As appropriate, procedures involving the use of commercially available kits and reagents are generally carried out in accordance with manufacturer-defined protocols and conditions unless otherwise noted.

As used herein, the singular forms “a,” “an”, and “the” include the plural referents unless the context clearly indicates otherwise. The terms “include”, “such as”, and the like are intended to convey inclusion without limitation, unless otherwise specifically indicated.

As used herein, the term “or” is generally employed in its usual sense including “and/or” unless the content clearly dictates otherwise. The term “and/or” means one or all of the listed elements or a combination of any two or more of the listed elements.

As used herein, the term “comprising” also specifically includes embodiments “consisting of” and “consisting essentially of” the recited elements, unless specifically indicated otherwise. The terms “comprising” and “having” may be used interchangeably herein.

As used herein, the term “about” indicates and encompasses an indicated value and a range above and below that value. In certain embodiments, the term “about” indicates the designated value±10%, ±5%, or ±1%. In certain embodiments, where applicable, the term “about” indicates the designated value(s)±one standard deviation of that value(s).

As used herein “association” preferably means that an anti-CRP sdAb of the invention “associates” i.e. binds with an CRP. As used herein “bind” or “binds” preferably means binding of an anti-CRP sdAb of the invention with CRP, e.g. in a sample from a subject, or in/on a subject. Binding or association of an anti-CRP sdAb with CRP may be detected in vitro, ex vivo or in vivo.

As used herein “subject” means an individual. In general, a subject in the sense of the invention can be a mammal, preferably a human. In the sense of the treatments and diagnoses of the invention, a subject is preferably a human. In the sense of the treatments and diagnoses of the invention, a subject may be a patient, such as a human or animal patient. In the sense of the method of producing an anti-CRP sdAb, a subject is preferably a camelid, more preferably a huarizo.

As used herein “measurement” in general means the detection and/or quantification of CRP. Thus, the term measurement may relate to a detection of CRP and/or the quantification of an CRP level. In all aspects of the invention, a measurement of an CRP level can be compared to a reference sample or reference value as described herein.

As used herein “contacting” in general means that two compounds are being brought into close proximity so that a reaction can happen. In the context of contacting a sample or a subject with an anti-CRP sdAb of the invention, the term “contacting” means that the anti-CRP sdAb is brought into close proximity with a sample comprising interstitial fluid (ISF), blood, urine, stool, serum, tear fluid, sweat, saliva in vitro or a subject's interstitial fluid (ISF), blood, urine, stool, serum, tear fluid, sweat, or saliva in vivo, which comprise CRP, thereby enabling binding/association of the anti-CRP sdAb with CRP.

The term “contacting” can also relate to administering a compound, e.g. a drug to a subject. In the sense of the apparatus per se of the present invention, the term “contacting” can mean that the apparatus is applied to a subject, e.g. a subject's skin. In addition, it can encompass that the apparatus is permanently or reversibly attached to or implanted into a subject. In the sense of the contacting steps performed by the apparatus of the invention, the term “contacting” means that an anti-CRP sdAb of the invention is brought into close proximity with an CRP, thereby enabling binding/association of the anti-CRP sdAb with CRP.

As used herein, terms like “variant thereof having up to [n] amino acid substitutions, means that the variant can have any of the concrete number of n amino acid substitutions. For example, if the “variant has up to 5 amino acid substitutions”, the variant can have 1, 2, 3, 4 or 5 amino acid substitutions.

As used herein “apparatus” may also be referred to as device.

The methods provided herein can be performed on a sample, such as a sample obtained from a subject. In some embodiments a step of obtaining a sample is not part of any one of the methods described herein. In some embodiments any of the methods described herein do not comprise a step of obtaining a sample from a subject, such as a human or animal.

In some embodiments any one of the methods or uses described herein can be in vitro and/or ex vivo or in vivo. In some embodiments any of the method steps described herein can be in vitro and/or ex vivo or in vivo.

The disclosures in context of the methods described herein are disclosed as corresponding use mutatis mutandis. The disclosures in context of the use described herein are disclosed as corresponding methods mutatis mutandis.

In one aspect, the methods of the present invention are not methods for treatment of the human or animal body by therapy. In a further aspect, the methods of the present invention are not processes for modifying the germ line genetic identity of human beings. In one aspect, the methods of the present invention are in vitro or ex vivo methods. In a further aspect, the methods of the present invention are not processes for modifying the genetic identity of animals which are likely to cause them suffering without any substantial medical benefit to man or animal. The methods and uses of the present invention can be in vivo, ex vivo or in vitro.

Biacore systems allow monitoring the interaction between molecules in real time using the phenomenon of surface plasmon resonance (SPR). This involves attaching one interacting partner to the surface of a sensor chip, and then passing a sample containing the second interaction partner over the surface. Binding of molecules to the sensor surface generates a response which is proportional to the bound mass. Accordingly, as used herein the term “SPR Biacore” means a SPR binding kinetic measurement. Both terms can be used interchangeably herein. Accordingly, as used herein “wherein the values are obtained by a SPR Biacore binding kinetic measurement” or “wherein the values are determined/assessed using a SPR Biacore binding kinetic measurement” and “SPR Biacore measurement” are equivalent to and can be interchangeably used with “wherein the values are obtained by/determined/assessed using a SPR binding kinetic measurement” and “SPR binding kinetic measurement”, respectively.

When referring herein to any measurement obtained, determined, performed “by a SPR Biacore” and similar formulations, the SPR Biacore is preferably a SPR Biacore T200. Furthermore, when referring herein to any measurements “obtained/determined/assessed by a SPR Biacore” and similar formulations, the measurements were done preferably according to the standard protocol provided by the manufacturer, such as in the “Biacore™ sensor surface handbook” published in 2022 and available on https://cdn.cytivalifesciences.com/api/public/content/digi-16475-pdf (availability as of 10.05.2024) which is incorporated herein in its entirety. An exemplary method to obtain measurements by SPR binding kinetic measurements is provided in Stenberg et al. 1991, which is incorporated herein in its entirety. An exemplary method to obtain measurements by a SPR Biacore or SPR binding kinetic measurements in general is provided in the appended examples. When referring herein to any measurements “obtained/determined by a SPR Biacore” and similar formulations, the measurement method preferably comprises:
a. immobilizing sdAbs of the invention on a SPR COOH-chip (e.g. CM5 sensor chip, Cytiva, Cat. 29149603) through EDC/NHS coupling, wherein coupling is done individually optimized for each sdAb based on its isoelectric point (pI) by altering pH with sodium acetate buffer,
b. setting the coupling to reach a similar target loading signal to provide coating density for all sdAbs to be measured (e.g. 100 response units),
c. diluting CRP in a running buffer to, e.g. 7-8, different CRP concentrations that can be measured on a SPR, e.g. a Biacore T200, (sample flow rate-30 μL/min), wherein the concentration range depends on the K_{D} of each sdAb (e.g. an estimate of which was obtained from Octet BLI screening),
d. obtaining a reference (buffer only) measurement for each sdAb
d. obtaining kinetic curves and applying them for fitting the 1:1 Langmuir binding model (e.g. using the Biacore Evaluation software 3.2.), wherein the measurement length is 300 sec for association and 900-1,000 sec for dissociation,
wherein the running buffer comprises 140 mM NaCl, 10 mM HEPES, 2 mM CaCl₂), 0.01% Tween20 (pH 8.0), or wherein the running buffer comprises 1×PBS±0.01% Tween20, pH 7.4,
wherein the sdAbs are provided in in 10 mM glycine pH 2.0,
preferably wherein the method is performed on a Biacore T200.

Unless otherwise stated herein the “SPR Biacore binding kinetic measurement” as used herein is performed according to standard manufacturer's conditions (as e.g. described in the “Biacore™ sensor surface handbook”) and/or according to Stenberg et al. In one preferred aspect, a running buffer can be used comprising the following components: 140 mM NaCl, 10 mM HEPES, 2 mM CaCl₂, 0.01% Tween20 (pH 8.0) or 1×PBS±0.01% Tween20, pH 7.4.

Bio-Layer Interferometry (BLI) is an optical analytical technique used to study biomolecular interactions in real-time. It works by measuring the interference pattern of white light reflected from two surfaces: a biosensor surface and an internal reference surface.

When biomolecules bind to the biosensor surface, the optical thickness at the surface changes, resulting in a change in the interference pattern. This change in the interference pattern is measured and used to determine the kinetics and affinity of the molecular interactions occurring on the biosensor surface.

BLI is commonly used for applications such as:
1. Characterizing biomolecular interactions: BLI can measure the binding kinetics, affinity constants, and thermodynamics of interactions between proteins, nucleic acids, small molecules, and other biomolecules.
2. Analyzing protein-protein interactions: BLI is particularly useful for studying protein-protein interactions, which are crucial for understanding biological processes and developing therapeutics.
3. Measuring protein-small molecule interactions: BLI can detect and quantify the binding of small molecules to proteins, which is important in drug discovery and development.
4. Epitope mapping: BLI can map the binding sites (epitopes) of antibodies on target proteins, aiding in the design of therapeutic antibodies and vaccine development.
5. Concentration analysis: BLI can be used to determine the concentration of biomolecules in solution by measuring the binding response to a known target.

BLI offers advantages such as label-free detection, low sample consumption, and the ability to analyze interactions in complex matrices like cell lysates or serum.

BLI is an optical biosensing technique that utilizes an integrated waveguide and interferometer to monitor biomolecular interactions in real-time. The core component is a biosensor, which consists of a linear optical waveguide and two measurement surfaces: a biological surface and an internal reference surface.

The biological surface is functionalized with specific capture molecules (e.g., proteins, nucleic acids, small molecules) that can bind to the analyte of interest. The reference surface is designed to account for any non-specific binding or bulk refractive index changes.

White light is coupled into the waveguide, where it propagates and reflects off the two surfaces, creating an interference pattern. The phase shift in the interference pattern is directly proportional to the thickness and refractive index of the biological layer on the sensor surface. As biomolecules bind to the capture molecules, the optical thickness of the biological layer changes, resulting in a shift in the interference pattern. This shift is detected and quantified by the interferometer, providing a real-time measurement of the binding kinetics and affinity between the analyte and the immobilized capture molecules.

The rate of binding (association rate constant, kₐ) and the rate of dissociation (dissociation rate constant, k_{d}) can be determined by fitting the binding curves to suitable mathematical models. The equilibrium dissociation constant (K_{D}), which is a measure of affinity, can be calculated from the ratio of k_{d}/kₐ.

BLI offers several advantages over other biosensing techniques, such as:
1. Label-free detection: No labeling of biomolecules is required, reducing the risk of altering their native structure and behavior.
2. High sensitivity: BLI can detect biomolecular interactions with picomolar to nanomolar sensitivity.
3. Wide molecular weight range: BLI can analyze interactions involving small molecules (<1 kDa) to large complexes (>1 MDa).
4. Real-time kinetics: BLI provides continuous monitoring of binding events, enabling the determination of kinetic rate constants.
5. Multiplexing capabilities: Multiple biosensors can be used simultaneously to study different interactions in parallel.

In Octet bio-layer interferometry (BLI) systems, one molecule is immobilized to a Dip and Read Biosensor and binding to a second molecule is measured. A change in the number of molecules bound to the end of the biosensor tip causes a shift in the interference pattern that is measured in real-time. Accordingly, as used herein the term “Octet BLI” means a BLI binding kinetic measurement. Both terms can be used interchangeably herein. Accordingly, as used herein “wherein the values are obtained by an Octet BLI binding kinetic measurement” or “wherein the values are determined/assessed using an Octet BLI kinetic measurement” and “Octet BLI measurement” are equivalent to and can be interchangeably used with “wherein the values are obtained by/determined/assessed using a BLI binding kinetic measurement” and “BLI binding kinetic measurement”, respectively.

When referring herein to any measurement obtained, determined, performed “by a Octet BLI” and similar formulations, the Octet BLI is preferably a ForteBio Octet RED96. Furthermore, when referring herein to any measurements “obtained/determined/assessed by a Octet BLI” and similar formulations, the measurements were done preferably according to the standard protocol provided by the manufacturer, such as in the “Octet® System Data Acquisition User Guide” Release 11.0 available on https://facilities.bioc.cam.ac.uk/files/media/data_acquisition_octet_web.pdf (availability as of 10.05.2024) which is incorporated herein in its entirety. An exemplary method to obtain measurements by an Octet BLI or BLI binding kinetic measurements in general is provided in the appended examples. When referring herein to any measurements “obtained/determined by a Octet BLI” and similar formulations, the measurement method preferably comprises:
a. immobilizing Biotinylated hCRP (e.g. Sigma, Cat 236603) on Streptavidin-coated sensors (e.g. Sartorius Cat 18-5019) for 80 sec with the biotinylated hCRP was at 10 μg/mL in the running buffer,
b. diluting purified sdAbs of the present invention in the running buffer to a range of concentrations, e.g. 100, 50, 25, 12.5, 6.3, 3.1, 1.6 nM, wherein the exact concentrations depend on the respective sdAb clones,
c. dipping the (Octet) BLI sensors into the wells of a 96-well plate containing the sdAb samples,
d. regenerating between readings using using 10 mM glycine buffer (pH 1.7), wherein the readings were done with the following parameters:
Association: 420 sec
Dissociation 600 sec
Assay temperature: 25° C.
Agitation at 1,000 rpm
e. repeating all measurements with reference sensors (without biotinylated hCRP) to measure background signal, which was subsequently subtracted from the binding signal of the sdAbs,
wherein the running buffer comprises 10 mM Tris, 140 nM NaCl, 2 nM CaCl₂ (pH 8.0), or wherein the running buffer comprises 140 mM NaCl, 10 mM HEPES, 2 mM CaCl₂, 0.01% Tween20 (pH 8.0),
preferably wherein the method is performed on a ForteBio Octet RED96.

Unless otherwise stated herein the “BLI binding kinetic measurement” as used herein is performed according to standard manufacturer's conditions (as e.g. described in the “Octet® System Data Acquisition User Guide”). In one preferred aspect, a running buffer can be used comprising the following components: 10 mM Tris, 140 nM NaCl, 2 nM CaCl₂) (pH 8.0). In another preferred aspect, a running buffer can be used comprising the following components: 140 mM NaCl, 10 mM HEPES, 2 mM CaCl₂, 0.01% Tween20 (pH 8.0).

### BRIEF DESCRIPTION OF THE DRAWINGS

FIG. 1: Modeling of the proportion of bound sdAbs for different association rate values kₐ. The solid black line, highlighted by an arrow, represents the modelled concentration change of CRP molecule in vivo (left y axis). The dotted lines represent the proportion of occupied sdAbs as predicted by the TEM for different values of association rate kₐ (right y axis). The values used to generate these plots are: low kₐ=0.3×10³ M⁻¹×s⁻¹., medium kₐ=1×10³ M⁻¹×s⁻¹, high kₐ=3×10³ M⁻¹×s⁻¹. The dissociation rate is kept constant, k_{d}=1×10−3 s⁻¹.

FIG. 2: Modeling of the proportion of bound sdAbs for different dissociation rate values kₐ. The solid black line, highlighted by an arrow, represents the modelled concentration change of CRP molecule in vivo (left y axis). The dotted lines represent the proportion of occupied sdAbs as predicted by the TEM for different values of dissociate rate kₐ (right y axis). The values used to generate these plots are: low kₐ=0.3×10−3 s⁻¹, medium kₐ=1×10−³ s⁻¹, high kₐ=3×10−3 s⁻¹. The association rate is kept constant, kₐ=1×10³ M⁻¹×s⁻¹.

FIG. 3: Schematic of the overall experimental procedure to raise and isolate anti-CRP sdAbs from an adult Huarizo.

FIG. 4: Assessment of the immune response on day 0, 47, 75 and 124 against human CRP by ELISA. Bound antibodies were detected using a goat-anti-llama-HRP IgG GaLPO.

FIG. 5: Generation of the sdAb-phagemid libraries of day 43, day 75 and day 124. A) Gel electrophoresis of 5 μl of the precipitated RNA samples on a 1% TBE agarose gel. The 28S and 18S ribosomal RNA bands are indicated. B) Amplification of the sdAb (~700 bp) and the VH (~900 bp) cDNA, analysed on a 1% TBE agarose gel. C) 10-fold dilutions of TG1 E. coli cells transformed with the sdAb libraries, spotted onto LB-agar plates supplemented with 100 μg/ml ampicillin and 2% glucose. D) Colony PCR of different clones from each transformation/library.

FIG. 6: Results of the first round of selections on CRP. A) Input phages were diluted up to 1×10\hat{ }10 and used to infect E. coli TG1 before spotting on selective LB agar plates. B) Results of the selection output on wells coated with indicated amount of directly coated CRP.

FIG. 7: Results of the second round of selections on CRP. A) Input phages were diluted up to 1×10\hat{ }10 and used to infect E. coli TG1 before spotting on selective LB agar plates. B) Results of the selection output on wells coated with indicated amount of directly coated CRP and with the indicated incubation times and washing conditions.

FIG. 8: Analysis of sequences from master plates SCR-1, SCR-2 and SCR-3 using the PipeBio software. A) sdAb annotation score indicated the percentages of successfully annotated sdAb genes in the dataset. B) Treemap indicating the relative size of the sdAb clusters found amongst master plates SCR-1, SCR-2 and SCR-3. Cluster number and CDR-H3 sequence are depicted. C) Cluster locations indicated in the master plates layouts, empty well: no correct/annotated sequence, light blue: unique sequence.

FIG. 9: Concentration standard curve established by Octet BLI used to derive anti-CRP sdAb concentration in periplasmic extracts.

FIG. 10: PageBlue-stained SDS-PAGE gel containing Ref) 1 μg of tagless reference sdAb, M) PageRuler™ Prestained Protein Ladder and 1 μg of the indicated sdAb.

FIG. 11: Assessing the binding of purified sdAb from masterplate SCR-1, SCR-2 and SCR-3 to immobilized CRP by ELISA. Bound sdAb were detected using rabbit-anti-sdAb, followed by donkey-anti-rabbitHRP and OPD as substrate.

FIG. 12: SPR Biacore binding kinetic measurements done for anti-CRP sdAb SCR-1A1. Curves selected for fitting. Gray scale curves indicate CRP binding signal at different concentrations, black curves indicate fitted model curves.

FIG. 13: SPR Biacore binding kinetic measurements done for anti-CRP sdAb SCR-2D1. Curves selected for fitting. Gray scale curves indicate CRP binding signal at different concentrations, black curves indicate fitted model curves.

FIG. 14: SPR Biacore binding kinetic measurements done for anti-CRP sdAb SCR-3A4. Curves selected for fitting. Gray scale curves indicate CRP binding signal at different concentrations, black curves indicate fitted model curves.

FIG. 15: SPR Biacore binding kinetic measurements done for anti-CRP sdAb SCR-3E4. Curves selected for fitting. Gray scale curves indicate CRP binding signal at different concentrations, black curves indicate fitted model curves.

FIG. 16: SPR Biacore binding kinetic measurements done for anti-CRP sdAb SCR-3H1. Curves selected for fitting. Gray scale curves indicate CRP binding signal at different concentrations, black curves indicate fitted model curves.

FIG. 17: SPR Biacore binding kinetic measurements done for anti-CRP sdAb SCR-3H6. Curves selected for fitting. Gray scale curves indicate CRP binding signal at different concentrations, black curves indicate fitted model curves.

FIG. 18: SPR Biacore binding kinetic measurements done for anti-CRP sdAb SCR-2B4. Curves selected for fitting. Gray scale curves indicate CRP binding signal at different concentrations, black curves indicate fitted model curves.

FIG. 19: SPR Biacore binding kinetic measurements done for anti-CRP sdAb SCR-2D2. Curves selected for fitting. Gray scale curves indicate CRP binding signal at different concentrations, black curves indicate fitted model curves.

FIG. 20: SPR Biacore binding kinetic measurements done for anti-CRP sdAb SCR-3A5. Curves selected for fitting. Gray scale curves indicate CRP binding signal at different concentrations, black curves indicate fitted model curves.

FIG. 21: SPR Biacore binding kinetic measurements done for anti-CRP sdAb SCR-3B7. Curves selected for fitting. Gray scale curves indicate CRP binding signal at different concentrations, black curves indicate fitted model curves.

FIG. 22: SPR Biacore binding kinetic measurements done for anti-CRP sdAb SCR-3E5. Curves selected for fitting. Gray scale curves indicate CRP binding signal at different concentrations, black curves indicate fitted model curves.

FIG. 23: SPR Biacore binding kinetic measurements done for anti-CRP sdAb SCR-3G7. Curves selected for fitting. Gray scale curves indicate CRP binding signal at different concentrations, black curves indicate fitted model curves.

The invention is illustrated in the following examples.

### Example 1: Generation of Anti-CRP sdAbs (VHHs)

Key abbreviations
BLI: Bio-Layer Interferometry
CRP: C-reactive protein
K_{D}: dissociation constant (affinity)=k_{d}/kₐ
k_{d}: kinetic dissociation rate constant (also k_{off}, or off-rate)
kₐ: kinetic association rate constant (also kₒₙ, or on-rate)
PBMCs: Peripheral Blood Mononuclear Cells
PBS: Phosphate-buffered saline
PBST: PBS with 0.05% Tween-20
MPBS: 4% milk powder in PBS
RT: Room temperature
TEM: Thermodynamic equilibrium model
VHH: heavy chain variable domain of a camelid single-chain antibody; also: single-domain antibody (sdAb)

### Material and Methods

### Analytical Modeling of CRP Dynamics

Gaussian functions were used to model CRP concentration dynamics that closely mimic signals measured in human serum during homeostasis and disease (Devran et al., 2012; Dinh et al., 2019; Hart et al., 2020; Herieka & Erridge, 2014; Keegan et al., 2020; Landry et al., 2017; Mavropoulou et al., 2020; Póvoa et al., 1998; Ridker et al., 2003; Ruiz-González et al., 2016; Sproston & Ashworth, 2018).

The CRP concentration equation was defined as follow:

$$C ( t ) = {C}_{baseline} + {C}_{high} \cdot {e}^{\frac{2 {( t - μ )}^{2}}{{w}^{2}}} \qquad Equation 1$$

With C_{baseline} and C_{high} the baseline and peak concentration values respectively, t the time interval, u the time interval at which the concentration value is maximum, and w reflecting the duration of the concentration peak. Multi-gaussian distributions were obtained by summing multiple Gaussian signals with different parameters μ. A typical CRP concentration was represented by the following input parameters: C_{baseline}=0.01 μM, C_{high}=0.4 μM, μ=24 h, w=14 h and a time interval t=1 min.

### Analytical Implementation of the Thermodynamic Equilibrium Model (TEM)

Binding kinetics between CRP and anti-CRP sdAb were approximated by the Langmuir absorption model (Kusnezow et al., 2006). This simple analytical formula describes the kinetic interactions between an analyte, here the CRP molecule, and a capture molecule, here respectively: the CRP antigen and the anti-CRP sdAbs.

According to this thermodynamic equilibrium model, the proportion of bound sdAbs S(t) can be expressed as:

$$S ( t ) = {S}_{e q} ( 1 - {e}^{- \frac{t}{τ}} ) \qquad Equation 2$$

$$With :$$

$${S}_{eq} = {S}_{max} \frac{C ( t )}{C ( t ) + {K}_{D}} \qquad Equation 3$$

$${K}_{D} = \frac{{k}_{d}}{{k}_{a}} , and \qquad Equation 4$$

$$τ = \frac{1}{{k}_{a} + {k}_{a} \cdot C ( t )} = \frac{1}{{k}_{a} ( {K}_{D} + C ( t ) )} \qquad Equation 5$$

$$\dot{S} ( 0 ) = {C}_{0} {k}_{a} {S}_{max} \qquad Equation 6$$

Where C(t) is the analyte concentration at time t, Sₘₐₓ the maximum proportion of bound active sites (i.e., 100%), S_{eq} the signal at equilibrium, kₐ the association rate, k_{d} the dissociation rate, K_{D} the dissociation constant, and t the reaction time constant. \dot{S}(0) indicates the initial reaction rate and C₀ a fixed concentration at time t=0.

This simplified model assumes no limitation from analyte diffusion. It implies that, for a dissociation constant K_{D} largely superior to the analyte concentration C(t), the readout signal is proportional to C(t) at any time t. When K_{D} approaches or exceeds C(t), a delay is introduced between the readout signal and the concentration dynamics, and the linear relationship does not hold anymore.

To calculate the proportion of bound sdAbs S(t) for a given CRP concentration target range, the limit of detection of the system was assumed to be equivalent to 0.01% of bound sdAbs, or 100 parts per million.

Immunization of a Huarizo with Recombinant Human CRP

One adult Huarizo was immunized with human CRP (Sigma, Cat. 236603) and GERBU FAMA adjuvant. Six successive immunizations were performed every 2 weeks (starting at day 0), injecting 50 μg CRP antigen plus adjuvant intramuscularly. Antigen and adjuvant were injected as a 1:1 (v/v) mixture except for the third and fifth injection where antigen and adjuvant were injected at the same time but at separate locations. On day 47, 75 and 124, serum and PBMCs were collected. PBMCs were used for total RNA isolation. RNA was precipitated in EtOH and NaAc and dissolved in RNase-free milli-Q.

### Testing the Huarizo's Immune Response Against Human CRP by ELISA

The immune response after immunization is determined by ELISA using sera from day 0 (before immunization), day 47, day 75 and day 124. The ELISA is done according to standard laboratory procedures. In short, MaxiSorp plates are coated overnight at 4° C. with 50 μl of antigen at 2 μg/ml in PBS. The next day, the antigen is removed, plates are washed with PBS and then blocked with 4% milk powder in PBS (MPBS). Next, the wells are incubated with serially diluted sera for 1 hour at room temperature. Bound antibodies in the sera are detected with rabbit-anti-sdAb, followed by donkey-anti-rabbit HRP. In between incubations, plates are washed with PBS with 0.05% Tween-20 (PBST) or PBS. Bound HRP-coupled antibodies are detected using o-phenylenediamine dihydrochloride (OPD) with H₂O₂ as substrate and converted substrate is measured at 490 nm wavelength.

Generating sdAb Phagemid Libraries

Integrity of the total RNA is determined by assessing the 28S and 18S rRNA on a 1% agarose gel. A total of 40 μg RNA is transcribed into cDNA using a reverse transcriptase reaction (Thermo Fisher Scientific), which is followed by purification using a PCR clean-up kit (Macherey Nagel). cDNAs encoding for heavy chain immunoglobulin domains (both conventional and heavy chain-only) are amplified by PCR using proprietary primer sets. This results in two types of DNA fragments (sizes of ~900 bp and ~700 bp) which represent the conventional and heavy chain-only antibody cDNAs respectively. The ~700 bp fragments (CH1-VHH of the heavy chain-only antibodies) are excised from the gel, purified and used as a template for a nested PCR that introduces an 5′ flanking SfiI restriction site. Amplified nested-PCR fragments are then purified as before and subsequently digested with SfiI and Eco91I. This results in ~400 bp gene fragments containing the sdAb genes, which are purified using a PCR clean-up kit (Machery Nagel). Digested sdAb genes are ligated in frame with genelII into the pQ81 phagemid vector and transformed into TG1 by electroporation. To assess the transformation efficiency, the transformed TG1 are titrated using 10-fold dilutions and then spotted on LB-agar plates supplemented with 100 μg/ml ampicillin and 2% glucose. The total number of transformants was estimated by counting colonies in the highest dilution and using the formula: Library size=(amount of colonies)*(dilution)*3 ml/0.005 ml (spotted volume). The insert frequency was determined by randomly picking 24 clones from each library and performing a colony PCR using primers that anneal in the pQ81 vector. In this PCR, successfully inserted sdAb fragments will yield PCR-fragments of ~700 bp, compared to a ~300 bp for empty plasmids. All libraries were stored at −80° C. in 2xYT medium supplemented with 20% glycerol, 2% glucose and 100 μg/ml ampicillin.

### Phage Production and Panning

Phages expressing the sdAbs via fusion to the minor coat protein pIII were based on the VCSM13 bacteriophage and were produced in E. coli strain TG1. For the first round of selections, phages were produced from the libraries in TG1 bacteria. TG1 containing the sdAb-phagemid libraries were diluted from glycerol stocks in 2xYT medium containing 2% glucose and 100 μg/ml ampicillin. The cultures were inoculated up to an OD600 of 0.05, while the number of bacteria of the inoculum is >10× the library size (>1×10\hat{ }8 bacteria inoculum). Inoculated cultures were grown at 37° C. for 2 hours to reach an OD600 of ~0.5. Subsequently, 7 ml of the cultures were infected with helper phage VCSM13 using a MOI (multiplicity of infection) of ~100. After 30 minutes, infected bacteria were spun down and resuspended into 50 ml fresh 2xYT medium supplemented with both ampicillin (100 μg/ml) and kanamycin (25 μg/ml). Phages were produced overnight at 37° C., shaking. For the second round of selections, phages were produced from rescued outputs. Overnight cultures from rescued outputs were first diluted a 100-fold in 5 ml fresh 2xYT medium supplemented with 2% glucose and 100 μg/ml ampicillin and grown for 2 hours until the logarithmic growth phase. Subsequently, these cultures were infected with helper phage and phages were produced overnight as described above. In general, produced phages were precipitated from the supernatant of the cultures using PEG-NaCl precipitation. Titers of the produced phages were calculated by serial dilution of the phage sample and infection of E. coli TG1.

### Phage-Display and Panning Selections

For the first round of selections, precipitated phages of each library were pre-blocked and applied to wells coated overnight at 4° C. with 100 μl CRP in 140 mM NaCl, 10 mM Tris, 2 mM CaCl₂), pH 8.0 at 5 μg/mL. The next day, unbound antigen was removed, the wells were washed three times with PBS and then blocked for 1 hour at room temperature (RT) with 200 μl of 4% MPBS. The amount of phages used was at least 10-fold of the transformation efficiency (estimated diversity) of the library. Phages were pre-blocked with 100 μl of 2% MPBS for 30 minutes at RT (phage dilution of 1:5). For the panning, the coated and blocked wells were incubated with the pre-blocked phages for 2 hours at RT. Upon extensive washing with PBS-Tween and PBS, bound phages were eluted with 0.1M TEA-solution, which was immediately neutralized with 1M Tris/HCl pH7.5. Eluted phages were allowed to infect TG1 bacteria (rescued output). In order to assess the size of the output, eluted phages were serially diluted, infected into TG1 and then spotted on LB-agar plates supplemented with 2% glucose and 100 μg/ml of ampicillin. For the second round of selections, phages produced from the outputs of the day 75 library were used to generate input titers. 1 μl of the precipitated and PBS-resuspended phages per selection condition were then pre-blocked in 100 μl of 2% MPBS (phage dilution of 1:100) and subsequently applied to CRP-coated and blocked wells at RT. To direct the panning towards selecting sdAb with a high on-rate, CRP was coated at increasingly high concentrations, namely 20, 2 and 0.2 μg/mL. To dispose of the extremely high affinity binders, a 5-minute pre-incubation on the coated CRP was done in some conditions. In addition, the phages were in some conditions incubated for a longer time period, 240 minutes as opposed to 120 minutes or 5 minutes. Lastly, to prevent loss of sdAb with higher off-rate, a shorter wash was applied with only 5 minutes of washing compared to >30 minutes of washing in some conditions. Eluted phages were rescued in TG1 and spotted on LB-agar plates as indicated above. All rescued outputs were stored at −80° C. as glycerol stocks. Single clones from the selection outputs were picked and combined in three 96-wells masterplates (192 sdAb clones in total), which were cultivated overnight at 37° C. in 2xYT medium supplemented with 2% glucose and 100 μg/ml ampicillin. Next, 20% glycerol was added and the plates stored at −80° C.

Production of Periplasmic Extracts from Masterplates

In order to test individual sdAb clones from masterplates for antigen-binding, periplasmic extracts were produced. Masterplates were duplicated into deep well plates containing 1 ml 2xYT medium supplemented with 0.1% glucose and 100 μg/ml ampicillin per well. These cultures were grown for 3 hours at 37° C. after which production of sdAb was induced with 1 mM of isopropyl-beta-D-thiogalactopyranoside (IPTG). The production of sdAb was conducted overnight at RT. Periplasmic extracts were prepared by collecting the bacteria by centrifugation, their resuspension into 120 μl of PBS, and a single overnight freeze-thaw cycle at −20° C. The next day, bacteria debris was spun down by centrifugation to yield the soluble periplasmic fraction containing the sdAb in the supernatant. Periplasmic extracts were either used for ELISA immediately or stored at −20° C.

### Periplasmic Extract ELISA

MaxiSorp plates were coated overnight at 4° C. with 50 μl of CRP antigen at 2 μg/ml in PBS. The next day, the antigen was removed, plates were washed and then blocked with 4% milk powder in PBS (MPBS). Next, the wells were incubated with 5× diluted periplasmic extracts for 1 hour at RT. Bound antibodies in the sera were detected with rabbit-anti-sdAb, followed by donkey-anti-rabbit-HRP. In between incubations, plates were washed with PBS with 0.05% Tween-20 (PBST) or PBS. Bound HRP-coupled antibodies were detected using o-phenylenediamine dihydrochloride (OPD) with H₂O₂ as substrate and converted substrate was measured at 490 nm wavelength.

Periplasmic Anti-CRP sdAb Binding Kinetics Screening Using Octet BLI

The assay running buffer used for all experiments described below was 140 mM NaCl, 10 mM HEPES, 2 mM CaCl₂, 0.01% Tween20 (pH 8.0).

Quantification of sdAbs in the Periplasmic Extracts

In order to determine the binding constants of the sdAbs, the concentration of the nanobodies in the periplasmic extracts had to be determined. The sdAbs contained a poly-histidine tag (His-tag) and their presence could be detected with Octet instrument by using anti-His sensors (Sartorius, Cat 18-5120). The quantification standard curve was determined using a control in PBS (pH 7.4) with 0.01% Tween. Sensors were regenerated in 10 mM glycine (pH 1.7). Periplasmic extracts containing anti-CRP sdAbs were diluted 1:7 in the running buffer prior to the measurements. The readings were done with the following parameters:
Association: 120 sec
Assay temperature: 25° C.
Agitation at 400 rpm
   Binding Assay (i.e., Kinetic Screening of Non-Purified sdAbs in Periplasmic Extracts)

hCRP (Sigma, Cat 236603) was biotinylated in house (ThermoFisher, Cat. 21435) and immobilized on Streptavidin coated sensors (Sartorius, Cat 18−5019). The sensor loading was done for 60 sec with the biotinylated CRP at 10 μg/mL in the running buffer. The running buffer used for the kinetic measurements was 10 mM Tris, 140 nM NaCl, 2 nM CaCl₂) (pH 8.0). Periplasmic extracts were diluted ~7-fold in the running buffer (30 μL periplasmic extract+170 μL buffer).

The Octet BLI sensors were dipped into the wells of 96-well plate that contained the diluted periplasmic extracts. Regeneration between readings was done using 10 mM glycine buffer (pH 1.7). The readings were done with the following parameters:
Association: 120 sec
Dissociation 120 or 180 sec
Assay temperature: 25° C.
Agitation at 1,000 rpm

All measurements were repeated with reference sensors (without CRP) to measure background signal, which was subsequently subtracted from the binding signal of the sdAbs.

E. coli Production and Purification of Anti-CRP sdAbs

Selected clones were transformed into the E. coli strain BL21. Pre-cultures were prepared by growing bacteria containing the selected sdAb in pQ81 overnight at 37° C. in 8 ml 2×YT medium supplemented with ampicillin (100 μg/ml) and 2% glucose. The next day, pre-cultures were diluted into 800 ml pre-warmed 2-YT medium and supplemented with 100 μg/ml ampicillin and 0.1% glucose. The bacteria are grown until OD600 of 0.6−0.9 at 37° C. before induction of the sdAb expression with 1 mM of IPTG. sdAbs were expressed for 4 hours at 37° C. and bacteria are then harvested by centrifugation. Bacteria pellets were resuspended into 30 ml PBS and frozen overnight at −20° C. Frozen bacteria were thawed at RT and spun down to separate the sdAb-containing soluble fraction from cell debris. sdAb were purified from the soluble fraction using immobilized metal affinity chromatography (IMAC) using the C-terminal His-tag on the sdAb and sepharose resin charged with cobalt. Bound sdAb were eluted with 150 mM imidazole, which is removed by repetitive dialysis against PBS. sdAb concentration determined by UV-VIS spectrometry at 280 nm wavelength. In addition, the purity and integrity of lug of purified sdAbs is assessed Coomassie blue staining of a 15% SDS-PAGE gel after electrophoresis.

ELISA for Apparent sdAb Binding Affinity to CRP (i.e., Kinetic Screening of Purified sdAbs)

MaxiSorp plates were coated with 2 μg/ml in PBS 50 μl/well antigen and incubated overnight. The next day, the antigen was removed, plates were washed with PBS and then blocked with 4% skimmed milk powder in PBS (MPBS). Next, the wells were incubated with sdAb dilution starting at 1000 nM 1:10 for 1 hour at RT. Bound antibodies were detected with rabbit-anti-sdAb, followed by donkey-anti-rabbit-HRP. In between incubations, plates were washed with PBS with 0.05% Tween-20 (PBST) or PBS. Bound HRP-coupled antibodies were detected using o-phenylenediamine dihydrochloride (OPD) with H₂O₂ as substrate and converted substrate is measured at 490 nm wavelength.

Anti-CRP Binding Kinetics Characterization of Purified sdAb Samples Using Octet BLI

hCRP (Sigma, Cat 236603) was biotinylated in house (ThermoFisher, Cat. 21435) and immobilized on Streptavidin coated sensors (Sartorius, Cat 18-5019). The sensor loading was done for 80 sec with the biotinylated CRP at 10 μg/mL in the running buffer. The running buffer used for the kinetic measurements was 10 mM Tris, 140 nM NaCl, 2 nM CaCl₂ (pH 8.0). Purified samples were diluted in the running buffer to 7 concentrations: 100, 50, 25, 12.5, 6.3, 3.1, 1.6 nM.

The Octet BLI sensors were dipped into the wells of 96-well plate that contained the samples. Regeneration between readings was done using 10 mM glycine buffer (pH 1.7). The readings were done with the following parameters:
Association: 420 sec
Dissociation 600 sec
Assay temperature: 25° C.
Agitation at 1,000 rpm

All measurements were repeated with reference sensors (without biotinylated CRP) to measure background signal, which was subsequently subtracted from the binding signal of the sdAbs.

Anti-CRP sdAb Binding Kinetics Characterization Using Surface Plasmon Resonance (SPR) Biacore (of Purified Samples)

sdAbs were immobilised on SPR COOH-chip (CM5 sensor chip, Cytiva, Cat. 29149603) through EDC/NHS coupling. Coupling was individually optimised for each sdAb on its isoelectric point (pI) by altering pH with sodium acetate buffer. Coupling was set to reach the similar target loading signal to provide coating density for all sdAbs (100 response units). CRP was diluted in the running buffer to 7-8 different CRP concentrations that were measured on a Biacore T200 (sample flow rate-30 μL/min), with the concentration range depending on the Kp of each sdAb (the estimate of which was obtained from Octet BLI screening). For each sdAb 4 reference (buffer only) measurements were conducted. The 4−6 kinetic curves were used for fitting the 1:1 Langmuir binding model using the Biacore Evaluation software 3.2. The measurement length was 300 sec for association, 900−1,000 sec for dissociation. sdAbs were regenerated in 10 mM glycine pH 2.0.

Calculation of Standard Deviations for kₒₙ and k_{off} Values

The standard deviation values provided for in Tables 7 and 11 have been calculated with three different methods. The standard deviation (SD) has been obtained by repeating BLI Octet and SPR Biacore binding kinetics measurements, respectively, as described herein and in section “Anti-CRP sdAb binding kinetics characterization using Octet BLI” below.

The standard deviation (Rich et al) (SD (Rich et al)) has been obtained considering the average standard deviation of different machines in the prior art. In particular, an average kₒₙ value of 1.40×10\hat{ }5 and a corresponding average standard deviation of 1.30×10\hat{ }5 has been extracted from Table 1 of Rich et al. Similarly, an average k_{off} value of 6.10×10\hat{ }−5 and a corresponding average standard deviation of 8.70×10\hat{ }−5 has been extracted from Table 1 of Rich et al. A standard deviation factor has been obtained by dividing the standard deviation by the corresponding average kₒₙ or k_{off} values. Mean values of the kₒₙ k_{off} measurement values of the sdAb obtained by BLI Octet and/or SPR Biacore binding kinetics then have been multiplied by the standard deviation factor to obtain the SD (Rich et al). The standard deviation factor obtained for the kₒₙ value was 9.29×10\hat{ }−1, the standard deviation factor obtained for the k_{off} value was 1.43.

The standard deviation (Conservative SD) corresponds to the maximum standard deviation value obtained from SD or SD (Rich et al) as described above.

### Results

Establishing a Predictive Thermodynamic Equilibrium Model to Tune the Selection of Anti-CRP sdAbs

A framework for the rational selection of anti-CRP sdAbs tuned towards the detection of CRP at physiologically relevant concentrations was designed. The target application ranges for CRP-based inflammation sensing in humans in vivo were derived from literature (Devran et al., 2012; Dinh et al., 2019; Hart et al., 2020; Herieka & Erridge, 2014; Keegan et al., 2020; Landry et al., 2017; Mavropoulou et al., 2020; Póvoa et al., 1998; Ridker et al., 2003; Ruiz-González et al., 2016; Sproston & Ashworth, 2018) and summarized in Table 1.

TABLE 1
CRP concentrations in humans during
homeostasis and disease.

| | Concentration in | |
|---|---|---|
| | a biological fluid | Example |
| Range | such as blood | indications/disease |
| Baseline | <0.01 μM | |
| Low | 0.01 μM-0.1 μM | Metabolic Syndrome |
| elevation | | (incl obesity) |
| | | CVD risk (low: 1 mg/ml, |
| | | interm: 1-3, high: >3) |
| | | Low self rated |
| | | physical health score |
| | | Active IBD |
| Moderate | 0.1 μM-1 μM | Cholecystitis |
| elevation | | Chronic obstructive |
| | | pulmonary disease (COPD) |
| | | Acute bronchitis |
| | | Sepsis |
| | | Active advanced cancer, |
| | | metastasis (>50 mg/ml) |
| | | Prediction of |
| | | Immunotherapy |
| | | efficiency |
| Severe | >1 μM | Pancreatitis |
| elevation | | Pneumonia |
| | | Sepsis related mortality |
| | | Severe infection (lung, |
| | | urinary, skin, GI, blood), |
| | | Rheumatologic causes |
| | | and/or Advanced tumors |

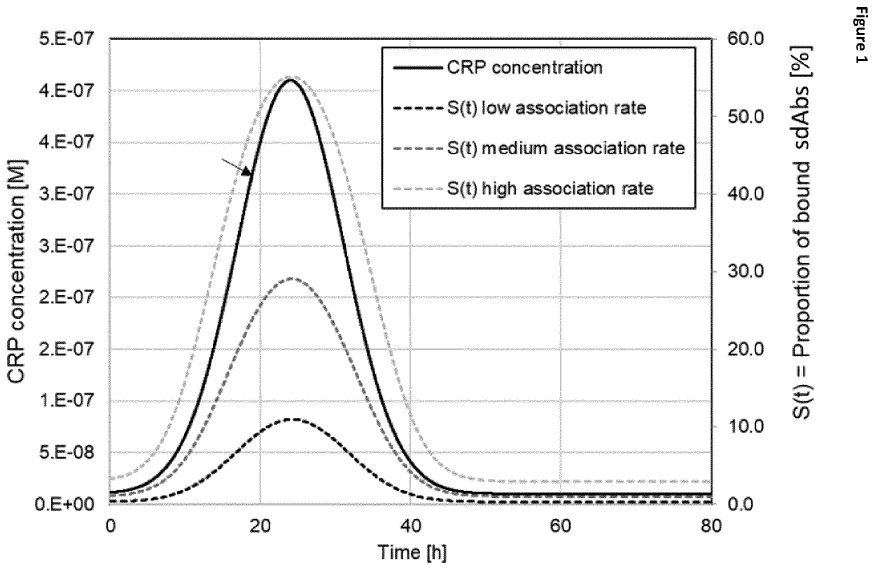

A typical CRP concentration dynamic is shown in FIG. 1. The sdAbs selection was based on a predictive analytical model, labelled hereafter as thermodynamic equilibrium model (TEM). The TEM was used to investigate the impact that varying kinetic binding rates, kₐ and k_{d}, had on the signal readout.

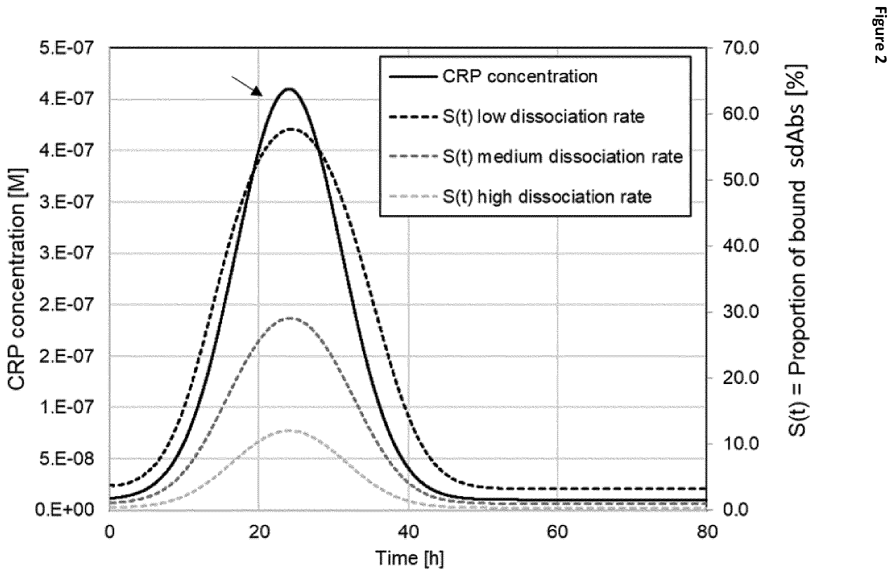

The implementation of the TEM demonstrated than for a fixed dissociation kinetic rate k_{d}, by increasing the value of the association rate kₐ, the proportion of occupied sdAbs S(t) also increased, and thus the signal readout (FIG. 1). However, for the largest value of kₐ explored, signal distortion is observed (i.e. the shape of the concentration peak is not conserved). For a fixed value of association rate kₐ, increasing the k_{d} resulted in a decreased signal readout amplitude but no significant modification of the signal shape (FIG. 2). Conversely, decreasing the k_{d} resulted in a large signal amplitude, but a greater distortion of the signal shape (FIG. 2). These first results suggest a non-linear relationship between the input CRP concentration C(t) and the proportion of bound sdAbs S(t) for some combinations of kₐ and k_{d} values. These non-linear effects arise from the fact that, for some kinetic rates combination, the modelled concentration of CRP C(t) is close to, or larger than the dissociation constant of the sdAb-CRP couple K_{D} (C(t)≤K_{D} in Equation 3).

Immunization of a Huarizo with Recombinant Human CRP to Raise Diverse Anti-CRP Antibody Responses

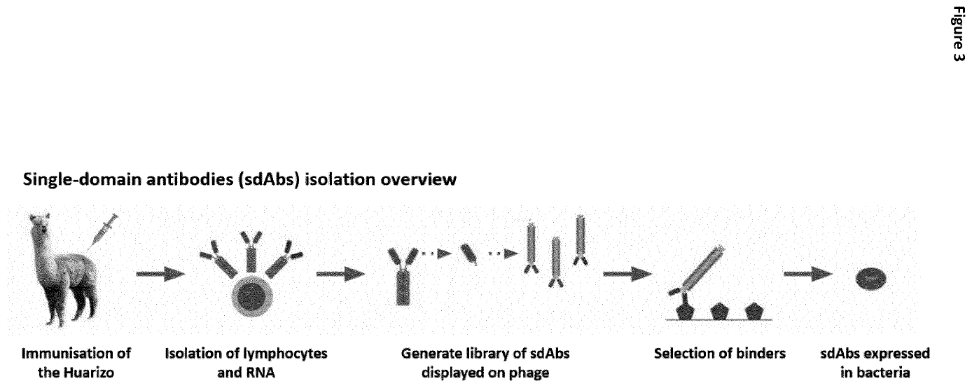

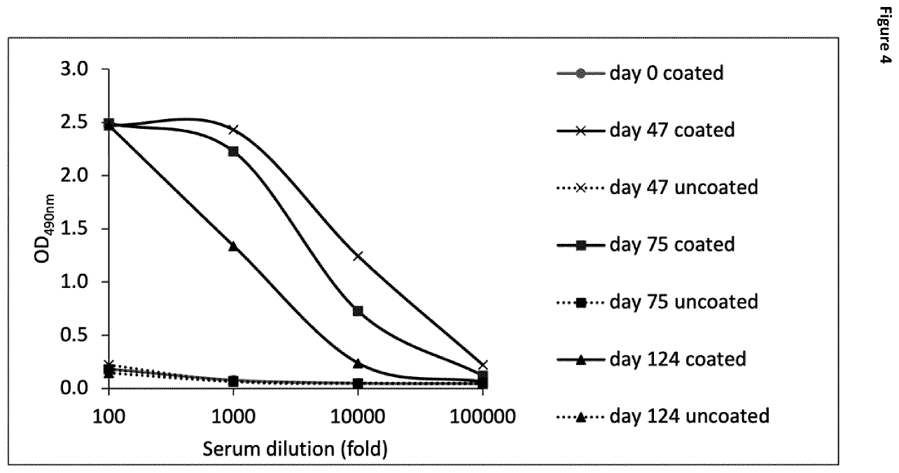

In order to raise a diverse set of anti-CRP sdAbs, from which to rationally select the ones with kₐ/k_{d} values relevant to measuring CRP levels measured in human serum during homeostasis and disease, natural immunization followed by phage-display and guided panning was performed (FIG. 3). One adult Huarizo was immunized six times with human CRP and GERBU FAMA adjuvant. To assess the immune response against CRP, a serial dilution of serum collected on day 0, 47, 75 and 124 was tested by ELISA (FIG. 4). The data shows a moderate immune response against CRP on either day 47 and 75, while a decreased response was observed on day 124.

Phage-Display and Guided Panning to Drive sdAb Selection Towards Relevant kₐ and kₐ

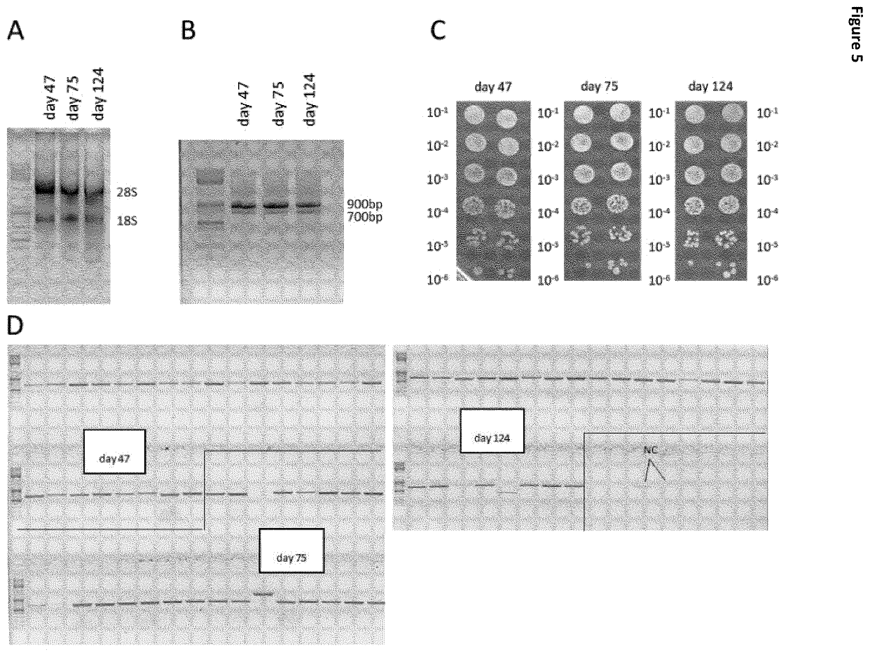

For the sdAb phagemid library construction, RNA integrity was confirmed by the clear visibility of intact 28S and 18S rRNA on an 1% agarose gel (FIG. 5A). Transcribed cDNA fragments of immunoglobulin domains (both conventional and heavy chain) were amplified using primers annealing at the IGH leader sequence region and the CH2 region (FIG. 5B). The 700 bp DNA fragment was excised and purified before being ligated into the pQ81 phagemid vector. The ligated library was transformed into TG1 E. coli by electroporation. This transformation was serially diluted to determine the number of transformants, which is indicative of the library size (FIG. 5C). The number of transformants was 1.2×10\hat{ }9 for the day 47 library, 1.8×10\hat{ }9 for the day 75 library and 1.8×10\hat{ }9 for the day 124 library (FIG. 5C). The insertion frequency was 100% for the day 43 library, 95% for the day 75 library and 95% the day 124 library (FIG. 5D). The results of the library construction are summarized in Table 2.

TABLE 2
Summary table of library construction

| | # of | Correct insertion | |
|---|---|---|---|
| Sample | transformants | frequency (%) | OD600 |
| Day 47 | 1.2 × 10\hat{ }9 | 100 | 99 |
| Day 75 | 1.8 × 10\hat{ }9 | 95 | 114 |
| Day 124 | 1.8 × 10\hat{ }9 | 95 | 102 |

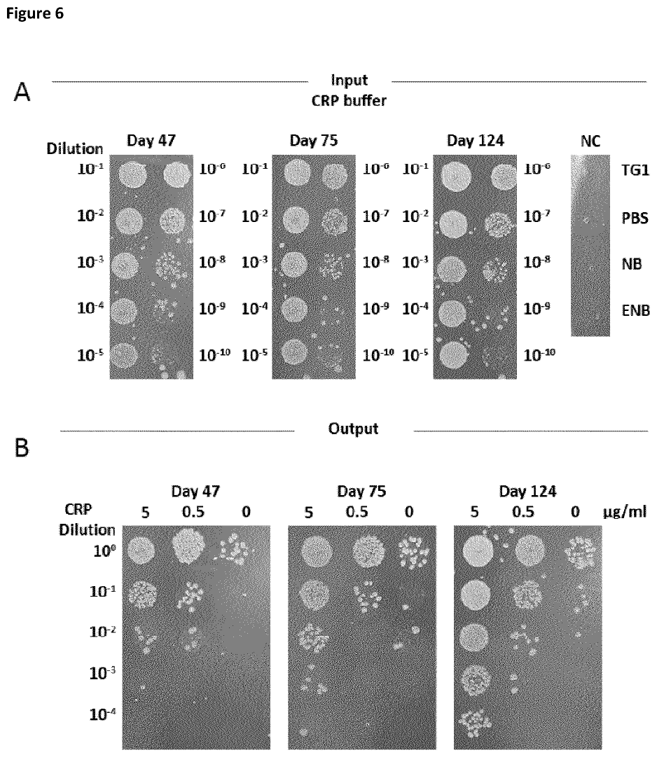

For the first round of phage-display panning selections, the amount of phages used corresponded to at least 10-fold of the transformation efficiency (estimated diversity) of the library (Table 3 and FIG. 6A).

TABLE 3
Input titers round 1 phage selection

| Library | Phage titer (cfu/ml) | Input titer (cfu/condition) |
|---|---|---|
| Day 47 | 7 × 10\hat{ }12 | 1.4 × 10\hat{ }11 |
| Day 75 | 4 × 10\hat{ }12 | 8.0 × 10\hat{ }10 |
| Day 124 | 6 × 10\hat{ }12 | 1.2 × 10\hat{ }11 |

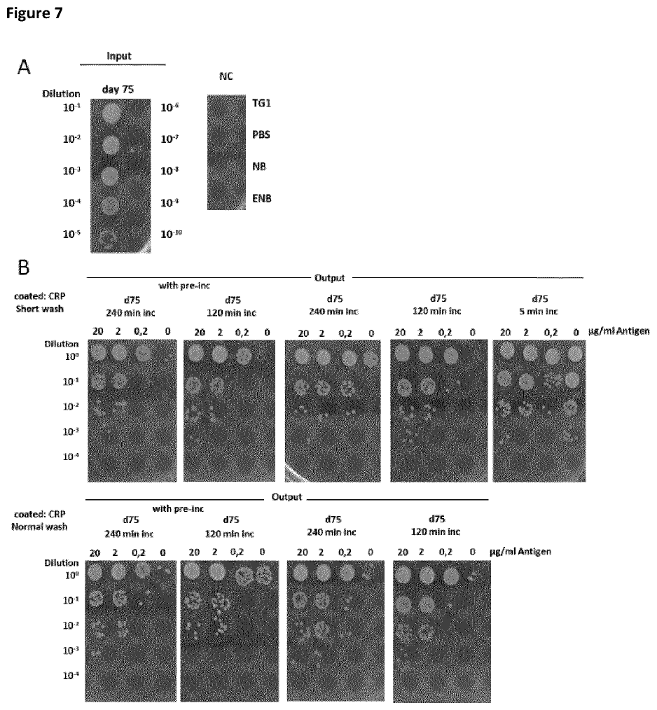

For the panning, the coated and blocked wells were incubated with the pre-blocked phages for 2 hours at RT. Upon extensive washing, bound phages were eluted and allowed to infect TG1 bacteria (rescued output). In order to assess the size of the output, eluted phages were serially diluted, infected into TG1 and then spotted on LB-agar. The outputs of these selections show that there was some enrichment of the phages from day 47. Day 75 showed more enrichment compared to uncoated wells and phages from day 124 showed most enrichment (FIG. 6B). As CRP was not immunized after day 75, most variety for CRP binding was expected from the day 75 library. Therefore, for the second round of selections, phages produced from the outputs of the day 75 library were used to generate input titers (Table 4 and FIG. 7A).

TABLE 4
Input titers round 2 phage selection

| Output | Phage titer (cfu/ml) | Input titer (cfu/condition) |
|---|---|---|
| Day 75 | 3.2 × 10\hat{ }10 | 3.2 × 10\hat{ }7 |

Based on the predictions retrieved from in silico modeling (TEM), the panning was directed towards the selection of sdAb clones with intermediate high on-rates and high off-rates allowing fast release of antigen at physiological concentrations of CRP. To direct the panning towards selecting sdAb with an intermediate high on-rate, CRP was coated at increasing concentrations. Extremely high affinity binders were disposed of by a non-standard 5-minute pre-incubation step. In addition to the standard incubation time of 120 minutes, the phages were then incubated for longer time periods in some conditions, namely 240 minutes, and for shorter time periods in other conditions, namely 5 minutes. Lastly, to prevent loss of sdAb with high off-rate, a shorter non-standard wash was applied in some conditions with only 5 minutes of washing as compared to the >30 minutes of washing usually performed. All conditions showed good output titration and enrichment of phages on the CRP coated wells (FIG. 7B). Eluted phages were rescued in TG1 and spotted on LB-agar plates from which single clones were picked and combined in three 96-wells masterplates (192 sdAb clones in total).

Sequence Analysis of sdAbs Identifies Clusters as Well as Unique sdAb Sequences

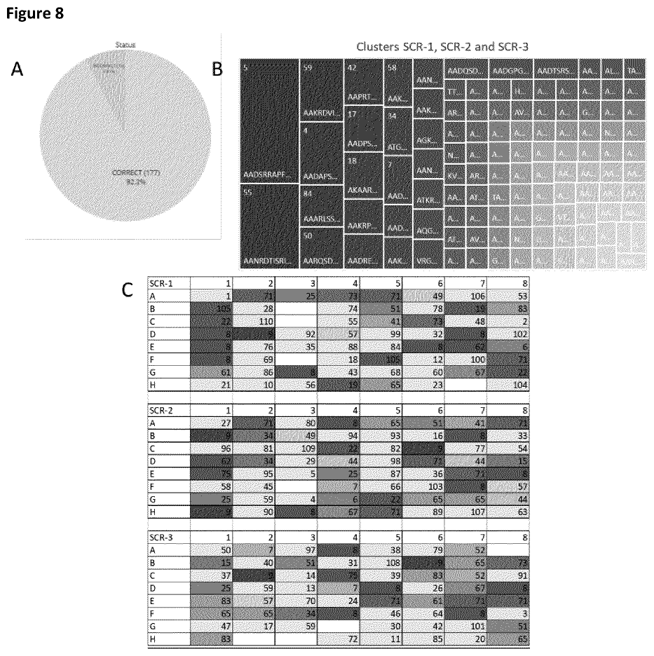

To determine the diversity of the sdAbs, all 3 master plates were sequenced by Sanger sequencing. The nucleic acid sequences were automatically analyzed and processed into sdAb amino acid sequences using the PipeBio Antibody Sequence Analysis platform. Then, the output sdAb sequences were annotated for sdAb sequence (FIG. 8A) and subsequently aligned and clustered using 80% CDR-H3 homology (FIGS. 8B and C).

Periplasmic Anti-CRP sdAb ELISA and Kinetics Screening Using Octet BLI to Characterize sdAb Binding Properties to Target Antigen (i.e., Non-Purified Periplasmic Samples Comprising Anti-CRP sdAbs were Measured Using Octet BLI for a Preliminary Assessment of the Binding Properties of the Respective sdAbs)

To evaluate binding to CRP, periplasmic extracts of all 192 sdAb clones were diluted and tested in an anti-CRP ELISA (Table 5). Performing a kinetic screening with non-purified sdAb samples (periplasmic extracts) in a high-throughput manner this early in the development process is unusual, but offers the opportunity to guide selection and further processing of sdAb clones at an early point towards desired kinetic properties.

TABLE 5
OD490 of periplasmic extract ELISA

| Clone | | Clone | | Clone | |
|---|---|---|---|---|---|
| name | OD490 | name | OD490 | name | OD490 |
| SCR-1A1 | 2.07 | SCR-2A1 | 1.80 | SCR-3A1 | 2.49 |
| SCR-1A2 | 2.09 | SCR-2A2 | 1.54 | SCR-3A2 | 2.51 |
| SCR-1A3 | 1.50 | SCR-2A3 | 1.58 | SCR-3A3 | 0.81 |
| SCR-1A4 | 2.01 | SCR-2A4 | 1.91 | SCR-3A4 | 1.92 |
| SCR-1A5 | 2.04 | SCR-2A5 | 1.78 | SCR-3A5 | 0.31 |
| SCR-1A6 | 2.52 | SCR-2A6 | 2.10 | SCR-3A6 | 2.33 |
| SCR-1A7 | 1.67 | SCR-2A7 | 1.63 | SCR-3A7 | 2.07 |
| SCR-1A8 | 2.15 | SCR-2A8 | 1.67 | SCR-3A8 | 1.84 |
| SCR-1B1 | 2.12 | SCR-2B1 | 1.65 | SCR-3B1 | 2.38 |
| SCR-1B2 | 1.85 | SCR-2B2 | 2.22 | SCR-3B2 | 2.56 |
| SCR-1B3 | 0.59 | SCR-2B3 | 2.33 | SCR-3B3 | 2.76 |
| SCR-1B4 | 1.55 | SCR-2B4 | 1.10 | SCR-3B4 | 2.40 |
| SCR-1B5 | 2.35 | SCR-2B5 | 1.19 | SCR-3B5 | 1.76 |
| SCR-1B6 | 2.35 | SCR-2B6 | 1.88 | SCR-3B6 | 2.39 |
| SCR-1B7 | 2.40 | SCR-2B7 | 1.76 | SCR-3B7 | 2.45 |
| SCR-1B8 | 2.20 | SCR-2B8 | 1.97 | SCR-3B8 | 2.03 |
| SCR-1C1 | 1.47 | SCR-2C1 | 1.54 | SCR-3C1 | 0.07 |
| SCR-1C2 | 1.80 | SCR-2C2 | 2.52 | SCR-3C2 | 2.23 |
| SCR-1C3 | 2.87 | SCR-2C3 | 1.42 | SCR-3C3 | 3.16 |
| SCR-1C4 | 2.02 | SCR-2C4 | 2.39 | SCR-3C4 | 2.28 |
| SCR-1C5 | 1.95 | SCR-2C5 | 2.01 | SCR-3C5 | 2.44 |
| SCR-1C6 | 1.99 | SCR-2C6 | 2.17 | SCR-3C6 | 0.93 |
| SCR-1C7 | 1.90 | SCR-2C7 | 2.23 | SCR-3C7 | 2.32 |
| SCR-1C8 | 2.22 | SCR-2C8 | 2.40 | SCR-3C8 | 0.19 |
| SCR-1D1 | 1.70 | SCR-2D1 | 0.53 | SCR-3D1 | 1.71 |
| SCR-1D2 | 1.94 | SCR-2D2 | 2.55 | SCR-3D2 | 2.41 |
| SCR-1D3 | 1.49 | SCR-2D3 | 1.70 | SCR-3D3 | 2.79 |
| SCR-1D4 | 1.65 | SCR-2D4 | 2.35 | SCR-3D4 | 2.78 |
| SCR-1D5 | 2.17 | SCR-2D5 | 2.10 | SCR-3D5 | 2.42 |
| SCR-1D6 | 2.66 | SCR-2D6 | 2.26 | SCR-3D6 | 2.57 |
| SCR-1D7 | 2.27 | SCR-2D7 | 2.27 | SCR-3D7 | 2.35 |
| SCR-1D8 | 1.82 | SCR-2D8 | 2.14 | SCR-3D8 | 2.07 |
| SCR-1E1 | 0.44 | SCR-2E1 | 1.67 | SCR-3E1 | 2.06 |
| SCR-1E2 | 1.68 | SCR-2E2 | 1.67 | SCR-3E2 | 2.31 |
| SCR-1E3 | 1.29 | SCR-2E3 | 2.51 | SCR-3E3 | 1.93 |
| SCR-1E4 | 2.03 | SCR-2E4 | 1.62 | SCR-3E4 | 1.59 |
| SCR-1E5 | 0.05 | SCR-2E5 | 1.62 | SCR-3E5 | 2.25 |
| SCR-1E6 | 2.51 | SCR-2E6 | 2.61 | SCR-3E6 | 2.27 |
| SCR-1E7 | 2.26 | SCR-2E7 | 2.24 | SCR-3E7 | 2.58 |
| SCR-1E8 | 2.44 | SCR-2E8 | 2.38 | SCR-3E8 | 2.51 |
| SCR-1F1 | 2.09 | SCR-2F1 | 1.66 | SCR-3F1 | 2.44 |
| SCR-1F2 | 2.43 | SCR-2F2 | 2.21 | SCR-3F2 | 2.40 |
| SCR-1F3 | 2.00 | SCR-2F3 | 2.38 | SCR-3F3 | 2.75 |
| SCR-1F4 | 2.04 | SCR-2F4 | 2.26 | SCR-3F4 | 2.52 |
| SCR-1F5 | 1.97 | SCR-2F5 | 1.98 | SCR-3F5 | 2.32 |
| SCR-1F6 | 0.06 | SCR-2F6 | 1.23 | SCR-3F6 | 2.53 |
| SCR-1F7 | 1.65 | SCR-2F7 | 1.63 | SCR-3F7 | 2.34 |
| SCR-1F8 | 2.33 | SCR-2F8 | 1.96 | SCR-3F8 | 2.60 |
| SCR-1G1 | 1.93 | SCR-2G1 | 2.04 | SCR-3G1 | 2.56 |
| SCR-1G2 | 1.83 | SCR-2G2 | 1.93 | SCR-3G2 | 2.34 |
| SCR-1G3 | 2.19 | SCR-2G3 | 2.55 | SCR-3G3 | 2.56 |
| SCR-1G4 | 2.09 | SCR-2G4 | 2.62 | SCR-3G4 | 2.17 |
| SCR-1G5 | 2.22 | SCR-2G5 | 1.76 | SCR-3G5 | 2.15 |
| SCR-1G6 | 1.75 | SCR-2G6 | 1.55 | SCR-3G6 | 0.08 |
| SCR-1G7 | 1.80 | SCR-2G7 | 2.14 | SCR-3G7 | 1.27 |
| SCR-1G8 | 1.88 | SCR-2G8 | 2.38 | SCR-3G8 | 2.71 |
| SCR-1H1 | 1.84 | SCR-2H1 | 2.04 | SCR-3H1 | 1.79 |
| SCR-1H2 | 1.99 | SCR-2H2 | 2.37 | SCR-3H2 | 2.30 |
| SCR-1H3 | 2.11 | SCR-2H3 | 1.87 | SCR-3H3 | 2.16 |
| SCR-1H4 | 2.31 | SCR-2H4 | 1.87 | SCR-3H4 | 2.40 |
| SCR-1H5 | 2.04 | SCR-2H5 | 2.03 | SCR-3H5 | 2.16 |
| SCR-1H6 | 1.94 | SCR-2H6 | 1.14 | SCR-3H6 | 1.92 |
| SCR-1H7 | 1.99 | SCR-2H7 | 1.56 | SCR-3H7 | 1.59 |
| SCR-1H8 | 1.11 | SCR-2H8 | 1.75 | SCR-3H8 | 2.11 |

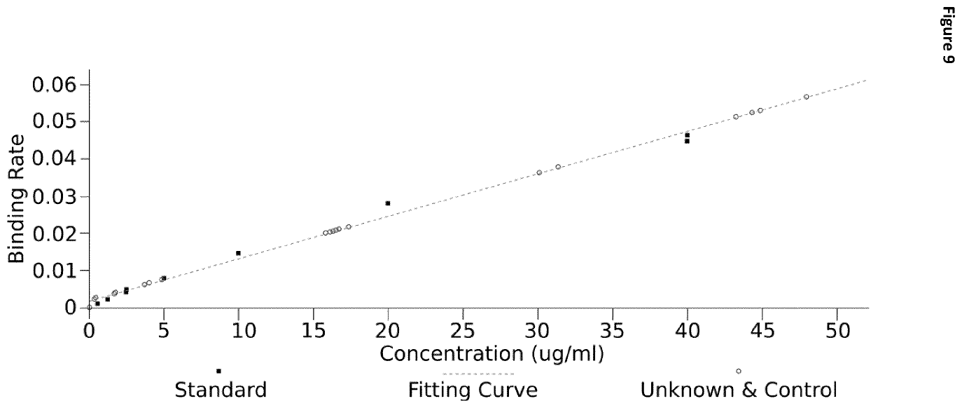

To evaluate the binding kinetics of each sdAb to CRP, periplasmic extracts of all 192 sdAb clones underwent kinetic screening using BLI Octet. First, a standard curve was first generated used to derive sdAb concentration in the periplasmic extracts (FIG. 9). Secondly, binding assays were performed by immobilizing biotinylated CRP on streptavidin-coated sensors and performing kinetic measurements on each sdAb. Of all 192 sdAb clones, the k_{d} of 121 sdAb clones could be evaluated (Table 6).

TABLE 6
BLI Octet screening results of the periplasmic extracts

| Clone name | Concentration [nM] | kₐ[s⁻¹M⁻¹] | k_{d} [s⁻¹] | K_{D} [M] |
|---|---|---|---|---|
| SCR-1A1 | 41.8 | 6.2E+05 | 9.3E−03 | 1.5E−08 |
| SCR-1A2 | 74.8 | 5.0E+05 | 5.6E−04 | 1.1E−09 |
| SCR-1A3 | NA | | 1.0E−03 | |
| SCR-1A4 | NA | | 2.8E−04 | |
| SCR-1A5 | NA | | 3.6E−04 | |
| SCR-1A6 | NA | | 6.7E−04 | |
| SCR-1A8 | NA | | 2.7E−03 | |
| SCR-1B1 | 70.5 | 3.7E+05 | 1.0E−03 | 2.7E−09 |
| SCR-1B4 | 69.7 | 6.1E+05 | 6.8E−04 | 1.1E−09 |
| SCR-1B8 | NA | | 4.1E−04 | |
| SCR-1C3 | NA | | 9.3E−04 | |
| SCR-1C6 | NA | | 3.6E−04 | |
| SCR-1D3 | 254.3 | 2.9E+05 | 2.7E−04 | 9.3E−10 |
| SCR-1D4 | 88.5 | 1.0E+06 | 8.8E−04 | 8.6E−10 |
| SCR-1D5 | 50.1 | 7.0E+05 | 3.3E−04 | 4.7E−10 |
| SCR-1D6 | NA | | 3.5E−04 | |
| SCR-1E1 | NA | | 6.2E−03 | |
| SCR-1E2 | 118.1 | 4.9E+05 | 7.5E−04 | 1.5E−09 |
| SCR-1E4 | 299.3 | 2.9E+05 | 5.2E−04 | 1.8E−09 |
| SCR-1E6 | NA | | 4.3E−04 | |
| SCR-1E7 | NA | | 4.8E−04 | |
| SCR-1F2 | 156.2 | 3.3E+05 | 9.1E−04 | 2.7E−09 |
| SCR-1F3 | NA | | 4.2E−03 | |
| SCR-1F5 | 53.9 | 1.3E+06 | 4.5E−04 | 3.4E−10 |
| SCR-1F7 | 59.0 | 1.3E+06 | 6.5E−04 | 5.2E−10 |
| SCR-1F8 | NA | | 1.9E−04 | |
| SCR-1G2 | 167.2 | 2.8E+05 | 6.8E−04 | 2.4E−09 |
| SCR-1G4 | 61.4 | 8.7E+05 | 5.7E−04 | 6.6E−10 |
| SCR-1G5 | NA | | 2.2E−04 | |
| SCR-1G6 | 73.1 | 7.6E+05 | 6.4E−04 | 8.3E−10 |
| SCR-1G7 | NA | | 5.5E−04 | |
| SCR-1H3 | 52.2 | 8.8E+05 | 5.7E−04 | 6.5E−10 |
| SCR-1H5 | 93.9 | 7.1E+05 | 3.9E−04 | 5.5E−10 |
| SCR-1H7 | NA | | 3.8E−04 | |
| SCR-1H8 | 91.7 | 1.6E+06 | 2.6E−03 | 1.7E−09 |
| SCR-2A2 | 85.8 | 5.4E+05 | 5.1E−04 | 9.3E−10 |
| SCR-2A3 | 85.2 | 6.8E+05 | 6.0E−04 | 8.8E−10 |
| SCR-2A5 | NA | | 5.6E−04 | |
| SCR-2A6 | NA | | 4.5E−04 | |
| SCR-2A7 | NA | | 1.3E−03 | |
| SCR-2A8 | NA | | 3.6E−04 | |
| SCR-2B2 | 102.9 | 2.2E+05 | 5.5E−04 | 2.5E−09 |
| SCR-2B4 | 86.5 | 6.2E+05 | 1.9E−03 | 3.1E−09 |
| SCR-2B5 | 32.9 | 7.2E+05 | 2.3E−03 | 3.1E−09 |
| SCR-2B7 | NA | | 3.6E−03 | |
| SCR-2B8 | NA | | 1.5E−03 | |
| SCR-2C1 | 88.4 | 6.0E+05 | 6.3E−04 | 1.1E−09 |
| SCR-2C2 | 109.9 | 3.4E+05 | 5.1E−04 | 1.5E−09 |
| SCR-2C5 | 54.9 | 1.1E+06 | 3.3E−04 | 3.1E−10 |
| SCR-2C7 | NA | | 5.1E−04 | |
| SCR-2C8 | NA | | 6.6E−04 | |
| SCR-2D1 | 190.9 | 4.1E+04 | 1.2E−03 | 2.9E−08 |
| SCR-2D2 | 39.4 | 5.7E+05 | 5.7E−04 | 1.0E−09 |
| SCR-2D5 | NA | | 3.4E−04 | |
| SCR-2D6 | 65.9 | 1.5E+06 | 6.1E−04 | 4.1E−10 |
| SCR-2D7 | NA | | 1.6E−03 | |
| SCR-2D8 | 38.3 | 6.4E+06 | 1.5E−03 | 2.4E−10 |
| SCR-2E1 | 152.9 | 2.7E+05 | 7.5E−04 | 2.8E−09 |
| SCR-2E2 | 90.9 | 1.8E+05 | 7.1E−04 | 3.9E−09 |
| SCR-2E3 | NA | | 5.8E−04 | |
| SCR-2E5 | NA | | 3.5E−04 | |
| SCR-2E7 | NA | | 4.8E−04 | |
| SCR-2E8 | NA | | 2.2E−03 | |
| SCR-2F1 | 57.2 | 4.3E+05 | 4.9E−04 | 1.2E−09 |
| SCR-2F3 | NA | | 1.0E−03 | |
| SCR-2F5 | NA | | 5.5E−04 | |
| SCR-2F6 | NA | | 1.7E−03 | |
| SCR-2F7 | NA | | 3.4E−03 | |
| SCR-2F8 | NA | | 5.5E−04 | |
| SCR-2G2 | NA | | 6.5E−04 | |
| SCR-2G3 | NA | | 1.5E−03 | |
| SCR-2G5 | NA | | 9.2E−04 | |
| SCR-2G6 | NA | | 5.1E−04 | |
| SCR-2G7 | NA | | 6.3E−04 | |
| SCR-2G8 | NA | | 1.5E−03 | |
| SCR-2H2 | NA | | 5.8E−04 | |
| SCR-2H4 | NA | | 5.6E−04 | |
| SCR-2H5 | NA | | 4.0E−04 | |
| SCR-2H6 | NA | | 4.1E−04 | |
| SCR-2H7 | NA | | 1.2E−03 | |
| SCR-2H8 | NA | | 6.0E−04 | |
| SCR-3A1 | NA | | 1.7E−03 | |
| SCR-3A3 | NA | | 5.6E−03 | |
| SCR-3A4 | 200.9 | 1.0E+06 | 8.6E−03 | 8.5E−09 |
| SCR-3A5 | 25.4 | 1.3E+06 | 2.2E−01 | 1.8E−07 |
| SCR-3A6 | NA | | 3.6E−04 | |
| SCR-3A7 | NA | | 2.6E−04 | |
| SCR-3A8 | 30.7 | 3.5E+06 | 5.3E−04 | 1.5E−10 |
| SCR-3B5 | 26.5 | 5.6E+06 | 1.9E−03 | 3.3E−10 |
| SCR-3B7 | 48.2 | 1.8E+06 | 3.7E−04 | 2.0E−10 |
| SCR-3B8 | NA | | 3.2E−04 | |
| SCR-3C4 | NA | | 5.1E−04 | |
| SCR-3C5 | NA | | 5.3E−04 | |
| SCR-3C6 | NA | | 2.1E−03 | |
| SCR-3C7 | NA | | 4.4E−04 | |
| SCR-3D2 | NA | | 7.7E−04 | |
| SCR-3D4 | 107.9 | 1.1E+06 | 1.2E−03 | 1.1E−09 |
| SCR-3D7 | NA | | 4.6E−04 | |
| SCR-3E1 | 50.9 | 2.6E+06 | 1.5E−03 | 5.7E−10 |
| SCR-3E2 | NA | | 6.2E−04 | |
| SCR-3E3 | NA | | 1.1E−03 | |
| SCR-3E4 | 168.9 | 3.3E+04 | 2.6E−04 | 7.8E−09 |
| SCR-3E5 | 35.8 | 2.7E+06 | 3.9E−04 | 1.5E−10 |
| SCR-3E6 | 195.9 | 2.0E+05 | 4.3E−04 | 2.1E−09 |
| SCR-3E7 | 82.8 | 6.0E+05 | 3.2E−04 | 5.2E−10 |
| SCR-3E8 | NA | | 4.0E−04 | |
| SCR-3F1 | 38.7 | 1.8E+06 | 9.3E−04 | 5.2E−10 |
| SCR-3F2 | 135.9 | 6.6E+05 | 8.1E−04 | 1.2E−09 |
| SCR-3F3 | 241.9 | 1.3E+05 | 7.0E−04 | 5.2E−09 |
| SCR-3F5 | 295.9 | 1.2E+05 | 2.5E−04 | 2.1E−09 |
| SCR-3F6 | NA | | 3.4E−04 | |
| SCR-3F8 | 65.4 | 1.1E+06 | 4.4E−04 | 4.1E−10 |
| SCR-3G3 | 39.5 | 1.8E+06 | 8.3E−04 | 4.5E−10 |
| SCR-3G4 | NA | | 5.3E−04 | |
| SCR-3G5 | NA | | 1.0E−02 | |
| SCR-3G7 | 72.5 | 3.9E+05 | 2.8E−03 | 7.3E−09 |
| SCR-3H1 | 26.9 | 4.6E+06 | 1.1E−03 | 2.4E−10 |
| SCR-3H2 | NA | | 7.7E−04 | |
| SCR-3H4 | NA | | 6.6E−04 | |
| SCR-3H6 | 45.1 | 2.4E+06 | 4.6E−04 | 1.9E−10 |
| SCR-3H8 | NA | | 4.1E−04 | |

Rational Selection of Anti-CRP sdAbs Based on Binding Characteristics and Sequence

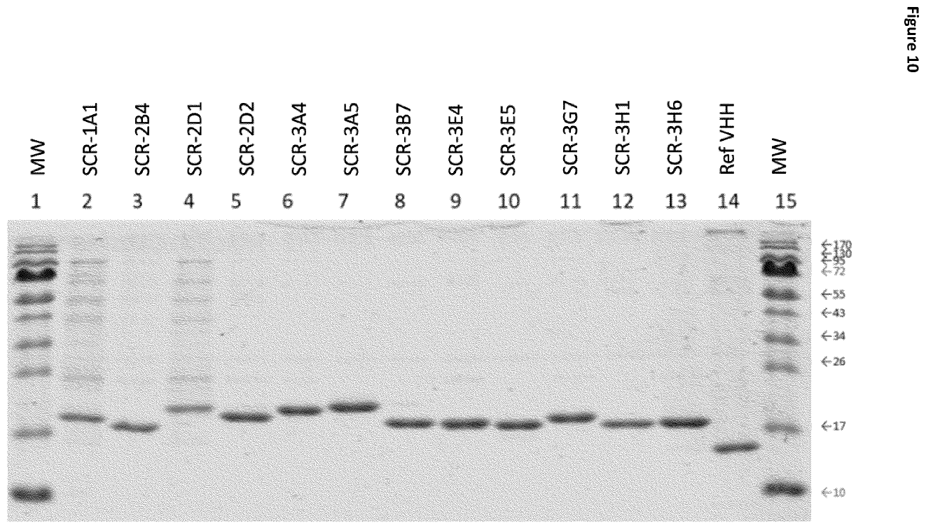

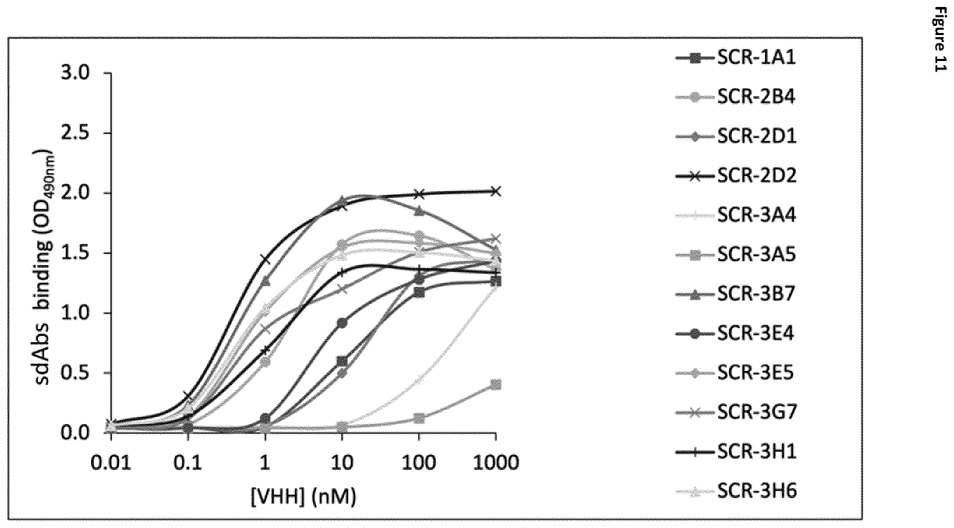

12 anti-CRP sdAb clones were selected based on ELISA results, Octet BLI screening results (Table 6) and sequence identity. The clones were selected such as to represent a variety of CDR sequences and to either represent high affinity binders and/or to fit the kₐ/k_{d} criteria defined by in silico modeling (TEM). Selected clones were transformed into the E. coli strain BL21, expressed and purified. The purity of those sdAbs was assessed by SDS-PAGE (FIG. 10) and their apparent binding affinity determined by ELISA (FIG. 11 and Table 7).

The rational selection was performed after the Octet screening of non-purified samples (see Table 6 above) and before running the Octet BLI and SPR Biacore with purified samples, see below Table 8.

TABLE 7
Estimated apparent affinities for CRP

| | Relative | Apparent |
|---|---|---|
| | Bmax | affinity for |
| Clone name | (OD) | CRP (nM) |
| SCR-1A1 | 0.6 | 10 |
| SCR-2B4 | 0.8 | 2 |
| SCR-2D1 | 0.7 | 20 |
| SCR-2D2 | 1 | 0.4 |
| SCR-3A4 | 0.6 | 200 |
| SCR-3A5 | 0.2 | 200 |
| SCR-3B7 | 1 | 0.5 |
| SCR-3E4 | 0.7 | 5 |
| SCR-3E5 | 0.75 | 0.5 |
| SCR-3G7 | 0.8 | 0.5 |
| SCR-3H1 | 0.65 | 0.5 |
| SCR-3H6 | 0.7 | 0.5 |

Anti-CRP sdAb Binding Kinetics Characterization Using Octet BLI

hCRP (Sigma, Cat 236603) was biotinylated in house (ThermoFisher, Cat. 21435) and immobilized on Streptavidin coated sensors (Sartorius, Cat 18−5019). The sensor loading was done for 80 sec with the biotinylated CRP at 10 μg/mL in the running buffer. Purified samples were diluted in the running buffer to 7 concentrations: 100, 50, 25, 12.5, 6.3, 3.1, 1.6 nM. The Octet BLI sensors were dipped into the wells of 96-well plate that contained the diluted samples.

The readings were done with the following parameters:
Association: 420 sec
Dissociation 600 sec
Assay temperature: 25° C.
Agitation at 1,000 rpm

All measurements were repeated with reference sensors (without CRP) to measure background signal, which was subsequently subtracted from the binding signal of the VHHs. The results are shown in Table 6 (screening of periplasmic samples) and Table 8 (purified samples).

TABLE 8
BLI Octet screening results of purified samples with standard deviations

| | | | SD (Rich | Conservative | | | SD (Rich | Conservative | |
|---|---|---|---|---|---|---|---|---|---|
| Clone | kₐ[s⁻¹M⁻¹] | SD | et al) | SD | k_{d} [s⁻¹] | SD | et al) | SD | K_{D} [M] |
| SCR-3H6 | 3.50E+05 | 2.36E+03 | 3.25E+05 | 3.25E+05 | 2.00E−05 | 3.39E−06 | 2.85E−05 | 2.85E−05 | 5.80E−11 |
| SCR-3E5 | 2.20E+05 | 1.09E+03 | 2.04E+05 | 2.04E+05 | 3.00E−05 | 2.65E−06 | 4.28E−05 | 4.28E−05 | 1.40E−10 |
| SCR-3B7 | 3.00E+05 | 8.37E+02 | 2.79E+05 | 2.79E+05 | 5.90E−05 | 1.41E−06 | 8.41E−05 | 8.41E−05 | 1.90E−10 |
| SCR-3H1 | 3.00E+05 | 1.58E+03 | 2.79E+05 | 2.79E+05 | 8.10E−05 | 2.73E−06 | 1.16E−04 | 1.16E−04 | 2.70E−10 |
| SCR-2D2 | 3.80E+05 | 4.34E+03 | 3.53E+05 | 3.53E+05 | 1.30E−04 | 5.53E−06 | 1.85E−04 | 1.85E−04 | 3.40E−10 |
| SCR-2B4 | 2.70E+05 | 1.80E+03 | 2.51E+05 | 2.51E+05 | 4.00E−04 | 3.44E−06 | 5.70E−04 | 5.70E−04 | 1.50E−09 |
| SCR-2D1 | 6.40E+04 | 4.22E+02 | 5.94E+04 | 5.94E+04 | 3.30E−04 | 2.48E−06 | 4.71E−04 | 4.71E−04 | 5.10E−09 |
| SCR-3E4 | 4.10E+04 | 3.31E+02 | 3.81E+04 | 3.81E+04 | 6.80E−04 | 3.27E−06 | 9.70E−04 | 9.70E−04 | 1.70E−08 |
| SCR-3A4 | 3.10E+05 | 1.48E+04 | 2.88E+05 | 2.88E+05 | 5.80E−03 | 8.61E−05 | 8.27E−03 | 8.27E−03 | 1.90E−08 |
| SCR-1A1 | 1.30E+05 | 3.02E+03 | 1.21E+05 | 1.21E+05 | 6.90E−03 | 4.29E−05 | 9.84E−03 | 9.84E−03 | 5.50E−08 |
| SCR-3G7 | 1.00E+05 | 4.84E+03 | 9.29E+04 | 9.29E+04 | 6.80E−03 | 8.57E−05 | 9.70E−03 | 9.70E−03 | 6.50E−08 |
| MIN | 4.10E+04 | 3.31E+02 | 3.81E+04 | 3.81E+04 | 2.00E−05 | 1.41E−06 | 2.85E−05 | 2.85E−05 | 5.80E−11 |
| MAX | 3.80E+05 | 1.48E+04 | 3.53E+05 | 3.53E+05 | 6.90E−03 | 8.61E−05 | 9.84E−03 | 9.84E−03 | 6.50E−08 |

Anti-CRP sdAb Binding Kinetics Characterization Using SPR Biacore

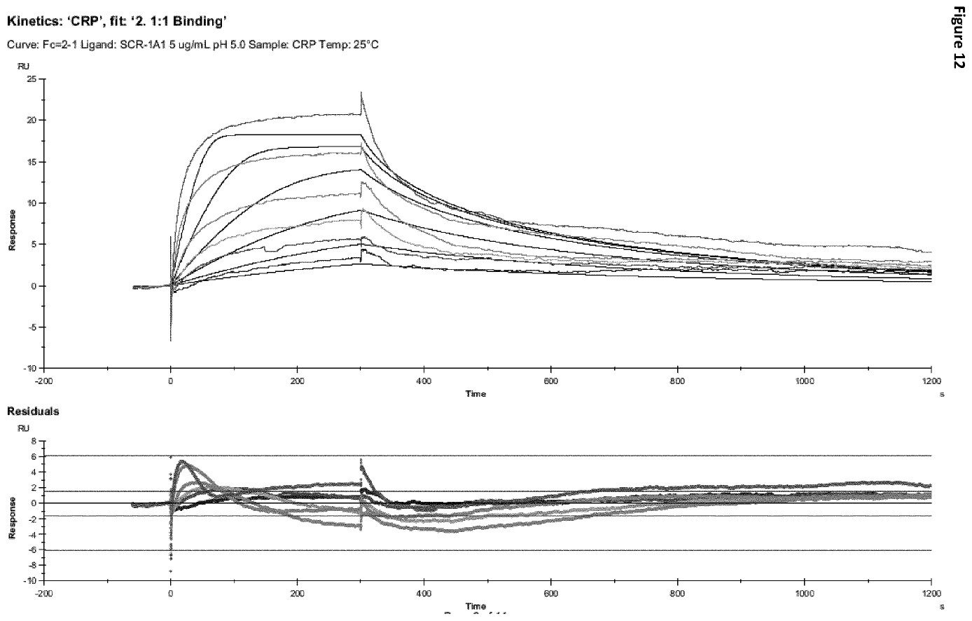

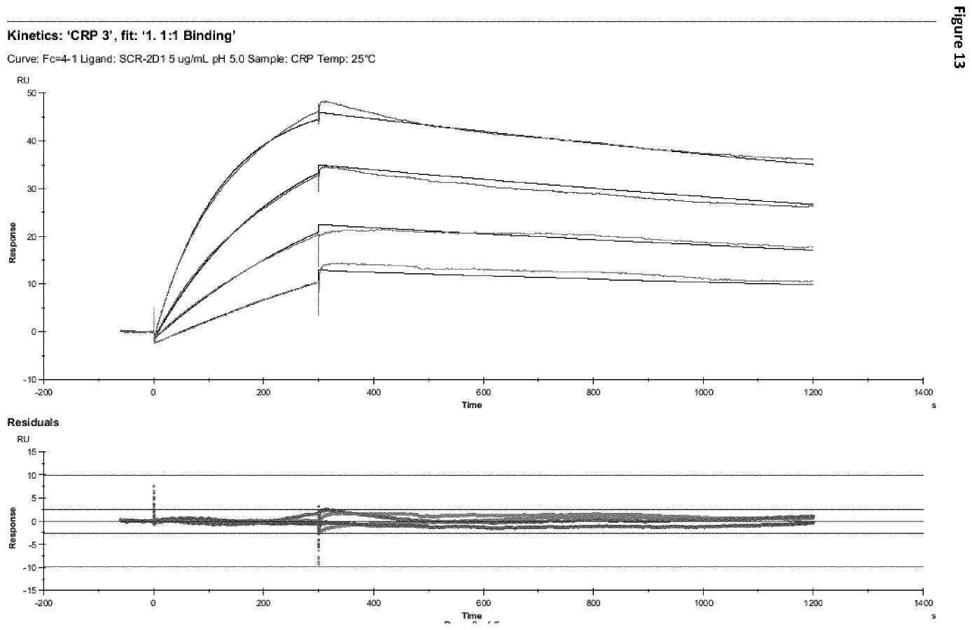

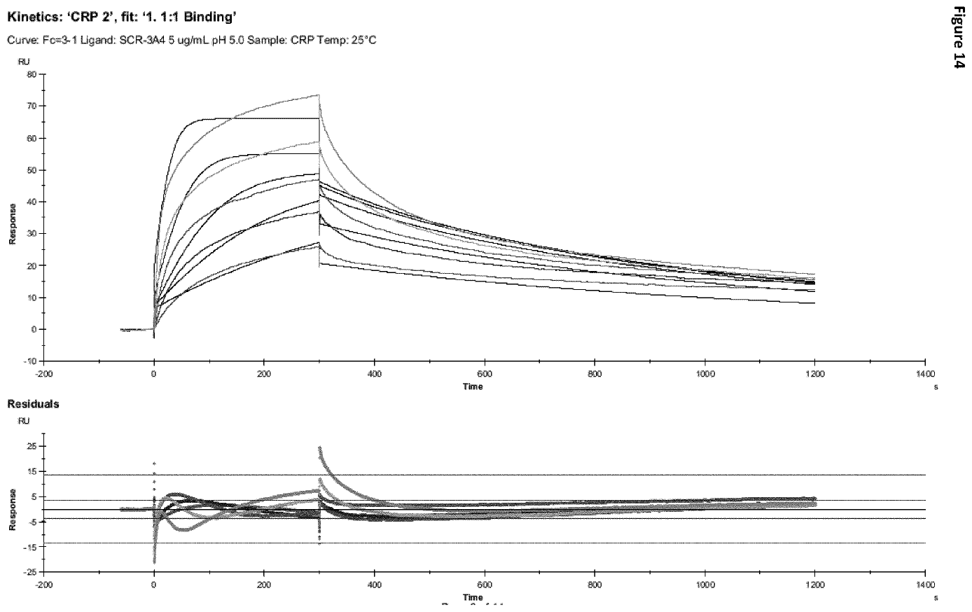

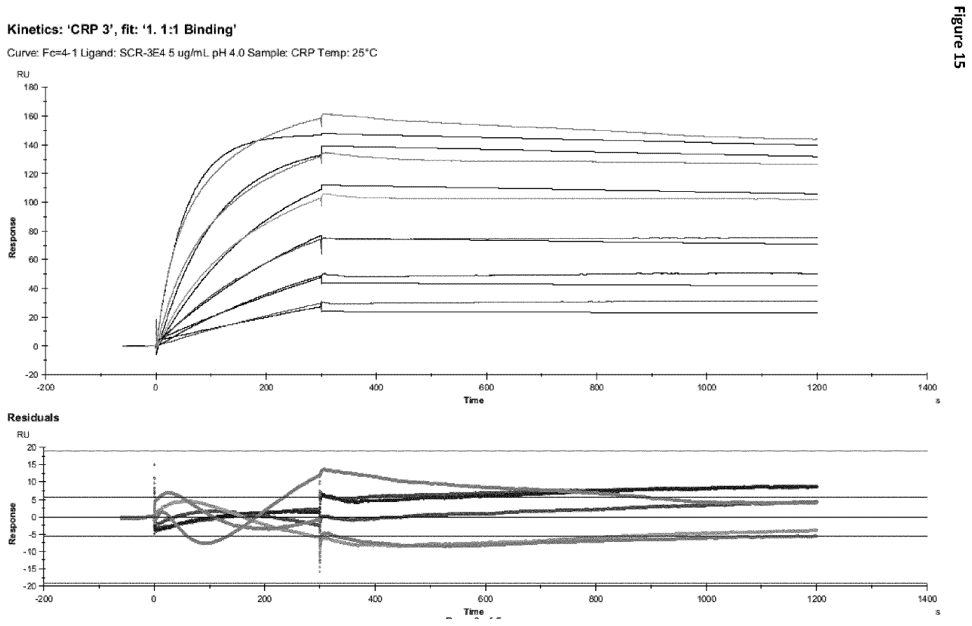

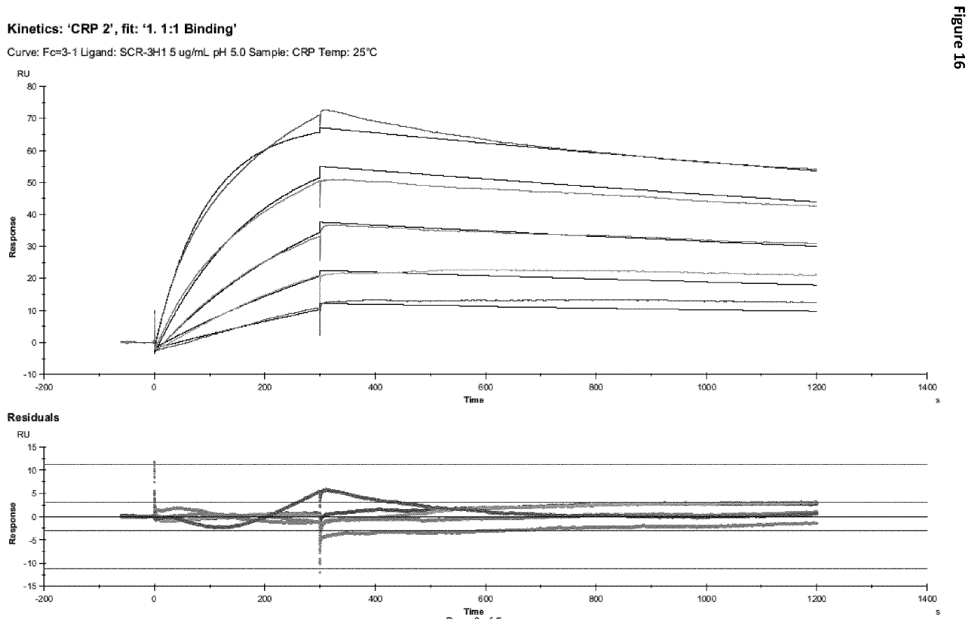

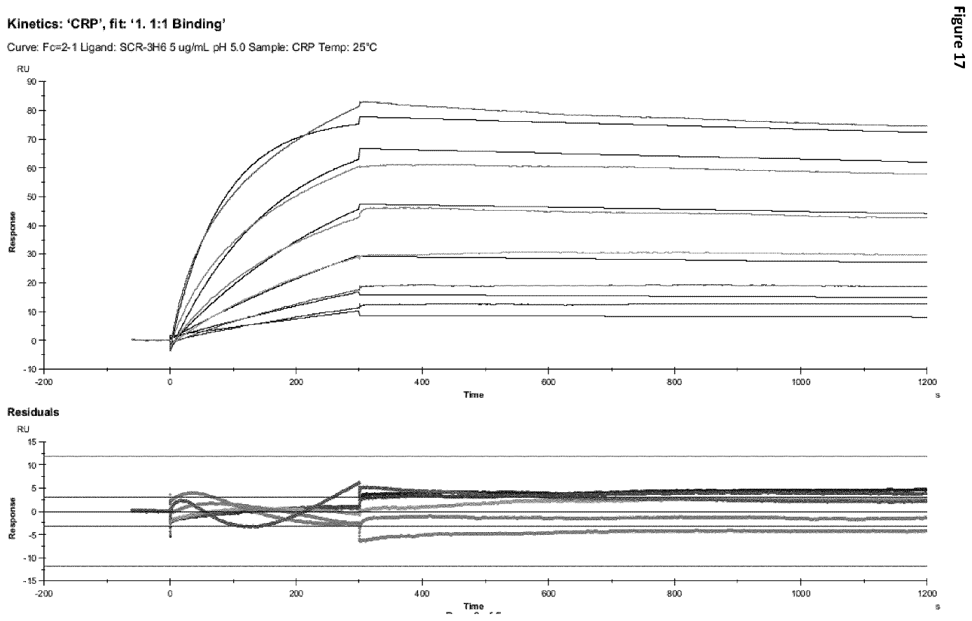

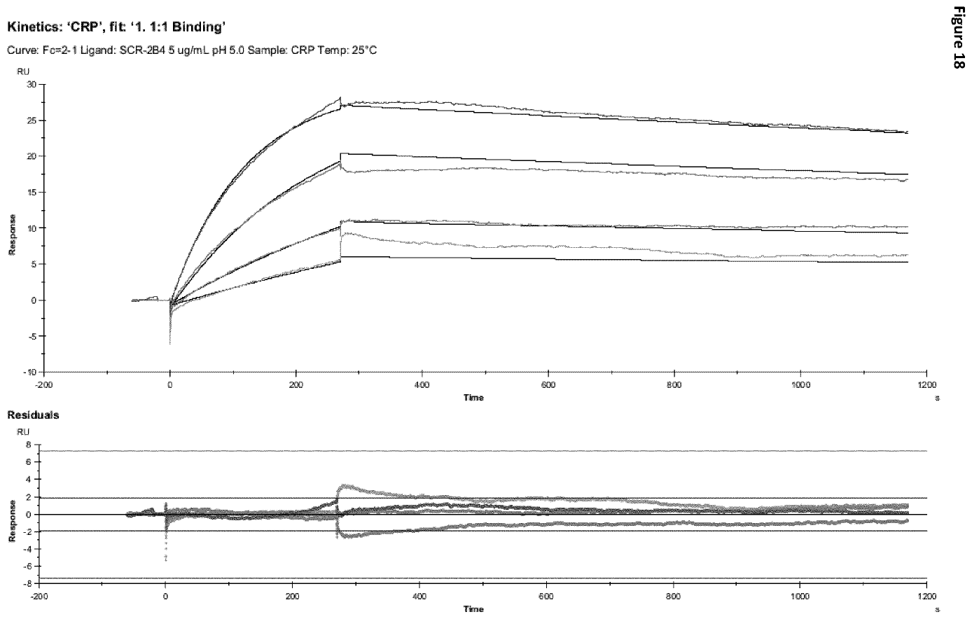

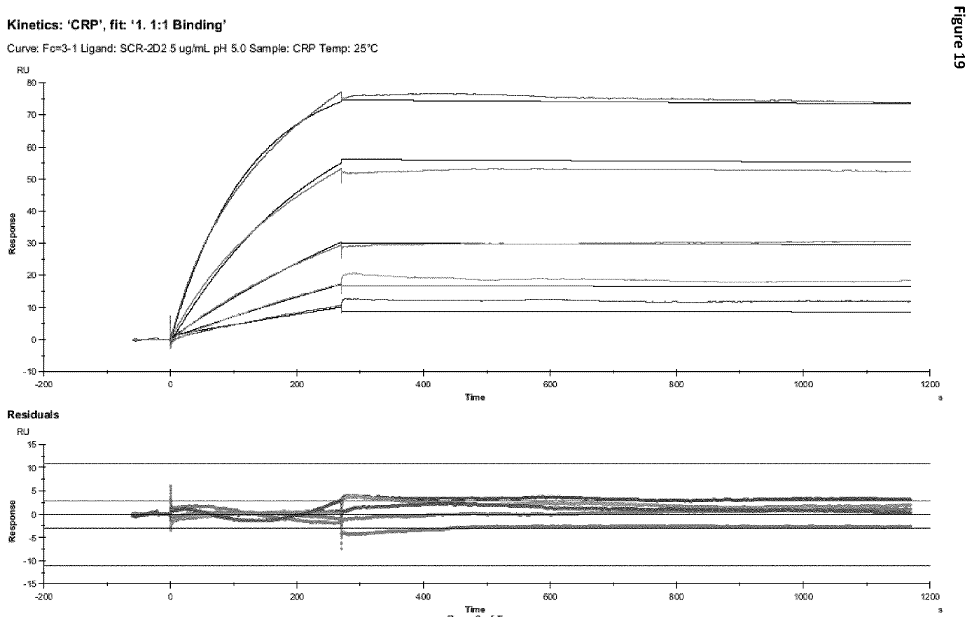

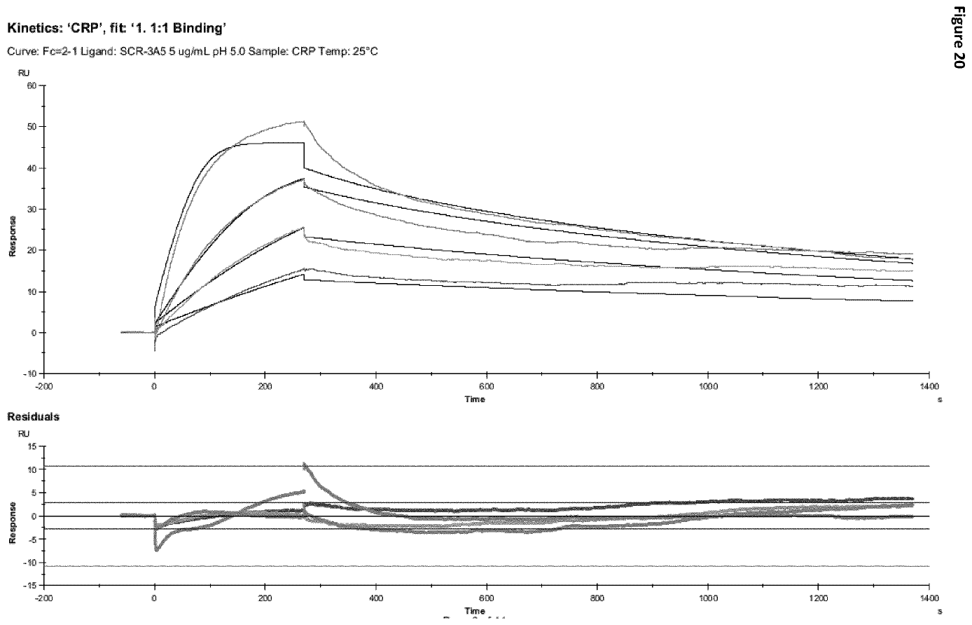

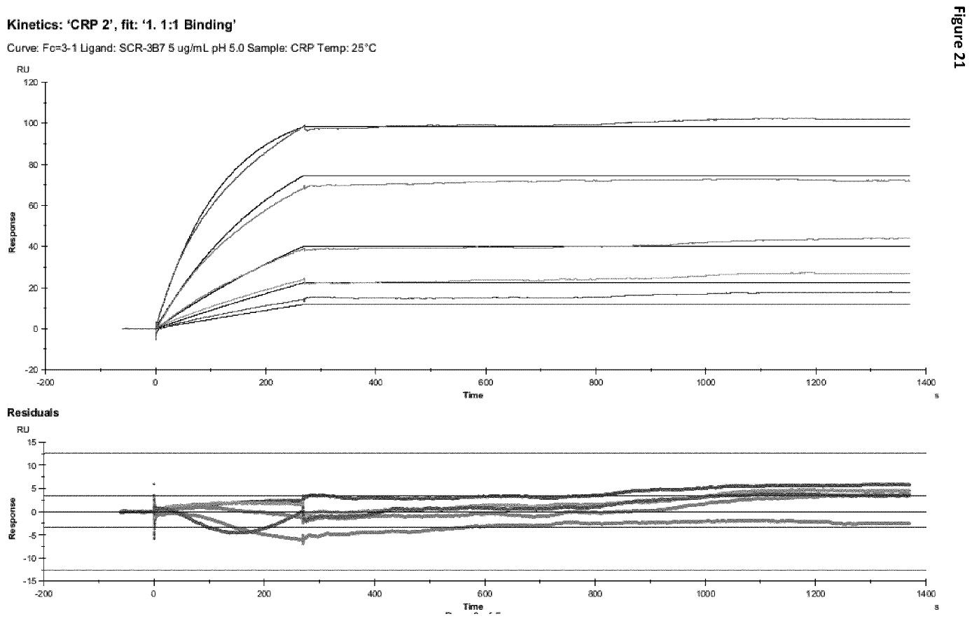

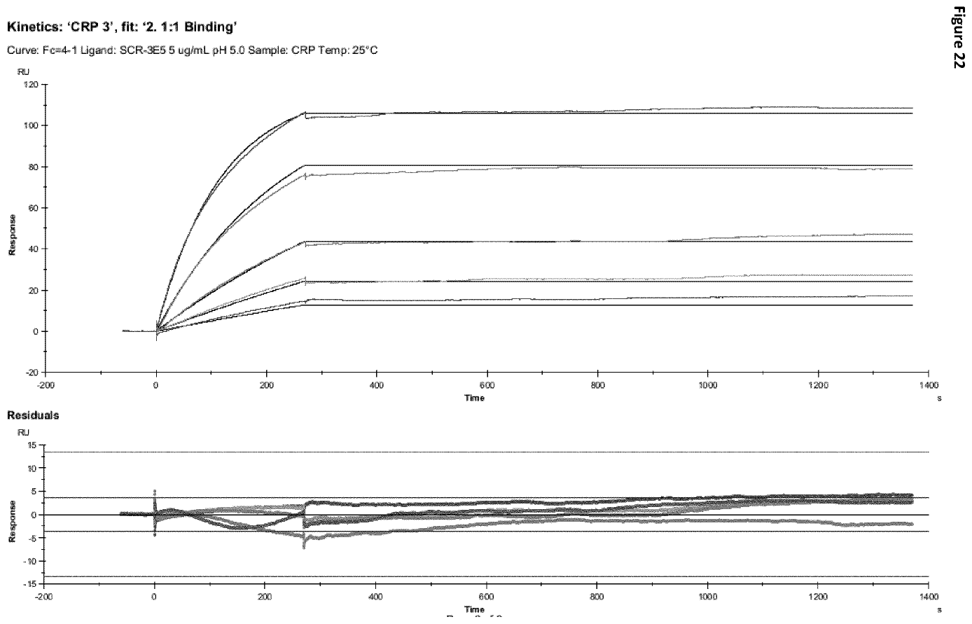

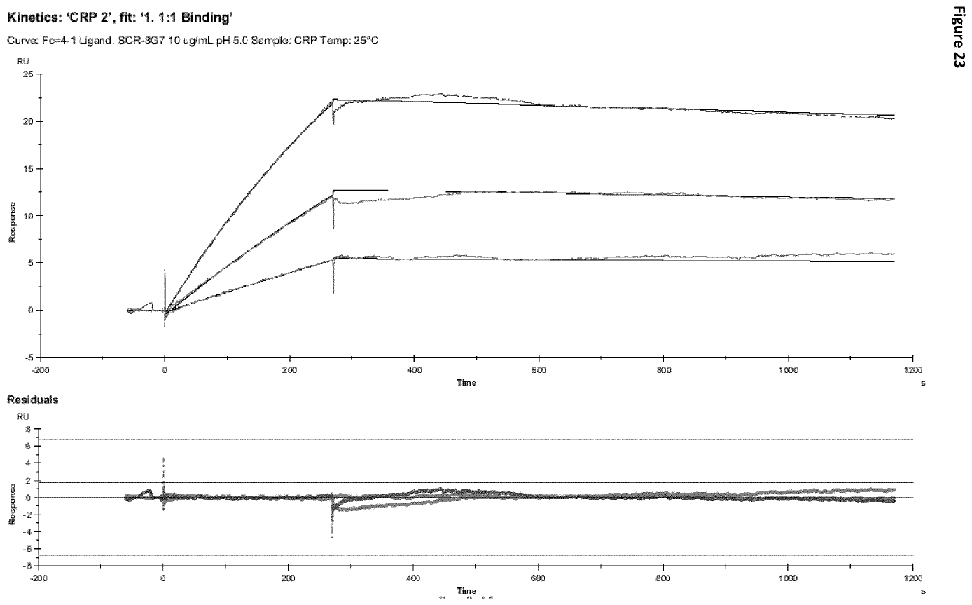

In order to characterize the binding kinetics of the 12 lead anti-CRP sdAbs, SPR Biacore measurements were performed. Experimental parameters of SPR Biacore characterization are plotted in Table 9, and results are plotted in Table 10. Individual kinetic binding curves for each sdAb are shown in FIG. 12-23.

TABLE 9
Experimental conditions to run SPR Biacore characterization
for each sdAb immobilised on the CM5 chip.

| | Concentration | | Target | | | |
|---|---|---|---|---|---|---|
| | used for | | immobilisation | Time for | Time for | Analyte |
| sdAb | immobilisation | pH used for | signal/actual | association | dissociation | concentrations |
| name | [μg/mL] | immobilisation | signal [r.u] | (sec) | (sec) | range [nM] |
| | | | | | | 8-point 2-fold |
| | | | | | | dilution: |
| SCR-1A1 | 5 | 5 | 100/159 | 300 | 900 | 200-1.56 |
| SCR-3A4 | 5 | 5 | 100/137 | 300 | 900 | 200-1.56 |
| SCR-3E4 | 5 | 4 | 100/145 | 300 | 900 | 200-1.56 |
| SCR-3H6 | 5 | 5 | 100/171 | 300 | 900 | 10-0.08 |
| SCR-3H1 | 5 | 5 | 100/162 | 300 | 900 | 10-0.08 |
| SCR-2D1 | 5 | 5 | 100/167 | 300 | 900 | 10-0.08 |
| | | | | | | 7-point 2-fold |
| | | | | | | dilution: |
| SCR-2B4 | 5 | 5 | 100/182 | 300 | 900 | 5-0.05 |
| SCR-2D2 | 5 | 5 | 100/153 | 300 | 900 | 5-0.05 |
| SCR-3G7 | 5 | 5 | 100/147 | 300 | 900 | 5-0.05 |
| SCR-3A5 | 5 | 5 | 100/179 | 300 | 1,000 | 5-0.05 |
| SCR-3B7 | 5 | 5 | 100/165 | 300 | 1,000 | 5-0.05 |
| SCR-3E5 | 5 | 5 | 100/163 | 300 | 1,000 | 5-0.05 |

TABLE 10
SPR Biacore binding kinetics of lead anti-CRP sdAb clones.

| Clone | kₐ[s⁻¹M⁻¹] | k_{d} [s⁻¹] | KD [nM] |
|---|---|---|---|
| SCR-3H6 | 2.4E6 | 8.1E−5 | 0.03 |
| SCR-3E5 | 1.7E6 | 3.2E−9* | 1.8E−6* |
| SCR-3B7 | 1.5E6 | 1.3E−7* | 7.6E−5* |
| SCR-3H1 | 2.1E6 | 2.5E−4 | 0.12 |
| SCR-2D2 | 1.6E6 | 2.0E−5 | 0.01 |
| SCR-2B4 | 1.7E6 | 1.7E−4 | 0.1 |
| SCR-2D1 | 8.0E5 | 3.0E−4 | 0.38 |
| SCR-3E4 | 3.8E5 | 6.2E−5 | 0.16 |
| SCR-3A4 | 2.7E6 | 1.9E−3 | 0.70 |
| SCR-1A1 | 1.3E6 | 1.3E−2 | 9.62 |
| SCR-3G7 | 6.0E5 | 9.8E−5* | 0.16* |
| SCR-3A5 | 1.7E7 | 1.3E−3 | 0.08 |

*K_{off} (i.e., k_{d}) too slow to be accurately calculated

During SPR Biacore measurements, the sdAbs are immobilized on the sensor surface by EDC/NHS chemistry, with CRP antigens flowing over the surface at different concentrations. Whereas during the Octet BLI measurements described above, the CRP antigens were immobilized and the anti-CRP sdAbs were present in solution. Accordingly, SPR Biacore more closely reflects an in vitro or in vivo biosensor setup (comprising immobilized anti-CRP sdAbs and CRP antigens in solution) as compared to Octet BLI. SPR Biacore measurements (Table 10) may thus be preferred herein compared to the BLI Octet measurements using purified sdAbs (Table 8), which in turn may be preferred herein compared to the BLI Octet measurements using periplasmic extract sdAbs (Table 6).

Accordingly, in the context of the present invention, when assessing binding affinities of the herein provided sdAbs, measurements performed on purified sdAbs (i.e., the values disclosed in Table 8 and Table 10) are preferred, whereas SPR Biacore measurements (i.e., the values disclosed in Table 10) are particularly preferred.

TABLE 11
SPR Biacore binding kinetics and standard deviations of lead anti-CRP sdAb clones

| | | | SD | Conservative | | | SD | Conservative | K_{D} |
|---|---|---|---|---|---|---|---|---|---|
| Clone | kₐ[s⁻¹M⁻¹] | SD | (Rich et al) | SD | k_{d} [s⁻¹] | SD | (Rich et al) | SD | [nM] |
| SCR-3H6 | 2.40E+06 | 4.90E+03 | 2.23E+06 | 2.23E+06 | 8.10E−05 | 2.69E−07 | 1.16E−04 | 1.16E−04 | 0.03 |
| SCR-3E5 | 1.70E+06 | 2.46E+03 | 1.58E+06 | 1.58E+06 | 3.20E−09 | 1.34E−07 | 4.56E−09 | 1.34E−07 | 1.8e−6* |
| SCR-3B7 | 1.50E+06 | 3.80E+03 | 1.39E+06 | 1.39E+06 | 1.30E−07 | 1.88E−07 | 1.85E−07 | 1.88E−07 | 7.6e−5* |
| SCR-3H1 | 2.10E+06 | 4.47E+03 | 1.95E+06 | 1.95E+06 | 2.50E−04 | 1.86E−06 | 3.57E−04 | 3.57E−04 | 0.12 |
| SCR-2D2 | 1.60E+06 | 3.80E+03 | 1.49E+06 | 1.49E+06 | 2.00E−05 | 1.79E−06 | 2.85E−05 | 2.85E−05 | 0.01 |
| SCR-2B4 | 1.70E+06 | 5.00E+03 | 1.58E+06 | 1.58E+06 | 1.70E−04 | 2.20E−06 | 2.42E−04 | 2.42E−04 | 0.1 |
| SCR-2D1 | 8.00E+05 | 1.26E+03 | 7.43E+05 | 7.43E+05 | 3.00E−04 | 1.20E−06 | 4.28E−04 | 4.28E−04 | 0.38 |
| SCR-3E4 | 3.80E+05 | 9.06E+02 | 3.53E+05 | 3.53E+05 | 6.20E−05 | 2.28E−06 | 8.84E−05 | 8.84E−05 | 0.16 |
| SCR-3A4 | 2.70E+06 | 4.47E+04 | 2.51E+06 | 2.51E+06 | 1.90E−03 | 2.91E−05 | 2.71E−03 | 2.71E−03 | 0.7 |
| SCR-1A1 | 1.30E+06 | 9.31E+03 | 1.21E+06 | 1.21E+06 | 1.30E−02 | 8.82E−05 | 1.85E−02 | 1.85E−02 | 9.62 |
| SCR-3G7 | 6.00E+05 | 6.24E+03 | 5.57E+05 | 5.57E+05 | 9.80E−05 | 1.11E−06 | 1.40E−04 | 1.40E−04 | 0.16* |
| SCR-3A5 | 1.70E+07 | 3.00E+05 | 1.58E+07 | 1.58E+07 | 1.30E−03 | 2.00E−05 | 1.85E−03 | 1.85E−03 | 0.08 |
| MIN | 3.80E+05 | 9.06E+02 | 3.53E+05 | 3.53E+05 | 3.20E−09 | 1.34E−07 | 4.56E−09 | 1.34E−07 | 1.00E−02 |
| MAX | 1.70E+07 | 3.00E+05 | 1.58E+07 | 1.58E+07 | 1.30E−02 | 8.82E−05 | 1.85E−02 | 1.85E−02 | 9.62E+00 |

*= the k_{off} too slow to be accurately calculated

Assessing the Impact of Custom Anti-CRP sdAbs Kinetic Rates on Different Biosensing Applications

For the inflammation levels considered in Table 1, the K_{D} of the anti-CRP sdAbs (Table 10) is superior to the minimum CRP concentration in blood, that is 0.01 μM (Table 1). The TEM predicts that, in this nonlinear range, the proportion of bound sdAbs at equilibrium is not proportional to the input CRP concentration, due to the saturation effects of the sensor surface (C(t)≤K_{D} in Equation 3). Specifically, the proportion of bound sdAbs at baseline CRP inflammation level is equal for instance to 50%, 93.4% and 99.7% for SCR-1A1, SCR-3A4, and SCR-3H6 respectively as shown in Table 12. This high to nearly complete occupancy of the sensor surface results from the kinetic rates of the considered sdAbs and the large, targeted analyte concentration. Beyond 100% occupancy, the sensor response will not change for increasing inflammation levels.

In the non-linear binding regime, while the proportion of bound sdAbs at equilibrium is not proportional to the input concentration, the initial reaction rate \dot{S}(0) increases linearly with the input concentration C₀ (Equation 6). For continuous CRP measurements, the medium of interest can thus be sampled at time to and the sensor signal measured at consecutive time steps t at the start of the association reaction. The input concentration C₀ can then be measured by dividing the initial slope of the measurements by a constant kₐSₘₐₓ determined previously through sensor calibration. For each sdAb, the time constant t, the time required for the signal to develop from zero to about 63% occupied sites (Kusnezow et al., 2006), as well as the proportion of occupied sites at equilibrium was calculated (Table 12). The sensor occupation can thus be probed before saturation, which depending on the biosensor system used can be done within a few milliseconds (Das et al. 2021). Given the time constant t is indeed in the order of tens (SCR-3A5) to hundreds of milliseconds (SCR-3H6, SCR-3A4, SCR-1A1, SCR-3E5, SCR-3B7, SCR-3H1, SCR-2D2, SCR-2B4) to seconds (e.g. SCR-2D1, SCR-3E4, SCR-3G7) even for CRP concentrations associated with severe inflammation, the herein provided anti-CRP sdAbs may be particularly advantageous for detecting and/or quantifying CRP in in vitro and/or in vivo diagnostic applications or for (continuous) monitoring of CRP concentration in a biological fluid (e.g, wherein the sdAbs are comprised in a biosensor). Accordingly, Table 12 demonstrates that the herein provided anti-CRP sdAbs are surprisingly able to detect and/or quantify CRP concentrations across throughout the entire inflammation range of CRP, e.g. in human blood. Thus, the herein provided sdAbs may be particularly useful in the (continuous) measurement of CRP concentrations (such as in biosensors).

Alternatively or additionally to using the sdAbs kinetic parameters to achieve continuous monitoring of sdAbs, repeated measurements could be achieved by recovering the sdAb-antigen complex e.g. by forced release through increasing temperature, inducing vibration, pH modifications, or changing the redox potential. Accordingly, forced release approaches may be employed in order to further enhance the ability of the herein provided sdAbs to continuously detect and/or quantify CRP across an even broader range of inflammation (e.g., across the full range of CRP concentrations detailed in Table 1) or finetune CRP detection within the relevant inflammation range. Further details on forced release approaches may be found in Govaert et al. 2012, Kunz et al. 2017, Kunz et al. 2019, Kunz et al. 2018, Gil et al. 2019, Renicke et al. 2013, Turton et al. 2014, Reverberi et al. 2007, Klaus and Deshmukh 2012, and Thompson et al. 2020, all of which are herewith incorporated by reference in their entirety. Alternatively, microfluidic approaches could be used in multi-sensor chips, where for each measurement, only a single dedicated sensor chip is flooded with sample and used for measurement. Such approaches are detailed e.g. in Glatz et al. 2022, Chen et al. 2023 and Pour et al. 2024, all of which are incorporated herein in their entirety.

For single shot measurements, the same technique can be applied. Additionally, point of care devices allow to dilute the analyzed sample. This approach would reduce the slope of the reaction rate (decrease C₀ in Equation 6) and allow to measure a higher number of datapoints to extract C₀. Alternatively, serial dilution could be used to reduce the input concentration and reach a linear binding regime between CRP and the sdAbs. The optimal dilution factor will depend on the kinetic rate of the sdAb used as target molecule and the extent of the targeted CRP concentration range.

TABLE 12
Time required for the signal to develop from zero to about 63% occupied sites (time constant τ) and proportion of the occupied
sites at the equilibrium of the sdAbs clones against the human inflammation ranges. The CRP concentrations are based on Table 1.

| | Concen- | Time constant τ [sec] | | | | | | | | | | | |
|---|---|---|---|---|---|---|---|---|---|---|---|---|---|
| Inflam- | tration | SC | SC | SC | SC | SC | SC | SC | SC | SC | SC | SC | SC |
| mation | in a biological | R- | R- | R- | R- | R- | R- | R- | R- | R- | R- | R- | R- |
| range | fluid, e.g. blood | 3H6 | 3A4 | 1A1 | 3E5 | 3B7 | 3H1 | 2D2 | 2B4 | 2D1 | 3E4 | 3G7 | 3A5 |
| Base- | 0.01 μM | 41.53 | 34.60 | 38.46 | 58.82 | 66.67 | 47.06 | 62.42 | 58.24 | 120.48 | 258.93 | 163.99 | 5.84 |
| line | (or less) | | | | | | | | | | | | |
| Low | 0.01 μM- | 41.53- | 34.60- | 38.46- | 58.82- | 66.67- | 47.06- | 62.42- | 58.24- | 120.48- | 258.93- | 163.99- | 5.84- |
| | 0.1 μM | 4.17 | 3.68 | 6.99 | 5.88 | 6.67 | 4.76 | 6.25 | 5.88 | 12.45 | 26.27 | 16.64 | 0.59 |
| Mod- | 0.1 μM- | 4.17- | 3.68- | 6.99- | 5.88- | 6.67- | 4.76- | 6.25- | 5.88- | 12.45- | 26.27- | 16.64- | 0.59- |
| erate | 1 μM | 0.42 | 0.37 | 0.76 | 0.59 | 0.67 | 0.48 | 0.62 | 0.59 | 1.25 | 2.63 | 1.67 | 0.06 |
| Se- | 1 μM | 0.42 | 0.37 | 0.76 | 0.59 | 0.67 | 0.48 | 0.62 | 0.59 | 1.25 | 2.63 | 1.67 | 0.06 |
| vere | (or more) | | | | | | | | | | | | |

| | Concen- | Proportion of occupied sites at equilibrium [%] | | | | | | | | | | | |
|---|---|---|---|---|---|---|---|---|---|---|---|---|---|
| Inflam- | tration | SC | SC | SC | SC | SC | SC | SC | SC | SC | SC | SC | SC |
| mation | in a biological | R- | R- | R- | R- | R- | R- | R- | R- | R- | R- | R- | R- |
| range | fluid, e.g. blood | 3H6 | 3A4 | 1A1 | 3E5 | 3B7 | 3H1 | 2D2 | 2B4 | 2D1 | 3E4 | 3G7 | 3A5 |
| Base- | 0.01 μM | 99.7 | 93.4 | 50.0 | 100.0 | 100.0 | 98.8 | 99.9 | 99.0 | 96.4 | 98.4 | 98.4 | 99.2 |
| line | (or less) | | | | | | | | | | | | |
| Low | 0.01 μM- | 99.7- | 93.4- | 50.0- | 100.0 | 100.0 | 98.8- | 99.9- | 99.0- | 96.4- | 98.4- | 98.4- | 99.2- |
| | 0.1 μM | 100.0 | 99.3 | 90.9 | | | 99.9 | 100.0 | 99.9 | 99.6 | 99.8 | 99.8 | 99.9 |
| Mod- | 0.1 μM- | 100.0 | 99.3- | 90.9- | 100.0 | 100.0 | 99.9 | 100.0 | 99.9 | 99.6- | 99.8- | 99.8- | 99.9 |
| erate | 1 μM | | 99.9 | 99.0 | | | | | | 100.0 | 100.0 | 100.0 | |
| Se- | 1 μM | 100 | 99.9 | 99.0 | 100.0 | 100.0 | 100.0 | 100.0 | 100.0 | 100.0 | 100.0 | 100.0 | 100.0 |
| vere | (or more) | | | | | | | | | | | | |

TABLE 13
Summary characteristics of exemplary sdAbs provided herein.
The column “Covered inflammation range” in the table below indicates
the respective inflammation range that a given sdAb can cover assuming
sensor occupation can be probed before saturation (as explained above
in relation to the data in Table 12). All other columns summarize
data also already detailed herein above.

| Clone | kₐ[s⁻¹M⁻¹] | k_{d} [s⁻¹] | K_{D} [nM] | Covered inflammation range |
|---|---|---|---|---|
| SCR-3H6 | 2.4E6 | 8.1E−5 | 0.03 | Baseline to severe |
| SCR-3E5 | 1.7E6 | 3.2E−9* | 1.8E−6* | Baseline to severe |
| SCR-3B7 | 1.5E6 | 1.3E−7* | 7.6E−5* | Baseline to severe |
| SCR-3H1 | 2.1E6 | 2.5E−4 | 0.12 | Baseline to severe |
| SCR-2D2 | 1.6E6 | 2.0E−5 | 0.01 | Baseline to severe |
| SCR-2B4 | 1.7E6 | 1.7E−4 | 0.1 | Baseline to severe |
| SCR-2D1 | 8.0E5 | 3.0E−4 | 0.38 | Baseline to severe |
| SCR-3E4 | 3.8E5 | 6.2E−5 | 0.16 | Baseline to severe |
| SCR-3A4 | 2.7E6 | 1.9E−3 | 0.70 | Baseline to severe |
| SCR-1A1 | 1.3E6 | 1.3E−2 | 9.62 | Baseline to severe |
| SCR-3G7 | 6.0E5 | 9.8E−5* | 0.16* | Baseline to severe |
| SCR-3A5 | 1.7E7 | 1.3E−3 | 0.08 | Baseline to severe |

*k^{d} too slow to be accurately calculated

### Example 2: Yeast Production and Purification of Anti-CRP sdAbs

Production is started from a single colony which is inoculated in 5 ml YP with 2% glucose and is grown for 24 hours at 30° C. at 200 rpm. The overnight culture is diluted 1:20 in 100 mL YNB with 2% glucose in a 2 L flask and grown for 24 hours to make a pre-culture. Addition of 750 mL YP, 100 mL 20% glucose, 50 mL 20% galactose to the 100 mL pre-culture starts the production which is continued for 72 hours. After the production ends OD600 is measured (dilute 1:50 in water before measuring). The culture should reach OD600>20. Cultures are spun down for 20 min at 4800 rpm in centrifuge and supernatant is directly after centrifugation poured gently into a clean bottle. The supernatant is filter sterilized using 0.45 μm bottle top filters on an autoclaved bottle. Take 30 μl culture supernatant (taken at the flame with a sterile pipette tip), add 10 μl 4X SDS-sample buffer and boil the samples at >95° C. for 5 min and load 10 μl of the samples on a 15% SDS-polyacrylamide gel next to a 1 μg reference sdAb to estimate the production level. An appropriate amount of supernatant containing sdAb is used for AKTA purification using a JSR protA column according to the manufacturers protocol.

### Example 3: SdAb Binding to CRP-PEPSCAN Peptide Epitope Mapping

The linear and CLIPS peptides are synthesized based on the amino acid sequence of human CRP using standard Fmoc-chemistry and deprotected using trifluoric acid with scavengers. For conformational mapping, the constrained peptides are synthesized on chemical scaffolds in order to reconstruct conformational epitopes, using Chemically Linked Peptides on Scaffolds (CLIPS) technology (Timmerman et al. 2007 (J. Mol. Recognit. 20:283-299)). For example, the single looped peptides are synthesized containing a dicysteine, which was cyclized by treating with alpha, alpha′-dibromoxylene and the size of the loop is varied by introducing cysteine residues at variable spacing. If other cysteines besides the newly introduced cysteines are present, they are replaced by cysteine-acetamydomethyl. The side-chains of the multiple cysteines in the peptides are coupled to CLIPS templates by reacting onto credit-card format polypropylene PEPSCAN cards (455 peptide formats/card) with a 0.5 mM solution of CLIPS template such as 1,3-bis(bromomethyl)benzene in ammonium bicarbonate (20 mM, pH 7.9)/acetonitrile (1: 1 (v/v)). The cards are gently shaken in the solution for 30 to 60 minutes while completely covered in solution. Finally, the cards are washed extensively with excess of H₂O and sonicated in disrupt-buffer containing 1% SDS/0.1% beta-mercaptoethanol in PBS (pH 7.2) at 70° C. for 30 minutes, followed by sonication in H₂O for another 45 minutes. The binding of sdAb to each peptide is tested in a PEPSCAN-based ELISA. The 455-well credit card format polypropylene cards containing the covalently linked peptides are incubated with primary antibody solution, for example, consisting of 1 μg/ml diluted in blocking solution, for example 4% horse serum, 5% ovalbumin (w/v) in PBS/1% Tween. After washing, the peptides are incubated with a 1/1000 dilution of antibody peroxidase conjugate for one hour at 25° C. After washing, the peroxidase substrate 2,2′-azino-di-3-ethylbenzthiazoline sulfonate (ABTS) and 2 μl of 3% H₂O₂ are added. After one hour, the color development is measured. The color development is quantified with a charge coupled device (CCD)—camera and an image processing system (as firstly described in Slootstra et al., 1996 (Mol. Divers. 1:87-96)). The raw data are optical values obtained by a CCD-camera calibrated to export absorption values compatible with a standard 96-well plate ELISA-reader. First the CCD-camera makes a picture of the card before peroxidase coloring and then again after the peroxidase coloring. These two pictures are subtracted from each other yielding a binding value for each peptide. This data is entered in the Peplab™ database for secure storage and retrieval.

All references cited herein are fully incorporated by reference. Having now fully described the invention, it will be understood by a person skilled in the art that the invention may be practiced within a wide and equivalent range of conditions, parameters and the like, without affecting the spirit or scope of the invention or any embodiment thereof.

### LIST OF REFERENCES

Chen, F.; Hu, Q.; Li, H.; Xie, Y.; Xiu, L.; Zhang, Y.; Guo, X.; Yin, K. Multiplex Detection of Infectious Diseases on Microfluidic Platforms. Biosensors 2023, 13, 410. https://doi.org/10.3390/bios13030410
Das J, Gomis S, Chen JB, Yousefi H, Ahmed S, Mahmud A, Zhou W, Sargent EH, Kelley SO. Reagentless biomolecular analysis using a molecular pendulum. Nat Chem. 2021 May; 13(5): 428-434. doi: 10.1038/s41557-021-00644-y. Epub 2021 Mar. 8. PMID: 33686229.
Devran, Ö., Karakurt, Z., Adigüzel, N., Güngör, G., Moçin, Ö. Y., Balcl, M. K., çelik, E., Saltürk, C., Taklr, H. B., Kargln, F., & Ylmaz, A. (2012). C-reactive protein as a predictor of mortality in patients affected with severe sepsis in intensive care unit. Multidisciplinary Respiratory Medicine, 7(1), 47. https://doi.org/10.1186/2049-6958-7-47
Dinh, K. M., Kaspersen, K. A., Mikkelsen, S., Pedersen, O. B., Petersen, M. S., Thørner, L. W., Hjalgrim, H., Rostgaard, K., Ullum, H., & Erikstrup, C. (2019). Low-grade inflammation is negatively associated with physical Health-Related Quality of Life in healthy individuals: Results from The Danish Blood Donor Study (DBDS). PLOS ONE, 14(3), e0214468. https://doi.org/10.1371/journal.pone.0214468
Gil, Agnieszka A., Evan M. Zhao, Maxwell Z. Wilson, Alexander G. Goglia, Cesar Carrasco-Lopez, José L. Avalos, and Jared E. Toettcher. 2019. “Optogenetic Control of Protein Binding Using Light-Switchable Nanobodies.” Preprint. Biochemistry. https://doi.org/10.1101/739201.
Glatz RT, Ates HC, Mohsenin H, Weber W, Dincer C. Designing electrochemical microfluidic multiplexed biosensors for on-site applications. Anal Bioanal Chem. 2022 September; 414(22): 6531-6540. doi: 10.1007/s00216-022-04210-4. Epub 2022 Jul. 6. PMID: 35794347; PMCID: PMC9411084.
Govaert, Jochen, Mireille Pellis, Nick Deschacht, Cécile Vincke, Katja Conrath, Serge Muyldermans, and Dirk Saerens. 2012. “Dual Beneficial Effect of Interloop Disulfide Bond for Single Domain Antibody Fragments.” Journal of Biological Chemistry 287(3): 1970-79.
Hart, P. C., Rajab, I. M., Alebraheem, M., & Potempa, L. A. (2020). C-Reactive Protein and Cancer-Diagnostic and Therapeutic Insights. Frontiers in Immunology, 11, 595835. https://doi.org/10.3389/fimmu.2020.595835
Herieka, M., & Erridge, C. (2014). High-fat meal induced postprandial inflammation. Molecular Nutrition & Food Research, 58 (1), 136-146. https://doi.org/10.1002/mnfr.201300104
HyTest (“HyTest, TechNotes: Human C-Reactive Protein (CRP)”, Retrieved from the Internet on May 1, 2022 at URL: https://shop.hytest.fi/spree/products/4177/CRP TechNotes.pdf)
Keegan, A., Ricciuti, B., Garden, P., Cohen, L., Nishihara, R., Adeni, A., Paweletz, C., Supplee, J., Janne, P. A., Severgnini, M., Awad, M. M., & Walt, D. R. (2020). Plasma IL-6 changes correlate to PD-1 inhibitor responses in NSCLC. Journal for ImmunoTherapy of Cancer, 8 (2), e000678. https://doi.org/10.1136/jitc-2020-000678
Klaus, T., Deshmukh, S. pH-responsive antibodies for therapeutic applications. J Biomed Sci 28, 11 (2021). https://doi.org/10.1186/s12929-021-00709-7
Kongsuphol, P., Ng, H. H., Pursey, J. P., Arya, S. K., Wong, C. C., Stulz, E., & Park, M. K. (2014). EIS-based biosensor for ultra-sensitive detection of TNF-α from non-diluted human serum. Biosensors & bioelectronics, 61, 274-279. https://doi.org/10.1016/j.bios.2014.05.017
Kunz, Patrick, Tilman Flock, Nicolas Soler, Moritz Zaiss, Cécile Vincke, Yann Sterckx, Damjana Kastelic, Serge Muyldermans, and Jörg D. Hoheisel. 2017. “Exploiting Sequence and Stability Information for Directing Nanobody Stability Engineering.” Biochimica et Biophysica Acta (BBA) General Subjects 1861 (9): 2196-2205. https://doi.org/10.1016/j.bbagen.2017.06.014.
Kunz, Patrick, Aurelio Ortale, Norbert Mücke, Katinka Zinner, and Jörg D Hoheisel. 2019. “Nanobody Stability Engineering by Employing the ΔTm Shift; a Comparison with Apparent Rate Constants of Heat-Induced Aggregation.” Protein Engineering, Design and Selection, July, gzz017. https://doi.org/10.1093/protein/gzz017.
Kunz, Patrick, Katinka Zinner, Norbert Mücke, Tanja Bartoschik, Serge Muyldermans, and Jörg D. Hoheisel. 2018. “The Structural Basis of Nanobody Unfolding Reversibility and Thermoresistance.” Scientific Reports 8(1): 7934. https://doi.org/10.1038/s41598-018-26338-z.
Kusnezow, W., Syagailo, Y. V., Rüffer, S., Klenin, K., Sebald, W., Hoheisel, J. D., Gauer, C., & Goychuk, I. (2006). Kinetics of antigen binding to antibody microspots: Strong limitation by to surface. PROTEOMICS, mass transport the 6(3), 794-803. https://doi.org/10.1002/pmic.200500149
Landry, A., Docherty, P., Ouellette, S., & Cartier, L. J. (2017). Causes and outcomes of markedly elevated C-reactive protein levels. Canadian Family Physician Medecin de Famille Canadien, 63(6), e316-e323.
Mavropoulou, E., Mechie, N.-C., Knoop, R., Petzold, G., Ellenrieder, V., Kunsch, S., Pilavakis, Y., & Amanzada, A. (2020). Association of serum interleukin-6 and soluble interleukin-2-receptor levels with disease activity status in patients with inflammatory bowel disease: A prospective observational PLOS ONE, study. 15 (5), e0233811. https://doi.org/10.1371/journal.pone.0233811
Oloketuyi, S., Bernedo, R., Christmann, A., Borkowska, J., Cazzaniga, G., Schuchmann, H. W., Niedziółkₐ-Jönsson, J., Szot-Karpińska, K., Kolmar, H., & de Marco, A. (2021). Native llama Nanobody Library Panning Performed by Phage and Yeast Display Provides Binders Suitable for C-Reactive Protein Detection. Biosensors, 11 (12), 496. https://doi.org/10.3390/bios11120496
Pour SRS, Calabria D, Emamiamin A, Lazzarini E, Pace A, Guardigli M, Zangheri M, Mirasoli M. Microfluidic-Based Non-Invasive Wearable Biosensors for Real-Time Monitoring of Sweat Biomarkers. Biosensors (Basel). 2024 Jan. 4;14(1): 29. doi: 10.3390/bios14010029. PMID: 38248406; PMCID: PMC10813635.
Póvoa, P., Almeida, E., Moreira, P., Fernandes, A., Mealha, R., Aragão, A., & Sabino, H. (1998). C-reactive protein as an indicator of sepsis. Intensive Care Medicine, 24 (10), 1052-1056. https://doi.org/10.1007/s001340050715
Renicke, Christian, Daniel Schuster, Svetlana Usherenko, Lars-Oliver Essen, and Christof Taxis. 2013. “A LOV2 Domain-Based Optogenetic Tool to Control Protein Degradation and Cellular Function.” Chemistry & Biology 20 (4): 619-26. https://doi.org/10.1016/j.chembiol.2013.03.005.
Reverberi, Roberto, and Lorenzo Reverberi. 2007. “Factors Affecting the Antigen-Antibody Reaction.” Blood Transfusion. https://doi.org/10.2450/2007.0047-07
Rich, Rebecca L., et al. “A global benchmark study using affinity-based biosensors.” Analytical biochemistry 386.2 (2009): 194-216.
Ridker, P. M., Buring, J. E., Cook, N. R., & Rifai, N. (2003). C-Reactive Protein, the Metabolic Syndrome, and Risk of Incident Cardiovascular Events. Circulation, 107 (3), 391-397. https://doi.org/10.1161/01.cir.0000055014.62083.05
Ruiz-González, A., Utrillo, L., Bielsa, S., Falguera, M., & Porcel, J. M. (2016). The Diagnostic Value of Serum C-Reactive Protein for Identifying Pneumonia in Hospitalized Patients with Acute Respiratory Symptoms. Journal of Biomarkers, 2016, 2198745. https://doi.org/10.1155/2016/2198745
Sproston, N. R., & Ashworth, J. J. (2018). Role of C-Reactive Protein at Sites of Inflammation and Infection. Frontiers in Immunology, 9, 754. https://doi.org/10.3389/fimmu.2018.00754
Stenberg, Esa, et al. “Quantitative determination of surface concentration of protein with surface plasmon resonance using radiolabeled proteins.” Journal of colloid and interface science 143.2 (1991): 513-526.
Thompson, I.A.P., Zheng, L., Eisenstein, M. et al. Rational design of aptamer switches with programmable pH response. Nat Commun 11, 2946 (2020). https://doi.org/10.1038/s41467-020-16808-2
Turton, David A., Hans Martin Senn, Thomas Harwood, Adrian J. Lapthorn, Elizabeth M. Ellis, and Klaas Wynne. 2014. “Terahertz Underdamped Vibrational Motion Governs Protein-Ligand Binding in Solution.” Nature Communications 5(1): 3999. https://doi.org/10.1038/ncomms4999.

Sequence listing:

SEQ ID NO: 1-full length SCR-1A1 sdAb
AVQLVESGGGLAQPGGSLRLSCVASGLMFSGYAMSWARQAPGKRLEWVS
GIYPDGSETYYADSVKGRFTISRDNAKNTLYLQMNSLEPEDTALYYCAK
DGVGWRRAGTPNLGCLNPPCDYWGQGTQVTVSS
SEQ ID NO: 2-SCR-1A1 FR1
AVQLVESGGGLAQPGGSLRLSCVAS
SEQ ID NO: 3-SCR-1A1 CDR1
GLMFSGYA
SEQ ID NO: 4-SCR-1A1 FR2
MSWARQAPGKRLEWVSG
SEQ ID NO: 5-SCR-1A1 CDR2
IYPDGSET
SEQ ID NO: 6-SCR-1A1 FR3
YYADSVKGRFTISRDNAKNTLYLQMNSLEPEDTALYYC
SEQ ID NO: 7-SCR-1A1 CDR3
AKDGVGWRRAGTPNLGCLNPPCDY
SEQ ID NO: 8-SCR-1A1 FR4
WGQGTQVTVSS
SEQ ID NO: 9-full length SCR-2B4 sdAb
EVQLVESGGGLVQPGGSLRLSCAASEFNLDSYSIVWFRQAPGKGREGIS
CISSGGSTTYRDSVKGRFTISRDNAKNTVYLQMNSLKPEDTAVYYCAAA
RICTYYSSTYDGWGQGTQVTVSS
SEQ ID NO: 10-SCR-2B4 FR1
EVQLVESGGGLVQPGGSLRLSCAAS
SEQ ID NO: 11-SCR-2B4 CDR1
EFNLDSYS
SEQ ID NO: 12-SCR-2B4 FR2
IVWFRQAPGKGREGISC
SEQ ID NO: 13-SCR-2B4 CDR2
ISSGGST
SEQ ID NO: 14-SCR-2B4 FR3
TYRDSVKGRFTISRDNAKNTVYLQMNSLKPEDTAVYYC
SEQ ID NO: 15-SCR-2B4 CDR3
AAARICTYYSSTYDG
SEQ ID NO: 16-SCR-2B4 FR4
WGQGTQVTVSS
SEQ ID NO: 17-full length SCR-2D1 sdAb
EVQLVESGGGLVQAGGSLRLSCAASGRTFSGYAMGWFRQAPGKERESVA
SISRSGPTTRYADSVKGRFTISRDNAKNTVYLDMDSLKPEDTAVYYCAG
KSDVIAVQATEYNYWGQGTQVTVSS
SEQ ID NO: 18-SCR-2D1 FR1
EVQLVESGGGLVQAGGSLRLSCAAS
SEQ ID NO: 19-SCR-2D1 CDR1
GRTFSGYA
SEQ ID NO: 20-SCR-2D1 FR2
MGWFRQAPGKERESVAS
SEQ ID NO: 21-SCR-2D1 CDR2
ISRSGPTT
SEQ ID NO: 22-SCR-2D1 FR3
RYADSVKGRFTISRDNAKNTVYLDMDSLKPEDTAVYYC
SEQ ID NO: 23-SCR-2D1 CDR3
AGKSDVIAVQATEYNY
SEQ ID NO: 24-SCR-2D1 FR4
WGQGTQVTVSS
SEQ ID NO: 25-full length SCR-2D2 sdAb
EVQLVESGGGLVQPGGSLRLSCAASGVTLAKNAIGWFRQAPGKEREGIS
CIRGSHGDTAYVDSVKGRFTISRDNTKNTVYLEMNNLKPEDTAVYYCAT
GDPQLRGGERCPYEYEYWGRGTQVTVSS
SEQ ID NO: 26-SCR-2D2 FR1
EVQLVESGGGLVQPGGSLRLSCAAS
SEQ ID NO: 27-SCR-2D2 CDR1
GVTLAKNA
SEQ ID NO: 28-SCR-2D2 FR2
IGWFRQAPGKEREGISC
SEQ ID NO: 29-SCR-2D2 CDR2
IRGSHGDT
SEQ ID NO: 30-SCR-2D2 FR3
AYVDSVKGRFTISRDNTKNTVYLEMNNLKPEDTAVYYC
SEQ ID NO: 31-SCR-2D2 CDR3
ATGDPQLRGGERCPYEYEY
SEQ ID NO: 32-SCR-2D2 FR4
WGRGTQVTVSS
SEQ ID NO: 33-full length SCR-3A4 sdAb
EVQLVESGGGSVQTGGSLRLSCAASGRTFSTYAMGWFRQAPGKEREFVA
AISRSGLNRFYADSVKGRFTIARDNAKNTVTLQMYSLKPEDTAVYYCAA
DSRRAPFDTPDRYMPRHYDDWGQGTQVTVSS
SEQ ID NO: 34-SCR-3A4 FR1
EVQLVESGGGSVQTGGSLRLSCAAS
SEQ ID NO: 35-SCR-3A4 CDR1
GRTFSTYA
SEQ ID NO: 36-SCR-3A4 FR2
MGWFRQAPGKEREFVAA
SEQ ID NO: 37-SCR-3A4 CDR2
ISRSGLNR
SEQ ID NO: 38-SCR-3A4 FR3
FYADSVKGRFTIARDNAKNTVTLQMYSLKPEDTAVYYC
SEQ ID NO: 39-SCR-3A4 CDR3
AADSRRAPFDTPDRYMPRHYDD
SEQ ID NO: 40-SCR-3A4 FR4
WGQGTQVTVSS
SEQ ID NO: 41-full length SCR-3A5 sdAb
EVQLVESGGGLVQAGDSLRLSCAASGRTFSGAAMVWFRQAPGKERERLA
AISWSGGSTWYADSVKGRFSISRDNAKNTVYLQMNSLKPEDTAVYICAA
HSGRDYYTLGEPYEPDYWGQGTQVTVSS
SEQ ID NO: 42-SCR-3A5 FR1
EVQLVESGGGLVQAGDSLRLSCAAS
SEQ ID NO: 43-SCR-3A5 CDR1
GRTFSGAA
SEQ ID NO: 44-SCR-3A5 FR2
MVWFRQAPGKERERLAA
SEQ ID NO: 45-SCR-3A5 CDR2
ISWSGGST
SEQ ID NO: 46-SCR-3A5 FR3
WYADSVKGRFSISRDNAKNTVYLQMNSLKPEDTAVYIC
SEQ ID NO: 47-SCR-3A5 CDR3
AAHSGRDYYTLGEPYEPDY
SEQ ID NO: 48-SCR-3A5 FR4
WGQGTQVTVSS
SEQ ID NO: 49-full length SCR-3B7 sdAb
EVQLVESGGGLVQAGGSLRLSCAASGRTFSNYVMGWFRQAPGKEREFVA
SIARSGPTTYYTDSVKGRFTISRDNAKNTVYLQMNSLRPADTALYYCAA
KRDVISRTAREYDYWGQGTQVTVSS
SEQ ID NO: 50-SCR-3B7 FR1
EVQLVESGGGLVQAGGSLRLSCAAS
SEQ ID NO: 51-SCR-3B7 CDR1
GRTFSNYV
SEQ ID NO: 52-SCR-3B7 FR2
MGWFRQAPGKEREFVAS
SEQ ID NO: 53-SCR-3B7 CDR2
IARSGPTT
SEQ ID NO: 54-SCR-3B7 FR3
YYTDSVKGRFTISRDNAKNTVYLQMNSLRPADTALYYC
SEQ ID NO: 55-SCR-3B7 CDR3
AAKRDVISRTAREYDY
SEQ ID NO: 56-SCR-3B7 FR4
WGQGTQVTVSS
SEQ ID NO: 57-full length SCR-3E4 sdAb
EVQLVESGGGLVQPGGSLRLFCAASGLTLDDYNVGWFRQAPGKELEGVA
CISRKDGSTYYADSVKGRFTISRDNAKNTVHLQMSSLKPEDTAVYRCVT
EGPGITTVQAMCDMLLSNYWGQGTQVTVSS
SEQ ID NO: 58-SCR-3E4 FR1
EVQLVESGGGLVQPGGSLRLFCAAS
SEQ ID NO: 59-SCR-3E4 CDR1
GLTLDDYN
SEQ ID NO: 60-SCR-3E4 FR2
VGWFRQAPGKELEGVAC
SEQ ID NO: 61-SCR-3E4 CDR2
ISRKDGST
SEQ ID NO: 62-SCR-3E4 FR3
YYADSVKGRFTISRDNAKNTVHLQMSSLKPEDTAVYRC
SEQ ID NO: 63-SCR-3E4 CDR3
VTEGPGITTVQAMCDMLLSNY
SEQ ID NO: 64-SCR-3E4 FR4
WGQGTQVTVSS
SEQ ID NO: 65-full length SCR-3E5 sdAb
VVQLVESGGGLVQAGGSLRLSCAASGRTLSSYAMGWFRQAPGKEREFVA
AVSGSGGTTYYADDSVKGRFTISRDNAKNTVSLQMNSLKPEDTAVYYCA
ANRDTISRIQYEYDYWGQGTQVTVSS
SEQ ID NO: 66-SCR-3E5 FR1
VVQLVESGGGLVQAGGSLRLSCAAS
SEQ ID NO: 67-SCR-3E5 CDR1
GRTLSSYA
SEQ ID NO: 68-SCR-3E5 FR2
MGWFRQAPGKEREFVAA
SEQ ID NO: 69-SCR-3E5 CDR2
VSGSGGTT
SEQ ID NO: 70-SCR-3E5 FR3
YYADDSVKGRFTISRDNAKNTVSLQMNSLKPEDTAVYYC
SEQ ID NO: 71-SCR-3E5 CDR3
AANRDTISRIQYEYDY
SEQ ID NO: 72-SCR-3E5 FR4
WGQGTQVTVSS
SEQ ID NO: 73-full length SCR-3G7 sdAb
EVQLVESGGGLVQVGGSLRLSCAASGRTFSTYDMGWYRQAPGKERERVA
AIRWRDSSTDYADSVQGRFTISRDNAKNTMYLQMDNLRPEDTAIYYCHA
GDYSGSYYRYDYWGQGTQVTVSS
SEQ ID NO: 74-SCR-3G7 FR1
EVQLVESGGGLVQVGGSLRLSCAAS
SEQ ID NO: 75-SCR-3G7 CDR1
GRTFSTYD
SEQ ID NO: 76-SCR-3G7 FR2
MGWYRQAPGKERERVAA
SEQ ID NO: 77-SCR-3G7 CDR2
IRWRDSST
SEQ ID NO: 78-SCR-3G7 FR3
DYADSVQGRFTISRDNAKNTMYLQMDNLRPEDTAIYYC
SEQ ID NO: 79-SCR-3G7 CDR3
HAGDYSGSYYRYDY
SEQ ID NO: 80-SCR-3G7 FR4
WGQGTQVTVSS
SEQ ID NO: 81-full length SCR-3H1 sdAb
EVQLVESGGGLVQAGDSLRLSCADSGRTFSSYAMGWFRQAPGKGREFVA
VISSGGSTIYGDSVKGRFTISRDNARNTGYLEVNSLKFEDTAVYYCAAA
RLSSRSPLDYRYWGQGTQVTVSS
SEQ ID NO: 82-SCR-3H1 FR1
EVQLVESGGGLVQAGDSLRLSCADS
SEQ ID NO: 83-SCR-3H1 CDR1
GRTFSSYA
SEQ ID NO: 84-SCR-3H1 FR2
MGWFRQAPGKGREFVAV
SEQ ID NO: 85-SCR-3H1 CDR2
ISSGGST
SEQ ID NO: 86-SCR-3H1 FR3
IYGDSVKGRFTISRDNARNTGYLEVNSLKFEDTAVYYC
SEQ ID NO: 87-SCR-3H1 CDR3
AAARLSSRSPLDYRY
SEQ ID NO: 88-SCR-3H1 FR4
WGQGTQVTVSS
SEQ ID NO: 89-full length SCR-3H6 sdAb
EVQLVESGGGLVQTGGSLRLSCAASGYSFSTYGMGWFRQAPGKQRELVA
AISSGGSTIYADSAKGRFTISRDNAKNTVYLQMNSLKPEDTAVYYCAGA
RLVSRSPAEYRGWGQGTQVTVSS
SEQ ID NO: 90-SCR-3H6 FR1
EVQLVESGGGLVQTGGSLRLSCAAS
SEQ ID NO: 91-SCR-3H6 CDR1
GYSFSTYG
SEQ ID NO: 92-SCR-3H6 FR2
MGWFRQAPGKQRELVAA
SEQ ID NO: 93-SCR-3H6 CDR2
ISSGGST
SEQ ID NO: 94-SCR-3H6 FR3
IYADSAKGRFTISRDNAKNTVYLQMNSLKPEDTAVYYC
SEQ ID NO: 95-SCR-3H6 CDR3
AGARLVSRSPAEYRG
SEQ ID NO: 96-SCR-3H6 FR4
WGQGTQVTVSS

TABLE 14
Table summarizing the herein disclosed sequences.

| SEQ ID NO: | sdAb | Fragment |
|---|---|---|
| 1 | SCR-1A1 | full length sdAb |
| 2 | SCR-1A1 | FR1 |
| 3 | SCR-1A1 | CDR1 |
| 4 | SCR-1A1 | FR2 |
| 5 | SCR-1A1 | CDR2 |
| 6 | SCR-1A1 | FR3 |
| 7 | SCR-1A1 | CDR3 |
| 8 | SCR-1A1 | FR4 |
| 9 | SCR-2B4 | full length sdAb |
| 10 | SCR-2B4 | FR1 |
| 11 | SCR-2B4 | CDR1 |
| 12 | SCR-2B4 | FR2 |
| 13 | SCR-2B4 | CDR2 |
| 14 | SCR-2B4 | FR3 |
| 15 | SCR-2B4 | CDR3 |
| 16 | SCR-2B4 | FR4 |
| 17 | SCR-2D1 | full length sdAb |
| 18 | SCR-2D1 | FR1 |
| 19 | SCR-2D1 | CDR1 |
| 20 | SCR-2D1 | FR2 |
| 21 | SCR-2D1 | CDR2 |
| 22 | SCR-2D1 | FR3 |
| 23 | SCR-2D1 | CDR3 |
| 24 | SCR-2D1 | FR4 |
| 25 | SCR-2D2 | full length sdAb |
| 26 | SCR-2D2 | FR1 |
| 27 | SCR-2D2 | CDR1 |
| 28 | SCR-2D2 | FR2 |
| 29 | SCR-2D2 | CDR2 |
| 30 | SCR-2D2 | FR3 |
| 31 | SCR-2D2 | CDR3 |
| 32 | SCR-2D2 | FR4 |
| 33 | SCR-3A4 | full length sdAb |
| 34 | SCR-3A4 | FR1 |
| 35 | SCR-3A4 | CDR1 |
| 36 | SCR-3A4 | FR2 |
| 37 | SCR-3A4 | CDR2 |
| 38 | SCR-3A4 | FR3 |
| 39 | SCR-3A4 | CDR3 |
| 40 | SCR-3A4 | FR4 |
| 41 | SCR-3A5 | full length sdAb |
| 42 | SCR-3A5 | FR1 |
| 43 | SCR-3A5 | CDR1 |
| 44 | SCR-3A5 | FR2 |
| 45 | SCR-3A5 | CDR2 |
| 46 | SCR-3A5 | FR3 |
| 47 | SCR-3A5 | CDR3 |
| 48 | SCR-3A5 | FR4 |
| 49 | SCR-3B7 | full length sdAb |
| 50 | SCR-3B7 | FR1 |
| 51 | SCR-3B7 | CDR1 |
| 52 | SCR-3B7 | FR2 |
| 53 | SCR-3B7 | CDR2 |
| 54 | SCR-3B7 | FR3 |
| 55 | SCR-3B7 | CDR3 |
| 56 | SCR-3B7 | FR4 |
| 57 | SCR-3E4 | full length sdAb |
| 58 | SCR-3E4 | FR1 |
| 59 | SCR-3E4 | CDR1 |
| 60 | SCR-3E4 | FR2 |
| 61 | SCR-3E4 | CDR2 |
| 62 | SCR-3E4 | FR3 |
| 63 | SCR-3E4 | CDR3 |
| 64 | SCR-3E4 | FR4 |
| 65 | SCR-3E5 | full length sdAb |
| 66 | SCR-3E5 | FR1 |
| 67 | SCR-3E5 | CDR1 |
| 68 | SCR-3E5 | FR2 |
| 69 | SCR-3E5 | CDR2 |
| 70 | SCR-3E5 | FR3 |
| 71 | SCR-3E5 | CDR3 |
| 72 | SCR-3E5 | FR4 |
| 73 | SCR-3G7 | full length sdAb |
| 74 | SCR-3G7 | FR1 |
| 75 | SCR-3G7 | CDR1 |
| 76 | SCR-3G7 | FR2 |
| 77 | SCR-3G7 | CDR2 |
| 78 | SCR-3G7 | FR3 |
| 79 | SCR-3G7 | CDR3 |
| 80 | SCR-3G7 | FR4 |
| 81 | SCR-3H1 | full length sdAb |
| 82 | SCR-3H1 | FR1 |
| 83 | SCR-3H1 | CDR1 |
| 84 | SCR-3H1 | FR2 |
| 85 | SCR-3H1 | CDR2 |
| 86 | SCR-3H1 | FR3 |
| 87 | SCR-3H1 | CDR3 |
| 88 | SCR-3H1 | FR4 |
| 89 | SCR-3H6 | full length sdAb |
| 90 | SCR-3H6 | FR1 |
| 91 | SCR-3H6 | CDR1 |
| 92 | SCR-3H6 | FR2 |
| 93 | SCR-3H6 | CDR2 |
| 94 | SCR-3H6 | FR3 |
| 95 | SCR-3H6 | CDR3 |
| 96 | SCR-3H6 | FR4 |

1. An anti-C-reactive protein (CRP) single-domain antibody (sdAb) comprising an on-rate constant (kₒₙ) of about or more than about 2.1×10\hat{ }⁵ s⁻¹M⁻¹, an off-rate constant (k_{off}) of about 2.5×10\hat{ }−9 s⁻¹−2×10\hat{ }−1 s⁻¹, and/or a dissociation constant (K_{D}) of about less than about 1×10\hat{ }−8 M, preferably wherein said sdAb is capable of detecting and/or quantifying CRP concentrations of about 0.01 μM or less to about 1 μM or more.

2. The anti-CRP sdAb of claim 1, wherein the sdAb comprises a first complementary determining region (CDR1), a second CDR (CDR2) and third CDR (CDR3), wherein:
(i) CDR1 comprises SEQ ID NO: 3 or a variant thereof having up to two amino acid substitutions, CDR2 comprises SEQ ID NO: 5 or a variant thereof having up to two amino acid substitutions, CDR3 comprises SEQ ID NO: 7 or a variant thereof having up to five amino acid substitutions; or
(ii) CDR1 comprises SEQ ID NO: 11 or a variant thereof having up to two amino acid substitutions, CDR2 comprises SEQ ID NO: 13 or a variant thereof having up to two amino acid substitutions, CDR3 comprises SEQ ID NO: 15 or a variant thereof having up to five amino acid substitutions; or
(iii) CDR1 comprises SEQ ID NO: 19 or a variant thereof having up to two amino acid substitutions, CDR2 comprises SEQ ID NO: 21 or a variant thereof having up to two amino acid substitutions, CDR3 comprises SEQ ID NO: 23 or a variant thereof having up to five amino acid substitutions; or
(iv) CDR1 comprises SEQ ID NO: 27 or a variant thereof having up to two amino acid substitutions, CDR2 comprises SEQ ID NO: 29 or a variant thereof having up to two amino acid substitutions, CDR3 comprises SEQ ID NO: 31 or a variant thereof having up to five amino acid substitutions; or
(v) CDR1 comprises SEQ ID NO: 35 or a variant thereof having up to two amino acid substitutions, CDR2 comprises SEQ ID NO: 37 or a variant thereof having up to two amino acid substitutions, CDR3 comprises SEQ ID NO: 39 or a variant thereof having up to five amino acid substitutions; or
(vi) CDR1 comprises SEQ ID NO: 43 or a variant thereof having up to two amino acid substitutions, CDR2 comprises SEQ ID NO: 45 or a variant thereof having up to two amino acid substitutions, CDR3 comprises SEQ ID NO: 47 or a variant thereof having up to five amino acid substitutions;
(vii) CDR1 comprises SEQ ID NO: 51 or a variant thereof having up to two amino acid substitutions, CDR2 comprises SEQ ID NO: 53 or a variant thereof having up to two amino acid substitutions, CDR3 comprises SEQ ID NO: 55 or a variant thereof having up to five amino acid substitutions; or
(viii) CDR1 comprises SEQ ID NO: 59 or a variant thereof having up to two amino acid substitutions, CDR2 comprises SEQ ID NO: 61 or a variant thereof having up to two amino acid substitutions, CDR3 comprises SEQ ID NO: 63 or a variant thereof having up to five amino acid substitutions; or
(ix) CDR1 comprises SEQ ID NO: 67 or a variant thereof having up to two amino acid substitutions, CDR2 comprises SEQ ID NO: 69 or a variant thereof having up to two amino acid substitutions, CDR3 comprises SEQ ID NO: 71 or a variant thereof having up to five amino acid substitutions; or
(x) CDR1 comprises SEQ ID NO: 75 or a variant thereof having up to two amino acid substitutions, CDR2 comprises SEQ ID NO: 77 or a variant thereof having up to two amino acid substitutions, CDR3 comprises SEQ ID NO: 79 or a variant thereof having up to five amino acid substitutions; or
(xi) CDR1 comprises SEQ ID NO: 83 or a variant thereof having up to two amino acid substitutions, CDR2 comprises SEQ ID NO: 85 or a variant thereof having up to two amino acid substitutions, CDR3 comprises SEQ ID NO: 87 or a variant thereof having up to five amino acid substitutions; or
(xii) CDR1 comprises SEQ ID NO: 91 or a variant thereof having up to two amino acid substitutions, CDR2 comprises SEQ ID NO: 93 or a variant thereof having up to two amino acid substitutions, CDR3 comprises SEQ ID NO: 95 or a variant thereof having up to five amino acid substitutions.

3. The anti-CRP sdAb of claim 1 or 2, wherein the sdAb comprises a first framework region (FR1), a second framework region (FR2) and third framework region (FR3), wherein:
(i) FR1 comprises SEQ ID NO: 2 or a variant thereof having up to six amino acid substitutions, FR2 comprises SEQ ID NO: 4 or a variant thereof having up to four amino acid substitutions, FR3 comprises SEQ ID NO: 6 or a variant thereof having up to ten amino acid substitutions, and FR4 comprises SEQ ID NO: 8 or a variant thereof having up to three amino acid substitutions; or
(ii) FR1 comprises SEQ ID NO: 10 or a variant thereof having up to six amino acid substitutions, FR2 comprises SEQ ID NO: 12 or a variant thereof having up to four amino acid substitutions, FR3 comprises SEQ ID NO: 14 or a variant thereof having up to ten amino acid substitutions, and FR4 comprises SEQ ID NO: 16 or a variant thereof having up to three amino acid substitutions; or
(iii) FR1 comprises SEQ ID NO: 18 or a variant thereof having up to six amino acid substitutions, FR2 comprises SEQ ID NO: 20 or a variant thereof having up to four amino acid substitutions, FR3 comprises SEQ ID NO: 22 or a variant thereof having up to ten amino acid substitutions, and FR4 comprises SEQ ID NO: 24 or a variant thereof having up to three amino acid substitutions; or
(iv) FR1 comprises SEQ ID NO: 26 or a variant thereof having up to six amino acid substitutions, FR2 comprises SEQ ID NO: 28 or a variant thereof having up to four amino acid substitutions, FR3 comprises SEQ ID NO: 30 or a variant thereof having up to ten amino acid substitutions, and FR4 comprises SEQ ID NO: 32 or a variant thereof having up to three amino acid substitutions; or
(v) FR1 comprises SEQ ID NO: 34 or a variant thereof having up to six amino acid substitutions, FR2 comprises SEQ ID NO: 36 or a variant thereof having up to four amino acid substitutions, FR3 comprises SEQ ID NO: 38 or a variant thereof having up to ten amino acid substitutions, and FR4 comprises SEQ ID NO: 40 or a variant thereof having up to three amino acid substitutions; or
(vi) FR1 comprises SEQ ID NO: 42 or a variant thereof having up to six amino acid substitutions, FR2 comprises SEQ ID NO: 44 or a variant thereof having up to four amino acid substitutions, FR3 comprises SEQ ID NO: 46 or a variant thereof having up to ten amino acid substitutions, and FR4 comprises SEQ ID NO: 48 or a variant thereof having up to three amino acid substitutions; or
(vii) FR1 comprises SEQ ID NO: 50 or a variant thereof having up to six amino acid substitutions, FR2 comprises SEQ ID NO: 52 or a variant thereof having up to four amino acid substitutions, FR3 comprises SEQ ID NO: 54 or a variant thereof having up to ten amino acid substitutions, and FR4 comprises SEQ ID NO: 56 or a variant thereof having up to three amino acid substitutions;
(viii) FR1 comprises SEQ ID NO: 58 or a variant thereof having up to six amino acid substitutions, FR2 comprises SEQ ID NO: 60 or a variant thereof having up to four amino acid substitutions, FR3 comprises SEQ ID NO: 62 or a variant thereof having up to ten amino acid substitutions, and FR4 comprises SEQ ID NO: 64 or a variant thereof having up to three amino acid substitutions; or
(ix) FR1 comprises SEQ ID NO: 66 or a variant thereof having up to six amino acid substitutions, FR2 comprises SEQ ID NO: 68 or a variant thereof having up to four amino acid substitutions, FR3 comprises SEQ ID NO: 70 or a variant thereof having up to ten amino acid substitutions, and FR4 comprises SEQ ID NO: 72 or a variant thereof having up to three amino acid substitutions; or
(x) FR1 comprises SEQ ID NO: 74 or a variant thereof having up to six amino acid substitutions, FR2 comprises SEQ ID NO: 76 or a variant thereof having up to four amino acid substitutions, FR3 comprises SEQ ID NO: 78 or a variant thereof having up to ten amino acid substitutions, and FR4 comprises SEQ ID NO: 80 or a variant thereof having up to three amino acid substitutions; or
(xi) FR1 comprises SEQ ID NO: 82 or a variant thereof having up to six amino acid substitutions, FR2 comprises SEQ ID NO: 84 or a variant thereof having up to four amino acid substitutions, FR3 comprises SEQ ID NO: 86 or a variant thereof having up to ten amino acid substitutions, and FR4 comprises SEQ ID NO: 88 or a variant thereof having up to three amino acid substitutions; or
(xii) FR1 comprises SEQ ID NO: 90 or a variant thereof having up to six amino acid substitutions, FR2 comprises SEQ ID NO: 92 or a variant thereof having up to four amino acid substitutions, FR3 comprises SEQ ID NO: 94 or a variant thereof having up to ten amino acid substitutions, and FR4 comprises SEQ ID NO: 96 or a variant thereof having up to three amino acid substitutions.

4. The anti-CRP sdAb of any one of claims 1 to 3, wherein the sdAb comprises any one of
(i) SEQ ID NO: 1 or a variant thereof having at least about 90% sequence identity to SEQ ID NO: 1; or
(ii) SEQ ID NO: 9 or a variant thereof having at least about 90% sequence identity to SEQ ID NO: 9; or
(iii) SEQ ID NO: 17 or a variant thereof having at least about 90% sequence identity to SEQ ID NO: 17; or
(iv) SEQ ID NO: 25 or a variant thereof having at least about 90% sequence identity to SEQ ID NO: 25; or
(v) SEQ ID NO: 33 or a variant thereof having at least about 90% sequence identity to SEQ ID NO: 33; or
(vi) SEQ ID NO: 41 or a variant thereof having at least about 90% sequence identity to SEQ ID NO: 41; or
(vii) SEQ ID NO: 49 or a variant thereof having at least about 90% sequence identity to SEQ ID NO: 49; or
(viii) SEQ ID NO: 57 or a variant thereof having at least about 90% sequence identity to SEQ ID NO: 57; or
(ix) SEQ ID NO: 65 or a variant thereof having at least about 90% sequence identity to SEQ ID NO: 65; or
(x) SEQ ID NO: 73 or a variant thereof having at least about 90% sequence identity to SEQ ID NO: 73; or
(xi) SEQ ID NO: 81 or a variant thereof having at least about 90% sequence identity to SEQ ID NO: 81; or
(xii)) SEQ ID NO: 89 or a variant thereof having at least about 90% sequence identity to SEQ ID NO: 89.

5. The anti-CRP sdAb of any one of claims 1-4, wherein the anti-CRP sdAb comprises an on-rate constant (kₒₙ) of about 3.80×10\hat{ }5 s⁻¹M⁻¹−1.70×10\hat{ }7 s⁻¹M⁻¹ and an off-rate constant (k_{off}) of about 3.20×10\hat{ }−9 s⁻¹−1.30×10\hat{ }−2 s⁻¹.

6. The anti-CRP sdAb of any one of claims 1-4, wherein the anti-CRP sdAb comprises an on-rate constant (kₒₙ) of about 3.30×10\hat{ }4 s⁻¹M⁻¹−4.60×10\hat{ }6 s⁻¹M⁻¹ and an off-rate constant (k_{off}) of about 2.60×10\hat{ }−4 s⁻¹−2.20×10\hat{ }−1 s⁻¹.

7. The anti-CRP sdAb of any one of claims 1-4, wherein the anti-CRP sdAb comprises an on-rate constant (kₒₙ) of about 4.10×10\hat{ }4 s⁻¹M⁻¹−3.80×10\hat{ }5 s⁻¹M⁻¹ and an off-rate constant (k_{off}) of about 2.00×10\hat{ }−5 s⁻¹−6.90×10\hat{ }−3 s⁻¹.

8. The anti-CRP sdAb of any one of claims 1-4, wherein the anti-CRP sdAb comprises an on-rate constant (kₒₙ) of about 3.80×10\hat{ }5 s⁻¹M⁻¹−1.70×10\hat{ }7 s⁻¹M⁻¹±9.06×10\hat{ }2 s⁻¹ M⁻¹−3.00×10\hat{ }5 s⁻¹M⁻¹ and an off-rate constant (k_{off}) of about 3.20×10\hat{ }−9 s⁻¹−1.30×10\hat{ }−2 s⁻¹±1.34×10\hat{ }−7 s⁻¹−8.82×10\hat{ }−5 s⁻¹ and optionally a K_{D} of about 1.00×10\hat{ }−2 M−9.62 M, optionally wherein the values are obtained by a SPR Biacore binding kinetic measurement, preferably wherein the sdAb is defined in any one of SEQ ID NOs: 1, 9, 17, 25, 33, 41, 49, 57, 65, 73, 81, 89.

9. The anti-CRP sdAb of any one of claims 1-4, wherein the anti-CRP sdAb comprises an on-rate constant (kₒₙ) of about 4.10×10\hat{ }4 s⁻¹M⁻¹−3.80×10\hat{ }5 s⁻¹M−1±3.31×10\hat{ }2 s⁻¹ M⁻¹−1.48×10\hat{ }4 s⁻¹M⁻¹ and an off-rate constant (k_{off}) of about 2.00×10\hat{ }−5 s⁻¹−6.90×10\hat{ }−3 s⁻¹=1.41×10\hat{ }−6 s⁻¹−8.61×10\hat{ }−5 s⁻¹ and optionally a K_{D} of about 5.80×10\hat{ }−11 M−6.50×10\hat{ }−8 M, optionally wherein the values are obtained by an Octet BLI binding kinetic measurement, preferably wherein the sdAb is defined in any one of SEQ ID NOs: 1, 9, 17, 25, 33, 49, 57, 65, 73, 81, 89.

10. The anti-CRP sdAb of any one of claims 1-9, wherein the anti-CRP sdAb continuously and reversibly binds CRP.

11. The anti-CRP sdAb of any one of claims 1-10, wherein the anti-CRP sdAb binds a second or further CRP after dissociating from a first CRP.

12. The anti-CRP sdAb of any one of claims 1-11, wherein the anti-CRP sdAb inhibits the biological function of CRP.

13. The anti-CRP sdAb of any one of claims 1-11, wherein the anti-CRP sdAb does not inhibit the biological function of CRP.

14. The anti-CRP sdAb of claim 12 or 13, wherein the biological function of CRP is CRP signalling or the CRP signalling pathway.

15. The anti-CRP sdAb of any one of claims 1-14, wherein the anti-CRP sdAb is conjugated to a biosensor electrode, such as a working electrode.

16. The anti-CRP sdAb of claim 15, wherein the anti-CRP sdAb is conjugated to a biosensor electrode via a linker, such as a nucleic acid.

17. The anti-CRP sdAb of claim 15 or 16, wherein the biosensor comprises one or more electrode, such as a working electrode and/or a counter electrode and/or a reference electrode, and a substrate.

18. The anti-CRP sdAb of any one of claims 15-17, wherein the biosensor comprises at least a working electrode and a substrate.

19. The anti-CRP sdAb of any one of claims 15-18, wherein the biosensor comprises an electrode comprising a conductive material such as carbon, metal, or metallic compound.

20. The anti-CRP sdAb of any one of claims 1-19, wherein the anti-CRP sdAb further comprises a detection label or tag.

21. The anti-CRP sdAb of any one of claims 1-19, wherein the anti-CRP sdAb does not comprise a detection label or tag.

22. A nucleic acid encoding the anti-CRP sdAb of any one of claims 1-21.

23. A vector comprising the nucleic acid of claim 22.

24. The vector of claim 23, wherein the vector is an expression vector.

25. A method for detecting and/or quantifying CRP in a sample, the method comprising:
(i) contacting a sample with the anti-CRP sdAb of any one of claims 1-21, and
(ii) detecting binding of the anti-CRP sdAb.

26. An in situ method for detecting and/or quantifying CRP in a subject, the method comprising (i) contacting a subject's biological fluid with the anti-CRP sdAb of any one of claims 1-21; and
(ii) detecting binding of the anti-CRP sdAb.

27. The method of claim 25 or 26, further comprising detecting the binding of the anti-CRP sdAb over time.

28. The method of any one of claims 25-27, further comprising detecting the repeated binding of the anti-CRP sdAb over time.

29. The method of any one of claims 25-28, wherein binding of the anti-CRP sdAb is detected by a biosensor.

30. The method of any one of claims 25-29, wherein the sample comprises a biological fluid, such as a bodily fluid, for example interstitial fluid (ISF), blood, urine, stool, serum, tear fluid, sweat or saliva; or wherein the biological fluid is a bodily fluid, for example interstitial fluid (ISF), blood, urine, stool, serum, tear fluid, sweat or saliva.

31. The method of any one of claims 25-30, wherein the sample comprises human interstitial fluid (ISF), blood, urine, stool, serum, tear, sweat or saliva, or wherein the biological fluid is a human bodily fluid, for example human interstitial fluid (ISF), blood, urine, stool, serum, tear fluid, sweat or saliva.

32. The method of any one of claims 25 and 27-29, wherein the sample comprises a sample from a biological production system such as a culture sample.

33. The anti-CRP sdAb of any one of claims 1-21 for use as a medicament.

34. The anti-CRP sdAb of any one of claims 1-21 for use in in vivo diagnosis.

35. The anti-CRP sdAb of any one of claims 1-21 for use in the treatment of a disease associated with an inflammation, optionally a systemic inflammation.

36. A method of treatment a disease associated with an inflammation, optionally a systemic inflammation comprising a step of administering the anti-CRP sdAb of any one of claims 1-21 to a subject in need thereof.

37. The anti-CRP sdAb for use of claim 35, or the method of claim 36, wherein the disease is associated with an increased production or release of inflammatory markers.

38. The anti-CRP sdAb for use of claim 35 or 37, or the method of claim 36 or 37, wherein the disease is associated with an increased production or release of CRP.

39. The anti-CRP sdAb for use of any one of claim 35 or 37-38, or the method of any one of claims 36-38, wherein the disease is rheumatoid arthritis, inflammatory bowel disease, IgA nephropathy, Psoriasis, lupus, multiple sclerosis, Endometriosis, Sepsis, cardiovascular disease, metabolic syndrome, hypertension, diabetes, cancer, bacterial or viral infections, COVID19, asthma, familial Mediterranean fever, Alzheimer's disease, Parkinson's diseases, diabetic macular edema (DME), diabetic retinopathy, uveitis, glaucoma, dry eye (e.g., dry eye disease or dry eye syndrome), allergic conjunctivitis, ocular pain, rhegmatogenous retinal detachment (RRD), age-related macular degeneration (AMD), proliferative diabetic retinopathy (PDR), retinal vein occlusion (RVO), neuromyelitis optica (NMO), corneal transplant, corneal abrasion, physical injury to the eye, non-alcoholic fat liver disease, chronic kidney disease, or tumour.

40. The anti-CRP sdAb of any one of claims 1-21 for use in a method of in vivo diagnosis of a disease associated with an inflammation, optionally a systemic inflammation, the method comprising:
(i) contacting a subject with the anti-CRP sdAb of any one of claims 1-21, and
(ii) detecting the binding of the anti-CRP sdAb.

41. A method of in vivo diagnosis of a disease associated with an inflammation, optionally a systemic inflammation, the method comprising:
(i) contacting a subject with the anti-CRP sdAb of any one of claims 1-21, and
(ii) detecting binding of the anti-CRP sdAb.

42. An ex vivo or in vitro method of monitoring the level of CRP comprising:
(i) contacting a sample of a subject with the anti-CRP sdAb of any one of claims 1-21, and
(ii) detecting binding of the anti-CRP sdAb.

43. A method of monitoring the level of CRP in a subject comprising:
(i) contacting the subject with the anti-CRP sdAb of any one of claims 1-21, and
(ii) detecting binding of the anti-CRP sdAb.

44. The anti-CRP sdAb for use of claim 35 or 37-40, or the method of any one of claim 36-39 or 41-43, wherein binding of the anti-CRP sdAb is repeatedly detected over time.

45. An apparatus comprising:
(i) the anti-CRP sdAb of any one of claims 1-21, and
(ii) a contacting unit for contacting a sample or a subject with the anti-CRP sdAb, and
(iii) a detecting unit for detecting and/or quantifying binding of the anti-CRP sdAb, and optionally
(iv) a dispensing unit for dispensing a drug, optionally wherein the drug is the anti-CRP sdAb of any one of claims 1-21.

46. The apparatus of claim 45 for use in the treatment or in vivo diagnosis of a disease associated with an inflammation, optionally a systemic inflammation.

47. The apparatus of claim 45 or the apparatus for use of claim 46, wherein the detecting unit for detecting and/or quantifying binding of the anti-CRP sdAb comprises a biosensor.

48. The apparatus or the apparatus for use of claim 47, wherein the biosensor comprises one or more electrode, such as a working electrode and/or a counter electrode and/or a reference electrode, and a substrate, optionally wherein the sdAb is conjugated to the working electrode.

49. The apparatus or the apparatus for use of claim 47 or 48, wherein the biosensor comprises at least a working electrode and a substrate.

50. The apparatus or the apparatus for use of any one of claims 47-49, wherein the biosensor comprises an electrode comprising a conductive material such as carbon, a metal or metallic compound.

51. The apparatus or the apparatus for use of any one of claims 48-50, wherein the anti-CRP sdAb is layered on the one or more electrode or conjugated to the one or more electrode, preferably the working electrode and optionally, wherein the anti-CRP sdAb is conjugated to said electrode via a linker.

52. The anti-CRP sdAb for use of claim 35 or 40, or the method of claim 36 or 41, or the apparatus for use of any one of claims 46-51, wherein the disease is associated with an increased production or release of inflammatory markers.

53. The anti-CRP sdAb for use of any one of claim 35, 40 or 52, or the method of any one of claim 36, 41 or 52, or the apparatus for use of any one of claims 46-52, wherein the disease is associated with an increased production or release of CRP.

54. The anti-CRP sdAb for use of any one of claim 35, 40 or 52-53, or the method of any one of claim 36, 41 or 52-53, or the apparatus of any one of claims 46-53, wherein the disease is any one of rheumatoid arthritis, inflammatory bowel disease, IgA nephropathy, Psoriasis, lupus, multiple sclerosis, Endometriosis, Sepsis, cardiovascular disease, metabolic syndrome, hypertension, diabetes, cancer, bacterial or viral infections, COVID19, asthma, familial Mediterranean fever, Alzheimer's disease, Parkinson's diseases, diabetic macular edema (DME), diabetic retinopathy, uveitis, glaucoma, dry eye (e.g., dry eye disease or dry eye syndrome), allergic conjunctivitis, ocular pain, rhegmatogenous retinal detachment (RRD), age-related macular degeneration (AMD), proliferative diabetic retinopathy (PDR), retinal vein occlusion (RVO), neuromyelitis optica (NMO), corneal transplant, corneal abrasion, physical injury to the eye, non-alcoholic fat liver disease, chronic kidney disease, or tumour.

55. A method of producing an anti-CRP sdAb comprising:
(i) contacting a subject with a recombinant human CRP, and
(ii) isolating a sdAb which successfully binds to CRP, and
(iii) selecting for an on-rate constant (kₒₙ) of about 3.30×10\hat{ }4 s⁻¹M⁻¹−1.70×10\hat{ }7 s⁻¹M⁻¹ and an off-rate constant (k_{off}) of about 3.20×10\hat{ }−9 s⁻¹−2.20×10\hat{ }−1 s⁻¹.

56. The method of claim 55, wherein the method comprises selecting for an on-rate constant (kₒₙ) of about 3.80×10\hat{ }5 s⁻¹M⁻¹−1.70×10\hat{ }7 s⁻¹M⁻¹ and an off-rate constant (k_{off}) of about 3.20×10\hat{ }−9 s⁻¹−1.30×10\hat{ }−2 s⁻¹.

57. The method of claim 55, wherein the method comprises selecting for an on-rate constant (kₒₙ) of about 3.30×10\hat{ }4 s⁻¹M⁻¹−4.60×10\hat{ }6 s⁻¹M⁻¹ and an off-rate constant (k_{off}) of about 2.60×10\hat{ }−4 s⁻¹−2.20×10\hat{ }−1 s⁻¹.

58. The method of claim 55, wherein the method comprises selecting for an on-rate constant (kₒₙ) of about 4.10×10\hat{ }4 s⁻¹M⁻¹−3.80×10\hat{ }5 s⁻¹M⁻¹ and an off-rate constant (k_{off}) of about 2.00×10\hat{ }−5 s⁻¹−6.90×10\hat{ }−3 s⁻¹.

59. The method of any one of claims 55-58, wherein the method further comprises a step of selecting for continuous and reversible binding to CRP.

60. The method of any one of claims 55-59, wherein the method further comprises a step of selecting for binding to a second or further CRP after dissociation from a first CRP.

61. The method of any one of claims 55-60, wherein the method further comprises a step of selecting for inhibiting the biological function of CRP or not inhibiting the biological function of CRP.

62. The method of claim 61, wherein the biological function of CRP is CRP signalling or the CRP signalling pathway.

63. An anti-CRP sdAb produced by the method of any one of claims 55-62.

64. A biosensor comprising the anti-CRP sdAb of any one of claims 1-21.

65. The biosensor of claim 64, wherein the biosensor comprises one or more biosensor electrodes.

66. The biosensor of claim 64 or 65, wherein the biosensor comprises a working electrode and a substrate

67. The biosensor of claim 66, wherein the biosensor further comprises a counter electrode and/or a reference electrode.

68. The biosensor of any one of claims 64-67, wherein the anti-CRP sdAb is conjugated to a biosensor electrode, optionally to a working electrode.

69. The biosensor of claim 68, wherein the anti-CRP sdAb is conjugated to a biosensor electrode, optionally to a working electrode, via a linker, such as a nucleic acid linker, amino acid/peptide linker, peptide nucleic acid linker, polymer linker, polyethylenglycol (PEG) linker, protein-tag linker, or conventional chemistry technique linker.

70. The biosensor of any one of claims 66-69, wherein the working electrode comprises a conductive material, such as carbon, metal or metallic compound.

71. The biosensor of any one of claims 64-70, wherein the biosensor, the anti-CRP sdAb or the linker comprises a detection label, a tag, or redox reporters/indicators.

72. The method of any one of claim 25-32, 36-39 or 41-44, or the anti-CRP sdAb for use of any one of claim 35, 37-40 or 44, wherein binding of the anti-CRP sdAb to CRP is detected and/or quantified by electrochemical ELISA.


SEQUENCE LISTING (111 sequences)

SEQ ID NO: 1 - protein, 131 aa, synthetic construct
  source 1..131: note = full length SCR-1A1 sdAb
AVQLVESGGGLAQPGGSLRLSCVASGLMFSGYAMSWARQAPGKRLEWVSGIYPDGSETYYADSVKGRFTISRDNAKNTLYLQMNSLEPEDTALYYCAKDGVGWRRAGTPNLGCLNPPCDYWGQGTQVTVSS

SEQ ID NO: 2 - protein, 25 aa, synthetic construct
  source 1..25: note = SCR-1A1 FR1
AVQLVESGGGLAQPGGSLRLSCVAS

SEQ ID NO: 3 - protein, 8 aa, synthetic construct
  source 1..8: note = SCR-1A1 CDR1
GLMFSGYA

SEQ ID NO: 4 - protein, 17 aa, synthetic construct
  source 1..17: note = SCR-1A1 FR2
MSWARQAPGKRLEWVSG

SEQ ID NO: 5 - protein, 8 aa, synthetic construct
  source 1..8: note = SCR-1A1 CDR2
IYPDGSET

SEQ ID NO: 6 - protein, 38 aa, synthetic construct
  source 1..38: note = SCR-1A1 FR3
YYADSVKGRFTISRDNAKNTLYLQMNSLEPEDTALYYC

SEQ ID NO: 7 - protein, 24 aa, synthetic construct
  source 1..24: note = SCR-1A1 CDR3
AKDGVGWRRAGTPNLGCLNPPCDY

SEQ ID NO: 8 - protein, 11 aa, synthetic construct
  source 1..11: note = SCR-1A1 FR4
WGQGTQVTVSS

SEQ ID NO: 9 - protein, 121 aa, synthetic construct
  source 1..121: note = full length SCR-2B4 sdAb
EVQLVESGGGLVQPGGSLRLSCAASEFNLDSYSIVWFRQAPGKGREGISCISSGGSTTYRDSVKGRFTISRDNAKNTVYLQMNSLKPEDTAVYYCAAARICTYYSSTYDGWGQGTQVTVSS

SEQ ID NO: 10 - protein, 25 aa, synthetic construct [sequence omitted]

SEQ ID NO: 11 - protein, 8 aa, synthetic construct
  source 1..8: note = SCR-2B4 CDR1
EFNLDSYS

SEQ ID NO: 12 - protein, 17 aa, synthetic construct
  source 1..17: note = SCR-2B4 FR2
IVWFRQAPGKGREGISC

SEQ ID NO: 13 - protein, 7 aa, synthetic construct
  source 1..7: note = SCR-2B4 CDR2
ISSGGST

SEQ ID NO: 14 - protein, 38 aa, synthetic construct
  source 1..38: note = SCR-2B4 FR3
TYRDSVKGRFTISRDNAKNTVYLQMNSLKPEDTAVYYC

SEQ ID NO: 15 - protein, 15 aa, synthetic construct
  source 1..15: note = SCR-2B4 CDR3
AAARICTYYSSTYDG

SEQ ID NO: 16 - protein, 11 aa, synthetic construct
  source 1..11: note = SCR-2B4 FR4
WGQGTQVTVSS

SEQ ID NO: 17 - protein, 123 aa, synthetic construct
  source 1..123: note = full length SCR-2D1 sdAb
EVQLVESGGGLVQAGGSLRLSCAASGRTFSGYAMGWFRQAPGKERESVASISRSGPTTRYADSVKGRFTISRDNAKNTVYLDMDSLKPEDTAVYYCAGKSDVIAVQATEYNYWGQGTQVTVSS

SEQ ID NO: 18 - protein, 25 aa, synthetic construct
  source 1..25: note = SCR-2D1 FR1
EVQLVESGGGLVQAGGSLRLSCAAS

SEQ ID NO: 19 - protein, 8 aa, synthetic construct
  source 1..8: note = SCR-2D1 CDR1
GRTFSGYA

SEQ ID NO: 20 - protein, 17 aa, synthetic construct
  source 1..17: note = SCR-2D1 FR2
MGWFRQAPGKERESVAS

SEQ ID NO: 21 - protein, 8 aa, synthetic construct
  source 1..8: note = SCR-2D1 CDR2
ISRSGPTT

SEQ ID NO: 22 - protein, 38 aa, synthetic construct
  source 1..38: note = SCR-2D1 FR3
RYADSVKGRFTISRDNAKNTVYLDMDSLKPEDTAVYYC

SEQ ID NO: 23 - protein, 16 aa, synthetic construct
  source 1..16: note = SCR-2D1 CDR3
AGKSDVIAVQATEYNY

SEQ ID NO: 24 - protein, 11 aa, synthetic construct
  source 1..11: note = SCR-2D1 FR4
WGQGTQVTVSS

SEQ ID NO: 25 - protein, 126 aa, synthetic construct
  source 1..126: note = full length SCR-2D2 sdAb
EVQLVESGGGLVQPGGSLRLSCAASGVTLAKNAIGWFRQAPGKEREGISCIRGSHGDTAYVDSVKGRFTISRDNTKNTVYLEMNNLKPEDTAVYYCATGDPQLRGGERCPYEYEYWGRGTQVTVSS

SEQ ID NO: 26 - protein, 25 aa, synthetic construct [sequence omitted]

SEQ ID NO: 27 - protein, 8 aa, synthetic construct
  source 1..8: note = SCR-2D2 CDR1
GVTLAKNA

SEQ ID NO: 28 - protein, 17 aa, synthetic construct
  source 1..17: note = SCR-2D2 FR2
IGWFRQAPGKEREGISC

SEQ ID NO: 29 - protein, 8 aa, synthetic construct
  source 1..8: note = SCR-2D2 CDR2
IRGSHGDT

SEQ ID NO: 30 - protein, 38 aa, synthetic construct
  source 1..38: note = SCR-2D2 FR3
AYVDSVKGRFTISRDNTKNTVYLEMNNLKPEDTAVYYC

SEQ ID NO: 31 - protein, 19 aa, synthetic construct
  source 1..19: note = SCR-2D2 CDR3
ATGDPQLRGGERCPYEYEY

SEQ ID NO: 32 - protein, 11 aa, synthetic construct
  source 1..11: note = SCR-2D2 FR4
WGRGTQVTVSS

SEQ ID NO: 33 - protein, 129 aa, synthetic construct
  source 1..129: note = full length SCR-3A4 sdAb
EVQLVESGGGSVQTGGSLRLSCAASGRTFSTYAMGWFRQAPGKEREFVAAISRSGLNRFYADSVKGRFTIARDNAKNTVTLQMYSLKPEDTAVYYCAADSRRAPFDTPDRYMPRHYDDWGQGTQVTVSS

SEQ ID NO: 34 - protein, 25 aa, synthetic construct
  source 1..25: note = SCR-3A4 FR1
EVQLVESGGGSVQTGGSLRLSCAAS

SEQ ID NO: 35 - protein, 8 aa, synthetic construct
  source 1..8: note = SCR-3A4 CDR1
GRTFSTYA

SEQ ID NO: 36 - protein, 17 aa, synthetic construct
  source 1..17: note = SCR-3A4 FR2
MGWFRQAPGKEREFVAA

SEQ ID NO: 37 - protein, 8 aa, synthetic construct
  source 1..8: note = SCR-3A4 CDR2
ISRSGLNR

SEQ ID NO: 38 - protein, 38 aa, synthetic construct
  source 1..38: note = SCR-3A4 FR3
FYADSVKGRFTIARDNAKNTVTLQMYSLKPEDTAVYYC

SEQ ID NO: 39 - protein, 22 aa, synthetic construct
  source 1..22: note = SCR-3A4 CDR3
AADSRRAPFDTPDRYMPRHYDD

SEQ ID NO: 40 - protein, 11 aa, synthetic construct
  source 1..11: note = SCR-3A4 FR4
WGQGTQVTVSS

SEQ ID NO: 41 - protein, 126 aa, synthetic construct
  source 1..126: note = full length SCR-3A5 sdAb
EVQLVESGGGLVQAGDSLRLSCAASGRTFSGAAMVWFRQAPGKERERLAAISWSGGSTWYADSVKGRFSISRDNAKNTVYLQMNSLKPEDTAVYICAAHSGRDYYTLGEPYEPDYWGQGTQVTVSS

SEQ ID NO: 42 - protein, 25 aa, synthetic construct
  source 1..25: note = SCR-3A5 FR1
EVQLVESGGGLVQAGDSLRLSCAAS

SEQ ID NO: 43 - protein, 8 aa, synthetic construct
  source 1..8: note = SCR-3A5 CDR1
GRTFSGAA

SEQ ID NO: 44 - protein, 17 aa, synthetic construct
  source 1..17: note = SCR-3A5 FR2
MVWFRQAPGKERERLAA

SEQ ID NO: 45 - protein, 8 aa, synthetic construct
  source 1..8: note = SCR-3A5 CDR2
ISWSGGST

SEQ ID NO: 46 - protein, 38 aa, synthetic construct
  source 1..38: note = SCR-3A5 FR3
WYADSVKGRFSISRDNAKNTVYLQMNSLKPEDTAVYIC

SEQ ID NO: 47 - protein, 19 aa, synthetic construct
  source 1..19: note = SCR-3A5 CDR3
AAHSGRDYYTLGEPYEPDY

SEQ ID NO: 48 - protein, 11 aa, synthetic construct
  source 1..11: note = SCR-3A5 FR4
WGQGTQVTVSS

SEQ ID NO: 49 - protein, 123 aa, synthetic construct
  source 1..123: note = full length SCR-3B7 sdAb
EVQLVESGGGLVQAGGSLRLSCAASGRTFSNYVMGWFRQAPGKEREFVASIARSGPTTYYTDSVKGRFTISRDNAKNTVYLQMNSLRPADTALYYCAAKRDVISRTAREYDYWGQGTQVTVSS

SEQ ID NO: 50 - protein, 25 aa, synthetic construct
  source 1..25: note = SCR-3B7 FR1
EVQLVESGGGLVQAGGSLRLSCAAS

SEQ ID NO: 51 - protein, 8 aa, synthetic construct
  source 1..8: note = SCR-3B7 CDR1
GRTFSNYV

SEQ ID NO: 52 - protein, 17 aa, synthetic construct
  source 1..17: note = SCR-3B7 FR2
MGWFRQAPGKEREFVAS

SEQ ID NO: 53 - protein, 8 aa, synthetic construct
  source 1..8: note = SCR-3B7 CDR2
IARSGPTT

SEQ ID NO: 54 - protein, 38 aa, synthetic construct
  source 1..38: note = SCR-3B7 FR3
YYTDSVKGRFTISRDNAKNTVYLQMNSLRPADTALYYC

SEQ ID NO: 55 - protein, 16 aa, synthetic construct
  source 1..16: note = SCR-3B7 CDR3
AAKRDVISRTAREYDY

SEQ ID NO: 56 - protein, 11 aa, synthetic construct
  source 1..11: note = SCR-3B7 FR4
WGQGTQVTVSS

SEQ ID NO: 57 - protein, 128 aa, synthetic construct
  source 1..128: note = full length SCR-3E4 sdAb
EVQLVESGGGLVQPGGSLRLFCAASGLTLDDYNVGWFRQAPGKELEGVACISRKDGSTYYADSVKGRFTISRDNAKNTVHLQMSSLKPEDTAVYRCVTEGPGITTVQAMCDMLLSNYWGQGTQVTVSS

SEQ ID NO: 58 - protein, 25 aa, synthetic construct
  source 1..25: note = SCR-3E4 FR1
EVQLVESGGGLVQPGGSLRLFCAAS

SEQ ID NO: 59 - protein, 8 aa, synthetic construct
  source 1..8: note = SCR-3E4 CDR1
GLTLDDYN

SEQ ID NO: 60 - protein, 17 aa, synthetic construct
  source 1..17: note = SCR-3E4 FR2
VGWFRQAPGKELEGVAC

SEQ ID NO: 61 - protein, 8 aa, synthetic construct
  source 1..8: note = SCR-3E4 CDR2
ISRKDGST

SEQ ID NO: 62 - protein, 38 aa, synthetic construct
  source 1..38: note = SCR-3E4 FR3
YYADSVKGRFTISRDNAKNTVHLQMSSLKPEDTAVYRC

SEQ ID NO: 63 - protein, 21 aa, synthetic construct
  source 1..21: note = SCR-3E4 CDR3
VTEGPGITTVQAMCDMLLSNY

SEQ ID NO: 64 - protein, 11 aa, synthetic construct
  source 1..11: note = SCR-3E4 FR4
WGQGTQVTVSS

SEQ ID NO: 65 - protein, 124 aa, synthetic construct
  source 1..124: note = full length SCR-3E5 sdAb
VVQLVESGGGLVQAGGSLRLSCAASGRTLSSYAMGWFRQAPGKEREFVAAVSGSGGTTYYADDSVKGRFTISRDNAKNTVSLQMNSLKPEDTAVYYCAANRDTISRIQYEYDYWGQGTQVTVSS

SEQ ID NO: 66 - protein, 25 aa, synthetic construct
  source 1..25: note = SCR-3E5 FR1
VVQLVESGGGLVQAGGSLRLSCAAS

SEQ ID NO: 67 - protein, 8 aa, synthetic construct
  source 1..8: note = SCR-3E5 CDR1
GRTLSSYA

SEQ ID NO: 68 - protein, 17 aa, synthetic construct
  source 1..17: note = SCR-3E5 FR2
MGWFRQAPGKEREFVAA

SEQ ID NO: 69 - protein, 8 aa, synthetic construct
  source 1..8: note = SCR-3E5 CDR2
VSGSGGTT

SEQ ID NO: 70 - protein, 39 aa, synthetic construct
  source 1..39: note = SCR-3E5 FR3
YYADDSVKGRFTISRDNAKNTVSLQMNSLKPEDTAVYYC

SEQ ID NO: 71 - protein, 16 aa, synthetic construct
  source 1..16: note = SCR-3E5 CDR3
AANRDTISRIQYEYDY

SEQ ID NO: 72 - protein, 11 aa, synthetic construct
  source 1..11: note = SCR-3E5 FR4
WGQGTQVTVSS

SEQ ID NO: 73 - protein, 121 aa, synthetic construct
  source 1..121: note = full length SCR-3G7 sdAb
EVQLVESGGGLVQVGGSLRLSCAASGRTFSTYDMGWYRQAPGKERERVAAIRWRDSSTDYADSVQGRFTISRDNAKNTMYLQMDNLRPEDTAIYYCHAGDYSGSYYRYDYWGQGTQVTVSS

SEQ ID NO: 74 - protein, 25 aa, synthetic construct
  source 1..25: note = SCR-3G7 FR1
EVQLVESGGGLVQVGGSLRLSCAAS

SEQ ID NO: 75 - protein, 8 aa, synthetic construct
  source 1..8: note = SCR-3G7 CDR1
GRTFSTYD

SEQ ID NO: 76 - protein, 17 aa, synthetic construct
  source 1..17: note = SCR-3G7 FR2
MGWYRQAPGKERERVAA

SEQ ID NO: 77 - protein, 8 aa, synthetic construct
  source 1..8: note = SCR-3G7 CDR2
IRWRDSST

SEQ ID NO: 78 - protein, 38 aa, synthetic construct
  source 1..38: note = SCR-3G7 FR3
DYADSVQGRFTISRDNAKNTMYLQMDNLRPEDTAIYYC

SEQ ID NO: 79 - protein, 14 aa, synthetic construct
  source 1..14: note = SCR-3G7 CDR3
HAGDYSGSYYRYDY

SEQ ID NO: 80 - protein, 11 aa, synthetic construct
  source 1..11: note = SCR-3G7 FR4
WGQGTQVTVSS

SEQ ID NO: 81 - protein, 121 aa, synthetic construct
  source 1..121: note = full length SCR-3H1 sdAb
EVQLVESGGGLVQAGDSLRLSCADSGRTFSSYAMGWFRQAPGKGREFVAVISSGGSTIYGDSVKGRFTISRDNARNTGYLEVNSLKFEDTAVYYCAAARLSSRSPLDYRYWGQGTQVTVSS

SEQ ID NO: 82 - protein, 25 aa, synthetic construct
  source 1..25: note = SCR-3H1 FR1
EVQLVESGGGLVQAGDSLRLSCADS

SEQ ID NO: 83 - protein, 8 aa, synthetic construct
  source 1..8: note = SCR-3H1 CDR1
GRTFSSYA

SEQ ID NO: 84 - protein, 17 aa, synthetic construct
  source 1..17: note = SCR-3H1 FR2
MGWFRQAPGKGREFVAV

SEQ ID NO: 85 - protein, 7 aa, synthetic construct
  source 1..7: note = SCR-3H1 CDR2
ISSGGST

SEQ ID NO: 86 - protein, 38 aa, synthetic construct
  source 1..38: note = SCR-3H1 FR3
IYGDSVKGRFTISRDNARNTGYLEVNSLKFEDTAVYYC

SEQ ID NO: 87 - protein, 15 aa, synthetic construct
  source 1..15: note = SCR-3H1 CDR3
AAARLSSRSPLDYRY

SEQ ID NO: 88 - protein, 11 aa, synthetic construct
  source 1..11: note = SCR-3H1 FR4
WGQGTQVTVSS

SEQ ID NO: 89 - protein, 121 aa, synthetic construct
  source 1..121: note = full length SCR-3H6 sdAb
EVQLVESGGGLVQTGGSLRLSCAASGYSFSTYGMGWFRQAPGKQRELVAAISSGGSTIYADSAKGRFTISRDNAKNTVYLQMNSLKPEDTAVYYCAGARLVSRSPAEYRGWGQGTQVTVSS

SEQ ID NO: 90 - protein, 25 aa, synthetic construct
  source 1..25: note = SCR-3H6 FR1
EVQLVESGGGLVQTGGSLRLSCAAS

SEQ ID NO: 91 - protein, 8 aa, synthetic construct
  source 1..8: note = SCR-3H6 CDR1
GYSFSTYG

SEQ ID NO: 92 - protein, 17 aa, synthetic construct
  source 1..17: note = SCR-3H6 FR2
MGWFRQAPGKQRELVAA

SEQ ID NO: 93 - protein, 7 aa, synthetic construct
  source 1..7: note = SCR-3H6 CDR2
ISSGGST

SEQ ID NO: 94 - protein, 38 aa, synthetic construct
  source 1..38: note = SCR-3H6 FR3
IYADSAKGRFTISRDNAKNTVYLQMNSLKPEDTAVYYC

SEQ ID NO: 95 - protein, 15 aa, synthetic construct
  source 1..15: note = SCR-3H6 CDR3
AGARLVSRSPAEYRG

SEQ ID NO: 96 - protein, 11 aa, synthetic construct
  source 1..11: note = SCR-3H6 FR4
WGQGTQVTVSS

SEQ ID NO: 97 - protein, 9 aa, synthetic construct
AADSRRAPF

SEQ ID NO: 98 - protein, 10 aa, synthetic construct
AANRDTISRI

SEQ ID NO: 99 - protein, 7 aa, synthetic construct
AAKRDVI

SEQ ID NO: 100 - protein, 6 aa, synthetic construct
AADAPS

SEQ ID NO: 101 - protein, 7 aa, synthetic construct
AAARLSS

SEQ ID NO: 102 - protein, 6 aa, synthetic construct
AARQSD

SEQ ID NO: 103 - protein, 5 aa, synthetic construct
AAPRT

SEQ ID NO: 104 - protein, 5 aa, synthetic construct
AADPS

SEQ ID NO: 105 - protein, 5 aa, synthetic construct
AKAAR

SEQ ID NO: 106 - protein, 5 aa, synthetic construct
AAKRP

SEQ ID NO: 107 - protein, 5 aa, synthetic construct
AADRE

SEQ ID NO: 108 - protein, 4 aa, synthetic construct
ATKR

SEQ ID NO: 109 - protein, 6 aa, synthetic construct
AADQSD

SEQ ID NO: 110 - protein, 6 aa, synthetic construct
AADGPG

SEQ ID NO: 111 - protein, 7 aa, synthetic construct
AADTSRS